(19) United States
(12) Patent Application Publication
(10) Pub. No.: US 2026/0137687 A1
(43) Pub. Date: May 21, 2026

(54) GTPase INHIBITORS AND USES THEREOF

(71) Applicant: The Regents of The University of California, Oakland, CA (US)

(72) Inventors: Ziyang Zhang, San Francisco, CA (US); Kevan M. Shokat, San Francisco, CA (US); Johannes Morstein, San Francisco, CA (US)

(21) Appl. No.: 19/105,304

(22) PCT Filed: Aug. 23, 2023
(86) PCT No.: PCT/US2023/072762

§ 371 Date: Feb. 20, 2025

Related U.S. Application Data
(60) Provisional application No. 63/400,620, filed on Aug. 24, 2022.

(51) Int. Cl. A61K 31/519; C07D 519/00

(52) U.S. Cl. CPC A61K 31/519; C07D 519/00

(57) ABSTRACT

Described herein, inter alia, are GTPase inhibitors and uses thereof.

### CROSS-REFERENCES TO RELATED APPLICATIONS

This application claims the benefit of U.S. Provisional Application No. 63/400,620, filed Aug. 24, 2022, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under K00 CA253758 awarded by the National Institutes of Health. The government has certain rights in the invention.

### REFERENCE TO AN ELECTRONIC SEQUENCE LISTING

The contents of the electronic sequence listing (048536-743001WO_Sequence_Listing_ST26.xml; Size: 13,874 bytes; and Date of Creation: Aug. 2, 2023) is hereby incorporated by reference in its entirety.

### BACKGROUND

KRAS mutations are one of the most common oncogenic drivers in human cancer. While small molecule inhibitors for the G12C mutant have been successfully developed, allele-specific inhibition for other KRAS hotspot mutants remain challenging. Disclosed herein, inter alia, are solutions to these and other problems in the art.

### BRIEF SUMMARY

In an aspect is provided a compound, or a pharmaceutically acceptable salt thereof, having the formula:

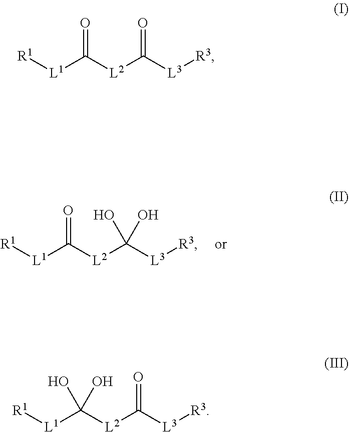

R¹ is a Switch II Binding Pocket binding moiety.
L¹ is a bond or divalent linker.
L² is a bond or substituted or unsubstituted alkylene.
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl.

Each X³ is independently —Cl, —Br, —I, or —F. The symbol n3 is an integer from 0 to 4. The symbols m3 and v3 are independently 1 or 2.

In an aspect is provided a pharmaceutical composition including a compound described herein, or a pharmaceutically acceptable salt thereof, and a pharmaceutically acceptable excipient.

In an aspect is provided a method of treating cancer in a subject in need thereof, the method including administering to the subject in need thereof a therapeutically effective amount of a compound described herein, or a pharmaceutically acceptable salt thereof.

In an aspect is provided a method of treating a K-Ras(G12R)-associated disease in a subject in need thereof, the method including administering to the subject in need thereof a therapeutically effective amount of a compound described herein, or a pharmaceutically acceptable salt thereof.

In an aspect is provided a method of treating an H-Ras(G12R)-associated disease in a subject in need thereof, the method including administering to the subject in need thereof a therapeutically effective amount of a compound described herein, or a pharmaceutically acceptable salt thereof.

In an aspect is provided a method of treating an N-Ras(G12R)-associated disease in a subject in need thereof, the method including administering to the subject in need thereof a therapeutically effective amount of a compound described herein, or a pharmaceutically acceptable salt thereof.

In an aspect is provided a method of modulating the level of activity of a Ras protein in a cell, the method including contacting the cell with an effective amount of a compound described herein, or a pharmaceutically acceptable salt thereof.

In an aspect is provided a method of attaching a compound to an arginine residue of a protein, the method including contacting said compound with the arginine residue, wherein the compound has the formula:

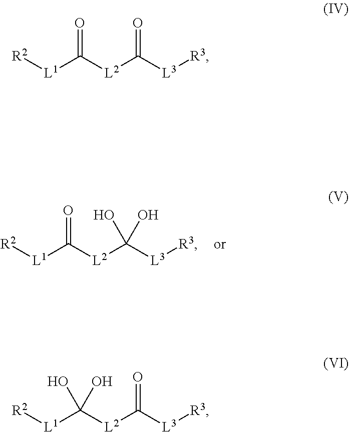

or a salt thereof. L¹, L², L³, and R³ are as described herein, including in embodiments. R² is a Switch II Binding Pocket binding moiety, a phosphatase PTP domain binding moiety, an SH2 domain binding moiety, a pseudokinase KSR domain binding moiety, or a pseudokinase STRADα domain binding moiety.

In an aspect is provided a method of attaching a compound to an arginine residue, the method including contacting the compound with the arginine residue, wherein the compound has the formula:

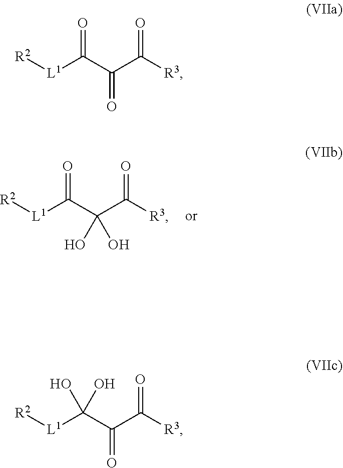

or a salt thereof. L¹ and R³ are as described herein, including in embodiments.

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOv₄NR^{4A}R^{4B}, —N^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —N^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl.

Each X⁴ is independently —Cl, —Br, —I, or —F. The symbol n4 is an integer from 0 to 4. The symbols m4 and v4 are independently 1 or 2.

In an aspect is provided a Switch II GTPase protein covalently bound to a compound described herein, or a salt thereof, wherein the compound is covalently bound to an arginine residue of the Switch II GTPase protein.

In an aspect is provided a compound covalently bound to an arginine residue, having the formula:

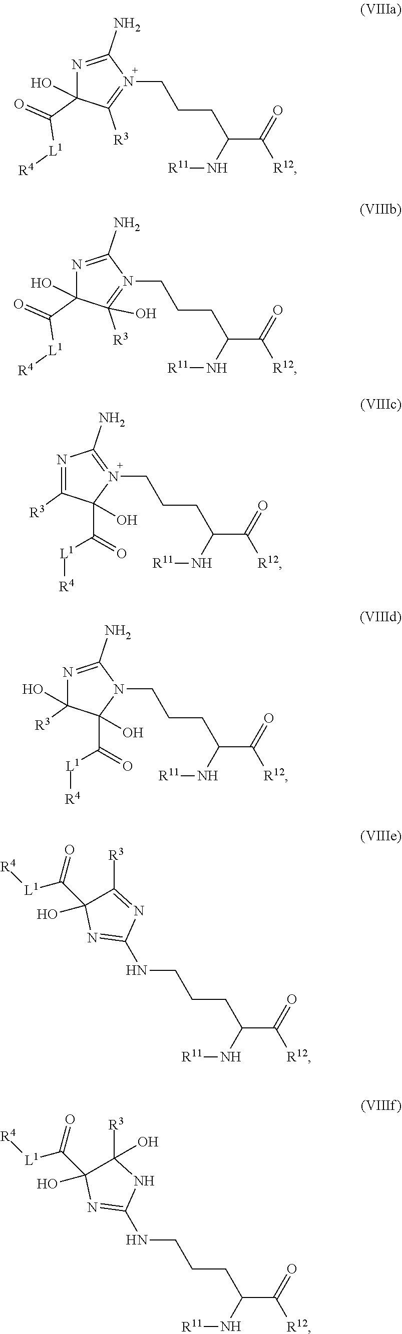

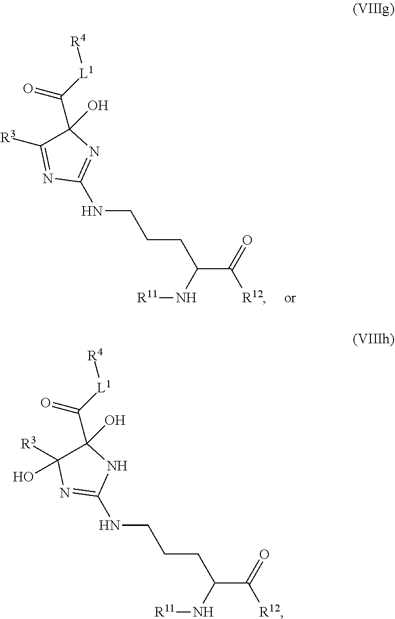

or a salt thereof. L¹, R³, and R⁴ are as described herein, including in embodiments. R¹¹ is hydrogen, —C(O)CH₃, or a first protein moiety. R¹² is —OH or a second protein moiety.

### BRIEF DESCRIPTION OF THE DRAWINGS

FIGS. 1A-1C. FIG. 1A: Synthesis of α,β-diketoamide 3. FIG. 1B: Scheme depicting the reaction between an arginine residue and an α,β-diketoamide. FIG. 1C: Intact protein mass spectra of K-Ras(G12R)·GDP and K-Ras(G12R)·GDP·3 adduct.

FIGS. 2A-2D. FIG. 2A: Time-dependent covalent modification of wildtype K-Ras and CysLight K-Ras(G12R) by compound 3 (50 μM). FIG. 2B: Reaction between K-Ras mutants and compound 3 (50 μM, 16 h). FIG. 2C: Reaction between K-Ras(G12R)·GDP and compound 3 (50 μM) at various pH. FIG. 2D: Differential scanning fluorimetry of K-Ras(G12R)·GDP and K-Ras(G12R)·GDP·3 adduct.

FIGS. 3A-3D. FIG. 3A: Crystal structure of K-Ras(G12R)·GDP·4 adduct. Fₒ-F_{c} omit map is depicted for compound 4 and arginine 12 in gray mesh (6=2.0). FIG. 3B: Scheme depicting the reaction between compound 4 and the Arg12 residue. FIG. 3C: Comparison of the structures of unliganded K-Ras(G12R)·GDP (PDB: 4QL3) and K-Ras(G12R)·GDP·4 adduct. FIG. 3D: Comparison of the structures of K-Ras(G12C)·GDP·MRTX849 (PDB: 6UT0) and K-Ras(G12R)·GDP·4 adduct.

FIGS. 4A-4B. FIG. 4A: Sos- or EDTA-mediated nucleotide exchange of K-Ras(G12R) and K-Ras(G12R)·3 adduct. FIG. 4B: Covalent modification of endogenous and exogenous K-Ras(G12R) in cell lysates.

FIGS. 5A-5C. Biochemical characterization of compound 4. FIG. 5A: Differential scanning fluorimetry of K-Ras(G12R)·GDP and its adduct with compound 4. FIG. 5B: Reaction between K-Ras(G12R)·GDP and compound 4 (50 μM) at various pH. FIG. 5C: Whole protein mass spectrometry of K-Ras(G12R)·GDP incubated with 100 μM 4 at pH 7.5 for 1 h and for 24 h.

FIGS. 6A-6B. Cellular activity of compound 3. FIG. 6A: Immunoblot analysis of phospho-ERK signaling in TCC-PAN-2 cells treated with compound 3. FIG. 6B: Growth inhibition of BaF3/G12R cells (±IL-3), TCC-Pan-2 cells, and A375 cells by compound 3.

FIG. 7. Growth inhibition of BaF3/G12R cells (±IL-3) by compound 4.

FIG. 8. Permeability of compound 3 assessed in a parallel artificial membrane permeability assay (PAMPA). Three permeability controls were included: Chloramphenicol (high), Diclofenac (medium), and Theophylline (low). MRTX849 was included as a reference compound.

FIG. 9. Direct targeting of arginines with 1,2-dicarbonyl compounds. Labelling conditions: 4 μM K-Ras Cyslight, 100 μM compound, 20 mM HEPES 7.5, 150 mM NaCl, 1 mM MgCl₂.

FIGS. 10A-10C. The diketone warhead is reactive with arginine. FIG. 10A: Chemical structure of ZZY-04-084. FIG. 10B: Src protein covalent labelling detected by mass spectrometry. FIG. 10C: Src protein covalent labelling detected by fluorescence.

FIG. 11. Overlaid crystal structures of c-Src bound to a related inhibitor and KSR2. Pseudokinases contain natural lysine to arginine mutations.

FIG. 12. Targeting a natural arginine in the VAIR motif in KSR.

FIGS. 13A-13B. Compounds selectively react with pseudokinase KSR. FIG. 13A: Chemical structures of ZZY-04-076, ZZY-04-084, ZZY-04-090, and ZZY-05-057. FIG. 13B: Experimental conditions: ˜10 ng/μL protein+10 μM compound, 23° C., 1 h.

FIG. 14. Targeting Arg692 in KSR.

FIG. 15. STRADα has two arginines in the ATP pocket. Compounds engage both binary and ternary complexes of STRADα but spares LKB1.

FIG. 16. Arg crosslinks with Cys but not Lys in the presence of 2,3-butanedione. SH2 domains and phosphatase active sites contain Arg-Cys diad. Experimental conditions: 0.1 M NAc-Arg, 0.1 M 2,3-butanedione, 0.1 M additive, 0.5 M pH 8.0 Na-phosphate buffer, 23° C., 24 h.

FIG. 17. Selected compounds modify SHP2 PTP domain, which contains ligandable arginine, in the assay tested.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O-is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

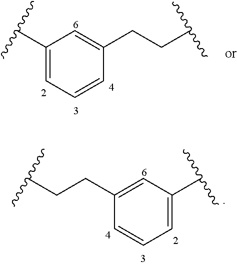 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, ˜NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₅ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1} μL⁴ may be substituted with one or more first substituent groups denoted by R^{L4.1}, L⁵ may be substituted with one or more first substituent groups denoted by R_{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R^{5.1} . . . ; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R³², R^{4.2}, R^{5.2} . . . R^{100.2}; R^{1A.2}, R^{2A.2}, R^{3A.2}, R^{4A.2}, R^{5A.2} . . . R^{100A.2}; R_{L1.2}, R_{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R_{L2.2}, R^{L3.2}, R^{L4.2}, R^{L5.2} . . . R^{100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R_{WW.2} is optionally substituted by one or more R^{WW.3}. By way of example when the R^{WW} group is phenyl substituted by R^{WW.1}, which is methyl, the methyl group may be further substituted to form groups including but not limited to:

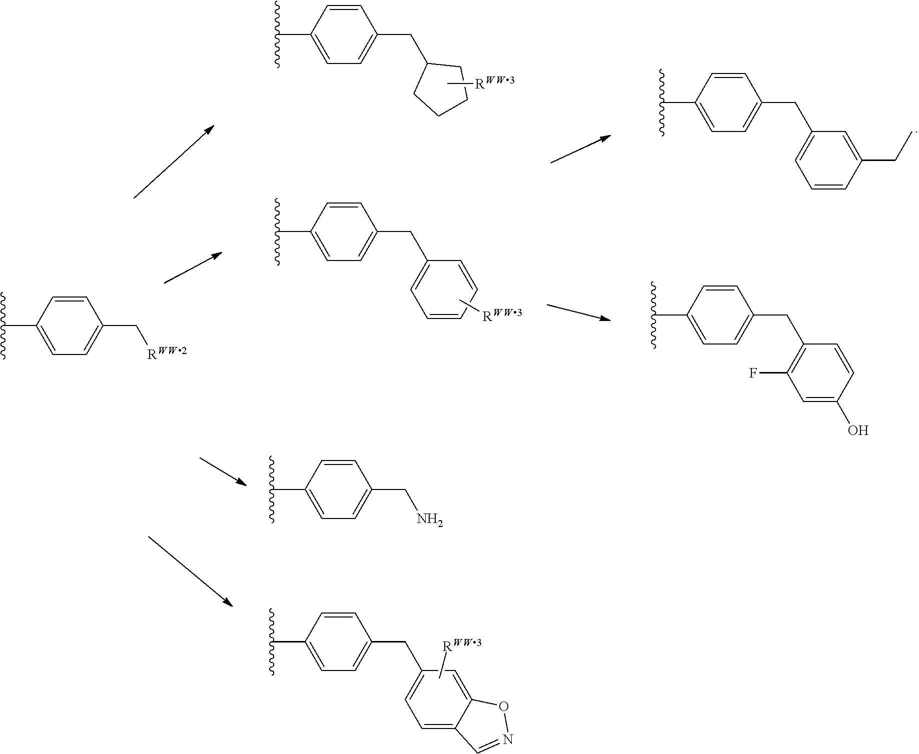

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as RWW²; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R_{100.2}, and R^{WW.3} is R^{100A.3}. Alternatively, in embodiments where R^{100A} and R^{100B} are optionally joined together to form an openly substituted ring, R^{WW.1} is R^{100B1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

As used herein, “biomolecule” is used in its customary sense and refers to a molecule found in nature or derivatives thereof, including macromolecules such as proteins, carbohydrates, lipids, and nucleic acids, as well as small molecules such as primary metabolites, secondary metabolites, and natural products. A biomolecule may be present as a moiety attached to the remainder of a compound. A biomolecule includes but is not limited to nucleic acids (e.g., DNA and RNA), peptide nucleic acids, sugars, peptides, proteins, antibodies, aptamers, lipids, small molecule affinity ligands (e.g., inhibitors, biotin, and haptens). A “biomolecular moiety” as used herein refers to a radical of a biomolecule.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments, inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is cancer (e.g., pancreatic cancer, lung cancer, colorectal cancer, melanoma, thyroid cancer, or urinary cancer). In embodiments, the disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “RASopathy” refers to a disease caused by germline mutations of genes encoding components of the RAS/MAPK signaling pathway. In embodiments, the RASopathy is a mosaic RASopathy. In embodiments, the RASopathy is a germline RASopathy. In embodiments, the RASopathy is a developmental syndrome. In embodiments, the RASopathy is Noonan syndrome. In embodiments, the RASopathy is epidermal nevus. In embodiments, the RASopathy is Schimmelpenning syndrome. In embodiments, the RASopathy is sebaceous nevus. In embodiments, the RASopathy is talipes equinovarus (e.g., congenital talipes equinovarus). In embodiments, the RASopathy is PIK3CA-related overgrowth syndrome (PROS). In embodiments, the RASopathy is PTEN-Hamartoma of the soft tissue (PHOST). In embodiments, the RASopathy is fibroadipose overgrowth, hemihyperplasia-multiple lipomatosis, congenital lipomatous overgrowth, vascular malformations, epidermal nevus, spinal and skeletal syndrome, macrodactyly syndrome, megalocephaly syndrome, or congenital diffuse infiltrative lipomatosis. In embodiments, the RASopathy is Klippel-Trenaunay syndrome (KTS), venous malformation, or lymphatic malformation. In embodiments, the RASopathy is capillary malformation-AV malformation syndrome. In embodiments, the RASopathy is autoimmune lymphoproliferative syndrome. In embodiments, the RASopathy is cardiofaciocutaneous syndrome. In embodiments, the RASopathy is hereditary gingival fibromatosis type 1. In embodiments, the RASopathy is neurofibromatosis type 1. In embodiments, the RASopathy is Costello syndrome. In embodiments, the RASopathy is Legius syndrome. In embodiments, the RASopathy is a disease as described in Hafner, C. et al. Cell Cycle 12(1), 43-50, which is herein incorporated by reference in its entirety for all purposes.

As used herein, the term “Switch II GTPase protein-associated disease” refers to any disease or condition caused by aberrant activity or signaling of a Switch II GTPase protein. In embodiments, the Switch II GTPase protein-associated disease is cancer (e.g., pancreatic cancer, lung cancer, colorectal cancer, melanoma, thyroid cancer, or urinary cancer).

As used herein, the term “K-Ras(G12R)-associated disease” refers to any disease or condition caused by aberrant activity or signaling of K-Ras(G12R). In embodiments, the K-Ras(G12R)-associated disease is cancer (e.g., pancreatic cancer, lung cancer, or colorectal cancer). In embodiments, the K-Ras(G12R)-associated disease is a RASopathy.

As used herein, the term “H-Ras(G12R)-associated disease” refers to any disease or condition caused by aberrant activity or signaling of H-Ras(G12R). In embodiments, the H-Ras(G12R)-associated disease is cancer. In embodiments, the H-Ras(G12R)-associated disease is a RASopathy.

As used herein, the term “N-Ras(G12R)-associated disease” refers to any disease or condition caused by aberrant activity or signaling of N-Ras(G12R). In embodiments, the N-Ras(G12R)-associated disease is cancer. In embodiments, the N-Ras(G12R)-associated disease is a RASopathy.

As used herein, the term “K-Ras(G13R)-associated disease” refers to any disease or condition caused by aberrant activity or signaling of K-Ras(G13R). In embodiments, the K-Ras(G13R)-associated disease is cancer. In embodiments, the K-Ras(G13R)-associated disease is a RASopathy.

As used herein, the term “H-Ras(G13R)-associated disease” refers to any disease or condition caused by aberrant activity or signaling of H-Ras(G13R). In embodiments, the H-Ras(G13R)-associated disease is cancer (e.g., melanoma). In embodiments, the H-Ras(G13R)-associated disease is a RASopathy.

As used herein, the term “N-Ras(G13R)-associated disease” refers to any disease or condition caused by aberrant activity or signaling of N-Ras(G13R). In embodiments, the N-Ras(G13R)-associated disease is cancer. In embodiments, the N-Ras(G13R)-associated disease is a RASopathy.

As used herein, the term “K-Ras(Q61R)-associated disease” refers to any disease or condition caused by aberrant activity or signaling of K-Ras(Q61R). In embodiments, the K-Ras(Q61R)-associated disease is cancer. In embodiments, the K-Ras(Q61R)-associated disease is a RASopathy.

As used herein, the term “H-Ras(Q61R)-associated disease” refers to any disease or condition caused by aberrant activity or signaling of H-Ras(Q61R). In embodiments, the H-Ras(Q61R)-associated disease is cancer (e.g., thyroid cancer or urinary cancer). In embodiments, the H-Ras(Q61R)-associated disease is a RASopathy.

As used herein, the term “N-Ras(Q61R)-associated disease” refers to any disease or condition caused by aberrant activity or signaling of N-Ras(Q61R). In embodiments, the N-Ras(Q61R)-associated disease is cancer (e.g., melanoma or thyroid cancer). In embodiments, the N-Ras(Q61R)-associated disease is a RASopathy.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Ph, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer or RASopathy) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer or RASopathy) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid side chain” refers to the side chain of an amino acid. For example, if an amino acid has the formula

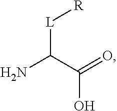

then -L-R is the amino acid side chain. As an example, L-arginine has the formula

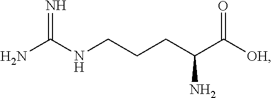 [SMILES: N=C(N)NCCC[C@H](N)C(=O)O]

and the L-arginine side chain is

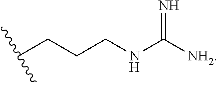 [SMILES: *CCCNC(=N)N]

The term “arginine” as used herein refers to the amino acid having the side chain

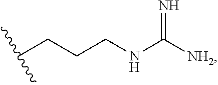 [SMILES: *CCCNC(=N)N]

or post-translational modifications thereof. In embodiments, the post-translational modification is methylation, citrullination, phosphorylation, ADP-ribosylation, carbonylation, or glycation. In embodiments, arginine is monomethylarginine (MMA). In embodiments, arginine is symmetric dimethylarginine (SDMA). In embodiments, arginine is asymmetric dimethylarginine (ADMA). In embodiments, arginine is 5-hydro-5-methyl-4-imidazolon-2-yl)-ornithine (MG-H1). In embodiments, arginine is carboxyethylarginine (CEA). In embodiments, arginine is carboxymethylarginine (CMA). In embodiments, arginine is as described in Slade, D. J. et al. Biopolymers 101(2), 133-143 (2014), which is herein incorporated by reference in its entirety for all purposes. In embodiments, arginine is a post-translationally modified arginine as set forth above. In embodiments, arginine is not post-translationally modified, and has the side chain

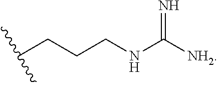 [SMILES: *CCCNC(=N)N]

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to G12 of K-Ras when the selected residue occupies the same essential spatial or other structural relationship as G12 of K-Ras. In some embodiments, where a selected protein is aligned for maximum homology with K-Ras, the position in the aligned selected protein aligning with G12 is said to correspond to G12. Instead of a primary sequence alignment, a three dimensional structural alignment can also be used, e.g., where the structure of the selected protein is aligned for maximum correspondence with K-Ras and the overall structures compared. In this case, an amino acid that occupies the same essential position as G12 in the structural model is said to correspond to the G12 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “Switch II” as used herein refers to a protein domain of a GTPase protein (e.g., Ras, K-Ras, H-Ras, or N-Ras) formed by residues corresponding to residues 60-76 of K-Ras, H-Ras, or N-Ras (e.g., K-Ras Switch II refers to residues 60-76 of K-Ras, H-Ras Switch II refers to residues 60-76 of H-Ras, N-Ras Switch II refers to residues 60-76 of N-Ras). A “Switch II Binding Pocket” is a cavity bound (the limits or boundaries of which are made), at least in part, by the amino acid residues that form Switch II. In some embodiments, a “Switch II Binding Pocket” is a cavity, in the GDP bound form of a GTPase protein (e.g., Ras, K-Ras, H-Ras, or N-Ras), bound (the limits or boundaries of which are made), at least in part, by the amino acid residues that form Switch II. A “Switch II Binding Pocket binding moiety” is a moiety of a compound (e.g., as described herein) that binds to the Switch II Binding Pocket.

The term “Switch II GTPase protein” as used herein refers to a GTPase protein including a Switch II. In embodiments, the Switch II GTPase protein includes a Switch II Binding Pocket. In embodiments, the Switch II GTPase protein is a Ras protein. In embodiments, the Switch II GTPase protein is K-Ras. In embodiments, the Switch II GTPase protein is H-Ras. In embodiments, the Switch II GTPase protein is N-Ras. In embodiments, the Switch II GTPase protein is ARF1 (e.g., UniProt P84077). In embodiments, the Switch II GTPase protein is ARF3 (e.g., UniProt P61204). In embodiments, the Switch II GTPase protein is ARF4 (e.g., UniProt P18085). In embodiments, the Switch II GTPase protein is ARF5 (e.g., UniProt P84085). In embodiments, the Switch II GTPase protein is ARF6 (e.g., UniProt P62330). In embodiments, the Switch II GTPase protein is TRIM23 (e.g., UniProt P36406). In embodiments, the Switch II GTPase protein is ARL1 (e.g., UniProt P40616). In embodiments, the Switch II GTPase protein is ARL2 (e.g., UniProt P36404). In embodiments, the Switch II GTPase protein is ARL3 (e.g., UniProt P36405). In embodiments, the Switch II GTPase protein is ARL4A (e.g., UniProt P40617). In embodiments, the Switch II GTPase protein is ARL4B. In embodiments, the Switch II GTPase protein is ARL5. In embodiments, the Switch II GTPase protein is ARL6 (e.g., UniProt Q9HOF7). In embodiments, the Switch II GTPase protein is ARL7. In embodiments, the Switch II GTPase protein is ARL8. In embodiments, the Switch II GTPase protein is ARL9 (e.g., UniProt Q6T311). In embodiments, the Switch II GTPase protein is ARL12. In embodiments, the Switch II GTPase protein is ARL11 (e.g., UniProt Q969Q4). In embodiments, the Switch II GTPase protein is ARF7. In embodiments, the Switch II GTPase protein is 339231 (e.g., UniProt Q0P5N6). In embodiments, the Switch II GTPase protein is DKFZp761. In embodiments, the Switch II GTPase protein is ARFRP1 (e.g., UniProt Q13795). In embodiments, the Switch II GTPase protein is ARFRP2 (e.g., UniProt Q9NXU5). In embodiments, the Switch II GTPase protein is ARL10A (e.g., UniProt Q8N8L6). In embodiments, the Switch II GTPase protein is ARL10B (e.g., UniProt Q96BM9). In embodiments, the Switch II GTPase protein is ARL10C. In embodiments, the Switch II GTPase protein is 344988. In embodiments, the Switch II GTPase protein is SARA1 (e.g., UniProt Q6FID4). In embodiments, the Switch II GTPase protein is SARA2.

The term “Switch II GTPase protein arginine residue” as used herein refers to an arginine residue of a Switch II GTPase protein. In embodiments, the Switch II GTPase protein arginine residue is an arginine residue corresponding to the 12 position of a Ras protein (e.g., K-Ras, H-Ras, or N-Ras). In embodiments, the Switch II GTPase protein arginine residue is an arginine residue corresponding to the 13 position of a Ras protein (e.g., K-Ras, H-Ras, or N-Ras). In embodiments, the Switch II GTPase protein arginine residue is an arginine residue corresponding to the 61 position of a Ras protein (e.g., K-Ras, H-Ras, or N-Ras). In embodiments, the Switch II GTPase protein arginine residue is a natural Switch II GTPase protein arginine residue. In embodiments, the Switch II GTPase protein arginine residue is a mutant Switch II GTPase protein arginine residue. In embodiments, the mutant Switch II GTPase protein arginine residue is arginine residue 12 of K-Ras(G12R), H-Ras(G12R), or N-Ras(G12R). In embodiments, the mutant Switch II GTPase protein arginine residue is arginine residue 13 of K-Ras(G13R), H-Ras(G13R), or N-Ras(G13R). In embodiments, the mutant Switch II GTPase protein arginine residue is arginine residue 61 of K-Ras(Q61R), H-Ras(Q61R), or N-Ras(Q61R).

The term “Ras” refers to one or more of the family of human Ras GTPase proteins (e.g. K-Ras, H-Ras, N-Ras), including homologs, isoforms, and functional fragments thereof.

The term “K-Ras” refers to the protein that in humans is encoded by the KRAS gene. The K-Ras protein is a GTPase, which converts guanosine triphosphate to guanosine diphosphate. A mutation in the K-Ras protein (e.g., an amino acid substitution) can result in various malignancies (e.g., lung adenocarcinoma, pancreatic cancer, or colorectal cancer). The term “K-Ras” may refer to the nucleotide sequence or protein sequence of human KRAS (e.g., Entrez 3845, UniProt P01116, RefSeq NM_004985.4, RefSeq NM_033360.3, RefSeq NP_004976.2, or RefSeq NP_203524.1). In embodiments, K-Ras has the following amino acid sequence:

(SEQ ID NO: 1)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQRVEDAFYTLVREIRQYRLKKISKEEKTPGCVKIKKCIIM.

The term “H-Ras” refers to the enzyme that in humans is encoded by the HRAS gene. The H-Ras protein is a GTPase, which converts guanosine triphosphate to guanosine diphosphate. Mutations in the H-Ras protein (e.g., an amino acid substitution) can result in various malignancies (e.g., bladder cancer, thyroid cancer, salivary duct carcinoma, epithelial carcinoma, or kidney cancer). The term “H-Ras” may refer to the nucleotide sequence or protein sequence of human HRAS (e.g., Entrez 3265, UniProt P01112, RefSeq NM_001130442.2, RefSeq NM_001318054.1, RefSeq NM_005343.3, RefSeq NM_00176795.4, RefSeq NP_001123914.1, RefSeq NP_001304983.1, RefSeq NP_005334.1, or RefSeq NP_789765.1). In embodiments, H-Ras has the following amino acid sequence:

(SEQ ID NO: 2)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHQYRE
QIKRVKDSDDVPMVLVGNKCDLAARTVESRQAQDLARSYGIPYIETSAK
TRQGVEDAFYTLVREIRQHKLRKLNPPDESGPGCMSCKCVLS.

The term “N-Ras” refers to the enzyme that in humans is encoded by the NRAS gene. The N-Ras protein is a GTPase, which converts guanosine triphosphate to guanosine diphosphate. The term “N-Ras” may refer to the nucleotide sequence or protein sequence of human NRAS (e.g., Entrez 4893, UniProt P01111, RefSeq NM_002524.4, or RefSeq NP_002515.1). In embodiments, N-Ras has the following amino acid sequence:

(SEQ ID NO: 3)
MTEYKLVVVGAGGVGKSALTIQLIQNHFVDEYDPTIEDSYRKQVVIDGE
TCLLDILDTAGQEEYSAMRDQYMRTGEGFLCVFAINNSKSFADINLYRE
QIKRVKDSDDVPMVLVGNKCDLPTRTVDTKQAHELAKSYGIPFIETSAK
TRQGVEDAFYTLVREIRQYRMKKLNSSDDGTQGCMGLPCVVM.

In embodiments, the Switch II Binding Pocket is bound at least in part by one or more of V7, V9, G10, P34, T58, G60, Q61, E62, E63, R68, Y71, M72, Y96, Q99, and/or 1100 of K-Ras or equivalent residues in homologous, related (e.g., H-Ras or N-Ras), or mutant Ras proteins. A compound as described herein (including embodiments, examples, and figures), which binds to amino acids that form or contacts amino acids that form the Switch II Binding Pocket is a “Switch II Binding Pocket binding compound” and a moiety of a compound that binds to amino acids that form or contacts amino acids that form the Switch II Binding Pocket is a “Switch II Binding Pocket binding moiety”.

In embodiments, a Switch II Binding Pocket binding compound or Switch II Binding Pocket binding moiety binds or contacts one amino acid that forms the Switch II Binding Pocket. In embodiments, a Switch II Binding Pocket binding compound or Switch II Binding Pocket binding moiety binds or contacts multiple amino acids that form the Switch II Binding Pocket. In embodiments, a Switch II Binding Pocket binding compound or Switch II-Binding Pocket binding moiety binds or contacts one amino acid selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts multiple K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts two K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts three K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts four K-Ras amino acids selected from amino acids in a mutant K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts five K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts six K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts seven K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts eight K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts nine K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts ten K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts eleven K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts twelve K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts thirteen K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts fourteen K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100. In embodiments, a Switch II Binding Pocket binding compound or Switch II Binding Pocket binding moiety binds or contacts fifteen K-Ras amino acids selected from amino acids in a mutant K-Ras (e.g., K-Ras(G12R), K-Ras(G13R), or K-Ras(Q61R)), related Ras (e.g., H-Ras, H-Ras(G12R), H-Ras(G13R), H-Ras(Q61R), N-Ras, N-Ras(G12R), N-Ras(G13R), or N-Ras(Q61R)), or homolog of K-Ras corresponding to K-Ras residues V7, V9, G10, P34, T58, G60, Q61, E62, E63, R68, Y71, M72, Y96, Q99, and 1100.

The term “phosphatase PTP domain” as used herein refers to a protein domain of a protein tyrosine phosphatase formed by residues corresponding to residues 1-279 of human PTP1B, which includes an arginine residue. In embodiments, the phosphatase PTP domain is as described in Andersen, J. N. et al. Mol. Cell Biol. 21(21), 7117-7136 (2001), which is herein incorporated by reference in its entirety for all purposes. A “phosphatase PTP domain binding moiety” is a moiety of a compound (e.g., as described herein) that binds to the phosphatase PTP domain.

The term “PTP1B” or “tyrosine-proteine phosphatase 1B” refers to a member of the protein tyrosine phosphatase family that in humans is encoded by the PTPN1 gene. The term “PTP1B” may refer to the nucleotide sequence or protein sequence of human PTP1B (e.g., Entrez 5770, UniProt P18031, RefSeq NM_002827.4, or RefSeq NP_002818.1). In embodiments, PTP1B has the following amino acid sequence:

(SEQ ID NO: 4)
MEMEKEFEQIDKSGSWAAIYQDIRHEASDFPCRVAKLPKNKNRNRYRDV
SPFDHSRIKLHQEDNDYINASLIKMEEAQRSYILTQGPLPNTCGHFWEM
VWEQKSRGVVMLNRVMEKGSLKCAQYWPQKEEKEMIFEDTNLKLTLISE
DIKSYYTVRQLELENLTTQETREILHFHYTTWPDFGVPESPASFLNFLF
KVRESGSLSPEHGPVVVHCSAGIGRSGTFCLADTCLLLMDKRKDPSSVD
IKKVLLEMRKFRMGLIQTADQLRFSYLAVIEGAKFIMGDSSVQDQWKEL
SHEDLEPPPEHIPPPPRPPKRILEPHNGKCREFFPNHQWVKEETQEDKD
CPIKEEKGSPLNAAPYGIESMSQDTEVRSRVVGGSLRGAQAASPAKGEP
SLPEKDEDHALSYWKPFLVNMCVATVLTAGAYLCYRFLFNSNT.

The term “SH2 domain” as used herein refers to a protein domain of a Src oncoprotein formed by residues corresponding to residues 151-248 of human Src oncoprotein, which includes an arginine residue. In embodiments, the SH2 domain is as described in Roskoski, R. Jr. Biochem. Biophys. Res. Commun. 324(4), 1155-1164 (2004), which is herein incorporated by reference in its entirety for all purposes. An “SH2 domain binding moiety” is a moiety of a compound (e.g., as described herein) that binds to the SH2 domain.

The term “Src” or “proto-oncogene tyrosine-protein kinase Src” refers to the non-receptor tyrosine kinase protein that in humans is encoded by the SRC gene. The term “Src” may refer to the nucleotide sequence or protein sequence of human Src (e.g., Entrez 6714, UniProt P12931, RefSeq NM_005417.4, RefSeq NM_198291.2, RefSeq NP_005408.1, or RefSeq NP_938033.1). In embodiments, Src has the following amino acid sequence:

(SEQ ID NO: 5)
MGSNKSKPKDASQRRRSLEPAENVHGAGGGAFPASQTPSKPASADGHRG
PSAAFAPAAAEPKLFGGFNSSDTVTSPQRAGPLAGGVTTFVALYDYESR
TETDLSFKKGERLQIVNNTEGDWWLAHSLSTGQTGYIPSNYVAPSDSIQ
AEEWYFGKITRRESERLLLNAENPRGTFLVRESETTKGAYCLSVSDFDN
AKGLNVKHYKIRKLDSGGFYITSRTQFNSLQQLVAYYSKHADGLCHRLT
TVCPTSKPQTQGLAKDAWEIPRESLRLEVKLGQGCFGEVWMGTWNGTTR
VAIKTLKPGTMSPEAFLQEAQVMKKLRHEKLVQLYAVVSEEPIYIVTEY
MSKGSLLDFLKGETGKYLRLPQLVDMAAQIASGMAYVERMNYVHRDLRA
ANILVGENLVCKVADFGLARLIEDNEYTARQGAKFPIKWTAPEAALYGR
FTIKSDVWSFGILLTELTTKGRVPYPGMVNREVLDQVERGYRMPCPPEC
PESLHDLMCQCWRKEPEERPTFEYLQAFLEDYFTSTEPQYQPGENL.

The term “pseudokinase KSR domain” or “pseudokinase kinase suppressor of Ras domain” as used herein refers to a protein domain of a KSR2 protein formed by residues corresponding to residues 634-950 of human KSR2 protein, which includes an arginine residue. In embodiments, the pseudokinase KSR domain is as described in Brennan, D. F. et al. Nature 472, 366-369 (2011) and Xing, L. et al. Bioorg. Med. Chem. 23(19), 6520-6527, which are herein incorporated by reference in their entirety for all purposes. A “pseudokinase KSR domain binding moiety” is a moiety of a compound (e.g., as described herein) that binds to the pseudokinase KSR domain.

The term “KSR2” or “kinase suppressor of Ras 2” refers to the protein that in humans is encoded by the KSR2 gene. The term “KSR2” may refer to the nucleotide sequence or protein sequence of human KSR2 (e.g., Entrez 283455, UniProt Q6VAB6, RefSeq NM_173598.6, or RefSeq NP_775869.4). In embodiments, KSR2 has the following amino acid sequence:

(SEQ ID NO: 6)
MDEENMTKSEEQQPLSLQKALQQCELVQNMIDLSISNLEGLRTKCATSN
DLTQKEIRTLESKLVKYFSRQLSCKKKVALQERNAELDGFPQLRHWFRI
VDVRKEVLEEISPGQLSLEDLLEMTDEQVCETVEKYGANREECARLNAS
LSCLRNVHMSGGNLSKQDWTIQWPTTETGKENNPVCPPEPTPWIRTHLS
QSPRVPSKCVQHYCHTSPTPGAPVYTHVDRLTVDAYPGLCPPPPLESGH
RSLPPSPRQRHAVRTPPRTPNIVTTVTPPGTPPMRKKNKLKPPGTPPPS
SRKLIHLIPGFTALHRSKSHEFQLGHRVDEAHTPKAKKKSKPLNLKIHS
SVGSCENIPSQQRSPLLSERSLRSFFVGHAPFLPSTPPVHTEANFSANT
LSVPRWSPQIPRRDLGNSIKHRFSTKYWMSQTCTVCGKGMLFGLKCKNC
KLKCHNKCTKEAPPCHLLIIHRGDPARLVRTESVPCDINNPLRKPPRYS
DLHISQTLPKTNKINKDHIPVPYQPDSSSNPSSTTSSTPSSPAPPLPPS
ATPPSPLHPSPQCTRQQKNFNLPASHYYKYKQQFIFPDVVPVPETPTRA
PQVILHPVTSNPILEGNPLLQIEVEPTSENEEVHDEAEESEDDFEEMNL
SLLSARSFPRKASQTSIFLQEWDIPFEQLEIGELIGKGRFGQVYHGRWH
GEVAIRLIDIERDNEDQLKAFKREVMAYRQTRHENVVLFMGACMSPPHL
AIITSLCKGRTLYSVVRDAKIVLDVNKTRQIAQEIVKGMGYLHAKGILH
KDLKSKNVFYDNGKVVITDFGLFSISGVLQAGRREDKLRIQNGWLCHLA
PEIIRQLSPDTEEDKLPFSKHSDVFALGTIWYELHAREWPFKTQPAEAI
IWQMGTGMKPNLSQIGMGKEISDILLFCWAFEQEERPTFTKLMDMLEKL
PKRNRRLSHPGHFWKSAEL.

The term “pseudokinase STRADα domain” as used herein refers to a protein domain of a STRADα protein formed by residues corresponding to residues 59-431 of the STRADα protein, which includes an arginine residue. In embodiments, the pseudokinase STRADα domain is as described in Zeqiraj, E. et al. PLOS Biology 7(6), e1000126 (2009) and Xing, L. et al. Bioorg. Med. Chem. 23(19), 6520-6527, which are herein incorporated by reference in their entirety for all purposes. A “pseudokinase STRADα domain binding moiety” is a moiety of a compound (e.g., as described herein) that binds to the pseudokinase STRADα domain.

The term “STRADα” or “STE20-related kinase adapter protein alpha” refers to the protein that in humans is encoded by the STRADA gene. The term “STRADα” may refer to the nucleotide sequence or protein sequence of human STRADα (e.g., Entrez 92335, UniProt Q7RTN6, RefSeq NM_001003786.2, RefSeq NM_001003787.2, RefSeq NM_001003788.2, RefSeq NM_001165969.1, RefSeq NM_001165970.1, RefSeq NM_153335.5, RefSeq NP_001003786.1, RefSeq NP_001003787.1, RefSeq NP_001003788.1, RefSeq NP_001159441.1, RefSeq NP_001159442.1, or RefSeq NP_699166.2). In embodiments, STRADα has the following amino acid sequence:

(SEQ ID NO: 7)
MSFLVSKPERIRRWVSEKFIVEGLRDLELFGEQPPGDTRRKTNDASSES
IASFSKQEVMSSFLPEGGCYELLTVIGKGFEDLMTVNLARYKPTGEYVT
VRRINLEACSNEMVTFLQGELHVSKLFNHPNIVPYRATFIADNELWVVT
SFMAYGSAKDLICTHFMDGMNELAIAYILQGVLKALDYIHHMGYVHRSV
KASHILISVDGKVYLSGLRSNLSMISHGQRQRVVHDFPKYSVKVLPWLS
PEVLQQNLQGYDAKSDIYSVGITACELANGHVPFKDMPATQMLLEKLNG
TVPCLLDTSTIPAEELTMSPSRSVANSGLSDSLTTSTPRPSNGDSPSHP
YHRTFSPHFHHFVEQCLQRNPDARPSASTLLNHSFFKQIKRRASEALPE
LLRPVTPITNFEGSQSQDHSGIFGLVTNLEELEVDDWEF.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward mutant K-Ras(G12R) would preferentially inhibit K-Ras(G12R) over other K-Ras proteins (e.g., wild type K-Ras)). In embodiments, a “Ras(G12R)-selective compound” refers to a compound (e.g., compound described herein) having selectivity towards Ras(G12R).

### II. Compounds

In an aspect is provided a compound, or a pharmaceutically acceptable salt thereof, having the formula:

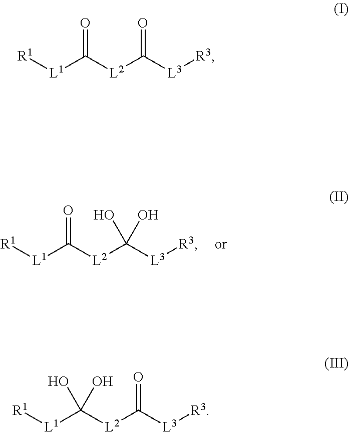

R¹ is a Switch II Binding Pocket binding moiety.

L¹ is a bond or divalent linker.

L² is a bond or substituted or unsubstituted alkylene (e.g., C₁-C₈, C₁-C₆, C₁-C₄, or C₁-C₂).

L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

Each X³ is independently —Cl, —Br, —I, or —F.

The symbol n3 is an integer from 0 to 4.

The symbols m3 and v3 are independently 1 or 2.

In embodiments, the compound has the formula:

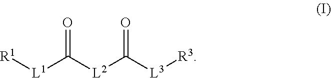

L¹, L², L³, R¹, and R³ are as described herein, including in embodiments.

In embodiments, the compound has the formula:

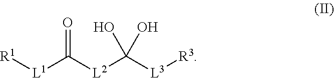

L¹, L², L³, R¹, and R³ are as described herein, including in embodiments.

In embodiments, the compound has the formula:

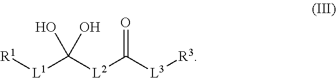

L¹, L², L³, R¹, and R³ are as described herein, including in embodiments.

In embodiments, a substituted L² (e.g., substituted alkylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is a bond or unsubstituted C₁-C₄ alkylene. In embodiments, L² is a bond. In embodiments, L² is unsubstituted C₁-C₄ alkylene. In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted isopropylene. In embodiments, L² is unsubstituted butylene. In embodiments, L² is unsubstituted n-butylene. In embodiments, L² is unsubstituted isobutylene. In embodiments, L² is unsubstituted tert-butylene.

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L³ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted C₁-C₆ alkylene, substituted or unsubstituted 2 to 6 membered heteroalkylene, substituted or unsubstituted C₃-C₅ cycloalkylene, substituted or unsubstituted 3 to 8 membered heterocycloalkylene, substituted or unsubstituted phenylene, or substituted or unsubstituted 5 to 10 membered heteroarylene.

In embodiments, L³ is a bond. In embodiments, L³ is —C(O)—.

In embodiments, a substituted R³⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³⁰ is substituted with a plurality of groups selected from substituent groups, size-limited substituent groups, and lower substituent groups; each substituent group, size-limited substituent group, and/or lower substituent group may optionally be different. In embodiments, when R³¹ is substituted, it is substituted with at least one substituent group. In embodiments, when R³⁰ is substituted, it is substituted with at least one size-limited substituent group. In embodiments, when R³⁰ is substituted, it is substituted with at least one lower substituent group.

In embodiments, R³⁰ is independently hydrogen. In embodiments, R³⁰ is independently unsubstituted C₁-C₄ alkyl. In embodiments, R³⁰ is independently unsubstituted methyl. In embodiments, R³⁰ is independently unsubstituted ethyl. In embodiments, R³⁰ is independently unsubstituted propyl. In embodiments, R³⁰ is independently unsubstituted n-propyl. In embodiments, R³⁰ is independently unsubstituted isopropyl. In embodiments, R³⁰ is independently unsubstituted butyl. In embodiments, R³⁰ is independently unsubstituted n-butyl. In embodiments, R³⁰ is independently unsubstituted isobutyl. In embodiments, R³⁰ is independently unsubstituted tert-butyl.

In embodiments, the compound has the formula:

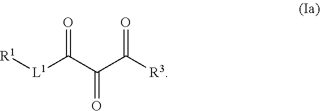

L¹, R¹, and R³ are as described herein, including in embodiments.

In embodiments, the compound has the formula:

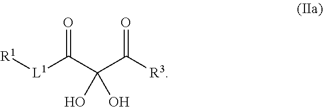

L¹, R¹, and R³ are as described herein, including in embodiments.

In embodiments, the compound has the formula:

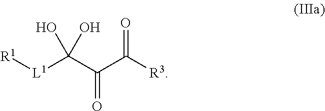

L¹, R¹, and R³ are as described herein, including in embodiments.

In embodiments, the compound has the formula:

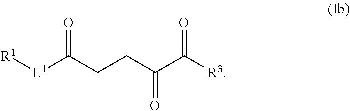

L¹, R¹, and R³ are as described herein, including in embodiments.

In embodiments, the compound has the formula:

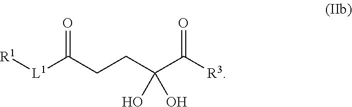

L¹, R¹, and R³ are as described herein, including in embodiments.

In embodiments, the compound has the formula:

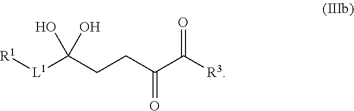

L¹, R¹, and R³ are as described herein, including in embodiments.

In embodiments, the compound has the formula:

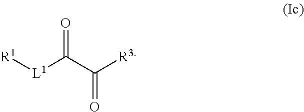

L¹, R¹, and R³ are as described herein, including in embodiments.

In embodiments, the compound has the formula:

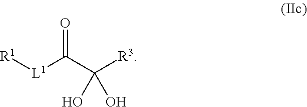

L¹, R¹, and R³ are as described herein, including in embodiments.

In embodiments, the compound has the formula:

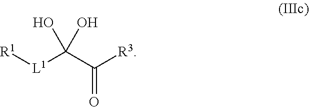

L¹, R¹, and R³ are as described herein, including in embodiments.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B}substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R³ is hydrogen or unsubstituted C₁-C₄ alkyl. In embodiments, R³ is hydrogen or unsubstituted methyl. In embodiments, R³ is hydrogen. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl.

In embodiments, R³ is halogen. In embodiments, R³ is —F. In embodiments, R³ is —Cl. In embodiments, R³ is —Br. In embodiments, R³ is —I. In embodiments, R³ is —CCl₃. In embodiments, R³ is —CBr₃. In embodiments, R³ is —CF₃. In embodiments, R³ is —CI₃. In embodiments, R³ is —CH₂Cl. In embodiments, R³ is —CH₂Br. In embodiments, R³ is —CH₂F. In embodiments, R³ is —CH₂I. In embodiments, R³ is —CHCl₂. In embodiments, R³ is —CHBr₂. In embodiments, R³ is —CHF₂. In embodiments, R³ is —CHI₂. In embodiments, R³ is —CN. In embodiments, R³ is —OH. In embodiments, R³ is —NH₂. In embodiments, R³ is —COOH. In embodiments, R³ is —CONH₂. In embodiments, R³ is —NO₂. In embodiments, R³ is —SH. In embodiments, R³ is —SO₃H. In embodiments, R³ is —OSO₃H. In embodiments, R³ is —SO₂NH₂. In embodiments, R³ is —NHNH₂. In embodiments, R³ is —ONH₂. In embodiments, R³ is —NHC(O)NHNH₂. In embodiments, R³ is —NHC(O)NH₂. In embodiments, R³ is —NHSO₂H. In embodiments, R³ is —NHC(O)H. In embodiments, R³ is —NHC(O)OH. In embodiments, R³ is-NHOH. In embodiments, R³ is —OCCl₃. In embodiments, R³ is —OCBr₃. In embodiments, R³ is —OCF₃. In embodiments, R³ is —OCI₃. In embodiments, R³ is —OCH₂Cl. In embodiments, R³ is —OCH₂Br. In embodiments, R³ is —OCH₂F. In embodiments, R³ is —OCH₂I. In embodiments, R³ is —OCHCl₂. In embodiments, R³ is —OCHBr₂. In embodiments, R³ is —OCHF₂. In embodiments, R³ is —OCHI₂. In embodiments, R³ is unsubstituted C₃-C₈ cycloalkyl. In embodiments, R³ is unsubstituted cyclopropyl. In embodiments, R³ is unsubstituted cyclobutyl. In embodiments, R³ is unsubstituted cyclopentyl. In embodiments, R³ is unsubstituted cyclohexyl. In embodiments, R³ is unsubstituted cycloheptyl. In embodiments, R³ is unsubstituted cyclooctyl. In embodiments, R³ is unsubstituted phenyl.

In embodiments, R^{3A} is hydrogen. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl.

In embodiments, R^{3B} is hydrogen. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl.

In embodiments, R^{3C} is hydrogen. In embodiments, R^{3C} is unsubstituted C₁-C₄ alkyl. In embodiments, R^{3C} is unsubstituted methyl. In embodiments, R^{3C} is unsubstituted ethyl. In embodiments, R^{3C} is unsubstituted propyl. In embodiments, R^{3C} is unsubstituted n-propyl. In embodiments, R^{3C} is unsubstituted isopropyl. In embodiments, R^{3C} is unsubstituted butyl. In embodiments, R^{3C} is unsubstituted n-butyl. In embodiments, R^{3C} is unsubstituted isobutyl. In embodiments, R^{3C} is unsubstituted tert-butyl.

In embodiments, R^{3D} is hydrogen. In embodiments, R^{3D} is unsubstituted C₁-C₄ alkyl. In embodiments, R^{3D} is unsubstituted methyl. In embodiments, R^{3D} is unsubstituted ethyl. In embodiments, R^{3D} is unsubstituted propyl. In embodiments, R^{3D} is unsubstituted n-propyl. In embodiments, R^{3D} is unsubstituted isopropyl. In embodiments, R^{3D} is unsubstituted butyl. In embodiments, R^{3D} is unsubstituted n-butyl. In embodiments, R^{3D} is unsubstituted isobutyl. In embodiments, R^{3D} is unsubstituted tert-butyl.

In embodiments, L¹ is -L¹⁰¹-L¹⁰²-L¹⁰³-L¹⁰⁴-L¹⁰⁵-.

In embodiments, L¹⁰¹ is connected directly to R¹.

L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR⁰³C(NH)NR¹⁰³—, —S(O)₂—, —NR¹⁰³S(O)₂—, —S(O)₂NR¹⁰³—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

Each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted L¹⁰¹ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰¹ is a bond. In embodiments, L¹⁰¹ is —C(O)—. In embodiments, L¹⁰¹ is —C(O)O—. In embodiments, L¹⁰¹ is —OC(O)—. In embodiments, L¹⁰¹ is —O—. In embodiments, L¹⁰¹ is —S—. In embodiments, L¹⁰¹ is —NR¹⁰¹—. In embodiments, L¹⁰¹ is —NH—. In embodiments, L¹⁰¹ is —C(O)NR¹⁰¹—. In embodiments, L¹⁰¹ is —C(O)NH—. In embodiments, L¹⁰¹ is —NR¹C(O)—. In embodiments, L¹⁰¹ is —NHC(O)—. In embodiments, L¹⁰¹ is —NR¹C(O)O—. In embodiments, L¹⁰¹ is —NHC(O)O—. In embodiments, L¹⁰¹ is —OC(O)NR¹⁰¹—. In embodiments, L¹⁰¹ is —OC(O)NH—. In embodiments, L¹⁰¹ is —NR¹⁰¹C(O)NR¹⁰¹—. In embodiments, L¹⁰¹ is —NHC(O)NH—. In embodiments, L¹⁰¹ is —NR¹⁰¹C(NH)NR^{1O1}—. In embodiments, L¹⁰¹ is —NHC(NH)NH—. In embodiments, L¹⁰¹ is —S(O)₂—. In embodiments, L¹⁰¹ is —NR¹⁰¹S(O)₂—. In embodiments, L¹⁰¹ is —NHS(O)₂—. In embodiments, L¹⁰¹ is —S(O)₂NR¹⁰¹—. In embodiments, L¹⁰¹ is —S(O)₂NH—. In embodiments, L¹⁰¹ is substituted or unsubstituted C₁-C₆ alkylene. In embodiments, L¹⁰¹ is substituted or unsubstituted methylene. In embodiments, L¹⁰¹ is substituted or unsubstituted ethylene. In embodiments, L¹⁰¹ is substituted or unsubstituted propylene. In embodiments, L¹⁰¹ is substituted or unsubstituted n-propylene. In embodiments, L¹⁰¹ is substituted or unsubstituted isopropylene. In embodiments, L¹⁰¹ is substituted or unsubstituted butylene. In embodiments, L¹⁰¹ is substituted or unsubstituted n-butylene. In embodiments, L¹⁰¹ is substituted or unsubstituted isobutylene. In embodiments, L¹⁰¹ is substituted or unsubstituted tert-butylene. In embodiments, L¹⁰¹ is substituted or unsubstituted pentylene. In embodiments, L¹⁰¹ is substituted or unsubstituted hexylene. In embodiments, L¹⁰¹ is substituted or unsubstituted 2 to 6 membered heteroalkylene. In embodiments, L¹⁰¹ is substituted or unsubstituted 3 to 8 membered heterocycloalkylene. In embodiments, L¹⁰¹ is substituted or unsubstituted azetidinyl. In embodiments, L¹⁰¹ is substituted or unsubstituted piperazinyl. In embodiments, L¹⁰¹ is

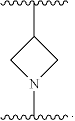 [SMILES: *C1CN(*)C1]

In embodiments, L¹⁰¹ is

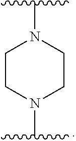 [SMILES: *N1CCN(*)CC1]

In embodiments, L¹⁰¹ is

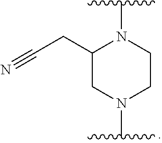 [SMILES: *N1CCN(*)C(CC#N)C1]

In embodiments, a substituted R¹⁰¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰¹ is substituted with a plurality of groups selected from substituent groups, size-limited substituent groups, and lower substituent groups; each substituent group, size-limited substituent group, and/or lower substituent group may optionally be different. In embodiments, when R¹⁰¹ is substituted, it is substituted with at least one substituent group. In embodiments, when R¹⁰¹ is substituted, it is substituted with at least one size-limited substituent group. In embodiments, when R¹⁰¹ is substituted, it is substituted with at least one lower substituent group.

In embodiments, R¹⁰¹ is independently hydrogen. In embodiments, R¹⁰¹ is independently unsubstituted C₁-C₄ alkyl. In embodiments, R¹⁰¹ is independently unsubstituted methyl. In embodiments, R¹⁰¹ is independently unsubstituted ethyl. In embodiments, R¹⁰¹ is independently unsubstituted propyl. In embodiments, R¹⁰¹ is independently unsubstituted n-propyl. In embodiments, R¹⁰¹ is independently unsubstituted isopropyl. In embodiments, R¹⁰¹ is independently unsubstituted butyl. In embodiments, R¹⁰¹ is independently unsubstituted n-butyl. In embodiments, R¹⁰¹ is independently unsubstituted isobutyl. In embodiments, R¹⁰¹ is independently unsubstituted tert-butyl.

In embodiments, a substituted L¹⁰² (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰² is a bond. In embodiments, L¹⁰² is —C(O)—. In embodiments, L¹⁰² is —C(O)O—. In embodiments, L¹⁰² is —OC(O)—. In embodiments, L¹⁰² is —O—. In embodiments, L¹⁰² is —S—. In embodiments, L¹⁰² is —NR¹⁰²—. In embodiments, L¹⁰² is —NH—. In embodiments, L¹⁰² is —C(O)NR¹⁰²—. In embodiments, L¹⁰² is —C(O)NH—. In embodiments, L¹⁰² is —NR¹⁰²C(O)—. In embodiments, L¹⁰² is —NHC(O)—. In embodiments, L¹⁰² is —NR¹⁰²C(O)O—. In embodiments, L¹⁰² is —NHC(O)O—. In embodiments, L¹⁰² is —OC(O)NR¹⁰²—. In embodiments, L¹⁰² is —OC(O)NH—. In embodiments, L¹⁰² is —NR¹⁰²C(O)NR¹⁰²—. In embodiments, L¹⁰² is —NHC(O)NH—. In embodiments, L¹⁰² is —NR¹⁰²C(NH)NR¹⁰²—. In embodiments, L¹⁰² is —NHC(NH)NH—. In embodiments, L¹⁰² is —S(O)₂—. In embodiments, L¹⁰² is —NR¹⁰²S(O)₂—. In embodiments, L¹⁰² is —NHS(O)₂—. In embodiments, L¹⁰² is —S(O)₂NR¹⁰²—. In embodiments, L¹⁰² is —S(O)₂NH—. In embodiments, L¹⁰² is substituted or unsubstituted C₁-C₆ alkylene. In embodiments, L¹⁰² is substituted or unsubstituted methylene. In embodiments, L¹⁰² is substituted or unsubstituted ethylene. In embodiments, L¹⁰² is substituted or unsubstituted propylene. In embodiments, L¹⁰² is substituted or unsubstituted n-propylene. In embodiments, L¹⁰² is substituted or unsubstituted isopropylene. In embodiments, L¹⁰² is substituted or unsubstituted butylene. In embodiments, L¹⁰² is substituted or unsubstituted n-butylene. In embodiments, L¹⁰² is substituted or unsubstituted isobutylene. In embodiments, L¹⁰² is substituted or unsubstituted tert-butylene. In embodiments, L¹⁰² is substituted or unsubstituted pentylene. In embodiments, L¹⁰² is substituted or unsubstituted hexylene. In embodiments, L¹⁰² is substituted or unsubstituted 2 to 6 membered heteroalkylene. In embodiments, L¹⁰² is substituted or unsubstituted phenylene.

In embodiments, a substituted R¹⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰² is substituted with a plurality of groups selected from substituent groups, size-limited substituent groups, and lower substituent groups; each substituent group, size-limited substituent group, and/or lower substituent group may optionally be different. In embodiments, when R¹⁰² is substituted, it is substituted with at least one substituent group. In embodiments, when R¹⁰² is substituted, it is substituted with at least one size-limited substituent group. In embodiments, when R¹⁰² is substituted, it is substituted with at least one lower substituent group.

In embodiments, R¹⁰² is independently hydrogen. In embodiments, R¹⁰² is independently unsubstituted C₁-C₄ alkyl. In embodiments, R¹⁰² is independently unsubstituted methyl. In embodiments, R¹⁰² is independently unsubstituted ethyl. In embodiments, R¹⁰² is independently unsubstituted propyl. In embodiments, R¹⁰² is independently unsubstituted n-propyl. In embodiments, R¹⁰² is independently unsubstituted isopropyl. In embodiments, R¹⁰² is independently unsubstituted butyl. In embodiments, R¹⁰² is independently unsubstituted n-butyl. In embodiments, R¹⁰² is independently unsubstituted isobutyl. In embodiments, R¹⁰² is independently unsubstituted tert-butyl.

In embodiments, a substituted L¹⁰³ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond. In embodiments, L¹⁰³ is —C(O)—. In embodiments, L¹⁰³ is —C(O)O—. In embodiments, L¹⁰³ is —OC(O)—. In embodiments, L¹⁰³ is —O—. In embodiments, L¹⁰³ is —S—. In embodiments, L¹⁰³ is —NR¹⁰³—. In embodiments, L¹⁰³ is —NH—. In embodiments, L¹⁰³ is —C(O)NR¹⁰³—. In embodiments, L¹⁰³ is —C(O)NH—. In embodiments, L¹⁰³ is —NR¹⁰³C(O)—. In embodiments, L¹⁰³ is —NHC(O)—. In embodiments, L¹⁰³ is —NR¹⁰³C(O)O—. In embodiments, L¹⁰³ is —NHC(O)O—. In embodiments, L¹⁰³ is —OC(O)NR¹⁰³—. In embodiments, L¹⁰³ is —OC(O)NH—. In embodiments, L¹⁰³ is —NR¹⁰³C(O)NR¹⁰³—. In embodiments, L¹⁰³ is —NHC(O)NH—. In embodiments, L¹⁰³ is —NR¹⁰³C(NH)NR¹⁰³—. In embodiments, L¹⁰³ is —NHC(NH)NH—. In embodiments, L¹⁰³ is —S(O)₂—. In embodiments, L¹⁰³ is —NR¹⁰³S(O)₂—. In embodiments, L¹⁰³ is —NHS(O)₂—. In embodiments, L¹⁰³ is —S(O)₂NR¹⁰³—. In embodiments, L¹⁰³ is —S(O)₂NH—. In embodiments, L¹⁰³ is substituted or unsubstituted C₁-C₆ alkylene. In embodiments, L¹⁰³ is substituted or unsubstituted methylene. In embodiments, L¹⁰³ is substituted or unsubstituted ethylene. In embodiments, L¹⁰³ is substituted or unsubstituted propylene. In embodiments, L¹⁰³ is substituted or unsubstituted n-propylene. In embodiments, L¹⁰³ is substituted or unsubstituted isopropylene. In embodiments, L¹⁰³ is substituted or unsubstituted butylene. In embodiments, L¹⁰³ is substituted or unsubstituted n-butylene. In embodiments, L¹⁰³ is substituted or unsubstituted isobutylene. In embodiments, L¹⁰³ is substituted or unsubstituted tert-butylene. In embodiments, L¹⁰³ is substituted or unsubstituted pentylene. In embodiments, L¹⁰³ is substituted or unsubstituted hexylene. In embodiments, L¹⁰³ is substituted or unsubstituted 2 to 6 membered heteroalkylene. In embodiments, L¹⁰³ is substituted or unsubstituted phenylene.

In embodiments, a substituted R¹⁰³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰³ is substituted with a plurality of groups selected from substituent groups, size-limited substituent groups, and lower substituent groups; each substituent group, size-limited substituent group, and/or lower substituent group may optionally be different. In embodiments, when R¹⁰³ is substituted, it is substituted with at least one substituent group. In embodiments, when R¹⁰³ is substituted, it is substituted with at least one size-limited substituent group. In embodiments, when R¹⁰³ is substituted, it is substituted with at least one lower substituent group.

In embodiments, R¹⁰³ is independently hydrogen. In embodiments, R¹⁰³ is independently unsubstituted C₁-C₄ alkyl. In embodiments, R¹⁰³ is independently unsubstituted methyl. In embodiments, R¹⁰³ is independently unsubstituted ethyl. In embodiments, R¹⁰³ is independently unsubstituted propyl. In embodiments, R¹⁰³ is independently unsubstituted n-propyl. In embodiments, R¹⁰³ is independently unsubstituted isopropyl. In embodiments, R¹⁰³ is independently unsubstituted butyl. In embodiments, R¹⁰³ is independently unsubstituted n-butyl. In embodiments, R¹⁰³ is independently unsubstituted isobutyl. In embodiments, R¹⁰³ is independently unsubstituted tert-butyl.

In embodiments, a substituted L¹⁰⁴ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰⁴ is substituted with a plurality of groups selected from substituent groups, size-limited substituent groups, and lower substituent groups; each substituent group, size-limited substituent group, and/or lower substituent group may optionally be different. In embodiments, when L¹⁰⁴ is substituted, it is substituted with at least one substituent group. In embodiments, when L¹⁰⁴ is substituted, it is substituted with at least one size-limited substituent group. In embodiments, when L¹⁰⁴ is substituted, it is substituted with at least one lower substituent group.

In embodiments, L¹⁰⁴ is a bond. In embodiments, L¹⁰⁴ is —C(O)—. In embodiments, L¹⁰⁴ is —C(O)O—. In embodiments, L¹⁰⁴ is —OC(O)—. In embodiments, L¹⁰⁴ is —O—. In embodiments, L¹⁰⁴ is —S—. In embodiments, L¹⁰⁴ is —NR¹⁰⁴—. In embodiments, L¹⁰⁴ is —NH—. In embodiments, L¹⁰⁴ is —C(O)NR¹⁰⁴—. In embodiments, L¹⁰⁴ is —C(O)NH—. In embodiments, L¹⁰⁴ is —NR¹⁰⁴C(O)—. In embodiments, L¹⁰⁴ is —NHC(O)—. In embodiments, L¹⁰⁴ is —NR¹⁰⁴C(O)O—. In embodiments, L¹⁰⁴ is —NHC(O)O—. In embodiments, L¹⁰⁴ is —OC(O)NR¹⁰⁴—. In embodiments, L¹⁰⁴ is —OC(O)NH—. In embodiments, L¹⁰⁴ is —NR¹⁰⁴C(O)NR¹⁰⁴—. In embodiments, L¹⁰⁴ is —NHC(O)NH—. In embodiments, L¹⁰⁴ is —NR¹⁰⁴C(NH)NR⁰⁴—. In embodiments, L¹⁰⁴ is —NHC(NH)NH—. In embodiments, L¹⁰⁴ is —S(O)₂—. In embodiments, L¹⁰⁴ is —NR¹⁰⁴S(O)₂—. In embodiments, L¹⁰⁴ is —NHS(O)₂—. In embodiments, L¹⁰⁴ is —S(O)₂NR¹⁰⁴—. In embodiments, L¹⁰⁴ is —S(O)₂NH—. In embodiments, L¹⁰⁴ is substituted or unsubstituted C₁-C₆ alkylene. In embodiments, L¹⁰⁴ is substituted or unsubstituted methylene. In embodiments, L¹⁰⁴ is substituted or unsubstituted ethylene. In embodiments, L¹⁰⁴ is substituted or unsubstituted propylene. In embodiments, L¹⁰⁴ is substituted or unsubstituted n-propylene. In embodiments, L¹⁰⁴ is substituted or unsubstituted isopropylene. In embodiments, L¹⁰⁴ is substituted or unsubstituted butylene. In embodiments, L¹⁰⁴ is substituted or unsubstituted n-butylene. In embodiments, L¹⁰⁴ is substituted or unsubstituted isobutylene. In embodiments, L¹⁰⁴ is substituted or unsubstituted tert-butylene. In embodiments, L¹⁰⁴ is substituted or unsubstituted pentylene. In embodiments, L¹⁰⁴ is substituted or unsubstituted hexylene. In embodiments, L¹⁰⁴ is substituted or unsubstituted 2 to 6 membered heteroalkylene. In embodiments, L¹⁰⁴ is substituted or unsubstituted phenylene.

In embodiments, a substituted R¹⁰⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰⁴ is substituted with a plurality of groups selected from substituent groups, size-limited substituent groups, and lower substituent groups; each substituent group, size-limited substituent group, and/or lower substituent group may optionally be different. In embodiments, when R¹⁰⁴ is substituted, it is substituted with at least one substituent group. In embodiments, when R¹⁰⁴ is substituted, it is substituted with at least one size-limited substituent group. In embodiments, when R¹⁰⁴ is substituted, it is substituted with at least one lower substituent group.

In embodiments, R¹⁰⁴ is independently hydrogen. In embodiments, R¹⁰⁴ is independently unsubstituted C₁-C₄ alkyl. In embodiments, R¹⁰⁴ is independently unsubstituted methyl. In embodiments, R¹⁰⁴ is independently unsubstituted ethyl. In embodiments, R¹⁰⁴ is independently unsubstituted propyl. In embodiments, R¹⁰⁴ is independently unsubstituted n-propyl. In embodiments, R¹⁰⁴ is independently unsubstituted isopropyl. In embodiments, R¹⁰⁴ is independently unsubstituted butyl. In embodiments, R¹⁰⁴ is independently unsubstituted n-butyl. In embodiments, R¹⁰⁴ is independently unsubstituted isobutyl. In embodiments, R¹⁰⁴ is independently unsubstituted tert-butyl.

In embodiments, a substituted L¹⁰⁵ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰⁵ is substituted with a plurality of groups selected from substituent groups, size-limited substituent groups, and lower substituent groups; each substituent group, size-limited substituent group, and/or lower substituent group may optionally be different. In embodiments, when L¹⁰⁵ is substituted, it is substituted with at least one substituent group. In embodiments, when L¹⁰⁵ is substituted, it is substituted with at least one size-limited substituent group. In embodiments, when L¹⁰⁵ is substituted, it is substituted with at least one lower substituent group.

In embodiments, L¹⁰⁵ is a bond. In embodiments, L¹⁰⁵ is —C(O)—. In embodiments, L¹⁰⁵ is —C(O)O—. In embodiments, L¹⁰⁵ is —OC(O)—. In embodiments, L¹⁰⁵ is —O—. In embodiments, L¹⁰⁵ is —S—. In embodiments, L¹⁰⁵ is —NR¹⁰⁵—. In embodiments, L¹⁰⁵ is —NH—. In embodiments, L¹⁰⁵ is —C(O)NR¹⁰⁵—. In embodiments, L¹⁰⁵ is —C(O)NH—. In embodiments, L¹⁰⁵ is —NR¹⁰⁵C(O)—. In embodiments, L¹⁰⁵ is —NHC(O)—. In embodiments, L¹⁰⁵ is —NR¹⁰⁵C(O)O—. In embodiments, L¹⁰⁵ is —NHC(O)O—. In embodiments, L¹⁰⁵ is —OC(O)NR¹⁰⁵—. In embodiments, L¹⁰⁵ is —OC(O)NH—. In embodiments, L¹⁰⁵ is —NR¹⁰⁵C(O)NR¹⁰⁵—. In embodiments, L¹⁰⁵ is —NHC(O)NH—. In embodiments, L¹⁰⁵ is —NR¹⁰⁵C(NH)NR¹⁰⁵—. In embodiments, L¹⁰⁵ is —NHC(NH)NH—. In embodiments, L¹⁰⁵ is —S(O)₂—. In embodiments, L¹⁰⁵ is —NR¹⁰⁵S(O)₂—. In embodiments, L¹⁰⁵ is —NHS(O)₂—. In embodiments, L¹⁰⁵ is —S(O)₂NR¹⁰⁵—. In embodiments, L¹⁰⁵ is —S(O)₂NH—. In embodiments, L¹⁰⁵ is substituted or unsubstituted C₁-C₆ alkylene. In embodiments, L¹⁰⁵ is substituted or unsubstituted methylene. In embodiments, L¹⁰⁵ is substituted or unsubstituted ethylene. In embodiments, L¹⁰⁵ is substituted or unsubstituted propylene. In embodiments, L¹⁰⁵ is substituted or unsubstituted n-propylene. In embodiments, L¹⁰⁵ is substituted or unsubstituted isopropylene. In embodiments, L¹⁰⁵ is substituted or unsubstituted butylene. In embodiments, L¹⁰⁵ is substituted or unsubstituted n-butylene. In embodiments, L¹⁰⁵ is substituted or unsubstituted isobutylene. In embodiments, L¹⁰⁵ is substituted or unsubstituted tert-butylene. In embodiments, L¹⁰⁵ is substituted or unsubstituted pentylene. In embodiments, L¹⁰⁵ is substituted or unsubstituted hexylene. In embodiments, L¹⁰⁵ is substituted or unsubstituted 2 to 6 membered heteroalkylene. In embodiments, L¹⁰⁵ is substituted or unsubstituted phenylene.

In embodiments, a substituted R¹⁰⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰⁵ is substituted with a plurality of groups selected from substituent groups, size-limited substituent groups, and lower substituent groups; each substituent group, size-limited substituent group, and/or lower substituent group may optionally be different. In embodiments, when R¹⁰⁵ is substituted, it is substituted with at least one substituent group. In embodiments, when R¹⁰⁵ is substituted, it is substituted with at least one size-limited substituent group. In embodiments, when R¹⁰⁵ is substituted, it is substituted with at least one lower substituent group.

In embodiments, R¹⁰⁵ is independently hydrogen. In embodiments, R¹⁰⁵ is independently unsubstituted C₁-C₄ alkyl. In embodiments, R¹⁰⁵ is independently unsubstituted methyl. In embodiments, R¹⁰⁵ is independently unsubstituted ethyl. In embodiments, R¹⁰⁵ is independently unsubstituted propyl. In embodiments, R¹⁰⁵ is independently unsubstituted n-propyl. In embodiments, R¹⁰⁵ is independently unsubstituted isopropyl. In embodiments, R¹⁰⁵ is independently unsubstituted butyl. In embodiments, R¹⁰⁵ is independently unsubstituted n-butyl. In embodiments, R¹⁰⁵ is independently unsubstituted isobutyl. In embodiments, R¹⁰⁵ is independently unsubstituted tert-butyl.

In embodiments, L¹ is a bond, substituted or unsubstituted 3 to 8 membered heterocycloalkylene, or substituted or unsubstituted phenylene. In embodiments, L¹ is substituted or unsubstituted 3 to 8 membered heterocycloalkylene. In embodiments, L¹ is substituted or unsubstituted piperazinylene. In embodiments, L¹ is

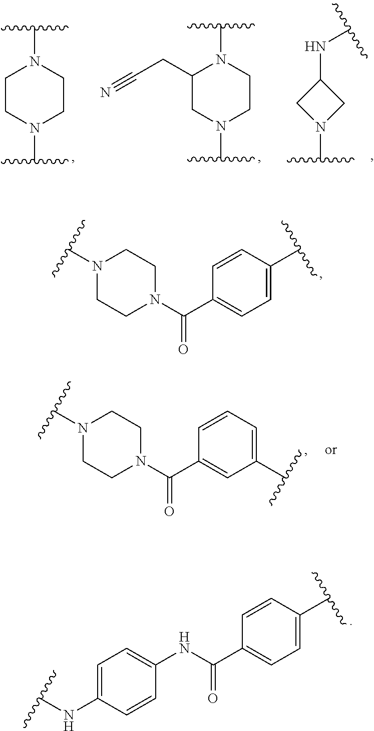

In embodiments, L¹ is

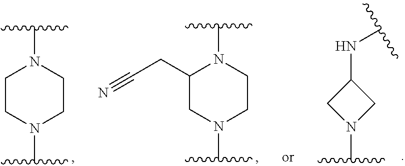

In embodiments, L¹ is

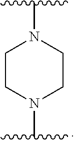 [SMILES: *N1CCN(*)CC1]

In embodiments, L¹ is

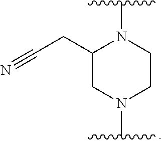 [SMILES: *N1CCN(*)C(CC#N)C1]

In embodiments, L¹ is

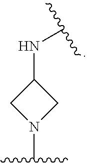 [SMILES: *NC1CN(*)C1]

In embodiments, L¹ is

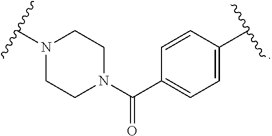 [SMILES: *c1ccc(C(=O)N2CCN(*)CC2)cc1]

In embodiments, L¹ is

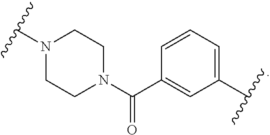 [SMILES: *c1cccc(C(=O)N2CCN(*)CC2)c1]

In embodiments, L¹ is

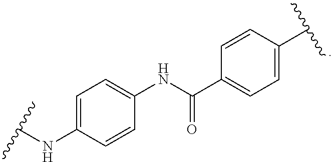 [SMILES: *Nc1ccc(NC(=O)c2ccc(*)cc2)cc1]

In embodiments, R¹ is -L¹⁰-R¹⁰.

L¹⁰ is a bond, —C(O)—, —C(O)O—, —OC(O)—, —O—, —S—, —NR¹⁰⁰—, —C(O)NR¹⁰⁰—, —NR¹⁰⁰C(O)—, —NR¹⁰⁰C(O)O—, —OC(O)NR¹⁰⁰—, —NR¹⁰⁰C(O)NR¹⁰⁰—, —NR¹⁰⁰C(NH)NR¹⁰⁰—, —S(O)₂—, —NR¹⁰⁰S(O)₂—, —S(O)₂NR¹⁰⁰—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{10A} and R^{10B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

Each X¹⁰ is independently —F, —Cl, —Br, or —I.

The symbol n10 is an integer from 0 to 4.

The symbols m10 and v10 are independently 1 or 2.

In embodiments, a substituted L¹⁰ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰ is substituted with a plurality of groups selected from substituent groups, size-limited substituent groups, and lower substituent groups; each substituent group, size-limited substituent group, and/or lower substituent group may optionally be different. In embodiments, when L¹⁰ is substituted, it is substituted with at least one substituent group. In embodiments, when L¹⁰ is substituted, it is substituted with at least one size-limited substituent group. In embodiments, when L¹⁰ is substituted, it is substituted with at least one lower substituent group.

In embodiments, L¹⁰ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁰ is a bond. In embodiments, L¹⁰ is —C(O)—. In embodiments, L¹⁰ is —C(O)O—. In embodiments, L¹⁰ is —OC(O)—. In embodiments, L¹⁰ is —O—. In embodiments, L¹⁰ is —S—. In embodiments, L¹⁰ is —NR¹⁰⁰—. In embodiments, L¹⁰ is —NH—. In embodiments, L¹⁰ is —C(O)NR¹⁰⁰—. In embodiments, L¹⁰ is —C(O)NH—. In embodiments, L¹⁰ is —NR¹⁰⁰C(O)—. In embodiments, L¹⁰ is —NHC(O)—. In embodiments, L¹⁰ is —NR¹⁰⁰C(O)O—. In embodiments, L¹⁰ is —NHC(O)O—. In embodiments, L¹⁰ is —OC(O)NR¹⁰⁰—. In embodiments, L¹⁰ is —OC(O)NH—. In embodiments, L¹⁰ is —NR¹⁰⁰C(O)NR¹⁰⁰—. In embodiments, L¹⁰ is —NHC(O)NH—. In embodiments, L¹⁰ is —NR¹⁰⁰C(NH)NR¹⁰⁰—. In embodiments, L¹⁰ is —NHC(NH)NH—. In embodiments, L¹⁰ is —S(O)₂—. In embodiments, L¹⁰ is —NR¹⁰⁰S(O)₂—. In embodiments, L¹⁰ is —NHS(O)₂—. In embodiments, L¹⁰ is —S(O)₂NR¹⁰⁰—. In embodiments, L¹⁰ is —S(O)₂NH—. In embodiments, L¹⁰ is substituted or unsubstituted C₁-C₆ alkylene. In embodiments, L¹⁰ is substituted or unsubstituted methylene. In embodiments, L¹⁰ is substituted or unsubstituted ethylene. In embodiments, L¹⁰ is substituted or unsubstituted propylene. In embodiments, L¹⁰ is substituted or unsubstituted n-propylene. In embodiments, L¹⁰ is substituted or unsubstituted isopropylene. In embodiments, L¹⁰ is substituted or unsubstituted butylene. In embodiments, L¹⁰ is substituted or unsubstituted n-butylene. In embodiments, L¹⁰ is substituted or unsubstituted isobutylene. In embodiments, L¹⁰ is substituted or unsubstituted tert-butylene. In embodiments, L¹⁰ is substituted or unsubstituted pentylene. In embodiments, L¹⁰ is substituted or unsubstituted hexylene. In embodiments, L¹⁰ is substituted or unsubstituted 2 to 6 membered heteroalkylene.

In embodiments, a substituted R¹⁰⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰⁰ is substituted with a plurality of groups selected from substituent groups, size-limited substituent groups, and lower substituent groups; each substituent group, size-limited substituent group, and/or lower substituent group may optionally be different. In embodiments, when R¹⁰⁰ is substituted, it is substituted with at least one substituent group. In embodiments, when R¹⁰⁰ is substituted, it is substituted with at least one size-limited substituent group. In embodiments, when R¹⁰⁰ is substituted, it is substituted with at least one lower substituent group.

In embodiments, R¹⁰⁰ is independently hydrogen. In embodiments, R¹⁰⁰ is independently unsubstituted C₁-C₄ alkyl. In embodiments, R¹⁰⁰ is independently unsubstituted methyl. In embodiments, R¹⁰⁰ is independently unsubstituted ethyl. In embodiments, R¹⁰⁰ is independently unsubstituted propyl. In embodiments, R¹⁰⁰ is independently unsubstituted n-propyl. In embodiments, R¹⁰⁰ is independently unsubstituted isopropyl. In embodiments, R¹⁰⁰ is independently unsubstituted butyl. In embodiments, R¹⁰⁰ is independently unsubstituted n-butyl. In embodiments, R¹⁰⁰ is independently unsubstituted isobutyl. In embodiments, R¹⁰⁰ is independently unsubstituted tert-butyl.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, a substituted R^{10A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, a substituted R^{10B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, a substituted ring formed when R^{10A} and R^{10B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{10A} and R^{10B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{10A} and R^{10B} substituents bonded to the same nitrogen atom are joined is substituted, it is substituted with at least one substituent group. In embodiments, when the substituted ring formed when R^{10A} and R^{10B} substituents bonded to the same nitrogen atom are joined is substituted, it is substituted with at least one size-limited substituent group. In embodiments, when the substituted ring formed when R^{10A} and R^{10B} substituents bonded to the same nitrogen atom are joined is substituted, it is substituted with at least one lower substituent group.

In embodiments, a substituted R^{10C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10C} is substituted with a plurality of groups selected from substituent groups, size-limited substituent groups, and lower substituent groups; each substituent group, size-limited substituent group, and/or lower substituent group may optionally be different. In embodiments, when R^{10C} is substituted, it is substituted with at least one substituent group. In embodiments, when R^{10C} is substituted, it is substituted with at least one size-limited substituent group. In embodiments, when R^{10C} is substituted, it is substituted with at least one lower substituent group.

In embodiments, a substituted R^{10D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10D} is substituted with a plurality of groups selected from substituent groups, size-limited substituent groups, and lower substituent groups; each substituent group, size-limited substituent group, and/or lower substituent group may optionally be different. In embodiments, when R^{10D} is substituted, it is substituted with at least one substituent group. In embodiments, when R^{10D} is substituted, it is substituted with at least one size-limited substituent group. In embodiments, when R^{10D} is substituted, it is substituted with at least one lower substituent group.

In embodiments, R¹⁰ is substituted or unsubstituted cycloalkyl, substituted or unsubstituted heterocycloalkyl, substituted or unsubstituted aryl, or substituted or unsubstituted heteroaryl. In embodiments, R¹⁰ is substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R¹⁰ is substituted C₃-C₈ cycloalkyl. In embodiments, R¹⁰ is substituted 3 to 8 membered heterocycloalkyl. In embodiments, R¹⁰ is substituted C₆-C₁₀ aryl. In embodiments, R¹⁰ is substituted phenyl. In embodiments, R¹⁰ is substituted 5 to 10 membered heteroaryl. In embodiments, R¹⁰ is substituted pyrimidyl. In embodiments, R¹⁰ is substituted tetrahydropyridopyrimidyl. In embodiments, R¹⁰ is substituted pyridopyrimidyl. In embodiments, R¹⁰ is substituted quinazolinyl. In embodiments, R¹⁰ is substituted pyrazolyl. In embodiments, R¹⁰ is substituted piperazinyl.

In embodiments, R^{10A} is hydrogen. In embodiments, R^{10A} is unsubstituted C₁-C₄ alkyl. In embodiments, R^{10A} is unsubstituted methyl. In embodiments, R^{10A} is unsubstituted ethyl. In embodiments, R^{10A} is unsubstituted propyl. In embodiments, R^{10A} is unsubstituted n-propyl. In embodiments, R^{10A} is unsubstituted isopropyl. In embodiments, R^{10A} is unsubstituted butyl. In embodiments, R^{10A} is unsubstituted n-butyl. In embodiments, R^{10A} is unsubstituted isobutyl. In embodiments, R^{10A} is unsubstituted tert-butyl.

In embodiments, R^{10B} is hydrogen. In embodiments, R^{10B} is unsubstituted C₁-C₄ alkyl. In embodiments, R^{10B} is unsubstituted methyl. In embodiments, R^{10B} is unsubstituted ethyl. In embodiments, R^{10B} is unsubstituted propyl. In embodiments, R^{10B} is unsubstituted n-propyl. In embodiments, R^{10B} is unsubstituted isopropyl. In embodiments, R^{10B} is unsubstituted butyl. In embodiments, R^{10B} is unsubstituted n-butyl. In embodiments, R^{10B} is unsubstituted isobutyl. In embodiments, R^{10B} is unsubstituted tert-butyl.

In embodiments, R^{10C} is hydrogen. In embodiments, R^{10C} is unsubstituted C₁-C₄ alkyl. In embodiments, R^{10C} is unsubstituted methyl. In embodiments, R^{10C} is unsubstituted ethyl. In embodiments, R^{10C} is unsubstituted propyl. In embodiments, R^{10C} is unsubstituted n-propyl. In embodiments, R^{10C} is unsubstituted isopropyl. In embodiments, R^{10C} is unsubstituted butyl. In embodiments, R^{10C} is unsubstituted n-butyl. In embodiments, R^{10C} is unsubstituted isobutyl. In embodiments, R^{10C} is unsubstituted tert-butyl.

In embodiments, R^{10D} is hydrogen. In embodiments, R^{10D} is unsubstituted C₁-C₄ alkyl. In embodiments, R^{10D} is unsubstituted methyl. In embodiments, R^{10D} is unsubstituted ethyl. In embodiments, R^{10D} is unsubstituted propyl. In embodiments, R^{10D} is unsubstituted n-propyl. In embodiments, R^{10D} is unsubstituted isopropyl. In embodiments, R^{10D} is unsubstituted butyl. In embodiments, R^{10D} is unsubstituted n-butyl. In embodiments, R^{10D} is unsubstituted isobutyl. In embodiments, R^{10D} is unsubstituted tert-butyl.

In embodiments, R¹ is

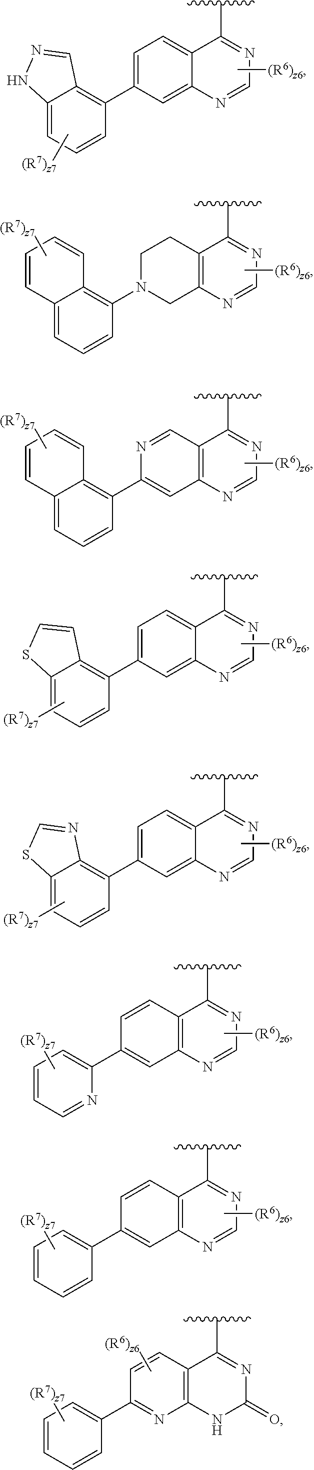

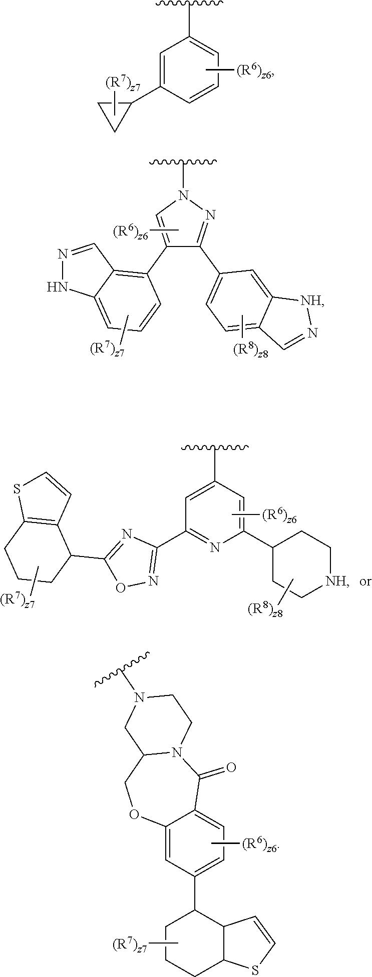

In embodiments, R¹ is

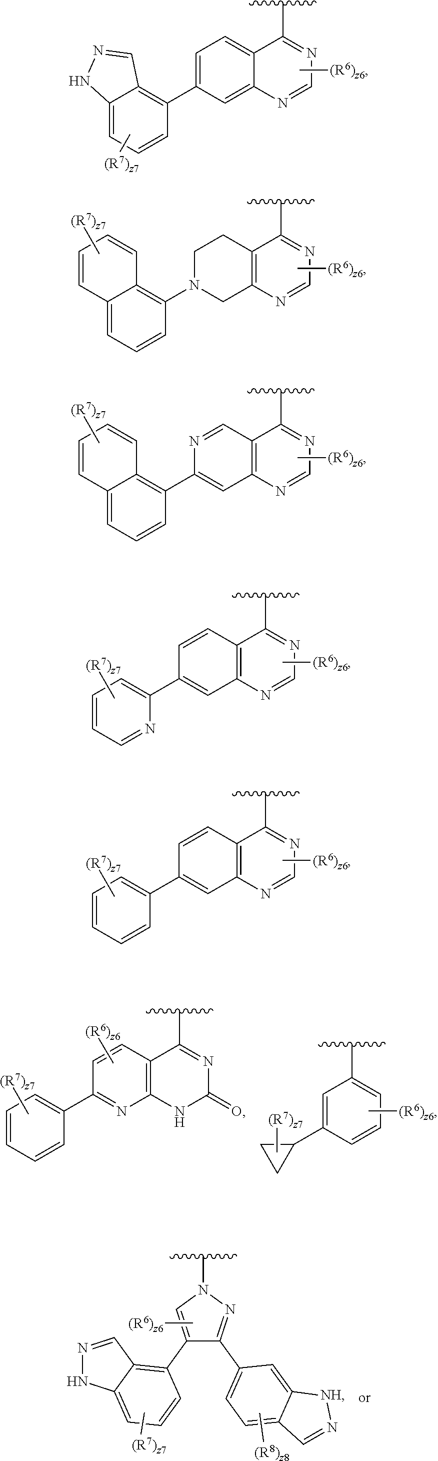

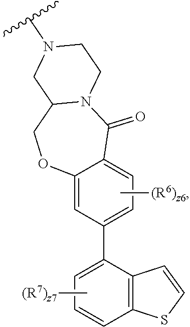

In embodiments, R¹ is

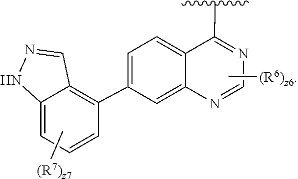

In embodiments, R¹ is

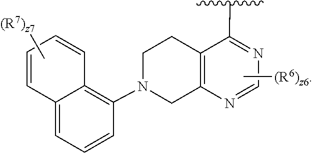

In embodiments, R¹ is

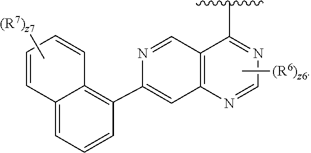

In embodiments, R¹ is

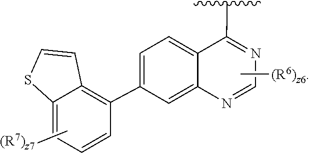

In embodiments, R¹ is

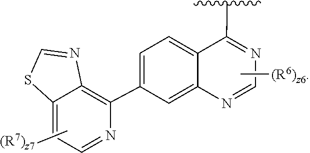

In embodiments, R¹ is

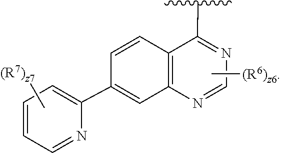

In embodiments, R¹ is

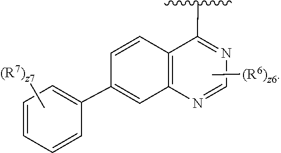

In embodiments, R¹ is

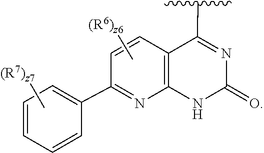

In embodiments, R¹ is

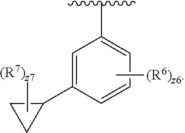

In embodiments, R¹ is

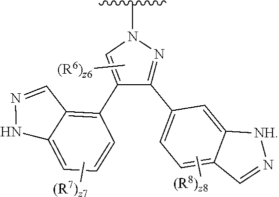

In embodiments, R¹ is

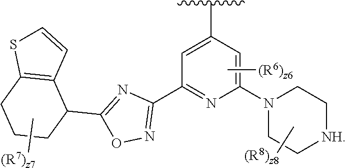

In embodiments, R¹ is

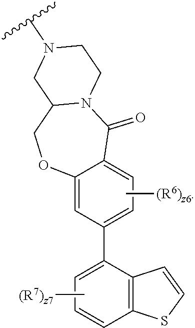

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z6 is an integer from 0 to 7.

The symbol z7 is an integer from 0 to 7.

The symbol z8 is an integer from 0 to 5.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁶ is independently oxo. In embodiments, R⁶ is independently halogen. In embodiments, R⁶ is independently —F. In embodiments, R⁶ is independently —Cl. In embodiments, R⁶ is independently —Br. In embodiments, R⁶ is independently —I. In embodiments, R⁶ is independently —CCl₃. In embodiments, R⁶ is independently —CBr₃. In embodiments, R⁶ is independently —CF₃. In embodiments, R⁶ is independently —CI₃. In embodiments, R⁶ is independently —CH₂Cl. In embodiments, R⁶ is independently —CH₂Br. In embodiments, R⁶ is independently —CH₂F. In embodiments, R⁶ is independently —CH₂I. In embodiments, R⁶ is independently —CHCl₂. In embodiments, R⁶ is independently —CHBr₂. In embodiments, R⁶ is independently —CHF₂. In embodiments, R⁶ is independently —CHI₂. In embodiments, R⁶ is independently —CN. In embodiments, R⁶ is independently —OH. In embodiments, R⁶ is independently —NH₂. In embodiments, R⁶ is independently —COOH. In embodiments, R⁶ is independently —CONH₂. In embodiments, R⁶ is independently —NO₂. In embodiments, R⁶ is independently —SH. In embodiments, R⁶ is independently —SO₃H. In embodiments, R⁶ is independently —OSO₃H. In embodiments, R⁶ is independently —SO₂NH₂. In embodiments, R⁶ is independently —NHNH₂. In embodiments, R⁶ is independently —ONH₂. In embodiments, R⁶ is independently —NHC(O)NHNH₂. In embodiments, R⁶ is independently —NHC(O)NH₂. In embodiments, R⁶ is independently —NHSO₂H. In embodiments, R⁶ is independently —NHC(O)H. In embodiments, R⁶ is independently —NHC(O)OH. In embodiments, R⁶ is independently —NHOH. In embodiments, R⁶ is independently —OCCl₃. In embodiments, R⁶ is independently —OCBr₃. In embodiments, R⁶ is independently —OCF₃. In embodiments, R⁶ is independently —OCI₃. In embodiments, R⁶ is independently —OCH₂Cl. In embodiments, R⁶ is independently —OCH₂Br. In embodiments, R⁶ is independently —OCH₂F. In embodiments, R⁶ is independently —OCH₂I. In embodiments, R⁶ is independently —OCHCl₂. In embodiments, R⁶ is independently —OCHBr₂. In embodiments, R⁶ is independently —OCHF₂. In embodiments, R⁶ is independently —OCHI₂. In embodiments, R⁶ is independently unsubstituted C₁-C₄ alkyl. In embodiments, R⁶ is independently unsubstituted methyl. In embodiments, R⁶ is independently unsubstituted ethyl. In embodiments, R⁶ is independently unsubstituted propyl. In embodiments, R⁶ is independently unsubstituted n-propyl. In embodiments, R⁶ is independently unsubstituted isopropyl. In embodiments, R⁶ is independently unsubstituted butyl. In embodiments, R⁶ is independently unsubstituted n-butyl. In embodiments, R⁶ is independently unsubstituted isobutyl. In embodiments, R⁶ is independently unsubstituted tert-butyl. In embodiments, R⁶ is independently substituted or unsubstituted 2 to 6 membered heteroalkyl. In embodiments, R⁶ is independently substituted 2 to 6 membered heteroalkyl. In embodiments, R⁶ is independently unsubstituted methoxy. In embodiments, R⁶ is independently unsubstituted ethoxy. In embodiments, R⁶ is independently unsubstituted propoxy. In embodiments, R⁶ is independently unsubstituted n-propoxy. In embodiments, R⁶ is independently unsubstituted isopropoxy. In embodiments, R⁶ is independently unsubstituted butoxy. In embodiments, R⁶ is independently unsubstituted n-butoxy. In embodiments, R⁶ is independently unsubstituted isobutoxy. In embodiments, R⁶ is independently unsubstituted tert-butoxy. In embodiments, R⁶ is independently substituted or unsubstituted 5 to 6 membered heteroaryl.

In embodiments, R⁶ is independently a halogen, —OH, unsubstituted C₁-C₄ alkyl, substituted 2 to 6 membered heteroalkyl, or substituted 5 to 6 membered heteroaryl. In embodiments, R⁶ is independently —F, —Cl, —OH, or unsubstituted methyl. In embodiments, R⁶ is independently a 2 to 6 membered heteroalkyl, substituted with substituted heterocycloalkyl or unsubstituted fused heterocycloalkyl. In embodiments, R⁶ is independently a substituted pyridyl.

In embodiments, z6 is 0. In embodiments, z6 is 1. In embodiments, z6 is 2. In embodiments, z6 is 3. In embodiments, z6 is 4. In embodiments, z6 is 5. In embodiments, z6 is 6. In embodiments, z6 is 7.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁷ is independently oxo. In embodiments, R⁷ is independently halogen. In embodiments, R⁷ is independently —F. In embodiments, R⁷ is independently —Cl. In embodiments, R⁷ is independently —Br. In embodiments, R⁷ is independently —I. In embodiments, R⁷ is independently —CCl₃. In embodiments, R⁷ is independently —CBr₃. In embodiments, R⁷ is independently —CF₃. In embodiments, R⁷ is independently —CI₃. In embodiments, R⁷ is independently —CH₂Cl. In embodiments, R⁷ is independently —CH₂Br. In embodiments, R⁷ is independently —CH₂F. In embodiments, R⁷ is independently —CH₂I. In embodiments, R⁷ is independently —CHCl₂. In embodiments, R⁷ is independently —CHBr₂. In embodiments, R⁷ is independently —CHF₂. In embodiments, R⁷ is independently —CHI₂. In embodiments, R⁷ is independently —CN. In embodiments, R⁷ is independently —OH. In embodiments, R⁷ is independently —NH₂. In embodiments, R⁷ is independently —COOH. In embodiments, R⁷ is independently —CONH₂. In embodiments, R⁷ is independently —NO₂. In embodiments, R⁷ is independently —SH. In embodiments, R⁷ is independently —SO₃H. In embodiments, R⁷ is independently —OSO₃H. In embodiments, R⁷ is independently —SO₂NH₂. In embodiments, R⁷ is independently —NHNH₂. In embodiments, R⁷ is independently —ONH₂. In embodiments, R⁷ is independently —NHC(O)NHNH₂. In embodiments, R⁷ is independently —NHC(O)NH₂. In embodiments, R⁷ is independently —NHSO₂H. In embodiments, R⁷ is independently —NHC(O)H. In embodiments, R⁷ is independently —NHC(O)OH. In embodiments, R⁷ is independently —NHOH. In embodiments, R⁷ is independently —OCCl₃. In embodiments, R⁷ is independently —OCBr₃. In embodiments, R⁷ is independently —OCF₃. In embodiments, R⁷ is independently —OCI₃. In embodiments, R⁷ is independently —OCH₂Cl. In embodiments, R⁷ is independently —OCH₂Br. In embodiments, R⁷ is independently —OCH₂F. In embodiments, R⁷ is independently —OCH₂I. In embodiments, R⁷ is independently —OCHCl₂. In embodiments, R⁷ is independently —OCHBr₂. In embodiments, R⁷ is independently —OCHF₂. In embodiments, R⁷ is independently —OCHI₂. In embodiments, R⁷ is independently unsubstituted C₁-C₄ alkyl. In embodiments, R⁷ is independently unsubstituted methyl. In embodiments, R⁷ is independently unsubstituted ethyl. In embodiments, R⁷ is independently unsubstituted propyl. In embodiments, R⁷ is independently unsubstituted n-propyl. In embodiments, R⁷ is independently unsubstituted isopropyl. In embodiments, R⁷ is independently unsubstituted butyl. In embodiments, R⁷ is independently unsubstituted n-butyl. In embodiments, R⁷ is independently unsubstituted isobutyl. In embodiments, R⁷ is independently unsubstituted tert-butyl. In embodiments, R⁷ is independently unsubstituted C₂-C₄ alkynyl. In embodiments, R⁷ is independently unsubstituted ethynyl. In embodiments, R⁷ is independently unsubstituted propynyl. In embodiments, R⁷ is independently unsubstituted butynyl. In embodiments, R⁷ is independently substituted or unsubstituted 2 to 6 membered heteroalkyl. In embodiments, R⁷ is independently unsubstituted methoxy. In embodiments, R⁷ is independently unsubstituted ethoxy. In embodiments, R⁷ is independently unsubstituted propoxy. In embodiments, R⁷ is independently unsubstituted n-propoxy. In embodiments, R⁷ is independently unsubstituted isopropoxy. In embodiments, R⁷ is independently unsubstituted butoxy. In embodiments, R⁷ is independently unsubstituted n-butoxy. In embodiments, R⁷ is independently unsubstituted isobutoxy. In embodiments, R⁷ is independently unsubstituted tert-butoxy.

In embodiments, R⁷ is independently a halogen, —CF₃, —CN, —OH, —NH₂, unsubstituted C₁-C₄ alkyl, unsubstituted C₂-C₄ alkynyl, unsubstituted 2 to 6 membered heteroalkyl, or unsubstituted C₃-C₈ cycloalkyl. In embodiments, R⁷ is independently —F, —Cl, —CF₃, —CN, —OH, —NH₂, unsubstituted methyl, unsubstituted ethynyl, unsubstituted methoxy, or unsubstituted cyclopropyl.

In embodiments, R⁷ is independently a halogen, —CF₃, —CN, —OH, —NH₂, unsubstituted C₁-C₄ alkyl, unsubstituted C₂-C₄ alkynyl, or unsubstituted C₃-C₅ cycloalkyl. In embodiments, R⁷ is independently —F, —Cl, —CF₃, —CN, —OH, —NH₂, unsubstituted methyl, unsubstituted ethynyl, or unsubstituted cyclopropyl.

In embodiments, z7 is 0. In embodiments, z7 is 1. In embodiments, z7 is 2. In embodiments, z7 is 3. In embodiments, z7 is 4. In embodiments, z7 is 5. In embodiments, z7 is 6. In embodiments, z7 is 7.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is independently halogen. In embodiments, R⁸ is independently —F. In embodiments, R⁸ is independently —Cl. In embodiments, R⁸ is independently —Br. In embodiments, R⁸ is independently —I. In embodiments, R⁸ is independently —CCl₃. In embodiments, R⁸ is independently —CBr₃. In embodiments, R⁸ is independently —CF₃. In embodiments, R⁸ is independently —CI₃. In embodiments, R⁸ is independently —CH₂Cl. In embodiments, R⁸ is independently —CH₂Br. In embodiments, R⁸ is independently —CH₂F. In embodiments, R⁸ is independently —CH₂I. In embodiments, R⁸ is independently —CHCl₂. In embodiments, R⁸ is independently —CHBr₂. In embodiments, R⁸ is independently —CIF₂. In embodiments, R⁸ is independently —CHI₂. In embodiments, R⁸ is independently —CN. In embodiments, R⁸ is independently —OH. In embodiments, R⁸ is independently —NH₂. In embodiments, R⁸ is independently —COOH. In embodiments, R⁸ is independently —CONH₂. In embodiments, R⁸ is independently —NO₂. In embodiments, R⁸ is independently —SH. In embodiments, R⁸ is independently —SO₃H. In embodiments, R⁸ is independently —OSO₃H. In embodiments, R⁸ is independently —SO₂NH₂. In embodiments, R⁸ is independently —NHNH₂. In embodiments, R⁸ is independently —ONH₂. In embodiments, R⁸ is independently —NHC(O)NHNH₂. In embodiments, R⁸ is independently —NHC(O)NH₂. In embodiments, R⁸ is independently —NHSO₂H. In embodiments, R⁸ is independently —NHC(O)H. In embodiments, R⁸ is independently —NHC(O)OH. In embodiments, R⁸ is independently —NHOH. In embodiments, R⁸ is independently —OCCl₃. In embodiments, R⁸ is independently —OCBr₃. In embodiments, R⁸ is independently —OCF₃. In embodiments, R⁸ is independently —OCI₃. In embodiments, R⁸ is independently —OCH₂Cl. In embodiments, R⁸ is independently —OCH₂Br. In embodiments, R⁸ is independently —OCH₂F. In embodiments, R⁸ is independently —OCH₂I. In embodiments, R⁸ is independently —OCHCl₂. In embodiments, R⁸ is independently —OCHBr₂. In embodiments, R⁸ is independently —OCHF₂. In embodiments, R⁸ is independently —OCHI₂. In embodiments, R⁸ is independently unsubstituted C₁-C₄ alkyl. In embodiments, R⁸ is independently unsubstituted methyl. In embodiments, R⁸ is independently unsubstituted ethyl. In embodiments, R⁸ is independently unsubstituted propyl. In embodiments, R⁸ is independently unsubstituted n-propyl. In embodiments, R⁸ is independently unsubstituted isopropyl. In embodiments, R⁸ is independently unsubstituted butyl. In embodiments, R⁸ is independently unsubstituted n-butyl. In embodiments, R⁸ is independently unsubstituted isobutyl. In embodiments, R⁸ is independently unsubstituted tert-butyl. In embodiments, R⁸ is independently substituted or unsubstituted 2 to 6 membered heteroalkyl. In embodiments, R⁸ is independently unsubstituted methoxy. In embodiments, R⁸ is independently unsubstituted ethoxy. In embodiments, R⁸ is independently unsubstituted propoxy. In embodiments, R⁸ is independently unsubstituted n-propoxy. In embodiments, R⁸ is independently unsubstituted isopropoxy. In embodiments, R⁸ is independently unsubstituted butoxy. In embodiments, R⁸ is independently unsubstituted n-butoxy. In embodiments, R⁸ is independently unsubstituted isobutoxy. In embodiments, R⁸ is independently unsubstituted tert-butoxy.

In embodiments, R⁸ is independently a halogen or unsubstituted C₁-C₄ alkyl. In embodiments, R⁸ is independently —Cl or unsubstituted methyl.

In embodiments, z8 is 0. In embodiments, z8 is 1. In embodiments, z8 is 2. In embodiments, z8 is 3. In embodiments, z8 is 4. In embodiments, z8 is 5.

In embodiments, R¹ is

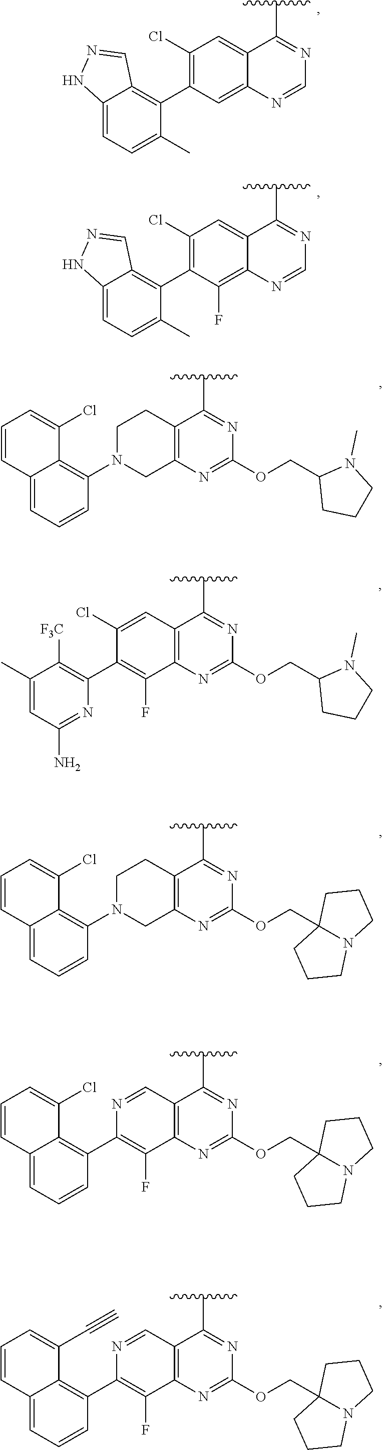

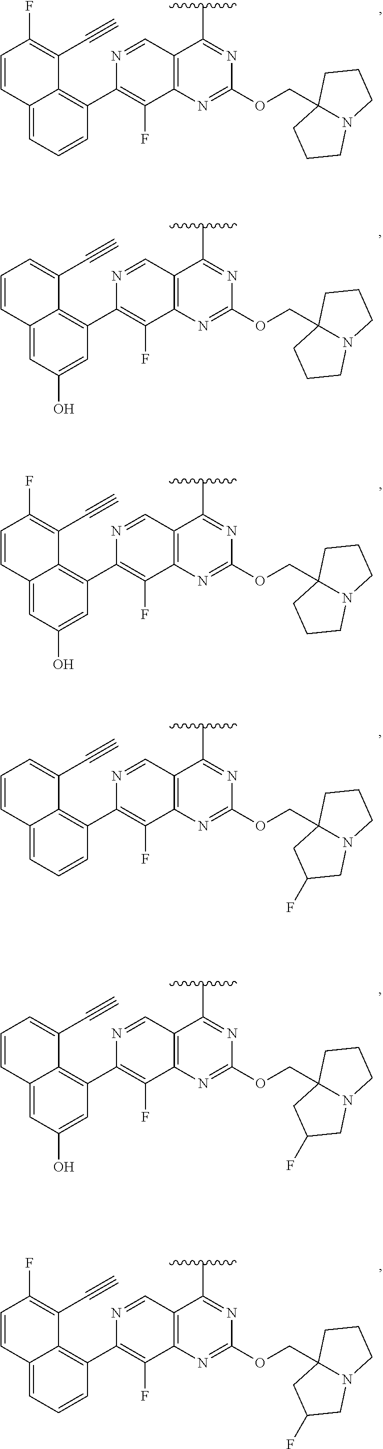 [SMILES: *c1nc(OCC23CCCN2CCC3)nc2c(F)c(-c3cccc4ccc(F)c(C#C)c34)ncc12 ; *c1nc(OCC23CCCN2CCC3)nc2c(F)c(-c3cc(O)cc4cccc(C#C)c34)ncc12 ; *c1nc(OCC23CCCN2CCC3)nc2c(F)c(-c3cc(O)cc4ccc(F)c(C#C)c34)ncc12 ; *c1nc(OCC23CCCN2CC(F)C3)nc2c(F)c(-c3cccc4cccc(C#C)c34)ncc12 ; *c1nc(OCC23CCCN2CC(F)C3)nc2c(F)c(-c3cc(O)cc4cccc(C#C)c34)ncc12 ; *c1nc(OCC23CCCN2CC(F)C3)nc2c(F)c(-c3cccc4ccc(F)c(C#C)c34)ncc12]

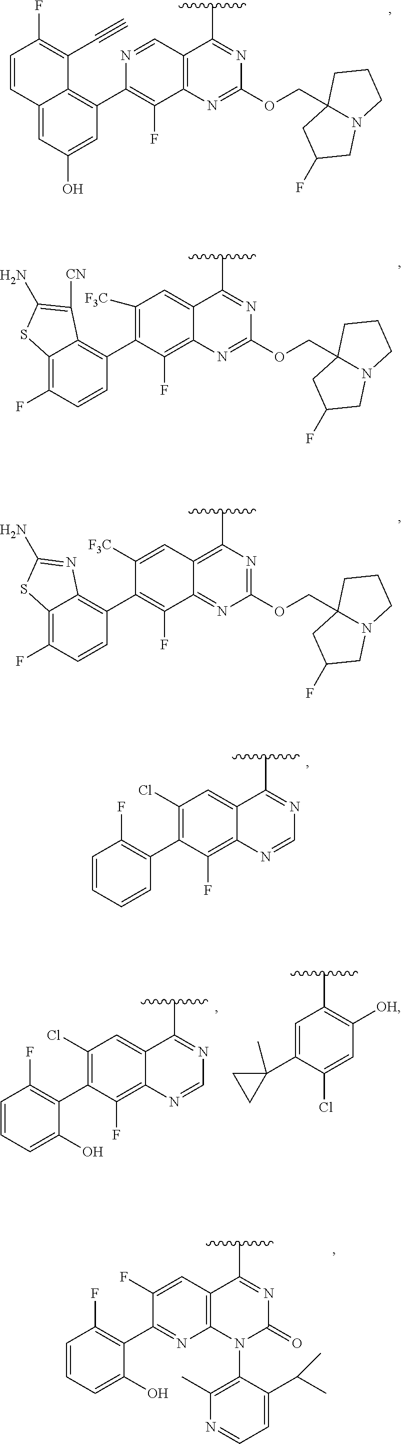

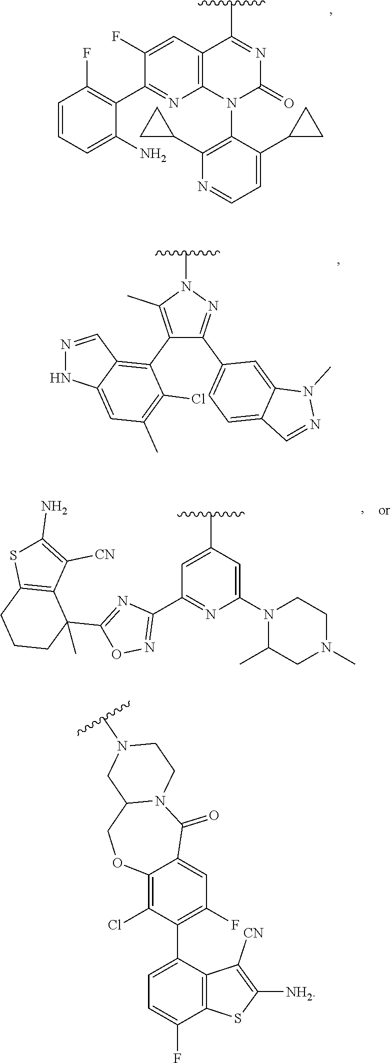 [SMILES: *c1nc(=O)n(-c2c(C3CC3)ccnc2C2CC2)c2nc(-c3c(N)cccc3F)c(F)cc12 ; *n1nc(-c2ccc3cnn(C)c3c2)c(-c2c(Cl)c(C)cc3[nH]ncc23)c1C ; *c1cc(-c2noc(C3(C)CCCc4sc(N)c(C#N)c43)n2)nc(N2CCN(C)CC2C)c1 ; *N1CCN2C(=O)c3cc(F)c(-c4ccc(F)c5sc(N)c(C#N)c45)c(Cl)c3OCC2C1]

In embodiments, R¹ is

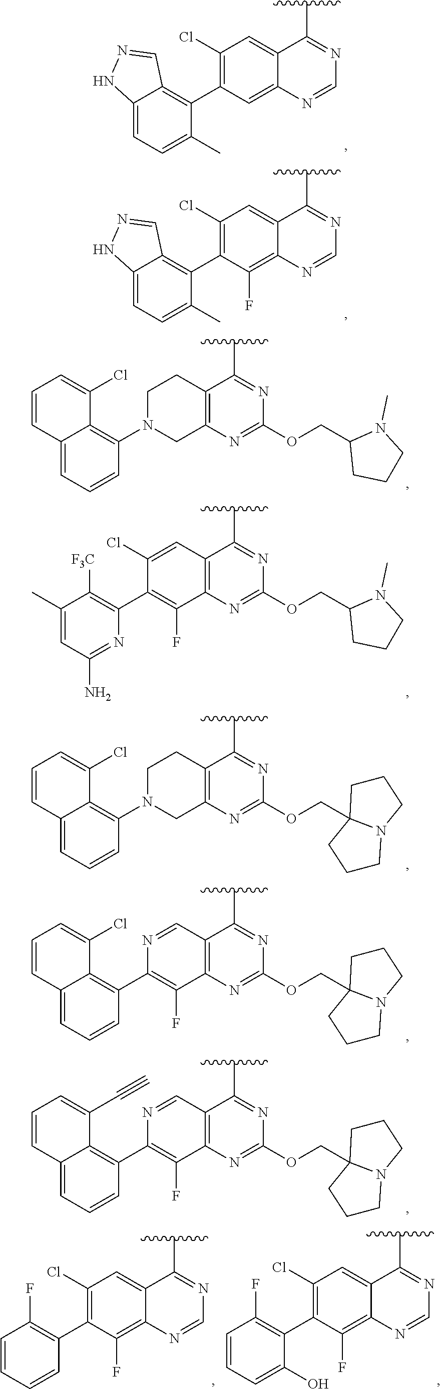

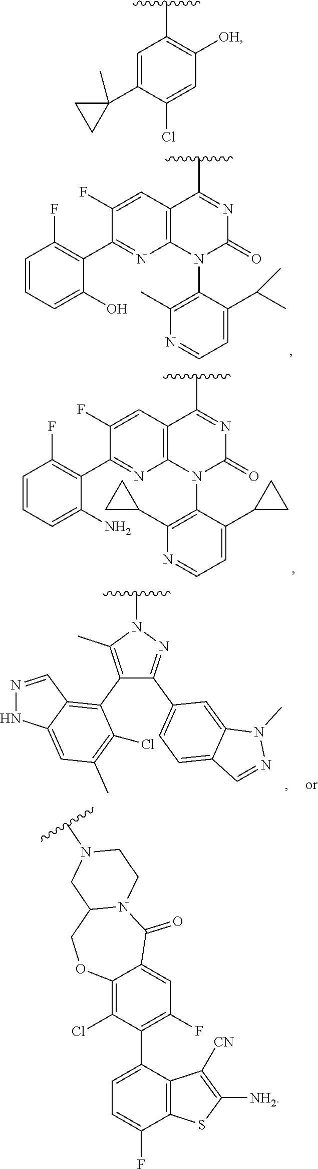

In embodiments, R¹ is

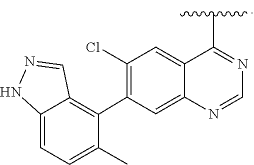 [SMILES: *c1ncnc2cc(-c3c(C)ccc4[nH]ncc34)c(Cl)cc12]

In embodiments, R¹ is

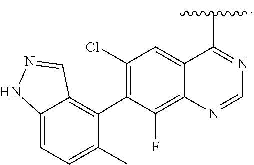 [SMILES: *c1ncnc2c(F)c(-c3c(C)ccc4[nH]ncc34)c(Cl)cc12]

In embodiments, R¹ is

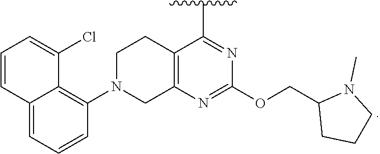 [SMILES: *c1nc(OCC2CCCN2C)nc2c1CCN(c1cccc3cccc(Cl)c13)C2]

In embodiments, R¹ is

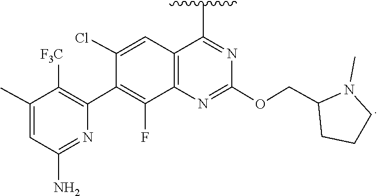 [SMILES: *c1nc(OCC2CCCN2C)nc2c(F)c(-c3nc(N)cc(C)c3C(F)(F)F)c(Cl)cc12]

In embodiments, R¹ is

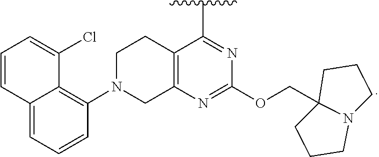 [SMILES: *c1nc(OCC23CCCN2CCC3)nc2c1CCN(c1cccc3cccc(Cl)c13)C2]

In embodiments, R¹ is

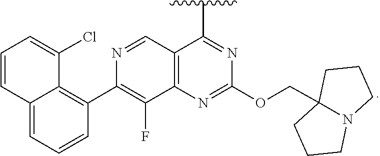 [SMILES: *c1nc(OCC23CCCN2CCC3)nc2c(F)c(-c3cccc4cccc(Cl)c34)ncc12]

In embodiments, R¹ is

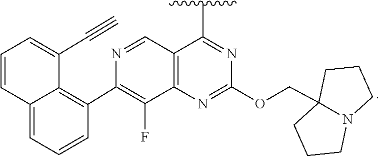 [SMILES: *c1nc(OCC23CCCN2CCC3)nc2c(F)c(-c3cccc4cccc(C#C)c34)ncc12]

In embodiments, R¹ is

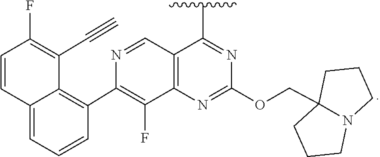 [SMILES: *c1nc(OCC23CCCN2CCC3)nc2c(F)c(-c3cccc4ccc(F)c(C#C)c34)ncc12]

In embodiments, R¹ is

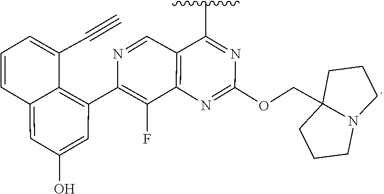 [SMILES: *c1nc(OCC23CCCN2CCC3)nc2c(F)c(-c3cc(O)cc4cccc(C#C)c34)ncc12]

In embodiments, R¹ is

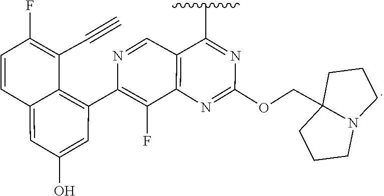 [SMILES: *c1nc(OCC23CCCN2CCC3)nc2c(F)c(-c3cc(O)cc4ccc(F)c(C#C)c34)ncc12]

In embodiments, R¹ is

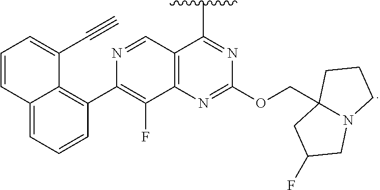 [SMILES: *c1nc(OCC23CCCN2CC(F)C3)nc2c(F)c(-c3cccc4cccc(C#C)c34)ncc12]

In embodiments, R¹ is

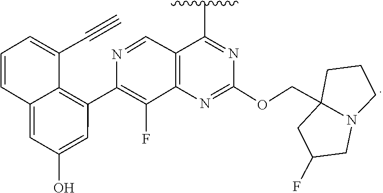 [SMILES: *c1nc(OCC23CCCN2CC(F)C3)nc2c(F)c(-c3cc(O)cc4cccc(C#C)c34)ncc12]

In embodiments, R¹ is

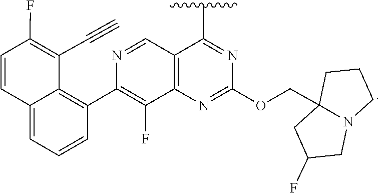 [SMILES: *c1nc(OCC23CCCN2CC(F)C3)nc2c(F)c(-c3cccc4ccc(F)c(C#C)c34)ncc12]

In embodiments, R¹ is

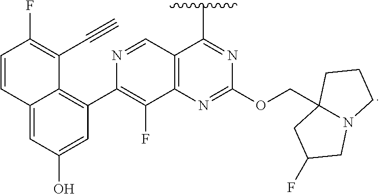 [SMILES: *c1nc(OCC23CCCN2CC(F)C3)nc2c(F)c(-c3cc(O)cc4ccc(F)c(C#C)c34)ncc12]

In embodiments, R¹ is

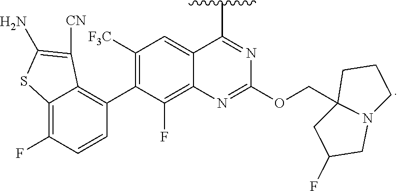 [SMILES: *c1nc(OCC23CCCN2CC(F)C3)nc2c(F)c(-c3ccc(F)c4sc(N)c(C#N)c34)c(C(F)(F)F)cc12]

In embodiments, R¹ is

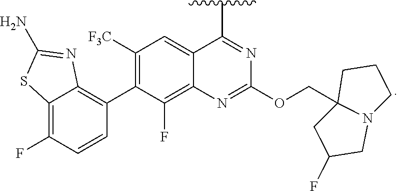 [SMILES: *c1nc(OCC23CCCN2CC(F)C3)nc2c(F)c(-c3ccc(F)c4sc(N)nc34)c(C(F)(F)F)cc12]

In embodiments, R¹ is

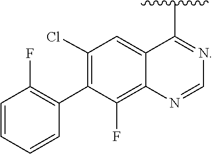 [SMILES: *c1ncnc2c(F)c(-c3ccccc3F)c(Cl)cc12]

In embodiments, R¹ is

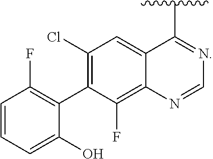 [SMILES: *c1ncnc2c(F)c(-c3c(O)cccc3F)c(Cl)cc12]

In embodiments, R¹ is

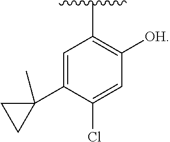 [SMILES: *c1cc(C2(C)CC2)c(Cl)cc1O]

In embodiments, R¹ is

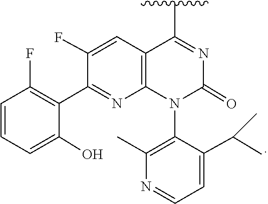 [SMILES: *c1nc(=O)n(-c2c(C(C)C)ccnc2C)c2nc(-c3c(O)cccc3F)c(F)cc12]

In embodiments, R¹ is

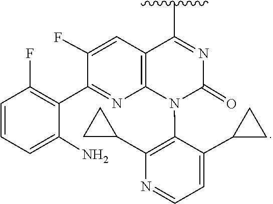 [SMILES: *c1nc(=O)n(-c2c(C3CC3)ccnc2C2CC2)c2nc(-c3c(N)cccc3F)c(F)cc12]

In embodiments, R¹ is

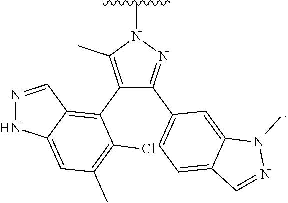 [SMILES: *n1nc(-c2ccc3cnn(C)c3c2)c(-c2c(Cl)c(C)cc3[nH]ncc23)c1C]

In embodiments, R¹ is

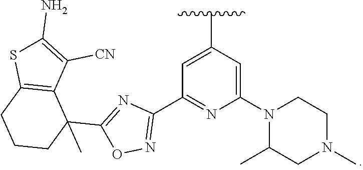 [SMILES: *c1cc(-c2noc(C3(C)CCCc4sc(N)c(C#N)c43)n2)nc(N2CCN(C)CC2C)c1]

In embodiments, R¹ is

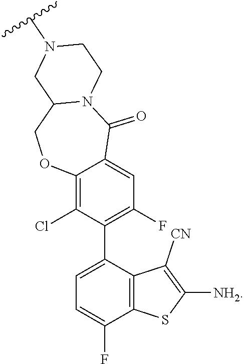 [SMILES: *N1CCN2C(=O)c3cc(F)c(-c4ccc(F)c5sc(N)c(C#N)c45)c(Cl)c3OCC2C1]

In embodiments, R¹ is a monovalent form of ARS-1620. In embodiments, R¹ is a monovalent form of

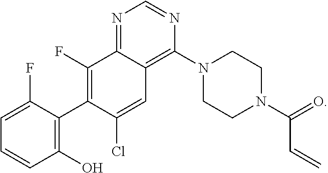 [SMILES: C=CC(=O)N1CCN(c2ncnc3c(F)c(-c4c(O)cccc4F)c(Cl)cc23)CC1]

In embodiments, R¹ is

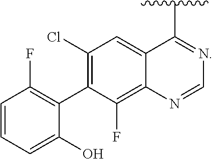 [SMILES: *c1ncnc2c(F)c(-c3c(O)cccc3F)c(Cl)cc12]

In embodiments, R¹ is a monovalent form of a portion of ARS-1620, wherein R¹ does not include the substituted piperazinyl moiety.

In embodiments, R¹ is a monovalent form of AMG-510. In embodiments, R¹ is a monovalent form of a compound as described in Canon, J. et al. Nature 575, 217-223 (2019), which is herein incorporated by reference in its entirety for all purposes. In embodiments, R¹ is a monovalent form of

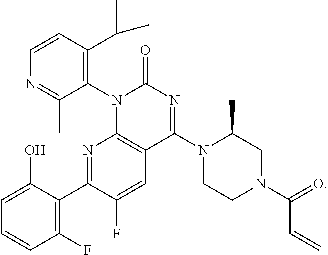 [SMILES: C=CC(=O)N1CCN(c2nc(=O)n(-c3c(C(C)C)ccnc3C)c3nc(-c4c(O)cccc4F)c(F)cc23)[C@@H](C)C1]

In embodiments, R¹ is

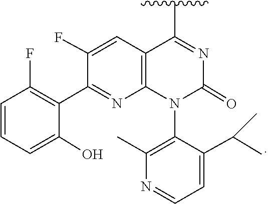 [SMILES: *c1nc(=O)n(-c2c(C(C)C)ccnc2C)c2nc(-c3c(O)cccc3F)c(F)cc12]

In embodiments, R¹ is a monovalent form of a portion of AMG-510, wherein R¹ does not include the substituted piperazinyl moiety or equivalent for compounds described in Canon, et al.

In embodiments, R¹ is a monovalent form of MRTX-849. In embodiments, R¹ is a monovalent form of a compound as described in Fell, J. B. et al. J. Med. Chem. 63, 6679-6693 (2020), which is herein incorporated by reference in its entirety for all purposes. In embodiments, R is a monovalent form of

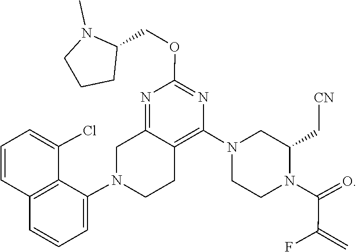 [SMILES: C=C(F)C(=O)N1CCN(c2nc(OC[C@@H]3CCCN3C)nc3c2CCN(c2cccc4cccc(Cl)c24)C3)C[C@@H]1CC#N]

In embodiments, R¹ is

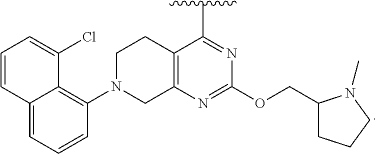 [SMILES: *c1nc(OCC2CCCN2C)nc2c1CCN(c1cccc3cccc(Cl)c13)C2]

In embodiments, R¹ is

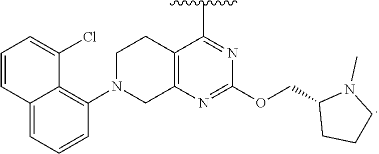 [SMILES: *c1nc(OC[C@H]2CCCN2C)nc2c1CCN(c1cccc3cccc(Cl)c13)C2]

In embodiments, R¹ is

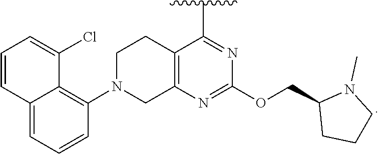 [SMILES: *c1nc(OC[C@@H]2CCCN2C)nc2c1CCN(c1cccc3cccc(Cl)c13)C2]

In embodiments, R¹ is a monovalent form of a portion of MRTX-849, wherein R¹ does not include the substituted piperazinyl moiety or equivalent for compounds described in Fell, et al.

In embodiments, R¹ is a monovalent form of GDC-6036. In embodiments, R¹ is a monovalent form of a compound as described in WO2020097537, which is herein incorporated by reference in its entirety for all purposes. In embodiments, R¹ is a monovalent form of

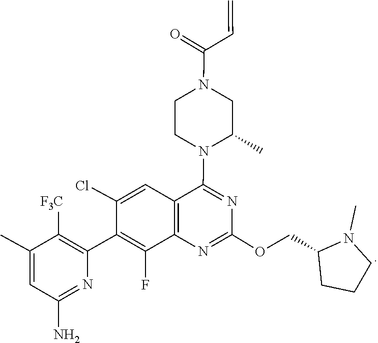 [SMILES: C=CC(=O)N1CCN(c2nc(OC[C@H]3CCCN3C)nc3c(F)c(-c4nc(N)cc(C)c4C(F)(F)F)c(Cl)cc23)[C@@H](C)C1]

In embodiments, R¹ is

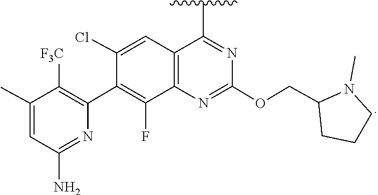 [SMILES: *c1nc(OCC2CCCN2C)nc2c(F)c(-c3nc(N)cc(C)c3C(F)(F)F)c(Cl)cc12]

In embodiments, R¹ is

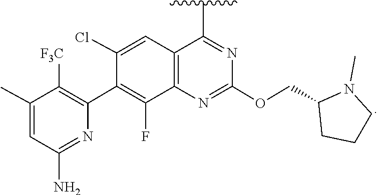 [SMILES: *c1nc(OC[C@H]2CCCN2C)nc2c(F)c(-c3nc(N)cc(C)c3C(F)(F)F)c(Cl)cc12]

In embodiments, R¹ is

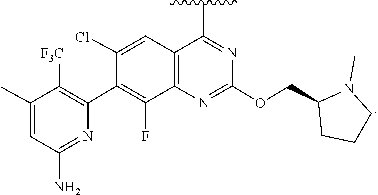 [SMILES: *c1nc(OC[C@@H]2CCCN2C)nc2c(F)c(-c3nc(N)cc(C)c3C(F)(F)F)c(Cl)cc12]

In embodiments, R¹ is a monovalent form of a portion of GDC-6036, wherein R¹ does not include the substituted piperazinyl moiety or equivalent for compounds described in WO2020097537.

In embodiments, R⁸ is a monovalent form of MRTX1133. In embodiments, R¹ is a monovalent form of a compound as described in Wang, X. et al. J. Med. Chem. 65, 3123-3133 (2022), which is herein incorporated by reference in its entirety for all purposes. In embodiments, R¹ is a monovalent form of

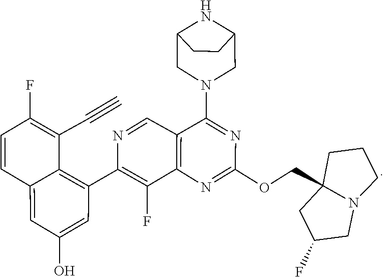 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

In embodiments, R¹ is

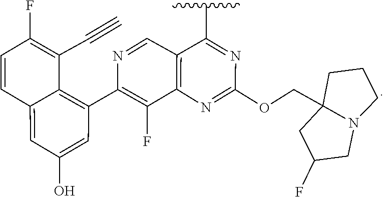 [SMILES: *c1nc(OCC23CCCN2CC(F)C3)nc2c(F)c(-c3cc(O)cc4ccc(F)c(C#C)c34)ncc12]

In embodiments, R¹ is

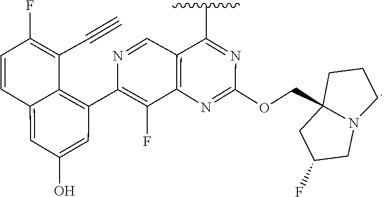 [SMILES: *c1nc(OC[C@@]23CCCN2C[C@H](F)C3)nc2c(F)c(-c3cc(O)cc4ccc(F)c(C#C)c34)ncc12]

In embodiments, R¹ is a monovalent form of a portion of MRTX1133, wherein R¹ does not include the diazabicyclooctanyl moiety or equivalent for compounds described in Wang, et al.

In embodiments, R¹ is a monovalent form of BBO-8520. In embodiments, R¹ is a monovalent form of putative BBO-8520. In embodiments, R¹ is a monovalent form of a compound as described in WO2023004102, which is herein incorporated by reference in its entirety for all purposes. In embodiments, R¹ is a monovalent form of

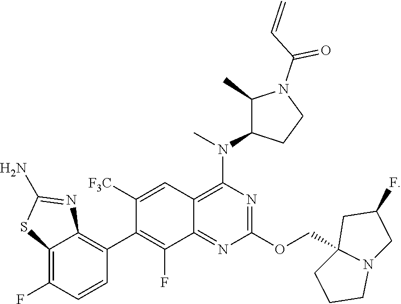

In embodiments, R¹ is

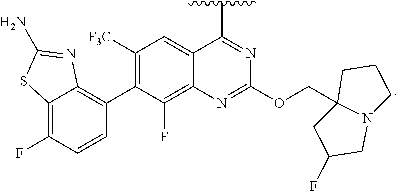 [SMILES: *c1nc(OCC23CCCN2CC(F)C3)nc2c(F)c(-c3ccc(F)c4sc(N)nc34)c(C(F)(F)F)cc12]

In embodiments, R¹ is

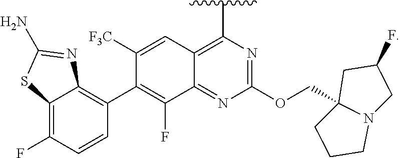

In embodiments, R¹ is

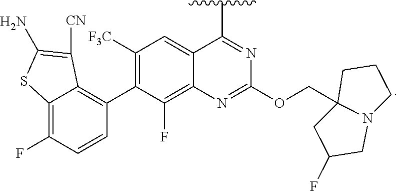 [SMILES: *c1nc(OCC23CCCN2CC(F)C3)nc2c(F)c(-c3ccc(F)c4sc(N)c(C#N)c34)c(C(F)(F)F)cc12]

In embodiments, R¹ is

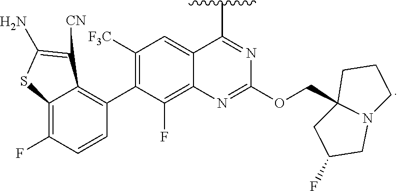

F In embodiments, R¹ is a monovalent form of a portion of BBO-8520, wherein R¹ does not include the

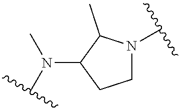 [SMILES: *N(C)C1CCN(*)C1C]

or equivalent for compounds described in WO2023004102.

In embodiments, R¹ is a monovalent form of JDQ-443. In embodiments, R¹ is a monovalent form of a compound as described in WO2021120890, which is herein incorporated by reference in its entirety for all purposes. In embodiments, R¹ is a monovalent form of

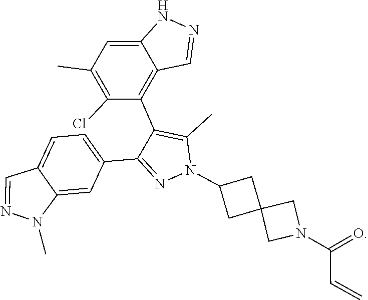 [SMILES: C=CC(=O)N1CC2(CC(n3nc(-c4ccc5cnn(C)c5c4)c(-c4c(Cl)c(C)cc5[nH]ncc45)c3C)C2)C1]

In embodiments, R¹ is

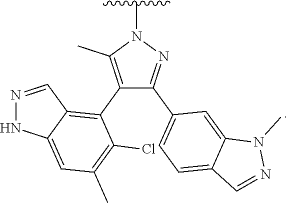 [SMILES: *n1nc(-c2ccc3cnn(C)c3c2)c(-c2c(Cl)c(C)cc3[nH]ncc23)c1C]

In embodiments, R¹ is a monovalent form of a portion of JDQ-443, wherein R¹ does not include the azaspiroheptanyl moiety or equivalent for compounds described in WO2021120890.

In embodiments, R¹ is a monovalent form of BI-0474. In embodiments, R⁸ is a monovalent form of a compound as described in Broker, J. et al. J. Med. Chem. 65, 14614-14629 (2022), which is herein incorporated by reference in its entirety for all purposes. In embodiments, R¹ is a monovalent form of

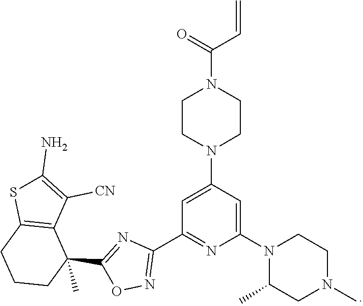 [SMILES: C=CC(=O)N1CCN(c2cc(-c3noc([C@@]4(C)CCCc5sc(N)c(C#N)c54)n3)nc(N3CCN(C)C[C@@H]3C)c2)CC1]

In embodiments, R¹ is

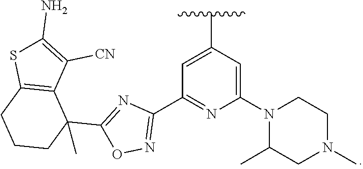 [SMILES: *c1cc(-c2noc(C3(C)CCCc4sc(N)c(C#N)c43)n2)nc(N2CCN(C)CC2C)c1]

In embodiments, R¹ is

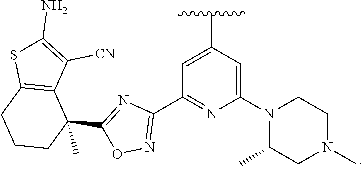 [SMILES: *c1cc(-c2noc([C@@]3(C)CCCc4sc(N)c(C#N)c43)n2)nc(N2CCN(C)C[C@@H]2C)c1]

In embodiments, R¹ is a monovalent form of a portion of BI-0474, wherein R¹ does not include the piperazinyl moiety or equivalent for compounds described in Bröker, et al.

In embodiments, R¹ is a monovalent form of a compound as described in WO2021118877, which is herein incorporated by reference in its entirety for all purposes. In embodiments, R¹ is

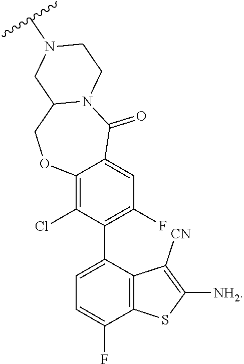 [SMILES: *N1CCN2C(=O)c3cc(F)c(-c4ccc(F)c5sc(N)c(C#N)c45)c(Cl)c3OCC2C1]

In embodiments, R¹ is a monovalent form of a portion of a compound described in WO2021118877, wherein R¹ does not include the acryloyl moiety or equivalent for compounds described in WO2021118877.

In embodiments, R¹ is a monovalent form of a compound as described in WO2021120045, which is herein incorporated by reference in its entirety for all purposes. In embodiments, R¹ is

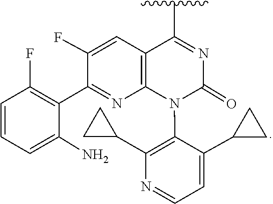 [SMILES: *c1nc(=O)n(-c2c(C3CC3)ccnc2C2CC2)c2nc(-c3c(N)cccc3F)c(F)cc12]

In embodiments, R¹ is a monovalent form of a portion of a compound described in WO2021120045, wherein R¹ does not include the substituted piperazinyl moiety or equivalent for compounds described in WO2021120045.

In embodiments, R¹ is a monovalent form of sotorasib. In embodiments, R¹ is a monovalent form of a compound as described in U.S. Pat. Nos. 10,519,146, 11,236,091, and 11,426,404, which are herein incorporated by reference in their entirety for all purposes. In embodiments, R¹ is a monovalent form of

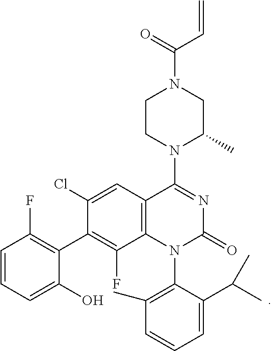 [SMILES: C=CC(=O)N1CCN(c2nc(=O)n(-c3c(C)cccc3C(C)C)c3c(F)c(-c4c(O)cccc4F)c(Cl)cc23)[C@@H](C)C1]

In embodiments, R¹ is

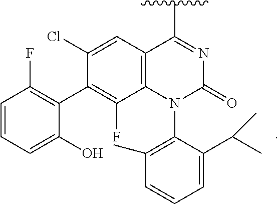 [SMILES: *c1nc(=O)n(-c2c(C)cccc2C(C)C)c2c(F)c(-c3c(O)cccc3F)c(Cl)cc12]

In embodiments, R¹ is a monovalent form of a portion of sotorasib, wherein R¹ does not include the substituted piperazinyl moiety or equivalent for compounds described in U.S. Pat. Nos. 10,519,146, 11,236,091, and 11,426,404.

In embodiments, R¹ is a monovalent form of adagrasib. In embodiments, R¹ is a monovalent form of a compound as described in WO 2021/037018, which is herein incorporated by reference in its entirety for all purposes. In embodiments, R¹ is a monovalent form of

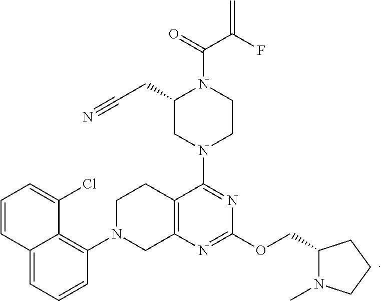 [SMILES: C=C(F)C(=O)N1CCN(c2nc(OC[C@@H]3CCCN3C)nc3c2CCN(c2cccc4cccc(Cl)c24)C3)C[C@@H]1CC#N]

In embodiments, R¹ is

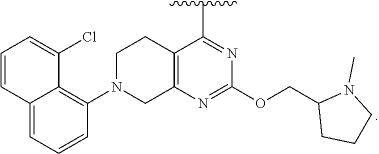 [SMILES: *c1nc(OCC2CCCN2C)nc2c1CCN(c1cccc3cccc(Cl)c13)C2]

In embodiments, R¹ is

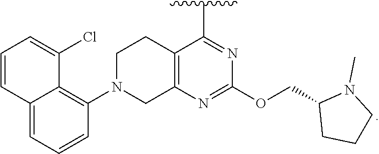 [SMILES: *c1nc(OC[C@H]2CCCN2C)nc2c1CCN(c1cccc3cccc(Cl)c13)C2]

In embodiments, R¹ is

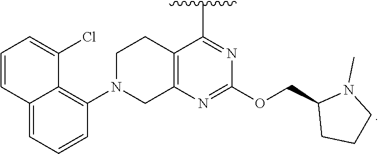 [SMILES: *c1nc(OC[C@@H]2CCCN2C)nc2c1CCN(c1cccc3cccc(Cl)c13)C2]

In embodiments, R¹ is a monovalent form of a portion of adagrasib, wherein R¹ does not include the substituted piperazinyl moiety or equivalent for compounds described in WO 2021/037018.

In embodiments, R¹ is a monovalent form of MRTX1257. In embodiments, R¹ is a monovalent form of a compound as described in US 2018/0072723, which is herein incorporated by reference in its entirety for all purposes. In embodiments, R¹ is a monovalent form of

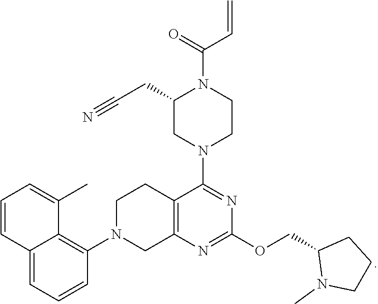 [SMILES: C=CC(=O)N1CCN(c2nc(OC[C@@H]3CCCN3C)nc3c2CCN(c2cccc4cccc(C)c24)C3)C[C@@H]1CC#N]

In embodiments, R¹ is

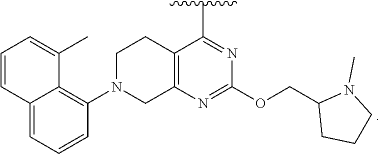 [SMILES: *c1nc(OCC2CCCN2C)nc2c1CCN(c1cccc3cccc(C)c13)C2]

In embodiments, R¹ is

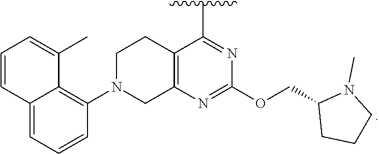 [SMILES: *c1nc(OC[C@H]2CCCN2C)nc2c1CCN(c1cccc3cccc(C)c13)C2]

In embodiments, R¹ is

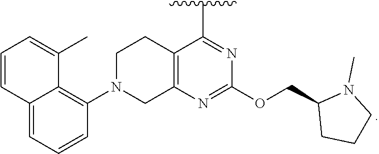 [SMILES: *c1nc(OC[C@@H]2CCCN2C)nc2c1CCN(c1cccc3cccc(C)c13)C2]

In embodiments, R¹ is a monovalent form of a portion of MRTX1257, wherein R¹ does not include the substituted piperazinyl moiety or equivalent for compounds described in US 2018/0072723.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R³² as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R⁶² substituent group is substituted with one or more third substituent groups denoted by R^{6.3} as explained in the definitions section above in the description of “first substituent group(s)”. In the above embodiments, R⁶, R^{6.1}, R⁶², and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2}, and R^{8.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸, R^{8.1}, R^{8.2}, and R^{8.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R^{10.1}, R^{10.2}, and R^{10.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10A}, R^{10A.1}, R^{10A.2}, and R^{10A.3} respectively.

In embodiments, when R^{10B} is substituted, R^{10B} is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10B}, R^{10B.1}, R^{10B.2}, and R^{10B.3} respectively.

In embodiments, when R^{10A} and R^{10B} substituents bonded to the same nitrogen atom are optionally joined to form a moiety that is substituted (e.g., a substituted heterocycloalkyl or substituted heteroaryl), the moiety is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A.1}, R^{10A.2}, and R^{10A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10A.1}, R^{10A.2}, and R^{10A.3}, respectively.

In embodiments, when R^{10A} and R^{10B} substituents bonded to the same nitrogen atom are optionally joined to form a moiety that is substituted (e.g., a substituted heterocycloalkyl or substituted heteroaryl), the moiety is substituted with one or more first substituent groups denoted by R^{10B} as explained in the definitions section above in the description of “first substituent group(s)”. In embodiments, when an R^{10B} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B.1}, R^{10B.2}, and R^{10B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10B.1}, R^{10B.2}, and R^{10B.3}, respectively.

In embodiments, when R^{10C} is substituted, R^{10C} is substituted with one or more first substituent groups denoted by R^{10C.1} as explained in the definitions section above in the description of “first substituent group(s)”. In embodiments, when an R^{10C.1} substituent group is substituted, the R^{10C.1} substituent group is substituted with one or more second substituent groups denoted by R^{10C.2} as explained in the definitions section above in the description of “first substituent group(s)”. In embodiments, when an R^{10C.2} substituent group is substituted, the R^{10C.2} substituent group is substituted with one or more third substituent groups denoted by R^{10C.3} as explained in the definitions section above in the description of “first substituent group(s)”. In the above embodiments, R^{10C}, R^{10C.1}, R^{10C.2}, and R^{10C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10C}, R^{10C.1}. R^{10C.2} and R^{10C.3} respectively.

In embodiments, when R^{10D} is substituted, R^{10D} is substituted with one or more first substituent groups denoted by R^{10D.1} as explained in the definitions section above in the description of “first substituent group(s)”. In embodiments, when an R^{10D.1} substituent group is substituted, the R^{10D.1} substituent group is substituted with one or more second substituent groups denoted by R^{10D.2} as explained in the definitions section above in the description of “first substituent group(s)”. In embodiments, when an R^{10D.2} substituent group is substituted, the R^{10D.2} substituent group is substituted with one or more third substituent groups denoted by R^{10D.3} as explained in the definitions section above in the description of “first substituent group(s)”. In the above embodiments, R^{10D}, R^{10D.1}, R^{10D.2}, and R^{10D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10D}, R^{10D.1}, R^{10D.2} and R^{10D.3} respectively.

In embodiments, when R²⁰ is substituted, R²⁰ is substituted with one or more first substituent groups denoted by R^{20.1} as explained in the definitions section above in the description of “first substituent group(s)”. In embodiments, when an R^{20.1} substituent group is substituted, the R^{20.1} substituent group is substituted with one or more second substituent groups denoted by R^{20.2} as explained in the definitions section above in the description of “first substituent group(s)”. In embodiments, when an R^{20.2} substituent group is substituted, the R^{20.2} substituent group is substituted with one or more third substituent groups denoted by R^{20.3} as explained in the definitions section above in the description of “first substituent group(s)”. In the above embodiments, R²⁰, R^{20.1}, R^{20.2}, and R^{20.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁰, R^{20.1}, R^{20.2}, and R^{20.3}, respectively.

In embodiments, when R^{20A} is substituted, R^{20A} is substituted with one or more first substituent groups denoted by R^{20A.1} as explained in the definitions section above in the description of “first substituent group(s)”. In embodiments, when an R^{20A.1} substituent group is substituted, the R^{20A.1} substituent group is substituted with one or more second substituent groups denoted by R^{20A.2} as explained in the definitions section above in the description of “first substituent group(s)”. In embodiments, when an R^{20A.2} substituent group is substituted, the R^{20A.2} substituent group is substituted with one or more third substituent groups denoted by R^{20A.3} as explained in the definitions section above in the description of “first substituent group(s)”. In the above embodiments, R^{20A}, R^{20A.1}, R^{20A.2}, and R^{20A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20A}, R^{20A.1}, R^{20A.2}, and R^{20A.3} respectively.

In embodiments, when R^{20B} is substituted, R^{20B} is substituted with one or more first substituent groups denoted by R^{20B.1} as explained in the definitions section above in the description of “first substituent group(s)”. In embodiments, when an R^{20B.1} substituent group is substituted, the R^{20B.1} substituent group is substituted with one or more second substituent groups denoted by R^{20B.2} as explained in the definitions section above in the description of “first substituent group(s)”. In embodiments, when an R^{20B.2} substituent group is substituted, the R^{20B.2} substituent group is substituted with one or more third substituent groups denoted by R^{20B.3} as explained in the definitions section above in the description of “first substituent group(s)”. In the above embodiments, R^{20B}, R^{20B.1}, R^{20B.2}, and R^{20B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20B}, R^{20B.1}, R^{20B.2}, and R^{20B.3}, respectively.

In embodiments, when R^{20A} and R^{20B} substituents bonded to the same nitrogen atom are optionally joined to form a moiety that is substituted (e.g., a substituted heterocycloalkyl or substituted heteroaryl), the moiety is substituted with one or more first substituent groups denoted by R^{20A.1} as explained in the definitions section above in the description of “first substituent group(s)”. In embodiments, when an R^{20A.1} substituent group is substituted, the R^{20A.1} substituent group is substituted with one or more second substituent groups denoted by R^{20A.2} as explained in the definitions section above in the description of “first substituent group(s)”. In embodiments, when an R^{20A.2} substituent group is substituted, the R^{20A.2} substituent group is substituted with one or more third substituent groups denoted by R^{20A.3} as explained in the definitions section above in the description of “first substituent group(s)”. In the above embodiments, R^{20A.1}, R^{20A.2}, and R^{20A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{20A.1}, R^{20A.2}, and R^{20A.3}, respectively.

In embodiments, when R^{20A} and R^{20B} substituents bonded to the same nitrogen atom are optionally joined to form a moiety that is substituted (e.g., a substituted heterocycloalkyl or substituted heteroaryl), the moiety is substituted with one or more first substituent groups denoted by R^{20B.1} as explained in the definitions section above in the description of “first substituent group(s)”. In embodiments, when an R^{20B.1} substituent group is substituted, the R^{20B.1} substituent group is substituted with one or more second substituent groups denoted by R^{20B.2} as explained in the definitions section above in the description of “first substituent group(s)”. In embodiments, when an R^{20B.2} substituent group is substituted, the R^{20B.2} substituent group is substituted with one or more third substituent groups denoted by R^{20B.3} as explained in the definitions section above in the description of “first substituent group(s)”. In the above embodiments, R^{20B.1}, R^{20B.2}, and R^{20B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{20B.1}, R^{20B.2}, and R^{20B.3}, respectively.

In embodiments, when R^{20C} is substituted, R^{20C} is substituted with one or more first substituent groups denoted by R^{20C.1} as explained in the definitions section above in the description of “first substituent group(s)”. In embodiments, when an R^{20C.1} substituent group is substituted, the R^{20C.1} substituent group is substituted with one or more second substituent groups denoted by R^{20C.2} as explained in the definitions section above in the description of “first substituent group(s)”. In embodiments, when an R^{20C.2} substituent group is substituted, the R^{20C.2} substituent group is substituted with one or more third substituent groups denoted by R^{20C.3} as explained in the definitions section above in the description of “first substituent group(s)”. In the above embodiments, R^{20C}, R^{20C.1}, R^{20C.2}, and R^{20C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20C}, R^{20C.1}, R^{20C.2}, and R^{20C.3} respectively.

In embodiments, when R^{20D} is substituted, R^{20D} is substituted with one or more first substituent groups denoted by R^{20D.1} as explained in the definitions section above in the description of “first substituent group(s)”. In embodiments, when an R^{20D.1} substituent group is substituted, the R^{20D.1} substituent group is substituted with one or more second substituent groups denoted by R^{20D.2} as explained in the definitions section above in the description of “first substituent group(s)”. In embodiments, when an R^{20D.2} substituent group is substituted, the R^{20D.2} substituent group is substituted with one or more third substituent groups denoted by R^{20D.3} as explained in the definitions section above in the description of “first substituent group(s)”. In the above embodiments, R^{20D}, R^{20D.1}, R^{20D.2}, and R^{20D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{20D}, R^{20D.1}, R^{20D.2} and R^{20D.3} respectively.

In embodiments, when R³⁰ is substituted, R³⁰ is substituted with one or more first substituent groups denoted by R^{30.1} as explained in the definitions section above in the description of “first substituent group(s)”. In embodiments, when an R^{30.1} substituent group is substituted, the R^{30.1} substituent group is substituted with one or more second substituent groups denoted by R^{30.2} as explained in the definitions section above in the description of “first substituent group(s)”. In embodiments, when an R^{30.2} substituent group is substituted, the R^{30.2} substituent group is substituted with one or more third substituent groups denoted by R^{30.3} as explained in the definitions section above in the description of “first substituent group(s)”. In the above embodiments, R³⁰, R^{30.1}, R^{30.2}, and R^{30.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³⁰, R^{30.1}, R^{30.2}, and R^{30.3}, respectively.

In embodiments, when R¹⁰⁰ is substituted, R¹⁰⁰ is substituted with one or more first substituent groups denoted by R^{100.1} as explained in the definitions section above in the description of “first substituent group(s)”. In embodiments, when an R^{100.1} substituent group is substituted, the R^{100.1} substituent group is substituted with one or more second substituent groups denoted by R^{100.2} as explained in the definitions section above in the description of “first substituent group(s)”. In embodiments, when an R^{100.2} substituent group is substituted, the R^{100.2} substituent group is substituted with one or more third substituent groups denoted by R^{100.3} as explained in the definitions section above in the description of “first substituent group(s)”. In the above embodiments, R¹⁰⁰, R^{100.1}, R^{100.2}, and R^{100.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰⁰, R^{100.1}, R^{100.2} and R^{100.3}, respectively.

In embodiments, when R¹⁰¹ is substituted, R¹⁰¹ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{101.2} as explained in the definitions section above in the description of “first substituent group(s)”. In embodiments, when an R^{101.2} substituent group is substituted, the R^{101.2} substituent group is substituted with one or more third substituent groups denoted by R^{101.3} as explained in the definitions section above in the description of “first substituent group(s)”. In the above embodiments, R¹⁰¹, R^{101.1}, R^{101.2}, and R^{101.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰¹, R^{101.1}, R^{101.2} and R^{101.3} respectively.

In embodiments, when R¹⁰² is substituted, R¹⁰² is substituted with one or more first substituent groups denoted by R^{102.1} as explained in the definitions section above in the description of “first substituent group(s)”. In embodiments, when an R^{102.1} substituent group is substituted, the R^{102.1} substituent group is substituted with one or more second substituent groups denoted by R^{102.2} as explained in the definitions section above in the description of “first substituent group(s)”. In embodiments, when an R^{102.2} substituent group is substituted, the R^{102.2} substituent group is substituted with one or more third substituent groups denoted by R^{102.3} as explained in the definitions section above in the description of “first substituent group(s)”. In the above embodiments, R¹⁰², R^{102.1}, R^{102.2}, and R^{102.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰², R^{102.1}, R^{102.2} and R^{102.3} respectively.

In embodiments, when R¹⁰³ is substituted, R¹⁰³ is substituted with one or more first substituent groups denoted by R^{103.1} as explained in the definitions section above in the description of “first substituent group(s)”. In embodiments, when an R^{103.1} substituent group is substituted, the R^{103.1} substituent group is substituted with one or more second substituent groups denoted by R^{103.2} as explained in the definitions section above in the description of “first substituent group(s)”. In embodiments, when an R^{103.2} substituent group is substituted, the R^{103.2} substituent group is substituted with one or more third substituent groups denoted by R^{103.3} as explained in the definitions section above in the description of “first substituent group(s)”. In the above embodiments, R¹⁰³, R^{103.1}, R^{103.2}, and R^{103.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰³, R^{103.1}, R^{103.2}, and R^{103.3}, respectively.

In embodiments, when R¹⁰⁴ is substituted, R¹⁰⁴ is substituted with one or more first substituent groups denoted by R^{104.1} as explained in the definitions section above in the description of “first substituent group(s)”. In embodiments, when an R^{104.1} substituent group is substituted, the R^{104.1} substituent group is substituted with one or more second substituent groups denoted by R^{104.2} as explained in the definitions section above in the description of “first substituent group(s)”. In embodiments, when an R^{104.2} substituent group is substituted, the R^{104.2} substituent group is substituted with one or more third substituent groups denoted by R^{104.3} as explained in the definitions section above in the description of “first substituent group(s)”. In the above embodiments, R¹⁰⁴, R^{104.1}, R^{104.2}, and R^{104.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰⁴, R^{104.1}, R^{104.2} and R^{104.3}, respectively.

In embodiments, when R¹⁰⁵ is substituted, R¹⁰⁵ is substituted with one or more first substituent groups denoted by R^{105.1} as explained in the definitions section above in the description of “first substituent group(s)”. In embodiments, when an R^{105.1} substituent group is substituted, the R^{105.1} substituent group is substituted with one or more second substituent groups denoted by R^{105.2} as explained in the definitions section above in the description of “first substituent group(s)”. In embodiments, when an R^{105.2} substituent group is substituted, the R^{105.2} substituent group is substituted with one or more third substituent groups denoted by R^{105.3} as explained in the definitions section above in the description of “first substituent group(s)”. In the above embodiments, R¹⁰⁵, R^{105.1}, R^{105.2}, and R^{105.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰⁵, R^{105.1}, R^{105.2}, and R^{105.3} respectively.

In embodiments, when R²⁰⁰ is substituted, R²⁰⁰ is substituted with one or more first substituent groups denoted by R^{200.1} as explained in the definitions section above in the description of “first substituent group(s)”. In embodiments, when an R^{200.1} substituent group is substituted, the R^{200.1} substituent group is substituted with one or more second substituent groups denoted by R^{200.2} as explained in the definitions section above in the description of “first substituent group(s)”. In embodiments, when an R^{200.2} substituent group is substituted, the R^{200.2} substituent group is substituted with one or more third substituent groups denoted by R^{200.3} as explained in the definitions section above in the description of “first substituent group(s)”. In the above embodiments, R²⁰⁰, R^{200.1}, R^{200.2}, and R^{200.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁰⁰, R^{200.1}, R^{200.2}, and R^{200.3} respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L², RL^{2.1}, R^{L2.2}, and R^{L2.3}, respectively.

In embodiments, when L³ is substituted, L³ is substituted with one or more first substituent groups denoted by R^{L3.1} as explained in the definitions section above in the description of “first substituent group(s)”. In embodiments, when an R^{L3.1} substituent group is substituted, the R^{L3.1} substituent group is substituted with one or more second substituent groups denoted by R^{L3.2} as explained in the definitions section above in the description of “first substituent group(s)”. In embodiments, when an R^{L3.2} substituent group is substituted, the R^{L3.2} substituent group is substituted with one or more third substituent groups denoted by R^{L33} as explained in the definitions section above in the description of “first substituent group(s)”. In the above embodiments, L³, R^{L3.1}, R^{L3.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L³, R^{L3.1}, R^{L3.2}, and R^{L33}, respectively.

In embodiments, when L¹⁰ is substituted, L¹⁰ is substituted with one or more first substituent groups denoted by R^{L10.1} as explained in the definitions section above in the description of “first substituent group(s)”. In embodiments, when an R^{L10.1} substituent group is substituted, the R^{L10.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L10.3} as explained in the definitions section above in the description of “first substituent group(s)”. In the above embodiments, L¹⁰, R^{L10.1}, R^{L10.2}, and R^{L10.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰, R^{L10.1}, R^{L1.2}, and R^{L10.3}, respectively.

In embodiments, when L²⁰ is substituted, L²⁰ is substituted with one or more first substituent groups denoted by R^{L20.1} as explained in the definitions section above in the description of “first substituent group(s)”. In embodiments, when an R^{L20.1} substituent group is substituted, the R^{L20.1} substituent group is substituted with one or more second substituent groups denoted by R^{L20.2} as explained in the definitions section above in the description of “first substituent group(s)”. In embodiments, when an R^{L20.2} substituent group is substituted, the R^{L20.2} substituent group is substituted with one or more third substituent groups denoted by R^{L20.3} as explained in the definitions section above in the description of “first substituent group(s)”. In the above embodiments, L²⁰, R^{L20.1}, R^{L20.2}, and R^{L20.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L²⁰, R^{L20.1}, R^{L20.2}, and R^{L20.3}, respectively.

In embodiments, when L¹⁰¹ is substituted, L¹⁰¹ is substituted with one or more first substituent groups denoted by R^{L101.1} as explained in the definitions section above in the description of “first substituent group(s)”. In embodiments, when an R^{L101.1} substituent group is substituted, the R^{L101.1} substituent group is substituted with one or more second substituent groups denoted by R^{L101.2} as explained in the definitions section above in the description of “first substituent group(s)”. In embodiments, when an R^{L101.2} substituent group is substituted, the R^{L101.2} substituent group is substituted with one or more third substituent groups denoted by R^{L101.3} as explained in the definitions section above in the description of “first substituent group(s)”. In the above embodiments, L¹⁰¹, R^{L101.1}, R^{L1001.2}, and R^{L101.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰¹, R^{101.1}, R^{L101.2}, and R^{L101.3}, respectively.

In embodiments, when L¹⁰² is substituted, L¹⁰² is substituted with one or more first substituent groups denoted by R^{L102.1} as explained in the definitions section above in the description of “first substituent group(s)”. In embodiments, when an R^{L102.1} substituent group is substituted, the R^{L102.1} substituent group is substituted with one or more second substituent groups denoted by R^{L102.2} as explained in the definitions section above in the description of “first substituent group(s)”. In embodiments, when an R^{L102.2} substituent group is substituted, the R^{L102.2} substituent group is substituted with one or more third substituent groups denoted by R^{L102.3} as explained in the definitions section above in the description of “first substituent group(s)”. In the above embodiments, L¹⁰², R^{L102.1}, R^{L102.2}, and R^{L102.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰², R^{102.1}, R^{L102.2}, and R^{L102.3}, respectively.

In embodiments, when L¹⁰³ is substituted, L¹⁰³ is substituted with one or more first substituent groups denoted by R^{L103.1} as explained in the definitions section above in the description of “first substituent group(s)”. In embodiments, when an R^{L103.1} substituent group is substituted, the R^{L103.1} substituent group is substituted with one or more second substituent groups denoted by R^{L103.2} as explained in the definitions section above in the description of “first substituent group(s)”. In embodiments, when an R^{L103.2} substituent group is substituted, the R^{L103.2} substituent group is substituted with one or more third substituent groups denoted by R^{L103.3} as explained in the definitions section above in the description of “first substituent group(s)”. In the above embodiments, L¹⁰³, R^{L103.1}, R^{L103.2}, and R^{L103.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰³, R^{L103.1}, R^{L103.2}, and R^{L103.3} respectively.

In embodiments, when L¹⁰⁴ is substituted, L¹⁰⁴ is substituted with one or more first substituent groups denoted by R^{L104.1} as explained in the definitions section above in the description of “first substituent group(s)”. In embodiments, when an R^{L104.1} substituent group is substituted, the R^{L104.1} substituent group is substituted with one or more second substituent groups denoted by R^{L104.2} as explained in the definitions section above in the description of “first substituent group(s)”. In embodiments, when an R_{L104.2} substituent group is substituted, the R^{L104.2} substituent group is substituted with one or more third substituent groups denoted by R^{L104.3} as explained in the definitions section above in the description of “first substituent group(s)”. In the above embodiments, L¹⁰⁴, R^{L104.1}, R^{L104.2}, and R^{L104.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰⁴, R^{L104.1}, R^{L104.2}, and R^{L104.3} respectively.

In embodiments, when L¹⁰⁵ is substituted, L¹⁰⁵ is substituted with one or more first substituent groups denoted by R^{105.1} as explained in the definitions section above in the description of “first substituent group(s)”. In embodiments, when an R^{105.1} substituent group is substituted, the R^{105.11} substituent group is substituted with one or more second substituent groups denoted by R^{L105.2} as explained in the definitions section above in the description of “first substituent group(s)”. In embodiments, when an R^{L105.2} substituent group is substituted, the R^{L105.2} substituent group is substituted with one or more third substituent groups denoted by R^{L105.3} as explained in the definitions section above in the description of “first substituent group(s)”. In the above embodiments, L¹⁰⁵, R^{L105.1}, R^{L105.2}, and R^{L105.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰⁵, R^{105.1}, R^{105.2}, and R^{L105.3}, respectively.

In embodiments, the compound has the formula:

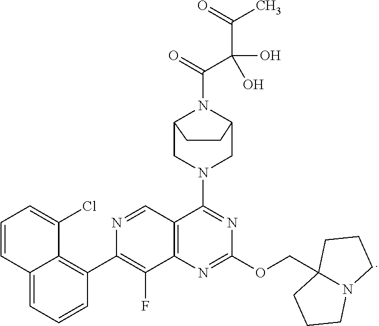 [SMILES: CC(=O)C(O)(O)C(=O)N1C2CCC1CN(c1nc(OCC34CCCN3CCC4)nc3c(F)c(-c4cccc5cccc(Cl)c45)ncc13)C2]

In embodiments, the compound has the formula:

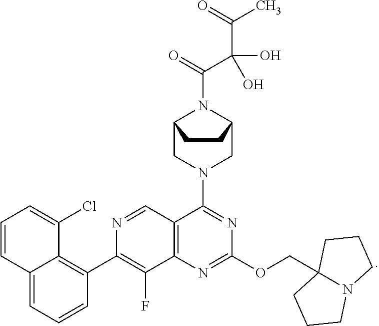

In embodiments, the compound has the formula:

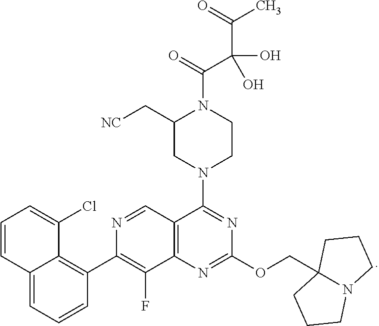 [SMILES: CC(=O)C(O)(O)C(=O)N1CCN(c2nc(OCC34CCCN3CCC4)nc3c(F)c(-c4cccc5cccc(Cl)c45)ncc23)CC1CC#N]

In embodiments, the compound has the formula:

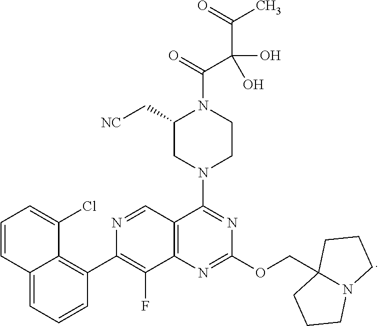 [SMILES: CC(=O)C(O)(O)C(=O)N1CCN(c2nc(OCC34CCCN3CCC4)nc3c(F)c(-c4cccc5cccc(Cl)c45)ncc23)C[C@@H]1CC#N]

In embodiments, the compound has the formula:

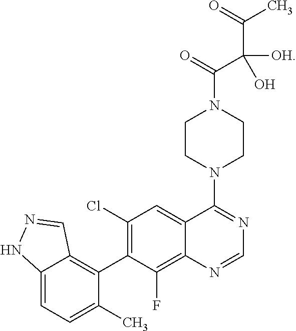 [SMILES: CC(=O)C(O)(O)C(=O)N1CCN(c2ncnc3c(F)c(-c4c(C)ccc5[nH]ncc45)c(Cl)cc23)CC1]

In embodiments, the compound has the formula:

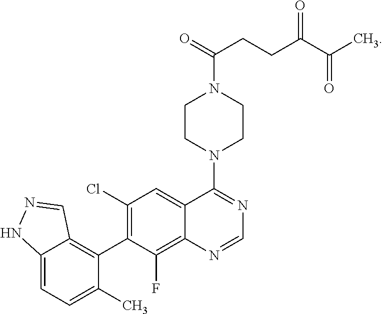 [SMILES: CC(=O)C(=O)CCC(=O)N1CCN(c2ncnc3c(F)c(-c4c(C)ccc5[nH]ncc45)c(Cl)cc23)CC1]

In embodiments, the compound has the formula:

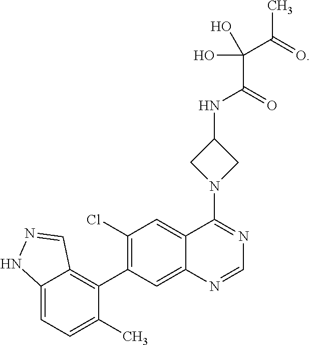 [SMILES: CC(=O)C(O)(O)C(=O)NC1CN(c2ncnc3cc(-c4c(C)ccc5[nH]ncc45)c(Cl)cc23)C1]

In embodiments,

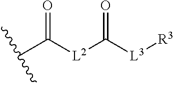

is capable of forming a covalent bond with a Switch II GTPase protein arginine residue. In embodiments, the Switch II GTPase protein arginine residue is a natural Switch II GTPase protein arginine residue. In embodiments, the Switch II GTPase protein arginine residue is a mutant Switch II GTPase protein arginine residue. In embodiments, the Switch II GTPase protein arginine residue is an arginine residue corresponding to the 12 position of a Ras protein (e.g., K-Ras, H-Ras, or N-Ras). In embodiments, the mutant Switch II GTPase protein arginine residue is arginine residue 12 of K-Ras(G12R), H-Ras(G12R), or N-Ras(G12R). In embodiments, the Switch II GTPase protein arginine residue is an arginine residue corresponding to the 13 position of a Ras protein (e.g., K-Ras, H-Ras, or N-Ras). In embodiments, the mutant Switch II GTPase protein arginine residue is arginine residue 13 of K-Ras(G13R), H-Ras(G13R), or N-Ras(G13R). In embodiments, the Switch II GTPase protein arginine residue is an arginine residue corresponding to the 61 position of a Ras protein (e.g., K-Ras, H-Ras, or N-Ras). In embodiments, the mutant Switch II GTPase protein arginine residue is arginine residue 61 of K-Ras(Q61R), H-Ras(Q61R), or N-Ras(Q61R).

In embodiments, the compound binds Ras(G12R) (e.g., K-Ras(G12R), H-Ras(G12R), or N-Ras(G12R)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12R) (e.g., K-Ras(G12R), H-Ras(G12R), or N-Ras(G12R)) nucleotide exchange (e.g., GDP for GTP or GTP for GDP) relative to the absence of the compound. In embodiments, the compound inhibits release of GDP from Ras(G12R) (e.g., K-Ras(G12R), H-Ras(G12R), or N-Ras(G12R)) relative to the absence of the compound. In embodiments, the compound inhibits binding of GDP to Ras(G12R) (e.g., K-Ras(G12R), H-Ras(G12R), or N-Ras(G12R)) relative to the absence of the compound. In embodiments, the compound inhibits binding of GTP to Ras(G12R) (e.g., K-Ras(G12R), H-Ras(G12R), or N-Ras(G12R)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G12R) (e.g., K-Ras(G12R), H-Ras(G12R), or N-Ras(G12R)) nucleotide exchange (e.g., GDP for GTP or GTP for GDP) relative to the absence of the compound. In embodiments, the compound increases release of GDP from Ras(G12R) (e.g., K-Ras(G12R), H-Ras(G12R), or N-Ras(G12R)) relative to the absence of the compound. In embodiments, the compound increases release of GTP from Ras(G12R) (e.g., K-Ras(G12R), H-Ras(G12R), or N-Ras(G12R)) relative to the absence of the compound. In embodiments, the compound increases binding of GDP to Ras(G12R) (e.g., K-Ras(G12R), H-Ras(G12R), or N-Ras(G12R)) relative to the absence of the compound. In embodiments, the compound inhibits binding of GTP to Ras(G12R) (e.g., K-Ras(G12R), H-Ras(G12R), or N-Ras(G12R)) relative to the absence of the compound. In embodiments, the compound modulates the binding of GTP and/or GDP to Ras(G12R) (e.g., K-Ras(G12R), H-Ras(G12R), or N-Ras(G12R)) compared to binding in the absence of the compound. In embodiments, the compound modulates the release of GTP and/or GDP from Ras(G12R) (e.g., K-Ras(G12R), H-Ras(G12R), or N-Ras(G12R)) compared to release in the absence of the compound. In embodiments, the compound modulates the ratio of the binding of GTP and GDP to Ras(G12R) (e.g., K-Ras(G12R), H-Ras(G12R), or N-Ras(G12R)) compared to the ratio in the absence of the compound. In embodiments, the compound modulates the ratio of the rate of release of GTP and GDP from Ras(G12R) (e.g., K-Ras(G12R), H-Ras(G12R), or N-Ras(G12R)) compared to the ratio in the absence of the compound. In embodiments, the compound binds Ras(G12R) (e.g., K-Ras(G12R), H-Ras(G12R), or N-Ras(G12R)) protein bound to GDP and, after release of the GDP, modulates the subsequent binding of GDP or GTP to the Ras bound to the compound. In embodiments, the compound binds Ras(G12R) (e.g., K-Ras(G12R), H-Ras(G12R), or N-Ras(G12R)) protein bound to GDP and, after release of the GDP, modulates the subsequent binding of GDP to the Ras bound to the compound. In embodiments, the compound binds Ras(G12R) (e.g., K-Ras(G12R), H-Ras(G12R), or N-Ras(G12R)) protein bound to GDP and after release of the GDP, modulates the subsequent binding of GTP to the Ras bound to the compound.

In embodiments, the compound binds Ras(G13R) (e.g., K-Ras(G13R), H-Ras(G13R), or N-Ras(G13R)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G13R) (e.g., K-Ras(G13R), H-Ras(G13R), or N-Ras(G13R)) nucleotide exchange (e.g., GDP for GTP or GTP for GDP) relative to the absence of the compound. In embodiments, the compound inhibits release of GDP from Ras(G13R) (e.g., K-Ras(G13R), H-Ras(G13R), or N-Ras(G13R)) relative to the absence of the compound. In embodiments, the compound inhibits binding of GDP to Ras(G13R) (e.g., K-Ras(G13R), H-Ras(G13R), or N-Ras(G13R)) relative to the absence of the compound. In embodiments, the compound inhibits binding of GTP to Ras(G13R) (e.g., K-Ras(G13R), H-Ras(G13R), or N-Ras(G13R)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(G13R) (e.g., K-Ras(G13R), H-Ras(G13R), or N-Ras(G13R)) nucleotide exchange (e.g., GDP for GTP or GTP for GDP) relative to the absence of the compound. In embodiments, the compound increases release of GDP from Ras(G13R) (e.g., K-Ras(G13R), H-Ras(G13R), or N-Ras(G13R)) relative to the absence of the compound. In embodiments, the compound increases release of GTP from Ras(G13R) (e.g., K-Ras(G13R), H-Ras(G13R), or N-Ras(G13R)) relative to the absence of the compound. In embodiments, the compound increases binding of GDP to Ras(G13R) (e.g., K-Ras(G13R), H-Ras(G13R), or N-Ras(G13R)) relative to the absence of the compound. In embodiments, the compound inhibits binding of GTP to Ras(G13R) (e.g., K-Ras(G13R), H-Ras(G13R), or N-Ras(G13R)) relative to the absence of the compound. In embodiments, the compound modulates the binding of GTP and/or GDP to Ras(G13R) (e.g., K-Ras(G13R), H-Ras(G13R), or N-Ras(G13R)) compared to binding in the absence of the compound. In embodiments, the compound modulates the release of GTP and/or GDP from Ras(G13R) (e.g., K-Ras(G13R), H-Ras(G13R), or N-Ras(G13R)) compared to release in the absence of the compound. In embodiments, the compound modulates the ratio of the binding of GTP and GDP to Ras(G13R) (e.g., K-Ras(G13R), H-Ras(G13R), or N-Ras(G13R)) compared to the ratio in the absence of the compound. In embodiments, the compound modulates the ratio of the rate of release of GTP and GDP from Ras(G13R) (e.g., K-Ras(G13R), H-Ras(G13R), or N-Ras(G13R)) compared to the ratio in the absence of the compound. In embodiments, the compound binds Ras(G13R) (e.g., K-Ras(G13R), H-Ras(G13R), or N-Ras(G13R)) protein bound to GDP and, after release of the GDP, modulates the subsequent binding of GDP or GTP to the Ras bound to the compound. In embodiments, the compound binds Ras(G13R) (e.g., K-Ras(G13R), H-Ras(G13R), or N-Ras(G13R)) protein bound to GDP and, after release of the GDP, modulates the subsequent binding of GDP to the Ras bound to the compound. In embodiments, the compound binds Ras(G13R) (e.g., K-Ras(G13R), H-Ras(G13R), or N-Ras(G13R)) protein bound to GDP and after release of the GDP, modulates the subsequent binding of GTP to the Ras bound to the compound.

In embodiments, the compound binds Ras(Q61R) (e.g., K-Ras(Q61R), H-Ras(Q61R), or N-Ras(Q61R)) behind Switch II. In embodiments, the compound modulates the conformation of Switch II. In embodiments, the compound inhibits (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(Q61R) (e.g., K-Ras(Q61R), H-Ras(Q61R), or N-Ras(Q61R)) nucleotide exchange (e.g., GDP for GTP or GTP for GDP) relative to the absence of the compound. In embodiments, the compound inhibits release of GDP from Ras(Q61R) (e.g., K-Ras(Q61R), H-Ras(Q61R), or N-Ras(Q61R)) relative to the absence of the compound. In embodiments, the compound inhibits binding of GDP to Ras(Q61R) (e.g., K-Ras(Q61R), H-Ras(Q61R), or N-Ras(Q61R)) relative to the absence of the compound. In embodiments, the compound inhibits binding of GTP to Ras(Q61R) (e.g., K-Ras(Q61R), H-Ras(Q61R), or N-Ras(Q61R)) relative to the absence of the compound. In embodiments, the compound increases (e.g., by about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000 fold or more) Ras(Q61R) (e.g., K-Ras(Q61R), H-Ras(Q61R), or N-Ras(Q61R)) nucleotide exchange (e.g., GDP for GTP or GTP for GDP) relative to the absence of the compound. In embodiments, the compound increases release of GDP from Ras(Q61R) (e.g., K-Ras(Q61R), H-Ras(Q61R), or N-Ras(Q61R)) relative to the absence of the compound. In embodiments, the compound increases release of GTP from Ras(Q61R) (e.g., K-Ras(Q61R), H-Ras(Q61R), or N-Ras(Q61R)) relative to the absence of the compound. In embodiments, the compound increases binding of GDP to Ras(Q61R) (e.g., K-Ras(Q61R), H-Ras(Q61R), or N-Ras(Q61R)) relative to the absence of the compound. In embodiments, the compound inhibits binding of GTP to Ras(Q61R) (e.g., K-Ras(Q61R), H-Ras(Q61R), or N-Ras(Q61R)) relative to the absence of the compound. In embodiments, the compound modulates the binding of GTP and/or GDP to Ras(Q61R) (e.g., K-Ras(Q61R), H-Ras(Q61R), or N-Ras(Q61R)) compared to binding in the absence of the compound. In embodiments, the compound modulates the release of GTP and/or GDP from Ras(Q61R) (e.g., K-Ras(Q61R), H-Ras(Q61R), or N-Ras(Q61R)) compared to release in the absence of the compound. In embodiments, the compound modulates the ratio of the binding of GTP and GDP to Ras(Q61R) (e.g., K-Ras(Q61R), H-Ras(Q61R), or N-Ras(Q61R)) compared to the ratio in the absence of the compound. In embodiments, the compound modulates the ratio of the rate of release of GTP and GDP from Ras(Q61R) (e.g., K-Ras(Q61R), H-Ras(Q61R), or N-Ras(Q61R)) compared to the ratio in the absence of the compound. In embodiments, the compound binds Ras(Q61R) (e.g., K-Ras(Q61R), H-Ras(Q61R), or N-Ras(Q61R)) protein bound to GDP and, after release of the GDP, modulates the subsequent binding of GDP or GTP to the Ras bound to the compound. In embodiments, the compound binds Ras(Q61R) (e.g., K-Ras(Q61R), H-Ras(Q61R), or N-Ras(Q61R)) protein bound to GDP and, after release of the GDP, modulates the subsequent binding of GDP to the Ras bound to the compound. In embodiments, the compound binds Ras(Q61R) (e.g., K-Ras(Q61R), H-Ras(Q61R), or N-Ras(Q61R)) protein bound to GDP and after release of the GDP, modulates the subsequent binding of GTP to the Ras bound to the compound.

In embodiments, the compound contacts the Switch II Binding Pocket of human K-Ras protein. In embodiments, the compound contacts a Switch II Binding Pocket amino acid corresponding to G60, Q61, D69, D92, H95, Y96, or Q99 of human K-Ras protein. In embodiments, the compound contacts a Switch II Binding Pocket amino acid corresponding to G60 of human K-Ras protein. In embodiments, the compound contacts a Switch II Binding Pocket amino acid corresponding to Q61 of human K-Ras protein. In embodiments, the compound contacts a Switch II Binding Pocket amino acid corresponding to D69 of human K-Ras protein. In embodiments, the compound contacts a Switch II Binding Pocket amino acid corresponding to D92 of human K-Ras protein. In embodiments, the compound contacts a Switch II Binding Pocket amino acid corresponding to H95 of human K-Ras protein. In embodiments, the compound contacts a Switch II Binding Pocket amino acid corresponding to Y96 of human K-Ras protein. In embodiments, the compound contacts a Switch II Binding Pocket amino acid corresponding to Q99 of human K-Ras protein.

In embodiments, the compound contacts the Switch II Binding Pocket of human H-Ras protein. In embodiments, the compound contacts a Switch II Binding Pocket amino acid corresponding to G60, Q61, D69, D92, Q95, Y96, or Q99 of human H-Ras protein. In embodiments, the compound contacts a Switch II Binding Pocket amino acid corresponding to G60 of human H-Ras protein. In embodiments, the compound contacts a Switch II Binding Pocket amino acid corresponding to Q61 of human H-Ras protein. In embodiments, the compound contacts a Switch II Binding Pocket amino acid corresponding to D69 of human H-Ras protein. In embodiments, the compound contacts a Switch II Binding Pocket amino acid corresponding to D92 of human H-Ras protein. In embodiments, the compound contacts a Switch II Binding Pocket amino acid corresponding to Q95 of human H-Ras protein. In embodiments, the compound contacts a Switch II Binding Pocket amino acid corresponding to Y96 of human H-Ras protein. In embodiments, the compound contacts a Switch II Binding Pocket amino acid corresponding to Q99 of human H-Ras protein.

In embodiments, the compound contacts the Switch II Binding Pocket of human N-Ras protein. In embodiments, the compound contacts a Switch II Binding Pocket amino acid corresponding to G60, Q61, D69, D92, L95, Y96, or Q99 of human N-Ras protein. In embodiments, the compound contacts a Switch II Binding Pocket amino acid corresponding to G60 of human N-Ras protein. In embodiments, the compound contacts a Switch II Binding Pocket amino acid corresponding to Q61 of human N-Ras protein. In embodiments, the compound contacts a Switch II Binding Pocket amino acid corresponding to D69 of human N-Ras protein. In embodiments, the compound contacts a Switch II Binding Pocket amino acid corresponding to D92 of human N-Ras protein. In embodiments, the compound contacts a Switch II Binding Pocket amino acid corresponding to L95 of human N-Ras protein. In embodiments, the compound contacts a Switch II Binding Pocket amino acid corresponding to Y96 of human N-Ras protein. In embodiments, the compound contacts a Switch II Binding Pocket amino acid corresponding to Q99 of human N-Ras protein.

In embodiments, the compound contacts a residue of K-Ras Switch II. In embodiments, the compound contacts a residue of H-Ras Switch II. In embodiments, the compound contacts a residue of N-Ras Switch II. In embodiments, wherein the compound contacts K-Ras (e.g., K-Ras(G12R), human K-Ras(G12R), K-Ras(G13R), human K-Ras(G13R), K-Ras(Q61R), human K-Ras(Q61R)), R¹ contacts V7, V9, G10, P34, T58, G60, Q61, E62, E63, R68, Y71, M72, Y96, Q99, or 1100. In embodiments, R¹ contacts at least one of G60, E62, or E63 of K-Ras (e.g., K-Ras(G12R), human K-Ras(G12R), K-Ras(G13R), human K-Ras(G13R), K-Ras(Q61R), human K-Ras(Q61R)). In embodiments, the compound does not contact the residues of K-Ras (e.g., K-Ras(G12R), human K-Ras(G12R), K-Ras(G13R), human K-Ras(G13R), K-Ras(Q61R), human K-Ras(Q61R)) that contact GTP. In embodiments, the compound does not contact the residues of K-Ras (e.g., K-Ras(G12R), human K-Ras(G12R), K-Ras(G13R), human K-Ras(G13R), K-Ras(Q61R), human K-Ras(Q61R)) that contact the guanine of GTP or GDP. In embodiments, the compound does not contact the residues of K-Ras (e.g., K-Ras(G12R), human K-Ras(G12R), K-Ras(G13R), human K-Ras(G13R), K-Ras(Q61R), human K-Ras(Q61R)) that contact GDP. In embodiments, R¹ contacts residues that contact Switch II in the GTP bound form of K-Ras (e.g., K-Ras(G12R), human K-Ras(G12R), K-Ras(G13R), human K-Ras(G13R), K-Ras(Q61R), human K-Ras(Q61R)). In embodiments, R¹ contacts residues that contact Switch II in the GDP bound form of K-Ras (e.g., K-Ras(G12R), human K-Ras(G12R), K-Ras(G13R), human K-Ras(G13R), K-Ras(Q61R), human K-Ras(Q61R)).

In embodiments, the compound binds a human Ras(G12R) (e.g., human K-Ras(G12R), human H-Ras(G12R), or human N-Ras(G12R)) protein-GDP complex more strongly than the compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 2-fold stronger than the compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 5-fold stronger than the compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 10-fold stronger than the compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 20-fold stronger than the compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 40-fold stronger than said compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 60-fold stronger than the compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 80-fold stronger than the compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 100-fold stronger than said compound binds a human Ras(G12R) (e.g., human K-Ras(G12R), human H-Ras(G12R), or human N-Ras(G12R)) protein-GTP complex under identical conditions. In embodiments, the compound binds a human Ras(G12R) (e.g., human K-Ras(G12R), human H-Ras(G12R), or human N-Ras(G12R)) protein-GDP complex at least 500-fold stronger than the compound binds a human Ras(G12R) (e.g., human K-Ras(G12R), human H-Ras(G12R), or human N-Ras(G12R)) protein-GTP complex under identical conditions.

In embodiments, the compound binds a human Ras(G13R) (e.g., human K-Ras(G13R), human H-Ras(G13R), or human N-Ras(G13R)) protein-GDP complex more strongly than the compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 2-fold stronger than the compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 5-fold stronger than the compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 10-fold stronger than the compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 20-fold stronger than the compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 40-fold stronger than said compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 60-fold stronger than the compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 80-fold stronger than the compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 100-fold stronger than said compound binds a human Ras(G13R) (e.g., human K-Ras(G13R), human H-Ras(G13R), or human N-Ras(G13R)) protein-GTP complex under identical conditions. In embodiments, the compound binds a human Ras(G13R) (e.g., human K-Ras(G13R), human H-Ras(G13R), or human N-Ras(G13R)) protein-GDP complex at least 500-fold stronger than the compound binds a human Ras(G13R) (e.g., human K-Ras(G13R), human H-Ras(G13R), or human N-Ras(G13R)) protein-GTP complex under identical conditions.

In embodiments, the compound binds a human Ras(Q61R) (e.g., human K-Ras(Q61R), human H-Ras(Q61R), or human N-Ras(Q61R)) protein-GDP complex more strongly than the compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 2-fold stronger than the compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 5-fold stronger than the compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 10-fold stronger than the compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 20-fold stronger than the compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 40-fold stronger than said compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 60-fold stronger than the compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 80-fold stronger than the compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 100-fold stronger than said compound binds a human Ras(Q61R) (e.g., human K-Ras(Q61R), human H-Ras(Q61R), or human N-Ras(Q61R)) protein-GTP complex under identical conditions. In embodiments, the compound binds a human Ras(Q61R) (e.g., human K-Ras(Q61R), human H-Ras(Q61R), or human N-Ras(Q61R)) protein-GDP complex at least 500-fold stronger than the compound binds a human Ras(Q61R) (e.g., human K-Ras(Q61R), human H-Ras(Q61R), or human N-Ras(Q61R)) protein-GTP complex under identical conditions.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound. In embodiments, the compound is a compound of formula (I), (Ia), (Ib), (Ic), (II), (IIa), (IIb), (IIc), (IIIa), (IIIb), or (IIIc). In embodiments, the compound is a compound of formula (I), (Ia), (Ib), (Ic), (II), (IIa), (IIb), (IIc), (IIIa), (IIIb), or (IIIc), including embodiments thereof.

### IV. Methods of Use

In an aspect is provided a method of treating cancer in a subject in need thereof, the method including administering to the subject in need thereof a therapeutically effective amount of a compound described herein, or a pharmaceutically acceptable salt thereof.

In embodiments, the cancer is pancreatic cancer. In embodiments, the cancer is lung cancer. In embodiments, the cancer is colorectal cancer. In embodiments, the cancer is melanoma. In embodiments, the cancer is thyroid cancer. In embodiments, the cancer is urinary cancer.

In an aspect is provided a method of treating a K-Ras(G12R)-associated disease in a subject in need thereof, the method including administering to the subject in need thereof a therapeutically effective amount of a compound described herein, or a pharmaceutically acceptable salt thereof. In embodiments, the K-Ras(G12R)-associated disease is cancer (e.g., pancreatic cancer, lung cancer, or colorectal cancer). In embodiments, the K-Ras(G12R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G12R)-associated disease in a subject in need thereof, the method including administering to the subject in need thereof a therapeutically effective amount of a compound described herein, or a pharmaceutically acceptable salt thereof. In embodiments, the H-Ras(G12R)-associated disease is cancer. In embodiments, the H-Ras(G12R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(G12R)-associated disease in a subject in need thereof, the method including administering to the subject in need thereof a therapeutically effective amount of a compound described herein, or a pharmaceutically acceptable salt thereof. In embodiments, the N-Ras(G12R)-associated disease is cancer. In embodiments, the N-Ras(G12R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(G13R)-associated disease in a subject in need thereof, the method including administering to the subject in need thereof a therapeutically effective amount of a compound described herein, or a pharmaceutically acceptable salt thereof. In embodiments, the K-Ras(G13R)-associated disease is cancer. In embodiments, the K-Ras(G13R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(G13R)-associated disease in a subject in need thereof, the method including administering to the subject in need thereof a therapeutically effective amount of a compound described herein, or a pharmaceutically acceptable salt thereof. In embodiments, the H-Ras(G13R)-associated disease is cancer (e.g., melanoma). In embodiments, the H-Ras(G13R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(G13R)-associated disease in a subject in need thereof, the method including administering to the subject in need thereof a therapeutically effective amount of a compound described herein, or a pharmaceutically acceptable salt thereof. In embodiments, the N-Ras(G13R)-associated disease is cancer. In embodiments, the N-Ras(G13R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating a K-Ras(Q61R)-associated disease in a subject in need thereof, the method including administering to the subject in need thereof a therapeutically effective amount of a compound described herein, or a pharmaceutically acceptable salt thereof. In embodiments, the K-Ras(Q61R)-associated disease is cancer. In embodiments, the K-Ras(Q61R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an H-Ras(Q61R)-associated disease in a subject in need thereof, the method including administering to the subject in need thereof a therapeutically effective amount of a compound described herein, or a pharmaceutically acceptable salt thereof. In embodiments, the H-Ras(Q61R)-associated disease is cancer (e.g., thyroid cancer or urinary cancer). In embodiments, the H-Ras(Q61R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of treating an N-Ras(Q61R)-associated disease in a subject in need thereof, the method including administering to the subject in need thereof a therapeutically effective amount of a compound described herein, or a pharmaceutically acceptable salt thereof. In embodiments, the N-Ras(Q61R)-associated disease is cancer (e.g., melanoma or thyroid cancer). In embodiments, the N-Ras(Q61R)-associated disease is a RASopathy (e.g., capillary malformation-AV malformation syndrome, autoimmune lymphoproliferative syndrome, cardiofaciocutaneous syndrome, hereditary gingival fibromatosis type 1, neurofibromatosis type 1, Noonan syndrome, Costello syndrome, or Legius syndrome).

In an aspect is provided a method of modulating the level of activity of a Ras protein in a cell, the method including contacting the cell with an effective amount of a compound described herein, or a pharmaceutically acceptable salt thereof.

In embodiments, the modulating of the activity includes modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, Ras subcellular localization, Ras post-translational processing, or Ras post-translational modifications.

In embodiments, the modulating of the activity is increasing GTPase activity. In embodiments, increasing GTPase activity results in reducing (e.g., inhibiting) Ras effector protein function. In embodiments, the modulating of the activity is increasing differential GDP binding to Ras over GTP binding to Ras. In embodiments, increasing preference for GDP binding to Ras over GTP binding to Ras results in reducing (e.g., inhibiting) Ras effector protein function. As used herein, a “Ras effector protein” is a protein that binds to the GTP-bound conformation of Ras and activates downstream signaling pathways. As used herein, a “Ras effector protein function” refers to a function of a Ras effector protein. In embodiments, the Ras effector protein is one or more of RAF, ARAF, BRAF, RAF1, PI3K, RalGDS, RGL2, PLCE1, RIN1, RASSF5, p120^{GAP}, RIN1, TIAM, Af6, Nore1, PLC, Grb14, Bry2, and Sin1. In embodiments, the Ras effector protein is as described in Rajalingam, K. et al. Biochimica et Biophysica Acta 1773 (2007) 1177-1195; and Kiel, C. et al. Biomolecules 11(2), 236 (2021), which are herein incorporated by reference in their entirety for all purposes. In embodiments, the Ras effector protein function is activation of the Ras/Raf/MEK/ERK pathway.

In embodiments, the modulating of the activity is reducing nucleotide exchange (e.g., GDP exchange for GTP). In embodiments, the modulating of the activity is reducing effector protein binding to Ras. In embodiments, the modulating of the activity is reducing Ras-mediated effector protein activation. In embodiments, the modulating of the activity is reducing guanine exchange factor (GEF) binding to Ras. In embodiments, the modulating of the activity is reducing GEF-facilitated nucleotide exchange. In embodiments, the modulating of the activity is reducing phosphate release (e.g., release of GTP hydrolysis product) from Ras. In embodiments, the modulating of the activity is reducing nucleotide release. In embodiments, the modulating of the activity is reducing nucleotide binding (e.g., GTP binding) to Ras. In embodiments, the modulating of the activity is reducing Ras subcellular localization (e.g., membrane localization). In embodiments, the modulating of the activity is reducing Ras post-translational processing. In embodiments, the modulating of the activity is reducing Ras post-translational modifications. In embodiments, the Ras post-translational modification is CaaX processing. In embodiments, the Ras post-translational modification is prenylation. In embodiments, the Ras post-translational modification is proteolysis. In embodiments, the Ras post-translational modification is carboxyl methylation. In embodiments, the Ras post-translational modification is palmitoylation. In embodiments, the Ras post-translational modification is phosphorylation. In embodiments, the Ras post-translational modification is RAS cysteine oxidation. In embodiments, the Ras post-translational modification is lysine modification (e.g., lysine ubiquitination, lysine SUMOylation, lysine acetylation, lysine methylation). In embodiments, the Ras post-translational modification is as described in Rajalingam, K. et al. Biochimica et Biophysica Acta 1773 (2007) 1177-1195; and Kiel, C. et al. Biomolecules 11(2), 236 (2021), which are herein incorporated by reference in their entirety for all purposes.

In embodiments, the modulating is increasing the activity of the Ras protein. In embodiments, the level of activity of the Ras protein is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the Ras protein is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the Ras protein is increased by about 1.5-fold relative to a control (e.g., absence of the compound). In embodiments, the level of activity of the Ras protein is increased by about 2-fold relative to a control (e.g., absence of the compound). In embodiments, the level of activity of the Ras protein is increased by about 5-fold relative to a control (e.g., absence of the compound). In embodiments, the level of activity of the Ras protein is increased by about 10-fold relative to a control (e.g., absence of the compound). In embodiments, the level of activity of the Ras protein is increased by about 25-fold relative to a control (e.g., absence of the compound). In embodiments, the level of activity of the Ras protein is increased by about 50-fold relative to a control (e.g., absence of the compound). In embodiments, the level of activity of the Ras protein is increased by about 100-fold relative to a control (e.g., absence of the compound). In embodiments, the level of activity of the Ras protein is increased by about 500-fold relative to a control (e.g., absence of the compound). In embodiments, the level of activity of the Ras protein is increased by about 1000-fold relative to a control (e.g., absence of the compound).

In embodiments, the level of activity of the Ras protein is increased by at least 1.5-fold relative to a control (e.g., absence of the compound). In embodiments, the level of activity of the Ras protein is increased by at least 2-fold relative to a control (e.g., absence of the compound). In embodiments, the level of activity of the Ras protein is increased by at least 5-fold relative to a control (e.g., absence of the compound). In embodiments, the level of activity of the Ras protein is increased by at least 10-fold relative to a control (e.g., absence of the compound). In embodiments, the level of activity of the Ras protein is increased by at least 25-fold relative to a control (e.g., absence of the compound). In embodiments, the level of activity of the Ras protein is increased by at least 50-fold relative to a control (e.g., absence of the compound). In embodiments, the level of activity of the Ras protein is increased by at least 100-fold relative to a control (e.g., absence of the compound). In embodiments, the level of activity of the Ras protein is increased by at least 500-fold relative to a control (e.g., absence of the compound). In embodiments, the level of activity of the Ras protein is increased by at least 1000-fold relative to a control (e.g., absence of the compound).

In embodiments, the modulating is reducing the activity of the Ras protein. In embodiments, the level of activity of the Ras protein is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the Ras protein is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the level of activity of the Ras protein is reduced by about 1.5-fold relative to a control (e.g., absence of the compound). In embodiments, the level of activity of the Ras protein is reduced by about 2-fold relative to a control (e.g., absence of the compound). In embodiments, the level of activity of the Ras protein is reduced by about 5-fold relative to a control (e.g., absence of the compound). In embodiments, the level of activity of the Ras protein is reduced by about 10-fold relative to a control (e.g., absence of the compound). In embodiments, the level of activity of the Ras protein is reduced by about 25-fold relative to a control (e.g., absence of the compound). In embodiments, the level of activity of the Ras protein is reduced by about 50-fold relative to a control (e.g., absence of the compound). In embodiments, the level of activity of the Ras protein is reduced by about 100-fold relative to a control (e.g., absence of the compound). In embodiments, the level of activity of the Ras protein is reduced by about 500-fold relative to a control (e.g., absence of the compound). In embodiments, the level of activity of the Ras protein is reduced by about 1000-fold relative to a control (e.g., absence of the compound).

In embodiments, the level of activity of the Ras protein is reduced by at least 1.5-fold relative to a control (e.g., absence of the compound). In embodiments, the level of activity of the Ras protein is reduced by at least 2-fold relative to a control (e.g., absence of the compound). In embodiments, the level of activity of the Ras protein is reduced by at least 5-fold relative to a control (e.g., absence of the compound). In embodiments, the level of activity of the Ras protein is reduced by at least 10-fold relative to a control (e.g., absence of the compound). In embodiments, the level of activity of the Ras protein is reduced by at least 25-fold relative to a control (e.g., absence of the compound). In embodiments, the level of activity of the Ras protein is reduced by at least 50-fold relative to a control (e.g., absence of the compound). In embodiments, the level of activity of the Ras protein is reduced by at least 100-fold relative to a control (e.g., absence of the compound). In embodiments, the level of activity of the Ras protein is reduced by at least 500-fold relative to a control (e.g., absence of the compound). In embodiments, the level of activity of the Ras protein is reduced by at least 1000-fold relative to a control (e.g., absence of the compound).

In an aspect is provided a method of reducing Ras protein-mediated activity in a cell, the method including contacting the cell with an effective amount of a compound described herein, or a pharmaceutically acceptable salt thereof.

In embodiments, Ras protein-mediated activity is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, Ras protein-mediated activity is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, Ras protein-mediated activity is reduced by about 1.5-fold relative to a control (e.g., absence of the compound). In embodiments, Ras protein-mediated activity is reduced by about 2-fold relative to a control (e.g., absence of the compound). In embodiments, Ras protein-mediated activity is reduced by about 5-fold relative to a control (e.g., absence of the compound). In embodiments, Ras protein-mediated activity is reduced by about 10-fold relative to a control (e.g., absence of the compound). In embodiments, Ras protein-mediated activity is reduced by about 25-fold relative to a control (e.g., absence of the compound). In embodiments, Ras protein-mediated activity is reduced by about 50-fold relative to a control (e.g., absence of the compound). In embodiments, Ras protein-mediated activity is reduced by about 100-fold relative to a control (e.g., absence of the compound). In embodiments, Ras protein-mediated activity is reduced by about 500-fold relative to a control (e.g., absence of the compound). In embodiments, Ras protein-mediated activity is reduced by about 1000-fold relative to a control (e.g., absence of the compound).

In embodiments, Ras protein-mediated activity is reduced by at least 1.5-fold relative to a control (e.g., absence of the compound). In embodiments, Ras protein-mediated activity is reduced by at least 2-fold relative to a control (e.g., absence of the compound). In embodiments, Ras protein-mediated activity is reduced by at least 5-fold relative to a control (e.g., absence of the compound). In embodiments, Ras protein-mediated activity is reduced by at least 10-fold relative to a control (e.g., absence of the compound). In embodiments, Ras protein-mediated activity is reduced by at least 25-fold relative to a control (e.g., absence of the compound). In embodiments, Ras protein-mediated activity is reduced by at least 50-fold relative to a control (e.g., absence of the compound). In embodiments, Ras protein-mediated activity is reduced by at least 100-fold relative to a control (e.g., absence of the compound). In embodiments, Ras protein-mediated activity is reduced by at least 500-fold relative to a control (e.g., absence of the compound). In embodiments, Ras protein-mediated activity is reduced by at least 1000-fold relative to a control (e.g., absence of the compound).

In embodiments, the Ras protein is a K-Ras protein. In embodiments, the Ras protein is a human K-Ras protein. In embodiments, human K-Ras protein contains a G12R mutation, a G13R mutation, or a Q61R mutation. In embodiments, human K-Ras protein contains a G12R mutation. In embodiments, human K-Ras protein contains a G13R mutation. In embodiments, human K-Ras protein contains a Q61R mutation.

In embodiments, the Ras protein is an H-Ras protein. In embodiments, the Ras protein is a human H-Ras protein. In embodiments, human H-Ras protein contains a G12R mutation, a G13R mutation, or a Q61R mutation. In embodiments, human H-Ras protein contains a G12R mutation. In embodiments, human H-Ras protein contains a G13R mutation. In embodiments, human H-Ras protein contains a Q61R mutation.

In embodiments, the Ras protein is an N-Ras protein. In embodiments, the Ras protein is a human N-Ras protein. In embodiments, human N-Ras protein contains a G12R mutation, a G13R mutation, or a Q61R mutation. In embodiments, human N-Ras protein contains a G12R mutation. In embodiments, human N-Ras protein contains a G13R mutation. In embodiments, human N-Ras protein contains a Q61R mutation.

### V. Methods of Making

In an aspect is provided a method of attaching a compound to an arginine residue of a protein, the method including contacting said compound with the arginine residue, wherein the compound has the formula:

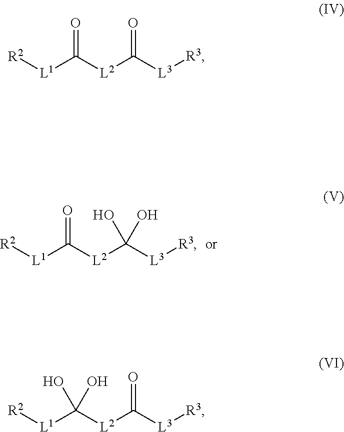

or a salt thereof. L¹, L², L³, and R³ are as described herein, including in embodiments.

R² is a Switch II Binding Pocket binding moiety, a phosphatase PTP domain binding moiety, an SH2 domain binding moiety, a pseudokinase KSR domain binding moiety, or a pseudokinase STRADα domain binding moiety.

In embodiments, the protein further includes additional arginine residues and none of the additional arginine residues react with the compound.

In embodiments, the protein is a Switch II GTPase protein. In embodiments, the Switch II GTPase protein is a Ras protein. In embodiments, the Ras protein is a K-Ras protein. In embodiments, the Ras protein is a human K-Ras protein. In embodiments, the human K-Ras protein contains a G12R mutation. In embodiments, the human K-Ras protein contains a G13R mutation. In embodiments, the human K-Ras protein contains a Q61R mutation. In embodiments, the Ras protein further includes additional arginine residues and none of the additional arginine residues react with the compound. In embodiments, the Ras protein is an H-Ras protein. In embodiments, the Ras protein is a human H-Ras protein. In embodiments, the human H-Ras protein contains a G12R mutation. In embodiments, the human H-Ras protein contains a G13R mutation. In embodiments, the human H-Ras protein contains a Q61R mutation. In embodiments, the Ras protein further comprises additional arginine residues and none of the additional arginine residues react with the compound. In embodiments, the Ras protein is an N-Ras protein. In embodiments, the Ras protein is a human N-Ras protein. In embodiments, the human N-Ras protein contains a G12R mutation. In embodiments, the human N-Ras protein contains a G13R mutation. In embodiments, the human N-Ras protein contains a Q61R mutation. In embodiments, the Ras protein further comprises additional arginine residues and none of the additional arginine residues react with the compound.

In embodiments, the compound has the formula:

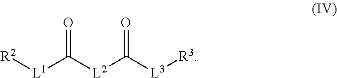

L¹, L², L³, R², and R³ are as described herein, including in embodiments.

In embodiments, the compound has the formula:

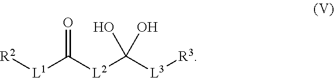

L¹, L², L³, R², and R³ are as described herein, including in embodiments.

In embodiments, the compound has the formula:

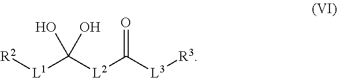

L¹, L², L³, R², and R³ are as described herein, including in embodiments.

In embodiments, the compound has the formula:

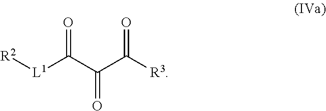

L¹, R², and R³ are as described herein, including in embodiments.

In embodiments, the compound has the formula:

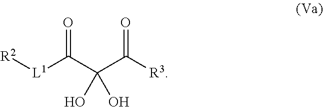

L¹, R², and R³ are as described herein, including in embodiments.

In embodiments, the compound has the formula:

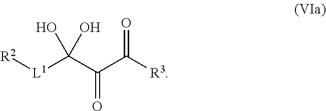

L¹, R², and R³ are as described herein, including in embodiments.

In embodiments, the compound has the formula:

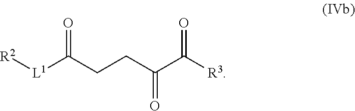

L¹, R², and R³ are as described herein, including in embodiments.

In embodiments, the compound has the formula:

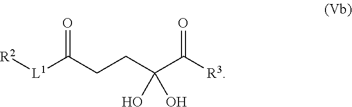

L¹, R², and R³ are as described herein, including in embodiments.

In embodiments, the compound has the formula:

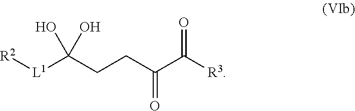

L¹, R², and R³ are as described herein, including in embodiments.

In embodiments, the compound has the formula:

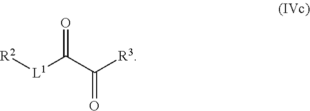

L¹, R², and R³ are as described herein, including in embodiments.

In embodiments, the compound has the formula:

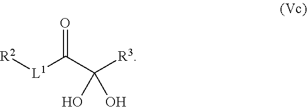

L¹, R², and R³ are as described herein, including in embodiments.

In embodiments, the compound has the formula:

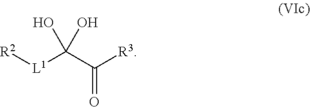

L¹, R², and R³ are as described herein, including in embodiments.

In embodiments, R² is a Switch II Binding Pocket binding moiety, as described herein, including in embodiments. In embodiments, R² is any value of R¹ as described herein, including in embodiments. In embodiments, R² is a phosphatase PTP domain binding moiety. In embodiments, R² is an SH2 domain binding moiety. In embodiments, R² is a pseudokinase KSR domain binding moiety. In embodiments, R² is a pseudokinase STRADα domain binding moiety.

In embodiments, R² is -L²⁰-R²⁰.

L²⁰ is a bond, —C(O)—, —C(O)O—, —OC(O)—, —O—, —S—, —NR²⁰⁰—, —C(O)NR²⁰⁰—, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene (e.g., C₁-C₅, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R²⁰ is hydrogen, halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, N(O)ₘ₂₀, —NR^{20A}R^{20B}, —C(O)R^{20C}, —C(O)OR^{20C}, —C(O)NR^{20A}R^{20B}, —OR^{20D}, —SR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

Each X²⁰ is independently —F, —Cl, —Br, or —I.

The symbol n20 is an integer from 0 to 4.

The symbols m20 and v20 are independently 1 or 2.

In embodiments, a substituted L²⁰ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L²⁰ is substituted with a plurality of groups selected from substituent groups, size-limited substituent groups, and lower substituent groups; each substituent group, size-limited substituent group, and/or lower substituent group may optionally be different. In embodiments, when L²⁰ is substituted, it is substituted with at least one substituent group. In embodiments, when L²⁰ is substituted, it is substituted with at least one size-limited substituent group. In embodiments, when L²⁰ is substituted, it is substituted with at least one lower substituent group.

In embodiments, L²⁰ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L²⁰ is a bond. In embodiments, L²⁰ is —C(O)—. In embodiments, L²⁰ is —C(O)O—. In embodiments, L²⁰ is —OC(O)—. In embodiments, L²⁰ is —O—. In embodiments, L²⁰ is —S—. In embodiments, L²⁰ is —NR²⁰⁰—. In embodiments, L²⁰ is —NH—. In embodiments, L²⁰ is —C(O)NR²⁰⁰—. In embodiments, L²⁰ is —C(O)NH—. In embodiments, L²⁰ is —NR²⁰⁰C(O)—. In embodiments, L²⁰ is —NHC(O)—. In embodiments, L²⁰ is —NR²⁰⁰C(O)O—. In embodiments, L²⁰ is —NHC(O)O—. In embodiments, L²⁰ is —OC(O)NR²⁰⁰—. In embodiments, L²⁰ is —OC(O)NH—. In embodiments, L²⁰ is —NR²⁰⁰C(O)NR²⁰⁰—. In embodiments, L²⁰ is —NHC(O)NH—. In embodiments, L²⁰ is —NR²⁰⁰C(NI)NR²⁰⁰—. In embodiments, L²⁰ is —NHC(NH)NH—. In embodiments, L²⁰ is —S(O)₂—. In embodiments, L²⁰ is —NR²⁰⁰S(O)₂—. In embodiments, L²⁰ is —NHS(O)₂—. In embodiments, L²⁰ is —S(O)₂NR²⁰⁰—. In embodiments, L²⁰ is —S(O)₂NH—. In embodiments, L²⁰ is substituted or unsubstituted C₁-C₆ alkylene. In embodiments, L²⁰ is substituted or unsubstituted methylene. In embodiments, L²⁰ is substituted or unsubstituted ethylene. In embodiments, L²⁰ is substituted or unsubstituted propylene. In embodiments, L²⁰ is substituted or unsubstituted n-propylene. In embodiments, L²⁰ is substituted or unsubstituted isopropylene. In embodiments, L²⁰ is substituted or unsubstituted butylene. In embodiments, L²⁰ is substituted or unsubstituted n-butylene. In embodiments, L²⁰ is substituted or unsubstituted isobutylene. In embodiments, L²⁰ is substituted or unsubstituted tert-butylene. In embodiments, L²⁰ is substituted or unsubstituted pentylene. In embodiments, L²⁰ is substituted or unsubstituted hexylene. In embodiments, L²⁰ is substituted or unsubstituted 2 to 6 membered heteroalkylene.

In embodiments, a substituted R²⁰⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰⁰ is substituted with a plurality of groups selected from substituent groups, size-limited substituent groups, and lower substituent groups; each substituent group, size-limited substituent group, and/or lower substituent group may optionally be different. In embodiments, when R²⁰⁰ is substituted, it is substituted with at least one substituent group. In embodiments, when R²⁰⁰ is substituted, it is substituted with at least one size-limited substituent group. In embodiments, when R²⁰⁰ is substituted, it is substituted with at least one lower substituent group.

In embodiments, R²⁰⁰ is independently hydrogen. In embodiments, R²⁰⁰ is independently unsubstituted C₁-C₄ alkyl. In embodiments, R²⁰⁰ is independently unsubstituted methyl. In embodiments, R²⁰⁰ is independently unsubstituted ethyl. In embodiments, R²⁰⁰ is independently unsubstituted propyl. In embodiments, R²⁰⁰ is independently unsubstituted n-propyl. In embodiments, R²⁰⁰ is independently unsubstituted isopropyl. In embodiments, R²⁰⁰ is independently unsubstituted butyl. In embodiments, R²⁰⁰ is independently unsubstituted n-butyl. In embodiments, R²⁰⁰ is independently unsubstituted isobutyl. In embodiments, R²⁰⁰ is independently unsubstituted tert-butyl.

In embodiments, a substituted R²⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰ is substituted with a plurality of groups selected from substituent groups, size-limited substituent groups, and lower substituent groups; each substituent group, size-limited substituent group, and/or lower substituent group may optionally be different. In embodiments, when R²⁰ is substituted, it is substituted with at least one substituent group. In embodiments, when R²⁰ is substituted, it is substituted with at least one size-limited substituent group. In embodiments, when R²⁰ is substituted, it is substituted with at least one lower substituent group.

In embodiments, a substituted R^{20A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20A} is substituted with a plurality of groups selected from substituent groups, size-limited substituent groups, and lower substituent groups; each substituent group, size-limited substituent group, and/or lower substituent group may optionally be different. In embodiments, when R^{20A} is substituted, it is substituted with at least one substituent group. In embodiments, when R^{20A} is substituted, it is substituted with at least one size-limited substituent group. In embodiments, when R^{20A} is substituted, it is substituted with at least one lower substituent group.

In embodiments, a substituted R^{20B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20B} is substituted with a plurality of groups selected from substituent groups, size-limited substituent groups, and lower substituent groups; each substituent group, size-limited substituent group, and/or lower substituent group may optionally be different. In embodiments, when R^{20B} is substituted, it is substituted with at least one substituent group. In embodiments, when R^{20B} is substituted, it is substituted with at least one size-limited substituent group. In embodiments, when R^{20B} is substituted, it is substituted with at least one lower substituent group.

In embodiments, a substituted ring formed when R^{20A} and R^{20B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{20A} and R^{20B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{20A} and R^{20B} substituents bonded to the same nitrogen atom are joined is substituted, it is substituted with at least one substituent group. In embodiments, when the substituted ring formed when R^{20A} and R^{20B} substituents bonded to the same nitrogen atom are joined is substituted, it is substituted with at least one size-limited substituent group. In embodiments, when the substituted ring formed when R^{20A} and R^{20B} substituents bonded to the same nitrogen atom are joined is substituted, it is substituted with at least one lower substituent group.

In embodiments, a substituted R^{20C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20C} is substituted with a plurality of groups selected from substituent groups, size-limited substituent groups, and lower substituent groups; each substituent group, size-limited substituent group, and/or lower substituent group may optionally be different. In embodiments, when R^{20C} is substituted, it is substituted with at least one substituent group. In embodiments, when R^{20C} is substituted, it is substituted with at least one size-limited substituent group. In embodiments, when R^{20C} is substituted, it is substituted with at least one lower substituent group.

In embodiments, a substituted R^{20D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20D} is substituted with a plurality of groups selected from substituent groups, size-limited substituent groups, and lower substituent groups; each substituent group, size-limited substituent group, and/or lower substituent group may optionally be different. In embodiments, when R^{20D} is substituted, it is substituted with at least one substituent group. In embodiments, when R^{20D} is substituted, it is substituted with at least one size-limited substituent group. In embodiments, when R^{20D} is substituted, it is substituted with at least one lower substituent group.

In embodiments, R²⁰ is substituted or unsubstituted cycloalkyl, substituted or unsubstituted heterocycloalkyl, substituted or unsubstituted aryl, or substituted or unsubstituted heteroaryl. In embodiments, R²⁰ is substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R²⁰ is substituted C₃-C₈ cycloalkyl. In embodiments, R²⁰ is substituted 3 to 8 membered heterocycloalkyl. In embodiments, R²⁰ is substituted C₆-C₁₀ aryl. In embodiments, R²⁰ is substituted phenyl. In embodiments, R²⁰ is substituted 5 to 20 membered heteroaryl. In embodiments, R²⁰ is substituted pyrimidyl. In embodiments, R²⁰ is substituted tetrahydropyridopyrimidyl. In embodiments, R²⁰ is substituted pyridopyrimidyl. In embodiments, R²⁰ is substituted quinazolinyl. In embodiments, R²⁰ is substituted pyrazolyl. In embodiments, R²⁰ is substituted piperazinyl.

In embodiments, R^{20A} is hydrogen. In embodiments, R^{20A} is unsubstituted C₁-C₄ alkyl. In embodiments, R^{20A} is unsubstituted methyl. In embodiments, R^{20A} is unsubstituted ethyl. In embodiments, R^{20A} is unsubstituted propyl. In embodiments, R^{20A} is unsubstituted n-propyl. In embodiments, R^{20A} is unsubstituted isopropyl. In embodiments, R^{20A} is unsubstituted butyl. In embodiments, R^{20A} is unsubstituted n-butyl. In embodiments, R^{20A} is unsubstituted isobutyl. In embodiments, R^{20A} is unsubstituted tert-butyl.

In embodiments, R^{20B} is hydrogen. In embodiments, R^{20B} is unsubstituted C₁-C₄ alkyl. In embodiments, R^{20B} is unsubstituted methyl. In embodiments, R^{20B} is unsubstituted ethyl. In embodiments, R^{20B} is unsubstituted propyl. In embodiments, R^{20B} is unsubstituted n-propyl. In embodiments, R^{20B} is unsubstituted isopropyl. In embodiments, R^{20B} is unsubstituted butyl. In embodiments, R^{20B} is unsubstituted n-butyl. In embodiments, R^{20B} is unsubstituted isobutyl. In embodiments, R^{20B} is unsubstituted tert-butyl.

In embodiments, R^{20C} is hydrogen. In embodiments, R^{20C} is unsubstituted C₁-C₄ alkyl. In embodiments, R^{20C} is unsubstituted methyl. In embodiments, R^{20C} is unsubstituted ethyl. In embodiments, R^{20C} is unsubstituted propyl. In embodiments, R^{20C} is unsubstituted n-propyl. In embodiments, R^{20C} is unsubstituted isopropyl. In embodiments, R^{20C} is unsubstituted butyl. In embodiments, R^{20C} is unsubstituted n-butyl. In embodiments, R^{20C} is unsubstituted isobutyl. In embodiments, R^{20C} is unsubstituted tert-butyl.

In embodiments, R^{20D} is hydrogen. In embodiments, R^{20D} is unsubstituted C₁-C₄ alkyl. In embodiments, R^{20D} is unsubstituted methyl. In embodiments, R^{20D} is unsubstituted ethyl. In embodiments, R^{20D} is unsubstituted propyl. In embodiments, R^{20D} is unsubstituted n-propyl. In embodiments, R^{20D} is unsubstituted isopropyl. In embodiments, R^{20D} is unsubstituted butyl. In embodiments, R^{20D} is unsubstituted n-butyl. In embodiments, R^{20D} is unsubstituted isobutyl. In embodiments, R^{20D} is unsubstituted tert-butyl.

In embodiments, R² is

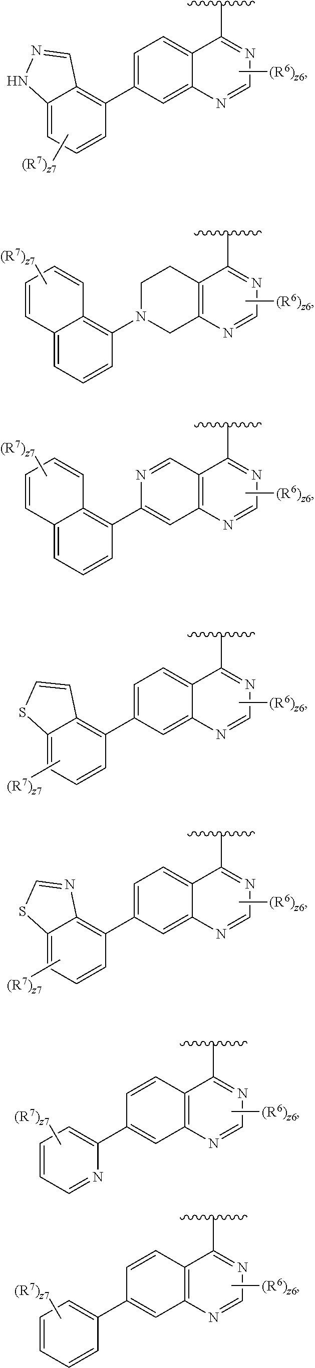

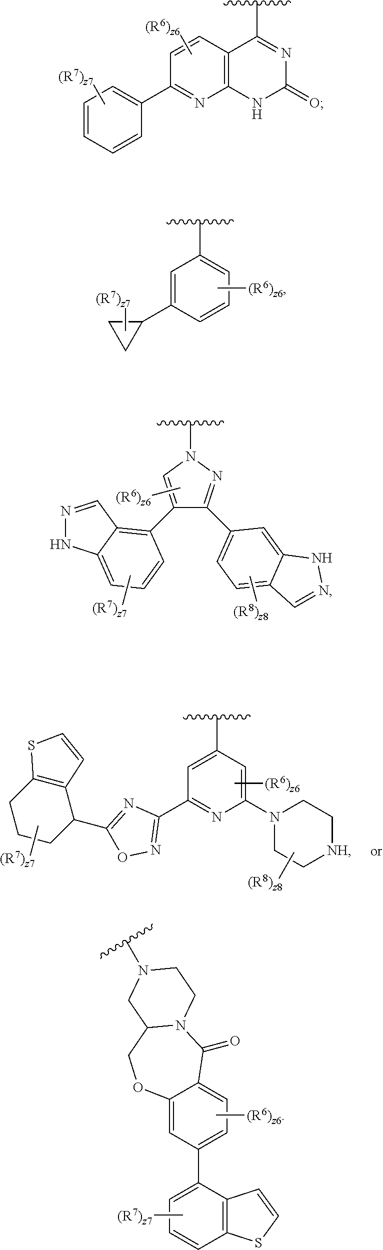

In embodiments, R² is

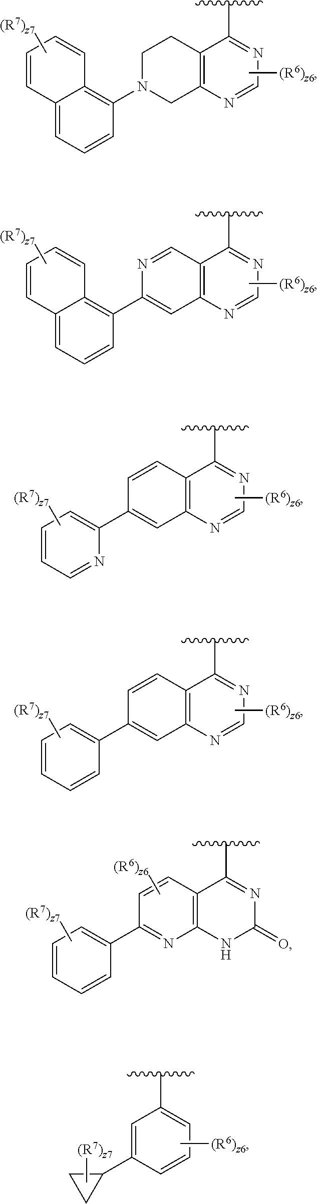

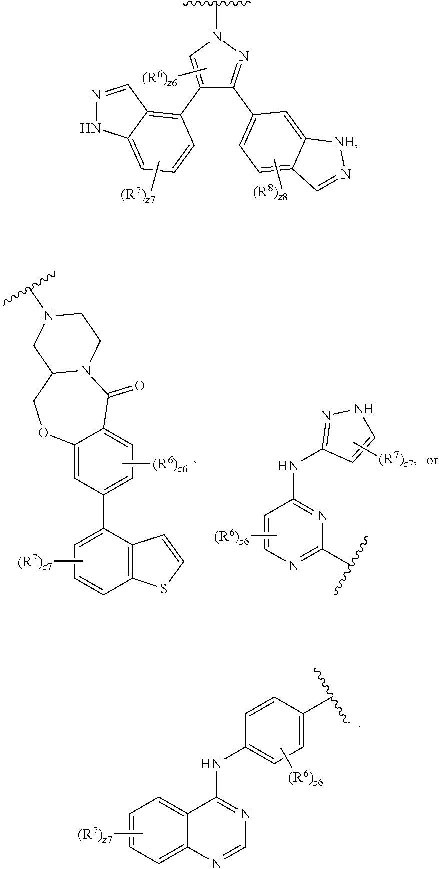

R⁶, z6, R⁷, z7, R⁸, and z8 are as described herein, including in embodiments. In embodiments, R² is

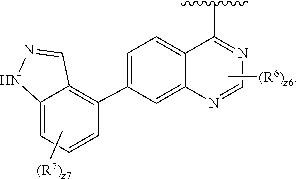

In embodiments, R² is

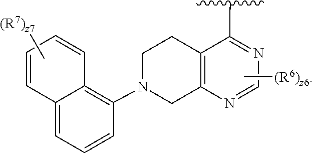

In embodiments, R² is

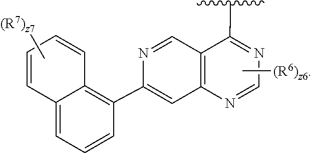

In embodiments, R² is

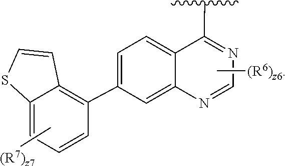

In embodiments, R² is

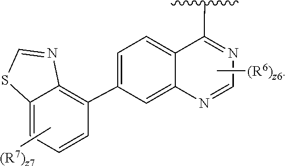

In embodiments, R² is

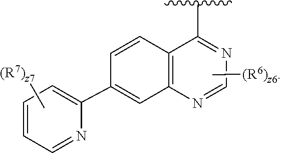

In embodiments, R² is

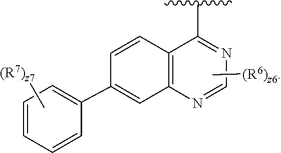

In embodiments, R² is

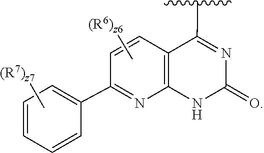

In embodiments, R² is

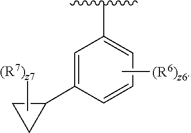

In embodiments, R² is

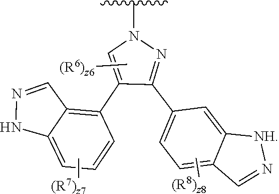

In embodiments, R² is

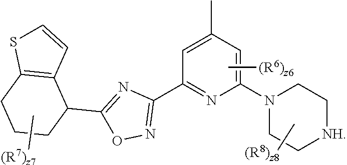

In embodiments, R² is

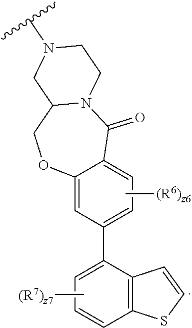

In embodiments, R² is

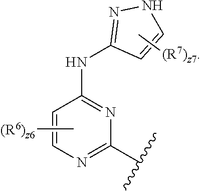

In embodiments, R² is

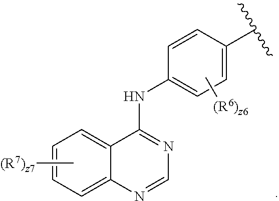

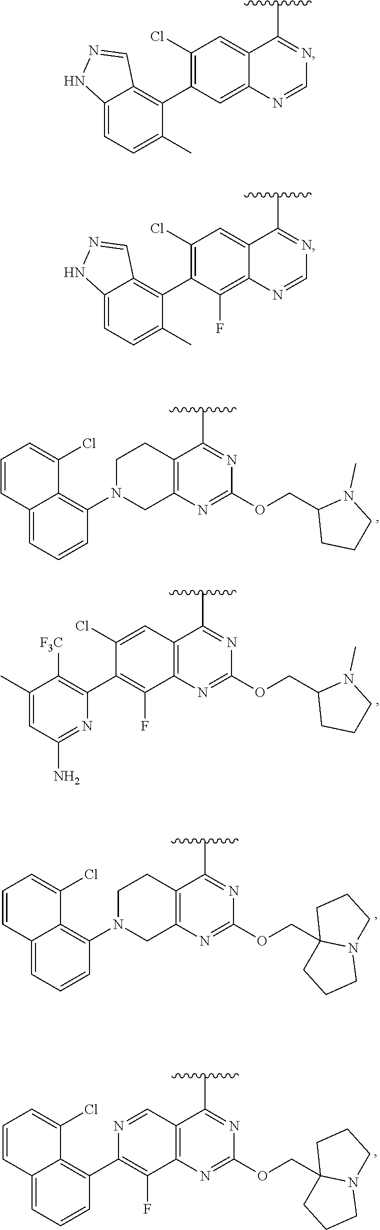 [SMILES: *c1ncnc2cc(-c3c(C)ccc4[nH]ncc34)c(Cl)cc12 ; *c1ncnc2c(F)c(-c3c(C)ccc4[nH]ncc34)c(Cl)cc12 ; *c1nc(OCC2CCCN2C)nc2c1CCN(c1cccc3cccc(Cl)c13)C2 ; *c1nc(OCC2CCCN2C)nc2c(F)c(-c3nc(N)cc(C)c3C(F)(F)F)c(Cl)cc12 ; *c1nc(OCC23CCCN2CCC3)nc2c1CCN(c1cccc3cccc(Cl)c13)C2 ; *c1nc(OCC23CCCN2CCC3)nc2c(F)c(-c3cccc4cccc(Cl)c34)ncc12]

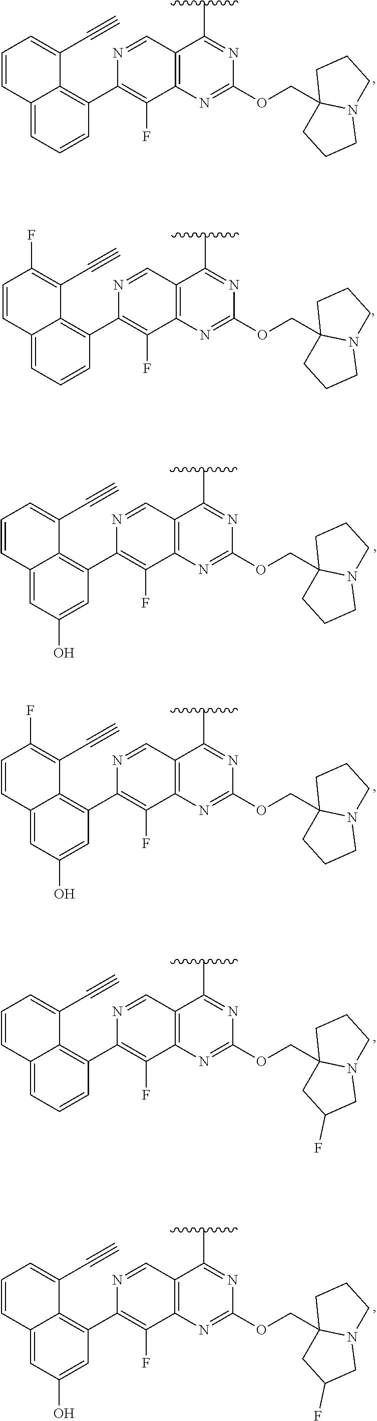 [SMILES: *c1nc(OCC23CCCN2CCC3)nc2c(F)c(-c3cccc4cccc(C#C)c34)ncc12 ; *c1nc(OCC23CCCN2CCC3)nc2c(F)c(-c3cccc4ccc(F)c(C#C)c34)ncc12 ; *c1nc(OCC23CCCN2CCC3)nc2c(F)c(-c3cc(O)cc4cccc(C#C)c34)ncc12 ; *c1nc(OCC23CCCN2CCC3)nc2c(F)c(-c3cc(O)cc4ccc(F)c(C#C)c34)ncc12 ; *c1nc(OCC23CCCN2CC(F)C3)nc2c(F)c(-c3cccc4cccc(C#C)c34)ncc12 ; *c1nc(OCC23CCCN2CC(F)C3)nc2c(F)c(-c3cc(O)cc4cccc(C#C)c34)ncc12]

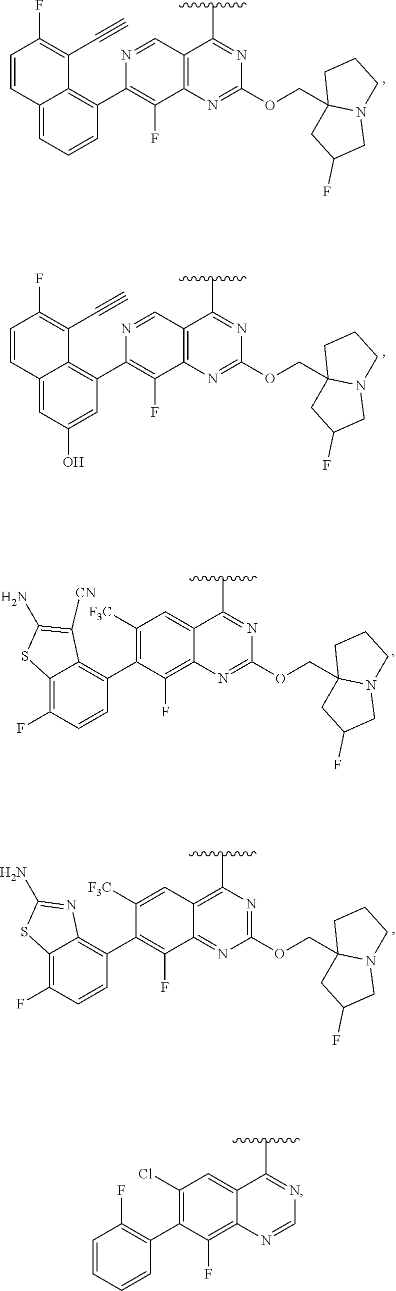 [SMILES: *c1nc(OCC23CCCN2CC(F)C3)nc2c(F)c(-c3cccc4ccc(F)c(C#C)c34)ncc12 ; *c1nc(OCC23CCCN2CC(F)C3)nc2c(F)c(-c3cc(O)cc4ccc(F)c(C#C)c34)ncc12 ; *c1nc(OCC23CCCN2CC(F)C3)nc2c(F)c(-c3ccc(F)c4sc(N)c(C#N)c34)c(C(F)(F)F)cc12 ; *c1nc(OCC23CCCN2CC(F)C3)nc2c(F)c(-c3ccc(F)c4sc(N)nc34)c(C(F)(F)F)cc12 ; *c1ncnc2c(F)c(-c3ccccc3F)c(Cl)cc12]

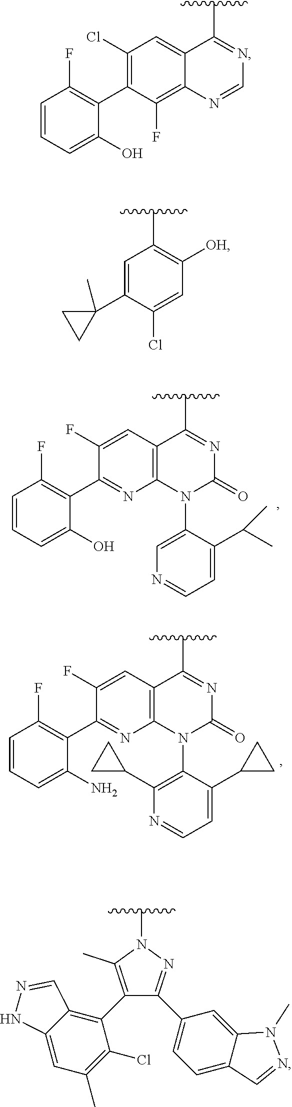

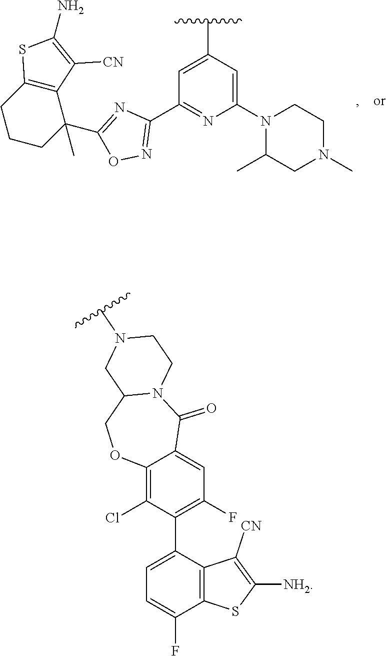 [SMILES: *c1cc(-c2noc(C3(C)CCCc4sc(N)c(C#N)c43)n2)nc(N2CCN(C)CC2C)c1 ; *N1CCN2C(=O)c3cc(F)c(-c4ccc(F)c5sc(N)c(C#N)c45)c(Cl)c3OCC2C1]

In embodiments, R² is

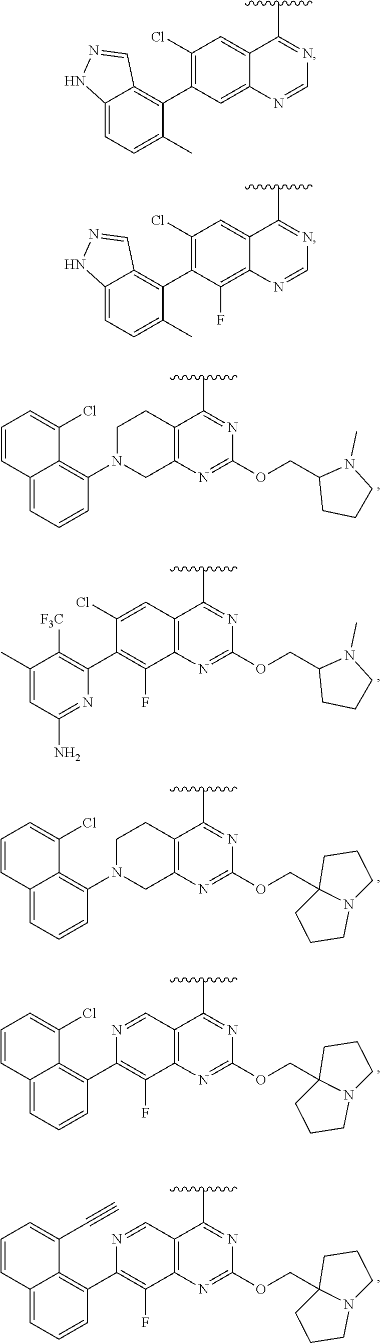

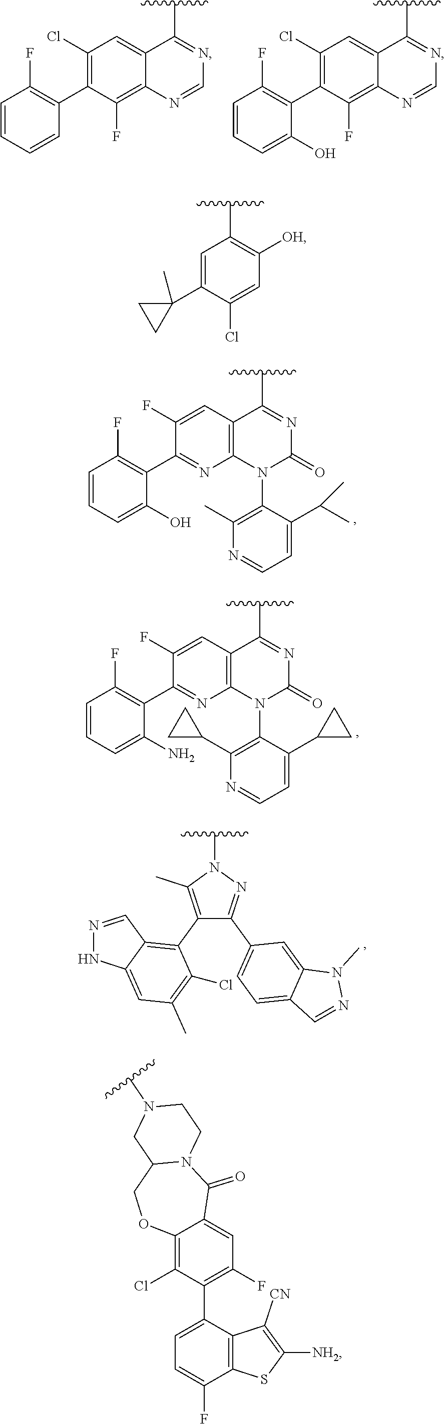

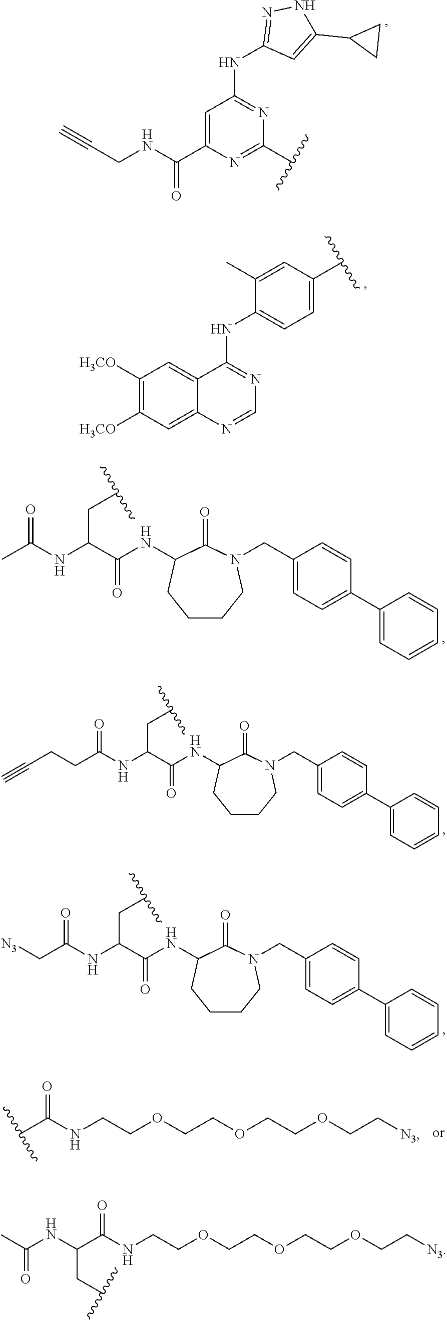

In embodiments, R² is

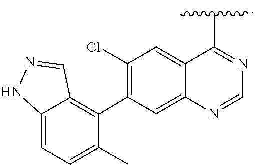 [SMILES: *c1ncnc2cc(-c3c(C)ccc4[nH]ncc34)c(Cl)cc12]

In embodiments, R² is

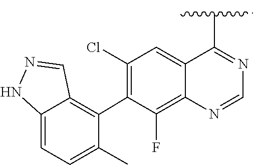 [SMILES: *c1ncnc2c(F)c(-c3c(C)ccc4[nH]ncc34)c(Cl)cc12]

In embodiments, R² is

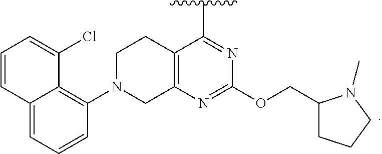 [SMILES: *c1nc(OCC2CCCN2C)nc2c1CCN(c1cccc3cccc(Cl)c13)C2]

In embodiments, R² is

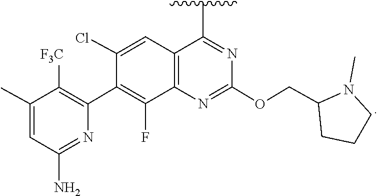 [SMILES: *c1nc(OCC2CCCN2C)nc2c(F)c(-c3nc(N)cc(C)c3C(F)(F)F)c(Cl)cc12]

In embodiments, R² is

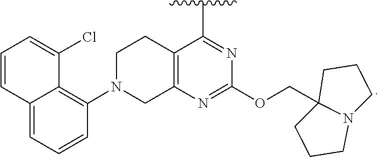 [SMILES: *c1nc(OCC23CCCN2CCC3)nc2c1CCN(c1cccc3cccc(Cl)c13)C2]

In embodiments, R² is

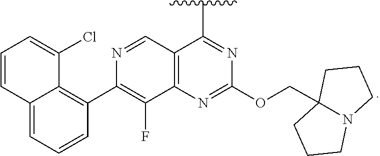 [SMILES: *c1nc(OCC23CCCN2CCC3)nc2c(F)c(-c3cccc4cccc(Cl)c34)ncc12]

In embodiments, R² is

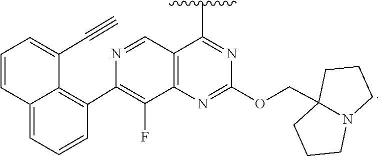 [SMILES: *c1nc(OCC23CCCN2CCC3)nc2c(F)c(-c3cccc4cccc(C#C)c34)ncc12]

In embodiments, R² is

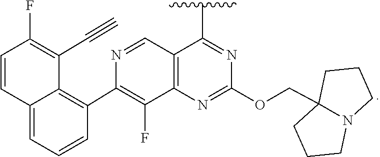 [SMILES: *c1nc(OCC23CCCN2CCC3)nc2c(F)c(-c3cccc4ccc(F)c(C#C)c34)ncc12]

In embodiments, R² is

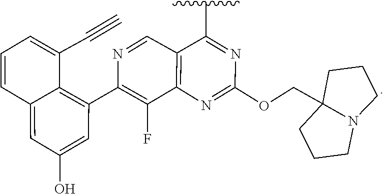 [SMILES: *c1nc(OCC23CCCN2CCC3)nc2c(F)c(-c3cc(O)cc4cccc(C#C)c34)ncc12]

In embodiments, R² is

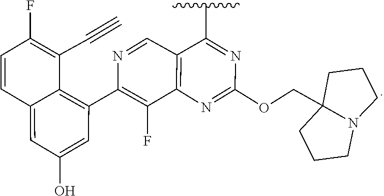 [SMILES: *c1nc(OCC23CCCN2CCC3)nc2c(F)c(-c3cc(O)cc4ccc(F)c(C#C)c34)ncc12]

In embodiments, R² is

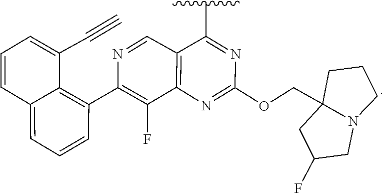 [SMILES: *c1nc(OCC23CCCN2CC(F)C3)nc2c(F)c(-c3cccc4cccc(C#C)c34)ncc12]

In embodiments, R² is

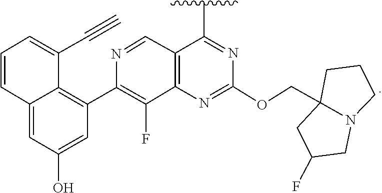 [SMILES: *c1nc(OCC23CCCN2CC(F)C3)nc2c(F)c(-c3cc(O)cc4cccc(C#C)c34)ncc12]

In embodiments, R² is

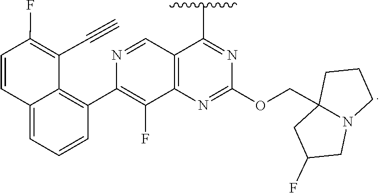 [SMILES: *c1nc(OCC23CCCN2CC(F)C3)nc2c(F)c(-c3cccc4ccc(F)c(C#C)c34)ncc12]

In embodiments, R² is

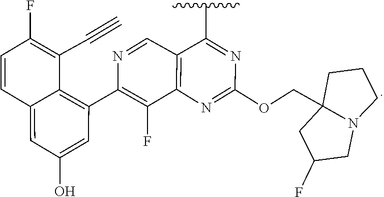 [SMILES: *c1nc(OCC23CCCN2CC(F)C3)nc2c(F)c(-c3cc(O)cc4ccc(F)c(C#C)c34)ncc12]

In embodiments, R² is

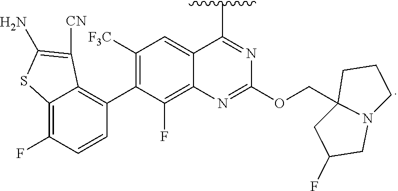 [SMILES: *c1nc(OCC23CCCN2CC(F)C3)nc2c(F)c(-c3ccc(F)c4sc(N)c(C#N)c34)c(C(F)(F)F)cc12]

In embodiments, R² is

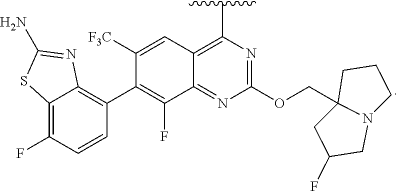 [SMILES: *c1nc(OCC23CCCN2CC(F)C3)nc2c(F)c(-c3ccc(F)c4sc(N)nc34)c(C(F)(F)F)cc12]

In embodiments, R² is

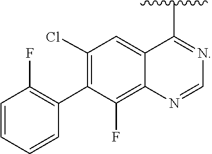 [SMILES: *c1ncnc2c(F)c(-c3ccccc3F)c(Cl)cc12]

In embodiments, R² is

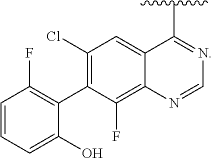 [SMILES: *c1ncnc2c(F)c(-c3c(O)cccc3F)c(Cl)cc12]

In embodiments, R² is

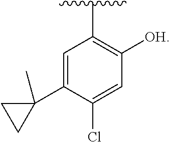 [SMILES: *c1cc(C2(C)CC2)c(Cl)cc1O]

In embodiments, R² is

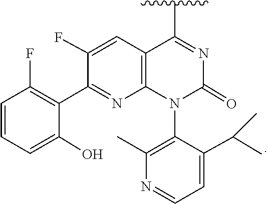 [SMILES: *c1nc(=O)n(-c2c(C(C)C)ccnc2C)c2nc(-c3c(O)cccc3F)c(F)cc12]

In embodiments, R² is

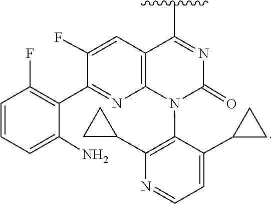 [SMILES: *c1nc(=O)n(-c2c(C3CC3)ccnc2C2CC2)c2nc(-c3c(N)cccc3F)c(F)cc12]

In embodiments, R² is

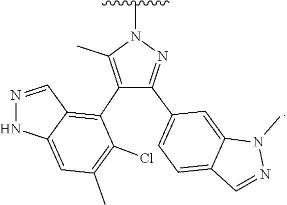 [SMILES: *n1nc(-c2ccc3cnn(C)c3c2)c(-c2c(Cl)c(C)cc3[nH]ncc23)c1C]

In embodiments, R² is

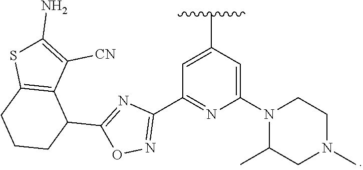 [SMILES: *c1cc(-c2noc(C3CCCc4sc(N)c(C#N)c43)n2)nc(N2CCN(C)CC2C)c1]

In embodiments, R² is

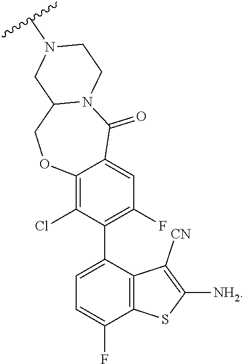 [SMILES: *N1CCN2C(=O)c3cc(F)c(-c4ccc(F)c5sc(N)c(C#N)c45)c(Cl)c3OCC2C1]

In embodiments, R² is

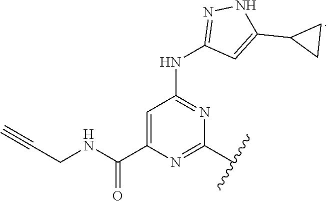 [SMILES: *c1nc(Nc2cc(C3CC3)[nH]n2)cc(C(=O)NCC#C)n1]

In embodiments, R² is

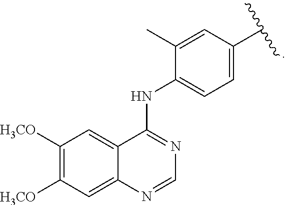 [SMILES: *c1ccc(Nc2ncnc3cc(OC)c(OC)cc23)c(C)c1]

In embodiments, R² is

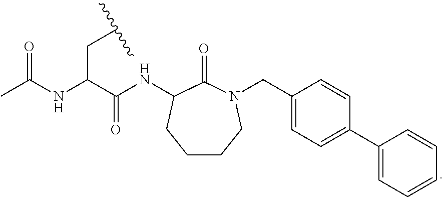 [SMILES: *CC(NC(C)=O)C(=O)NC1CCCCN(Cc2ccc(-c3ccccc3)cc2)C1=O]

In embodiments, R² is

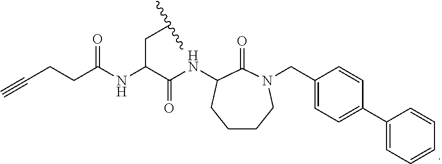 [SMILES: *CC(NC(=O)CCC#C)C(=O)NC1CCCCN(Cc2ccc(-c3ccccc3)cc2)C1=O]

In embodiments, R² is

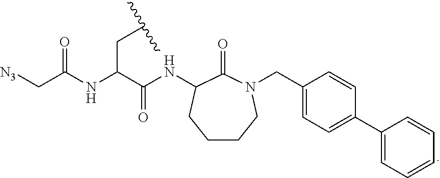 [SMILES: *CC(NC(=O)CN=[N+]=[N-])C(=O)NC1CCCCN(Cc2ccc(-c3ccccc3)cc2)C1=O]

In embodiments, R² is

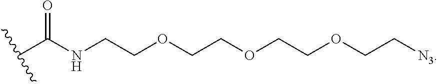 [SMILES: *C(=O)NCCOCCOCCOCCN=[N+]=[N-]]

In embodiments, R² is

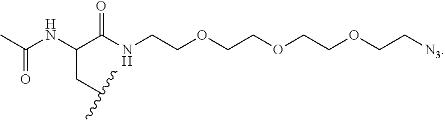 [SMILES: *CC(NC(C)=O)C(=O)NCCOCCOCCOCCN=[N+]=[N-]]

In embodiments, the compound has the formula:

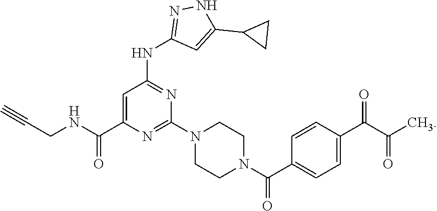 [SMILES: C#CCNC(=O)c1cc(Nc2cc(C3CC3)[nH]n2)nc(N2CCN(C(=O)c3ccc(C(=O)C(C)=O)cc3)CC2)n1]

In embodiments, the compound has the formula:

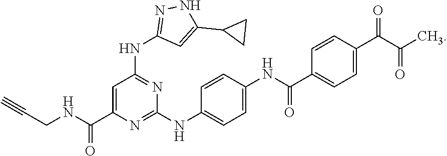 [SMILES: C#CCNC(=O)c1cc(Nc2cc(C3CC3)[nH]n2)nc(Nc2ccc(NC(=O)c3ccc(C(=O)C(C)=O)cc3)cc2)n1]

In embodiments, the compound has the formula:

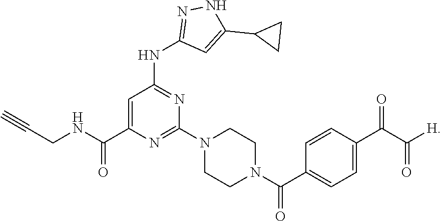

In embodiments, the compound has the formula:

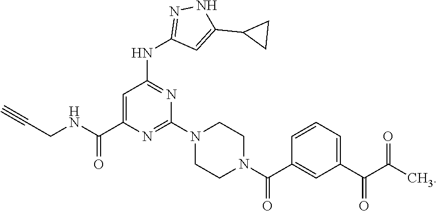 [SMILES: C#CCNC(=O)c1cc(Nc2cc(C3CC3)[nH]n2)nc(N2CCN(C(=O)c3cccc(C(=O)C(C)=O)c3)CC2)n1]

In embodiments, the compound has the formula:

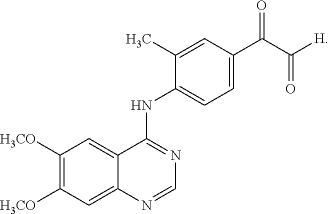

In embodiments, the compound has the formula:

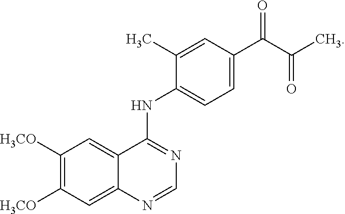 [SMILES: COc1cc2ncnc(Nc3ccc(C(=O)C(C)=O)cc3C)c2cc1OC]

In embodiments, the compound has the formula:

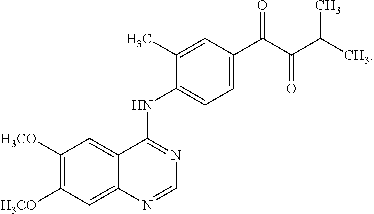 [SMILES: COc1cc2ncnc(Nc3ccc(C(=O)C(=O)C(C)C)cc3C)c2cc1OC]

In embodiments, the compound has the formula:

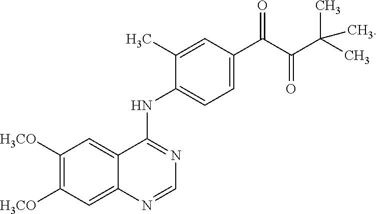 [SMILES: COc1cc2ncnc(Nc3ccc(C(=O)C(=O)C(C)(C)C)cc3C)c2cc1OC]

In embodiments, the compound has the formula:

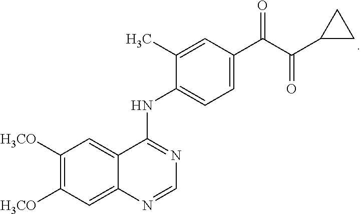 [SMILES: COc1cc2ncnc(Nc3ccc(C(=O)C(=O)C4CC4)cc3C)c2cc1OC]

In embodiments, the compound has the formula:

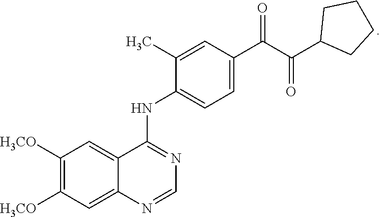 [SMILES: COc1cc2ncnc(Nc3ccc(C(=O)C(=O)C4CCCC4)cc3C)c2cc1OC]

In embodiments, the compound has the formula:

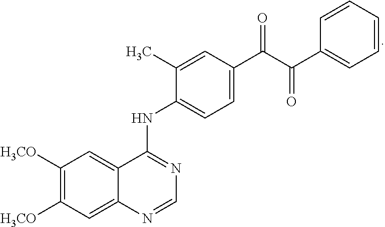 [SMILES: COc1cc2ncnc(Nc3ccc(C(=O)C(=O)c4ccccc4)cc3C)c2cc1OC]

In embodiments, the compound has the formula:

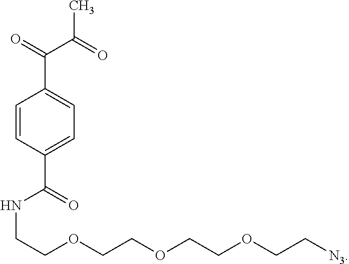 [SMILES: CC(=O)C(=O)c1ccc(C(=O)NCCOCCOCCOCCN=[N+]=[N-])cc1]

In embodiments, the compound has the formula:

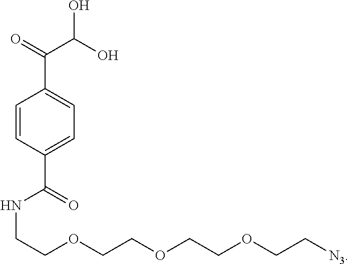 [SMILES: [N-]=[N+]=NCCOCCOCCOCCNC(=O)c1ccc(C(=O)C(O)O)cc1]

In embodiments, the compound has the formula:

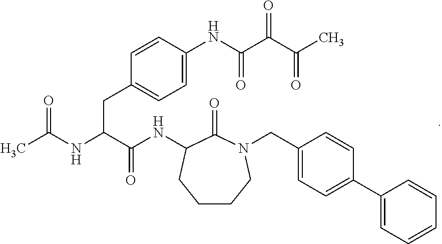 [SMILES: CC(=O)NC(Cc1ccc(NC(=O)C(=O)C(C)=O)cc1)C(=O)NC1CCCCN(Cc2ccc(-c3ccccc3)cc2)C1=O]

In embodiments, the compound has the formula:

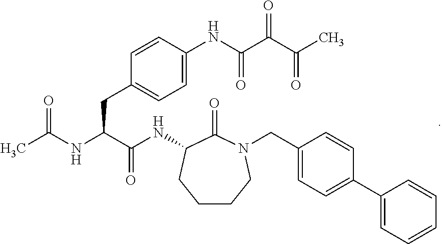 [SMILES: CC(=O)N[C@@H](Cc1ccc(NC(=O)C(=O)C(C)=O)cc1)C(=O)N[C@H]1CCCCN(Cc2ccc(-c3ccccc3)cc2)C1=O]

In embodiments, the compound has the

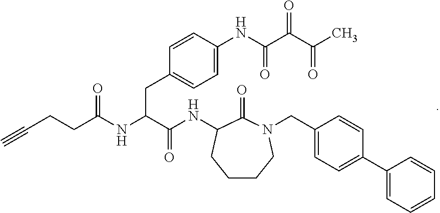 [SMILES: C#CCCC(=O)NC(Cc1ccc(NC(=O)C(=O)C(C)=O)cc1)C(=O)NC1CCCCN(Cc2ccc(-c3ccccc3)cc2)C1=O]

In embodiments, the compound has the formula:

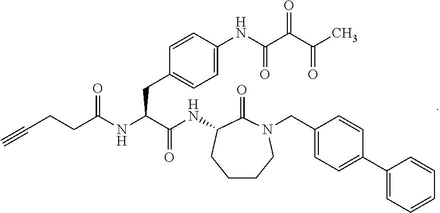 [SMILES: C#CCCC(=O)N[C@@H](Cc1ccc(NC(=O)C(=O)C(C)=O)cc1)C(=O)N[C@H]1CCCCN(Cc2ccc(-c3ccccc3)cc2)C1=O]

In embodiments, the compound has the formula:

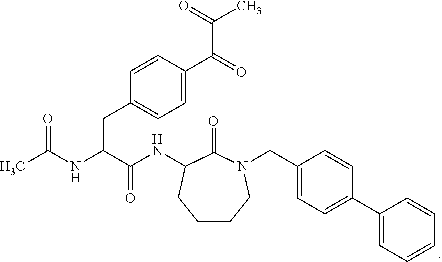 [SMILES: CC(=O)NC(Cc1ccc(C(=O)C(C)=O)cc1)C(=O)NC1CCCCN(Cc2ccc(-c3ccccc3)cc2)C1=O]

In embodiments, the compound has the formula:

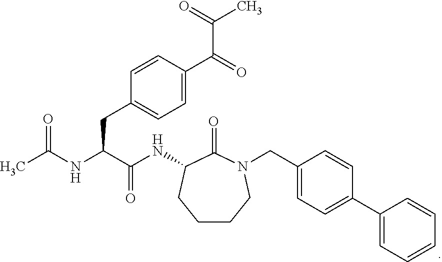 [SMILES: CC(=O)N[C@@H](Cc1ccc(C(=O)C(C)=O)cc1)C(=O)N[C@H]1CCCCN(Cc2ccc(-c3ccccc3)cc2)C1=O]

In embodiments, the compound has the formula:

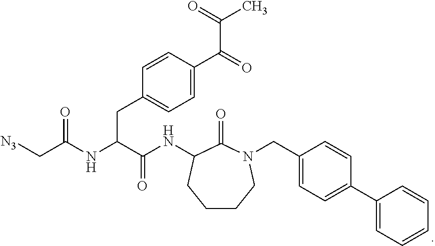 [SMILES: CC(=O)C(=O)c1ccc(CC(NC(=O)CN=[N+]=[N-])C(=O)NC2CCCCN(Cc3ccc(-c4ccccc4)cc3)C2=O)cc1]

In embodiments, the compound has the formula:

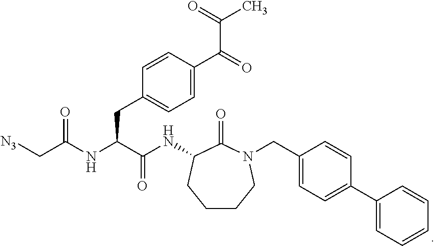 [SMILES: CC(=O)C(=O)c1ccc(C[C@H](NC(=O)CN=[N+]=[N-])C(=O)N[C@H]2CCCCN(Cc3ccc(-c4ccccc4)cc3)C2=O)cc1]

In embodiments, the compound has the formula:

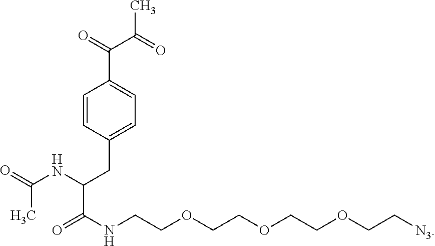 [SMILES: CC(=O)NC(Cc1ccc(C(=O)C(C)=O)cc1)C(=O)NCCOCCOCCOCCN=[N+]=[N-]]

In embodiments, the compound has the formula:

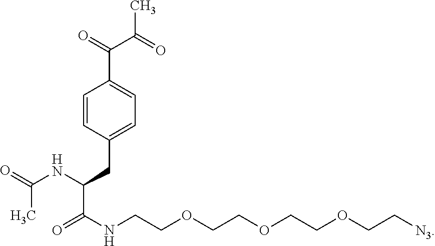 [SMILES: CC(=O)N[C@@H](Cc1ccc(C(=O)C(C)=O)cc1)C(=O)NCCOCCOCCOCCN=[N+]=[N-]]

In embodiments, the compound has the formula:

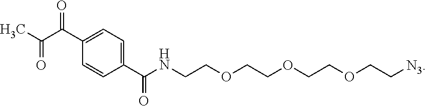 [SMILES: CC(=O)C(=O)c1ccc(C(=O)NCCOCCOCCOCCN=[N+]=[N-])cc1]

In embodiments, the compound has the formula:

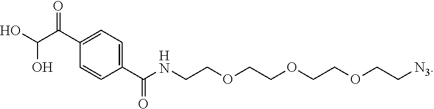 [SMILES: [N-]=[N+]=NCCOCCOCCOCCNC(=O)c1ccc(C(=O)C(O)O)cc1]

In embodiments, the compound is as described herein.

In an aspect is provided a method of attaching a compound to an arginine residue, the method including contacting the compound with the arginine residue, wherein the compound has the formula:

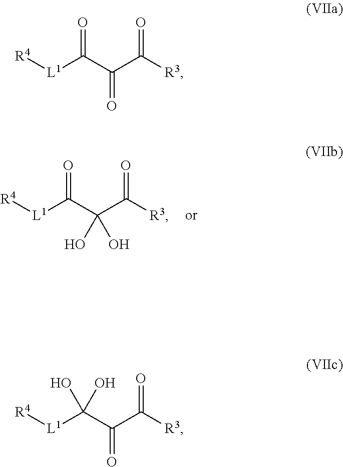

or a salt thereof. L¹ and R³ are as described herein, including in embodiments.

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —N^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —N^{4A}C(O)OR^{4C}, —NR^{4A}OR^{4C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

Each X⁴ is independently —Cl, —Br, —I, or —F.

The symbol n4 is an integer from 0 to 4.

The symbols m4 and v4 are independently 1 or 2.

In embodiments, the arginine residue forms part of a protein.

In embodiments, the arginine residue is attached to a biomolecule or a portion of a biomolecule. In embodiments, the biomolecule is a Switch II GTPase protein. In embodiments, the Switch II GTPase protein is a Ras protein. In embodiments, the Ras protein is a K-Ras protein. In embodiments, the Ras protein is a human K-Ras protein. In embodiments, the human K-Ras protein contains a G12R mutation. In embodiments, the human K-Ras protein contains a G13R mutation. In embodiments, the human K-Ras protein contains a Q61R mutation. In embodiments, the Ras protein further includes additional arginine residues and none of the additional arginine residues react with the compound. In embodiments, the Ras protein is an H-Ras protein. In embodiments, the Ras protein is a human H-Ras protein. In embodiments, the human H-Ras protein contains a G12R mutation. In embodiments, the human H-Ras protein contains a G13R mutation. In embodiments, the human H-Ras protein contains a Q61R mutation. In embodiments, the Ras protein further comprises additional arginine residues and none of the additional arginine residues react with the compound. In embodiments, the Ras protein is an N-Ras protein. In embodiments, the Ras protein is a human N-Ras protein. In embodiments, the human N-Ras protein contains a G12R mutation. In embodiments, the human N-Ras protein contains a G13R mutation. In embodiments, the human N-Ras protein contains a Q61R mutation. In embodiments, the Ras protein further comprises additional arginine residues and none of the additional arginine residues react with the compound.

In embodiments, the compound has the formula:

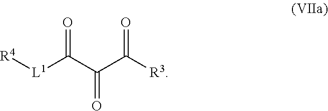

L¹, R³, and R⁴ are as described herein, including in embodiments.

In embodiments, the compound has the formula:

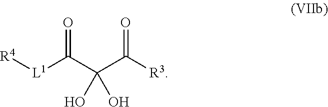

L¹, R³, and R⁴ are as described herein, including in embodiments.

In embodiments, the compound has the formula:

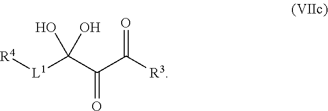

L¹, R³, and R⁴ are as described herein, including in embodiments.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴ is hydrogen. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CCl₃. In embodiments, R⁴ is —CBr₃. In embodiments, R⁴ is —CF₃. In embodiments, R⁴ is —CI₃. In embodiments, R⁴ is —CH₂Cl. In embodiments, R⁴ is —CH₂Br. In embodiments, R⁴ is —CH₂F. In embodiments, R⁴ is —CH₂I. In embodiments, R⁴ is —CHCl₂. In embodiments, R⁴ is —CHBr₂. In embodiments, R⁴ is —CHF₂. In embodiments, R⁴ is —CHI₂. In embodiments, R⁴ is —CN. In embodiments, R⁴ is —OH. In embodiments, R⁴ is —NH₂. In embodiments, R⁴ is —COOH. In embodiments, R⁴ is —CONH₂. In embodiments, R⁴ is —NO₂. In embodiments, R⁴ is —SH. In embodiments, R⁴ is —SO₃H. In embodiments, R⁴ is —OSO₃H. In embodiments, R⁴ is —SO₂NH₂. In embodiments, R⁴ is —NHNH₂. In embodiments, R⁴ is —ONH₂. In embodiments, R⁴ is —NHC(O)NHNH₂. In embodiments, R⁴ is —NHC(O)NH₂. In embodiments, R⁴ is —NHSO₂H. In embodiments, R⁴ is —NHC(O)H. In embodiments, R⁴ is —NHC(O)OH. In embodiments, R⁴ is-NHOH. In embodiments, R⁴ is —OCCl₃. In embodiments, R⁴ is —OCBr₃. In embodiments, R⁴ is —OCF₃. In embodiments, R⁴ is —OCI₃. In embodiments, R⁴ is —OCH₂Cl. In embodiments, R⁴ is —OCH₂Br. In embodiments, R⁴ is —OCH₂F. In embodiments, R⁴ is —OCH₂I. In embodiments, R⁴ is —OCHCl₂. In embodiments, R⁴ is —OCHBr₂. In embodiments, R⁴ is —OCHF₂. In embodiments, R⁴ is —OCHI₂. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl.

In embodiments, R⁴ is a biomolecular moiety. In embodiments, R⁴ is any value of R¹ or R² as described herein, including in embodiments.

In embodiments, R^{4A} is hydrogen. In embodiments, R^{4A} is unsubstituted C₁-C₄ alkyl. In embodiments, R^{4A} is unsubstituted methyl. In embodiments, R^{4A} is unsubstituted ethyl. In embodiments, R^{4A} is unsubstituted propyl. In embodiments, R^{4A} is unsubstituted n-propyl. In embodiments, R^{4A} is unsubstituted isopropyl. In embodiments, R^{4A} is unsubstituted butyl. In embodiments, R^{4A} is unsubstituted n-butyl. In embodiments, R^{4A} is unsubstituted isobutyl. In embodiments, R^{4A} is unsubstituted tert-butyl.

In embodiments, R^{4B} is hydrogen. In embodiments, R^{4B} is unsubstituted C₁-C₄ alkyl. In embodiments, R^{4B} is unsubstituted methyl. In embodiments, R^{4B} is unsubstituted ethyl. In embodiments, R^{4B} is unsubstituted propyl. In embodiments, R^{4B} is unsubstituted n-propyl. In embodiments, R^{4B} is unsubstituted isopropyl. In embodiments, R^{4B} is unsubstituted butyl. In embodiments, R^{4B} is unsubstituted n-butyl. In embodiments, R^{4B} is unsubstituted isobutyl. In embodiments, R^{4B} is unsubstituted tert-butyl.

In embodiments, R^{4C} is hydrogen. In embodiments, R^{4C} is unsubstituted C₁-C₄ alkyl. In embodiments, R^{4C} is unsubstituted methyl. In embodiments, R^{4C} is unsubstituted ethyl. In embodiments, R^{4C} is unsubstituted propyl. In embodiments, R^{4C} is unsubstituted n-propyl. In embodiments, R^{4C} is unsubstituted isopropyl. In embodiments, R^{4C} is unsubstituted butyl. In embodiments, R^{4C} is unsubstituted n-butyl. In embodiments, R^{4C} is unsubstituted isobutyl. In embodiments, R^{4C} is unsubstituted tert-butyl.

In embodiments, R^{4D} is hydrogen. In embodiments, R^{4D} is unsubstituted C₁-C₄ alkyl. In embodiments, R^{4D} is unsubstituted methyl. In embodiments, R^{4D} is unsubstituted ethyl. In embodiments, R^{4D} is unsubstituted propyl. In embodiments, R^{4D} is unsubstituted n-propyl. In embodiments, R^{4D} is unsubstituted isopropyl. In embodiments, R^{4D} is unsubstituted butyl. In embodiments, R^{4D} is unsubstituted n-butyl. In embodiments, R^{4D} is unsubstituted isobutyl. In embodiments, R^{4D} is unsubstituted tert-butyl.

### VI. Protein Compositions

In an aspect is provided a Switch II GTPase protein covalently bound to a compound described herein, or a salt thereof, wherein the compound is covalently bound to an arginine residue of the Switch II GTPase protein.

In embodiments, the compound is reversibly covalently bound to an arginine residue of the Switch II GTPase protein. In embodiments, the compound is irreversibly covalently bound to an arginine residue of the Switch II GTPase protein.

In embodiments, the Switch II GTPase protein is a Ras protein. In embodiments, the Ras protein is a K-Ras protein. In embodiments, the Ras protein is a human K-Ras protein. In embodiments, human K-Ras protein contains a G12R mutation. In embodiments, the compound is covalently bonded to arginine residue 12. In embodiments, the compound is covalently bonded to an arginine residue corresponding to arginine residue 12. In embodiments, human K-Ras protein contains a G13R mutation. In embodiments, the compound is covalently bonded to arginine residue 13. In embodiments, the compound is covalently bonded to an arginine residue corresponding to arginine residue 13. In embodiments, human K-Ras protein contains a Q61R mutation. In embodiments, the compound is covalently bonded to arginine residue 61. In embodiments, the compound is covalently bonded to an arginine residue corresponding to arginine residue 61. In embodiments, the Ras protein further includes additional arginine residues and none of the additional arginine residues react with the compound.

In embodiments, the Ras protein is an H-Ras protein. In embodiments, the Ras protein is a human H-Ras protein. In embodiments, human H-Ras protein contains a G12R mutation. In embodiments, the compound is covalently bonded to arginine residue 12. In embodiments, the compound is covalently bonded to an arginine residue corresponding to arginine residue 12. In embodiments, human H-Ras protein contains a G13R mutation. In embodiments, the compound is covalently bonded to arginine residue 13. In embodiments, the compound is covalently bonded to an arginine residue corresponding to arginine residue 13. In embodiments, human H-Ras protein contains a Q61R mutation. In embodiments, the compound is covalently bonded to arginine residue 61. In embodiments, the compound is covalently bonded to an arginine residue corresponding to arginine residue 61. In embodiments, the Ras protein further comprises additional arginine residues and none of the additional arginine residues react with the compound.

In embodiments, the Ras protein is an N-Ras protein. In embodiments, the Ras protein is a human N-Ras protein. In embodiments, human N-Ras protein contains a G12R mutation. In embodiments, the compound is covalently bonded to arginine residue 12. In embodiments, the compound is covalently bonded to an arginine residue corresponding to arginine residue 12. In embodiments, human N-Ras protein contains a G13R mutation. In embodiments, the compound is covalently bonded to arginine residue 13. In embodiments, the compound is covalently bonded to an arginine residue corresponding to arginine residue 13. In embodiments, human N-Ras protein contains a Q61R mutation. In embodiments, the compound is covalently bonded to arginine residue 61. In embodiments, the compound is covalently bonded to an arginine residue corresponding to arginine residue 61. In embodiments, the Ras protein further comprises additional arginine residues and none of the additional arginine residues react with the compound.

In embodiments, the Ras (e.g., K-Ras, H-Ras, or N-Ras) protein is covalently bonded (e.g., reversibly or irreversibly) to a portion (e.g., fragment, moiety, moiety of a fragment) of a compound described herein.

In embodiments, a Ras(G12R) (e.g., human K-Ras(G12R), human H-Ras(G12R), or human N-Ras(G12R)) protein is covalently bonded to a compound (e.g., compound described herein or a portion of a compound described herein). In embodiments, the Ras(G12R) (e.g., human K-Ras(G12R), human H-Ras(G12R), or human N-Ras(G12R)) protein is irreversibly covalently bonded to a compound (e.g., compound described herein or a portion of a compound described herein). In embodiments, the Ras(G12R) (e.g., human K-Ras(G12R), human H-Ras(G12R), or human N-Ras(G12R)) protein is reversibly covalently bonded to a compound (e.g., compound described herein or a portion of a compound described herein). In embodiments, the Ras(G12R) (e.g., human K-Ras(G12R), human H-Ras(G12R), or human N-Ras(G12R)) protein is covalently bonded to a portion of a compound (e.g., compound described herein). In embodiments, the Ras(G12R) (e.g., human K-Ras(G12R), human H-Ras(G12R), or human N-Ras(G12R)) protein is irreversibly covalently bonded to a portion of a compound described herein. In embodiments, the Ras(G12R) (e.g., human K-Ras(G12R), human H-Ras(G12R), or human N-Ras(G12R)) protein is reversibly covalently bonded to a portion of a compound described herein. In embodiments, the compound described herein is bonded to an arginine residue (e.g., G12R of human K-Ras(G12R) or arginine corresponding to G12R of human K-Ras(G12R)) of the Ras(G12R) (e.g., human K-Ras(G12R), human H-Ras(G12R), or human N-Ras(G12R)) protein.

In embodiments, a Ras(G13R) (e.g., human K-Ras(G13R), human H-Ras(G13R), or human N-Ras(G13R)) protein is covalently bonded to a compound (e.g., compound described herein or a portion of a compound described herein). In embodiments, the Ras(G13R) (e.g., human K-Ras(G13R), human H-Ras(G13R), or human N-Ras(G13R)) protein is irreversibly covalently bonded to a compound (e.g., compound described herein or a portion of a compound described herein). In embodiments, the Ras(G13R) (e.g., human K-Ras(G13R), human H-Ras(G13R), or human N-Ras(G13R)) protein is reversibly covalently bonded to a compound (e.g., compound described herein or a portion of a compound described herein). In embodiments, the Ras(G13R) (e.g., human K-Ras(G13R), human H-Ras(G13R), or human N-Ras(G13R)) protein is covalently bonded to a portion of a compound (e.g., compound described herein). In embodiments, the Ras(G13R) (e.g., human K-Ras(G13R), human H-Ras(G13R), or human N-Ras(G13R)) protein is irreversibly covalently bonded to a portion of a compound described herein. In embodiments, the Ras(G13R) (e.g., human K-Ras(G13R), human H-Ras(G13R), or human N-Ras(G13R)) protein is reversibly covalently bonded to a portion of a compound described herein. In embodiments, the compound described herein is bonded to an arginine residue (e.g., G13R of human K-Ras(G13R) or arginine corresponding to G13R of human K-Ras(G13R)) of the Ras(G13R) (e.g., human K-Ras(G13R), human H-Ras(G13R), or human N-Ras(G13R)) protein.

In embodiments, a Ras(Q61R) (e.g., human K-Ras(Q61R), human H-Ras(Q61R), or human N-Ras(Q61R)) protein is covalently bonded to a compound (e.g., compound described herein or a portion of a compound described herein). In embodiments, the Ras(Q61R) (e.g., human K-Ras(Q61R), human H-Ras(Q61R), or human N-Ras(Q61R)) protein is irreversibly covalently bonded to a compound (e.g., compound described herein or a portion of a compound described herein). In embodiments, the Ras(Q61R) (e.g., human K-Ras(Q61R), human H-Ras(Q61R), or human N-Ras(Q61R)) protein is reversibly covalently bonded to a compound (e.g., compound described herein or a portion of a compound described herein). In embodiments, the Ras(Q61R) (e.g., human K-Ras(Q61R), human H-Ras(Q61R), or human N-Ras(Q61R)) protein is covalently bonded to a portion of a compound (e.g., compound described herein). In embodiments, the Ras(Q61R) (e.g., human K-Ras(Q61R), human H-Ras(Q61R), or human N-Ras(Q61R)) protein is irreversibly covalently bonded to a portion of a compound described herein. In embodiments, the Ras(Q61R) (e.g., human K-Ras(Q61R), human H-Ras(Q61R), or human N-Ras(Q61R)) protein is reversibly covalently bonded to a portion of a compound described herein. In embodiments, the compound described herein is bonded to a arginine residue (e.g., Q61R of human K-Ras(Q61R) or arginine corresponding to Q61R of human K-Ras(Q61R)) of the Ras(Q61R) (e.g., human K-Ras(Q61R), human H-Ras(Q61R), or human N-Ras(Q61R)) protein.

In embodiments, the covalently modified Ras (e.g., K-Ras, H-Ras, or N-Ras) protein has a modulated activity relative to a control, wherein the activity is selected from GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, Ras (e.g., K-Ras, H-Ras, or N-Ras) subcellular localization, Ras (e.g., K-Ras, H-Ras, or N-Ras) post-translational processing, and Ras (e.g., K-Ras, H-Ras, or N-Ras) post-translational modifications.

In an aspect is provided a compound covalently bound to an arginine residue, having the formula:

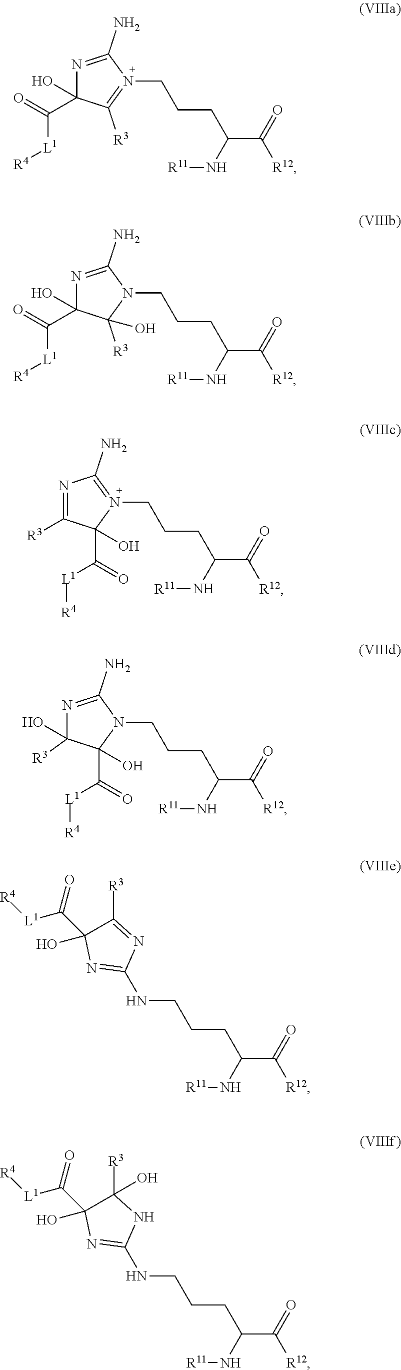

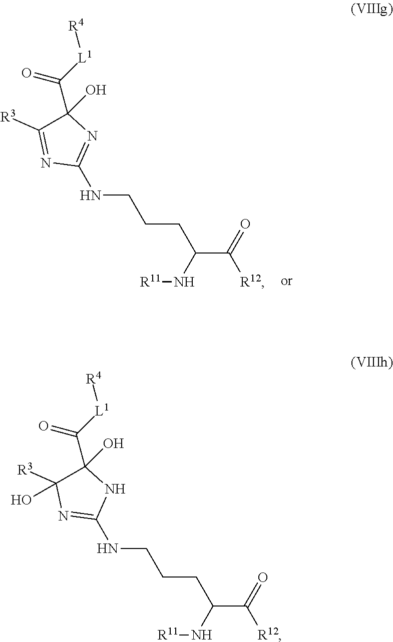

or a salt thereof. L¹, R³, and R⁴ are as described herein, including in embodiments.

R¹¹ is hydrogen, —C(O)CH₃, or a first protein moiety. In embodiments, R¹¹ is hydrogen. In embodiments, R¹¹ is —C(O)CH₃. In embodiments, R¹¹ is a first protein moiety.

R¹² is —OH or a second protein moiety. In embodiments, R¹² is —OH. In embodiments, R¹² is a second protein moiety.

In embodiments, the compound covalently bound to the arginine residue has the formula:

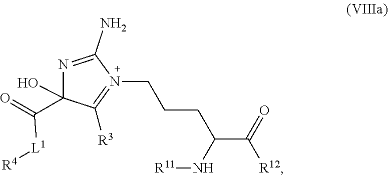

wherein L¹, R³, R⁴, R¹¹, and R¹² are as described herein, including in embodiments. In embodiments, the compound covalently bound to the arginine residue has the formula:

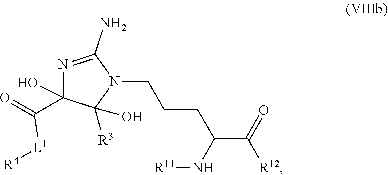

wherein L¹, R³, R⁴, R¹¹, and R¹² are as described herein, including in embodiments. In embodiments, the compound covalently bound to the arginine residue has the formula:

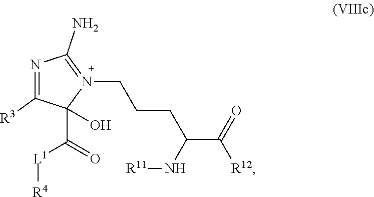

wherein L¹, R³, R⁴, R¹¹, and R¹² are as described herein, including in embodiments. In embodiments, the compound covalently bound to the arginine residue has the formula:

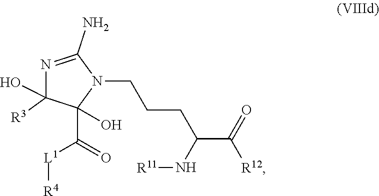

wherein L¹, R³, R⁴, R¹¹, and R¹² are as described herein, including in embodiments. In embodiments, the compound covalently bound to the arginine residue has the formula:

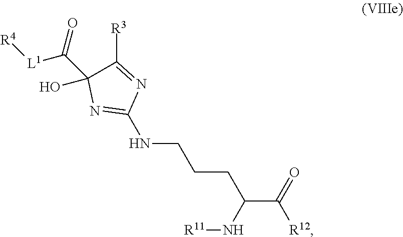

wherein L¹, R³, R⁴, R¹¹, and R¹² are as described herein, including in embodiments. In embodiments, the compound covalently bound to the arginine residue has the formula:

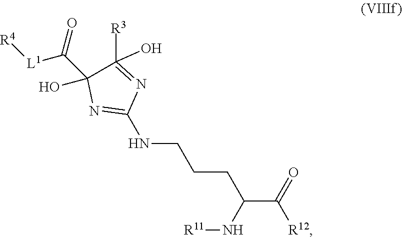

wherein L¹, R³, R⁴, R¹¹, and R¹² are as described herein, including in embodiments. In embodiments, the compound covalently bound to the arginine residue has the formula:

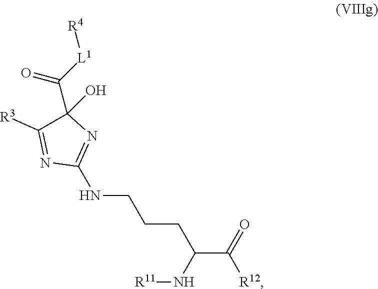

wherein L¹, R³, R⁴, R¹¹, and R¹² are as described herein, including in embodiments. In embodiments, the compound covalently bound to the arginine residue has the formula:

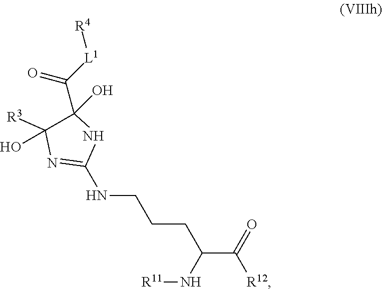

wherein L¹, R³, R⁴, R¹¹, and R¹² are as described herein, including in embodiments.

In embodiments, the first protein moiety and the second protein moiety together form a single protein. In embodiments, the single protein includes additional arginine residues that are not covalently bound to a compound to form a reacted arginine having the formula (VIa), (VIb), (VIc), (VId), (VIe), (VIf), (VIg), or (VIh). In embodiments, the single protein is a Switch II GTPase protein. In embodiments, the single protein is a Ras protein. In embodiments, the single protein is a K-Ras(G12R) (e.g., human K-Ras(G12R)) protein. In embodiments, the single protein is an H-Ras(G12R) (e.g., human H-Ras(G12R)) protein. In embodiments, the single protein is an N-Ras(G12R) (e.g., human N-Ras(G12R)) protein. In embodiments, the single protein is a K-Ras(G13R) (e.g., human K-Ras(G13R)) protein. In embodiments, the single protein is an H-Ras(G13R) (e.g., human H-Ras(G13R)) protein. In embodiments, the single protein is an N-Ras(G13R) (e.g., human N-Ras(G13R)) protein. In embodiments, the single protein is a K-Ras(Q61R) (e.g., human K-Ras(Q61R)) protein. In embodiments, the single protein is an H-Ras(Q61R) (e.g., human H-Ras(Q61R)) protein. In embodiments, the single protein is an N-Ras(Q61R) (e.g., human N-Ras(Q61R)) protein. In embodiments, the single protein is a phosphatase protein. In embodiments, the single protein is a Src oncoprotein. In embodiments, the single protein is a pseudokinase KSR (e.g., KSR2) protein. In embodiments, the single protein is a pseudokinase STRADα protein.

In embodiments, the arginine residue is a Switch II Binding Pocket arginine residue. In embodiments, the arginine residue is a phosphatase PTP domain arginine residue. In embodiments, the arginine residue is an SH2 domain binding moiety arginine residue. In embodiments, the arginine residue is a pseudokinase KSR domain binding moiety arginine residue. In embodiments, the arginine residue is a pseudokinase STRADα domain binding moiety arginine residue.

### VII. Embodiments

Embodiment P1. A compound, or a pharmaceutically acceptable salt thereof, having the formula:

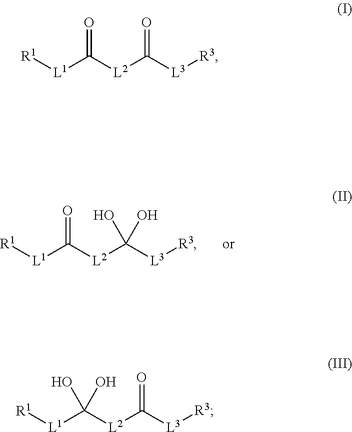

wherein
R¹ is a Switch II Binding Pocket binding moiety;
L¹ is a bond or divalent linker;
L² is a bond or substituted or unsubstituted alkylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is hydrogen, halogen, —CX³₃, —CHX³₂, —CH₂X³, —OCX³₃, —OCH₂X³, —OCHX³₂, —CN, —SOₙ₃R^{3D}, —SOv₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A} and R^{3B} substituents bonded to the same nitrogen atom may optionally be joined to form a substituted or unsubstituted heterocycloalkyl or substituted or unsubstituted heteroaryl;
each X³ is independently —Cl, —Br, —I, or —F;
n3 is an integer from 0 to 4; and
m3 and v3 are independently 1 or 2.

Embodiment P2. The compound of embodiment P1, wherein L² is a bond or unsubstituted C₁-C₄ alkylene.

Embodiment P3. The compound of one of embodiments P1 to P2, wherein L³ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P4. The compound of one of embodiments P1 to P2, wherein L³ is a bond.

Embodiment P5. The compound of one of embodiments P1 to P2, wherein L³ is —C(O)—.

Embodiment P6. The compound of embodiment P1, having the formula:

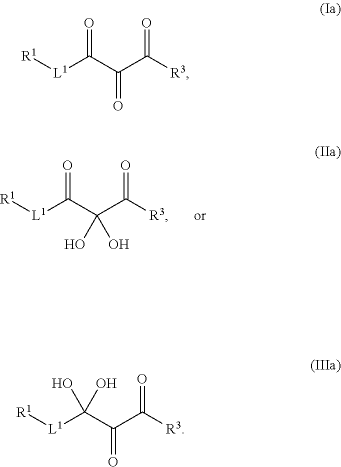

Embodiment P7. The compound of embodiment P1, having the formula:

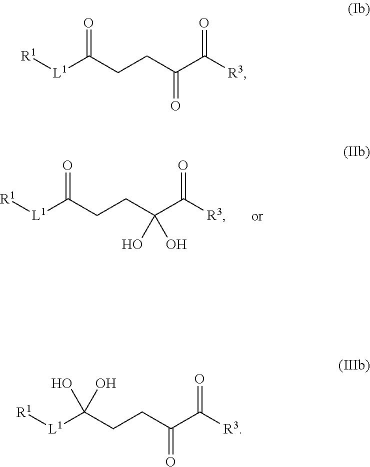

Embodiment P8. The compound of embodiment P1, having the formula:

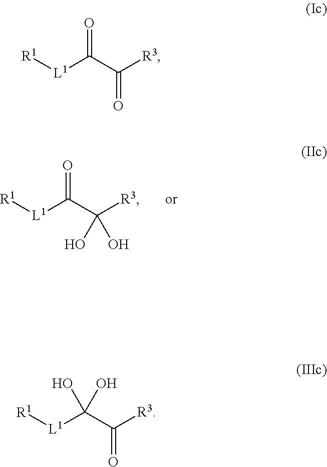

Embodiment P9. The compound of one of embodiments P1 to P8, wherein R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P10. The compound of one of embodiments P1 to P8, wherein R³ is hydrogen or unsubstituted C₁-C₄ alkyl.

Embodiment P11. The compound of one of embodiments P1 to P8, wherein R³ is hydrogen or unsubstituted methyl.

Embodiment P12. The compound of one of embodiments P1 to P11, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P13. The compound of one of embodiments P1 to P12, wherein L¹ is substituted or unsubstituted 3 to 8 membered heterocycloalkylene.

Embodiment P14. The compound of one of embodiments P1 to P12, wherein L¹ is

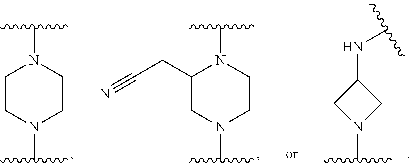

Embodiment P15. The compound of one of embodiments P1 to P14, wherein R¹ is -L¹⁰-R¹⁰;
L¹⁰ is a bond, —C(O)—, —C(O)O—, —OC(O)—, —O—, —S—, —NR¹⁰⁰—, —C(O)NR¹⁰⁰—, —NR¹⁰⁰C(O)—, —NR¹⁰⁰C(O)O—, —OC(O)NR¹⁰⁰—, —NR¹⁰⁰C(O)NR¹⁰⁰—, —NR¹⁰⁰C(NH)NR¹⁰⁰—, —S(O)₂—, —NR¹⁰⁰S(O)₂—, —S(O)₂NR¹⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
each X¹⁰ is independently —F, —Cl, —Br, or —I;
n10 is an integer from 0 to 4; and
m10 and v10 are independently 1 or 2.

Embodiment P16. The compound of one of embodiments P1 to P14, wherein R¹ is

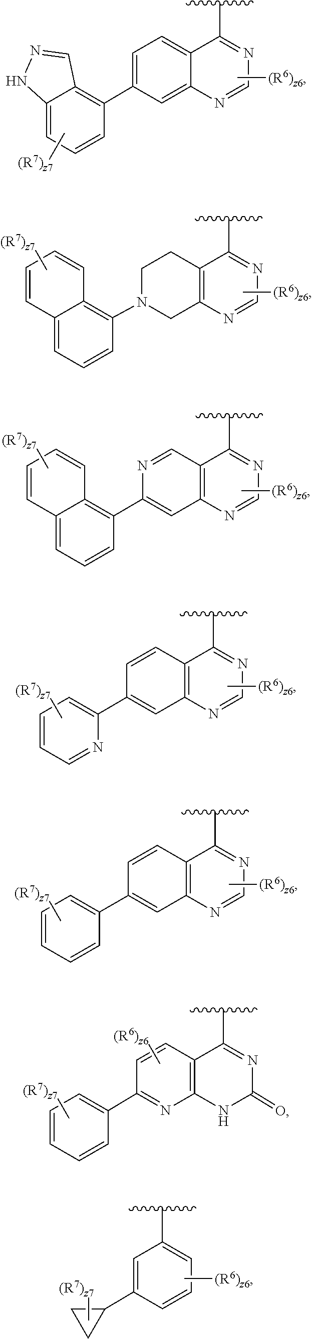

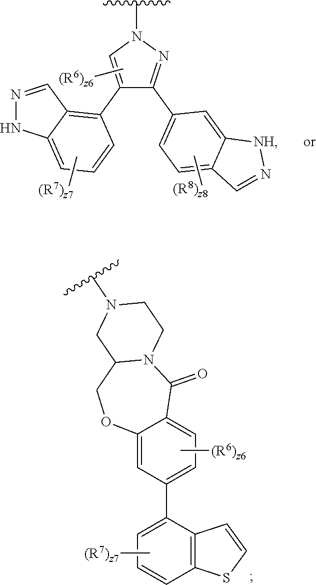

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

Embodiment P17. The compound of embodiment P16, wherein R⁶ is independently a halogen, —OH, unsubstituted C₁-C₄ alkyl, substituted 2 to 6 membered heteroalkyl, or substituted 5 to 6 membered heteroaryl.

Embodiment P18. The compound of embodiment P16, wherein R⁶ is independently —F, —Cl, —OH, or unsubstituted methyl.

Embodiment P19. The compound of embodiment P16, wherein R⁶ is independently a 2 to 6 membered heteroalkyl, substituted with substituted heterocycloalkyl or unsubstituted fused heterocycloalkyl.

Embodiment P20. The compound of embodiment P16, wherein R⁶ is independently a substituted pyridyl.

Embodiment P21. The compound of one of embodiments P16 to P20, wherein z6 is 1, 2, or 3.

Embodiment P22. The compound of one of embodiments P16 to P21, wherein R⁷ is independently a halogen, —CF₃, —CN, —OH, —NH₂, unsubstituted C₁-C₄ alkyl, or unsubstituted C₂-C₄ alkynyl.

Embodiment P23. The compound of one of embodiments P16 to P21, wherein R⁷ is independently —F, —Cl, —CF₃, —CN, —OH, —NH₂, unsubstituted methyl, or unsubstituted ethynyl.

Embodiment P24. The compound of one of embodiments P16 to P23, wherein z7 is 1, 2, or 3.

Embodiment P25. The compound of one of embodiments P16 to P24, wherein R⁸ is independently a halogen or unsubstituted C₁-C₄ alkyl.

Embodiment P26. The compound of one of embodiments P16 to P24, wherein R⁸ is independently —Cl or unsubstituted methyl.

Embodiment P27. The compound of one of embodiments P16 to P26, wherein z8 is 1.

Embodiment P28. The compound of one of embodiments P1 to P14, wherein R¹ is

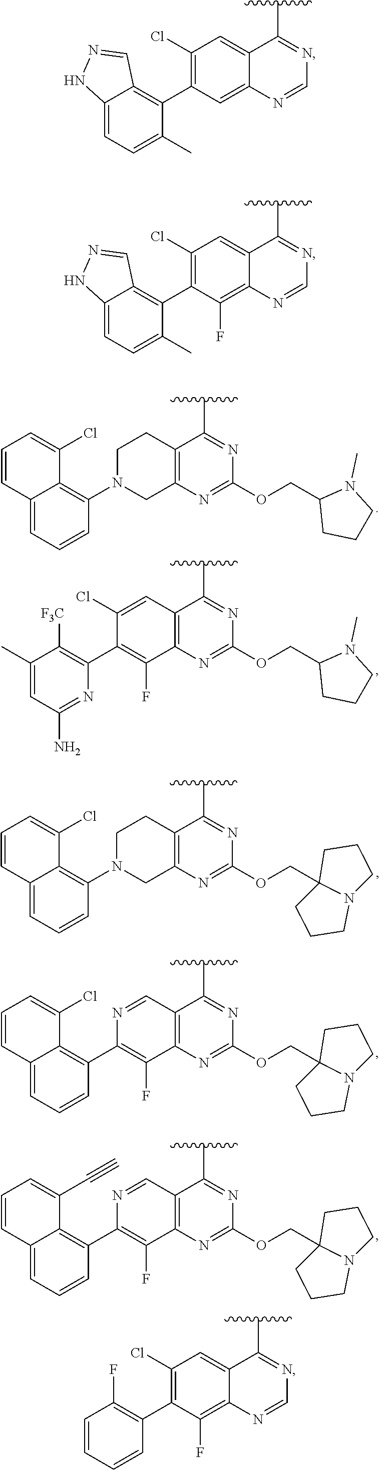

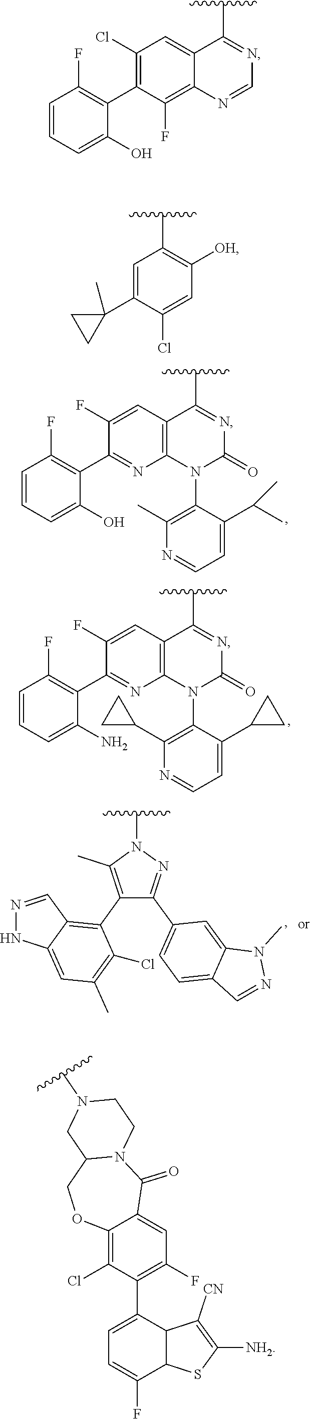

Embodiment P29. The compound of embodiment P1, having the formula:

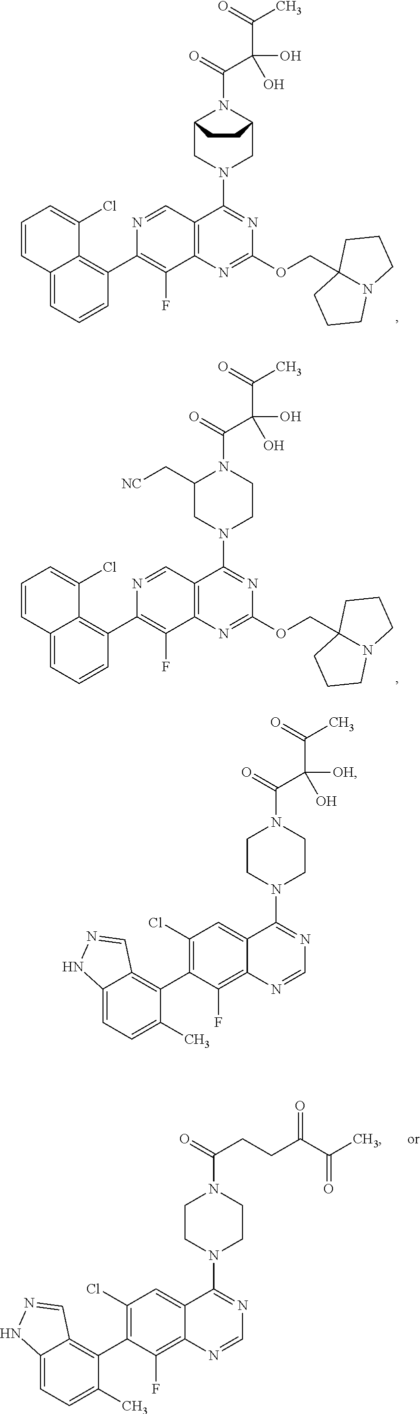

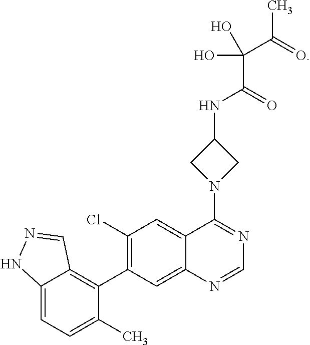 [SMILES: CC(=O)C(O)(O)C(=O)NC1CN(c2ncnc3cc(-c4c(C)ccc5[nH]ncc45)c(Cl)cc23)C1]

Embodiment P30. A pharmaceutical composition comprising a pharmaceutically acceptable excipient and a compound of one of embodiments P1 to P29, or a pharmaceutically acceptable salt thereof.

Embodiment P31. A method of treating cancer in a subject in need thereof, said method comprising administering to the subject in need thereof a therapeutically effective amount of a compound of one of embodiments P1 to P29, or a pharmaceutically acceptable salt thereof.

Embodiment P32. The method of embodiment P31, wherein the cancer is pancreatic cancer, lung cancer, or colorectal cancer.

Embodiment P33. A method of modulating the level of activity of a Ras protein in a cell, said method comprising contacting the cell with an effective amount of a compound of one of embodiments P1 to P29, or a pharmaceutically acceptable salt thereof.

Embodiment P34. The method of embodiment P33, wherein said modulating of said activity comprises modulating GTPase activity, nucleotide exchange, differential GDP or GTP binding, effector protein binding, effector protein activation, guanine exchange factor (GEF) binding, GEF-facilitated nucleotide exchange, phosphate release, nucleotide release, nucleotide binding, Ras subcellular localization, Ras post-translational processing, or Ras post-translational modifications.

Embodiment P35. The method of one of embodiments P33 to P34, wherein said modulating is increasing the activity of said Ras protein.

Embodiment P36. The method of one of embodiments P33 to P34, wherein said modulating is reducing the activity of said Ras protein.

Embodiment P37. The method of one of embodiments P33 to P36, wherein said Ras protein is a human K-Ras protein.

Embodiment P38. The method of embodiment P37, wherein said human K-Ras protein contains a G12R mutation, a G13R mutation, or a Q61R mutation.

Embodiment P39. The method of one of embodiments P33 to P36, wherein said Ras protein is a human H-Ras protein.

Embodiment P40. The method of embodiment P39, wherein said human H-Ras protein contains a G12R mutation, a G13R mutation, or a Q61R mutation.

Embodiment P41. The method of one of embodiments P33 to P36, wherein said Ras protein is a human N-Ras protein.

Embodiment P42. The method of embodiment P41, wherein said human N-Ras protein contains a G12R mutation, a G13R mutation, or a Q61R mutation.

Embodiment P43. A Switch II GTPase protein covalently bound to a compound of one of embodiments P1 to P29, or a salt thereof, wherein said compound is covalently bound to an arginine residue of said Switch II GTPase protein.

Embodiment P44. The covalently modified Switch II GTPase protein of embodiment P43, wherein said compound is reversibly covalently bound to an arginine residue of said Switch II GTPase protein.

Embodiment P45. The covalently modified Switch II GTPase protein of embodiment P43, wherein said compound is irreversibly covalently bound to an arginine residue of said Switch II GTPase protein.

Embodiment P46. The covalently modified Switch II GTPase protein of one of embodiments P43 to P45, wherein said Switch II GTPase protein is a human K-Ras protein.

Embodiment P47. The covalently modified Switch II GTPase protein of embodiment P46, wherein said human K-Ras protein contains a G12R mutation.

Embodiment P48. The covalently modified Switch II GTPase protein of embodiment P47, wherein said compound is covalently bonded to arginine residue 12.

Embodiment P49. The covalently modified Switch II GTPase protein of one of embodiments P43 to P45, wherein said Switch II GTPase protein is a human H-Ras protein.

Embodiment P50. The covalently modified Switch II GTPase protein of embodiment P49, wherein said human H-Ras protein contains a G12R mutation.

Embodiment P51. The covalently modified Switch II GTPase protein of embodiment P50, wherein said compound is covalently bonded to arginine residue 12.

Embodiment P52. The covalently modified Switch II GTPase protein of one of embodiments P43 to P45, wherein said Switch II GTPase protein is a human N-Ras protein.

Embodiment P53. The covalently modified Switch II GTPase protein of embodiment P52, wherein said human N-Ras protein contains a G12R mutation.

Embodiment P54. The covalently modified Switch II GTPase protein of embodiment P53, wherein said compound is covalently bonded to arginine residue 12.

Embodiment P55. A method of attaching a compound to an arginine residue of a protein, said method comprising contacting said compound with said arginine residue, wherein said compound has the formula:

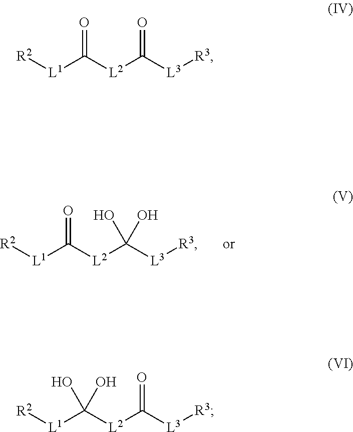

or a salt thereof, wherein
R² is a Switch II Binding Pocket binding moiety, a phosphatase PTP domain binding moiety, an SH2 domain binding moiety, a pseudokinase KSR domain binding moiety, or a pseudokinase STRADα domain binding moiety;
L¹ is a bond or divalent linker; and
L² is a bond or substituted or unsubstituted alkylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A} and R^{3B} substituents bonded to the same nitrogen atom may optionally be joined to form a substituted or unsubstituted heterocycloalkyl or substituted or unsubstituted heteroaryl; each X³ is independently —Cl, —Br, —I, or —F;
n3 is an integer from 0 to 4; and
m3 and v3 are independently 1 or 2.

Embodiment P56. The method of embodiment P55, wherein the protein further comprises additional arginine residues and none of the additional arginine residues react with the compound.

Embodiment P57. The method of one of embodiments P55 to P56, wherein L² is a bond or unsubstituted C₁-C₄ alkylene.

Embodiment P58. The method of one of embodiments P55 to P57, wherein L³ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P59. The method of one of embodiments P55 to P57, wherein L³ is a bond.

Embodiment P60. The method of one of embodiments P55 to P57, wherein L³ is —C(O)—.

Embodiment P61. The method of embodiment P55, wherein the compound has the formula:

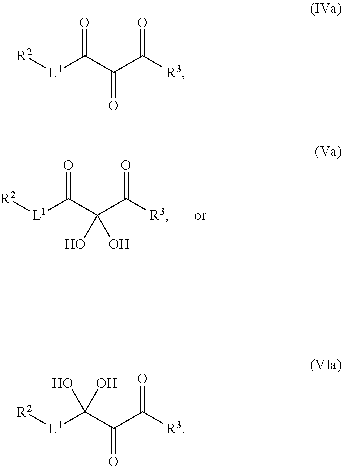

Embodiment P62. The method of embodiment P55, wherein the compound has the formula:

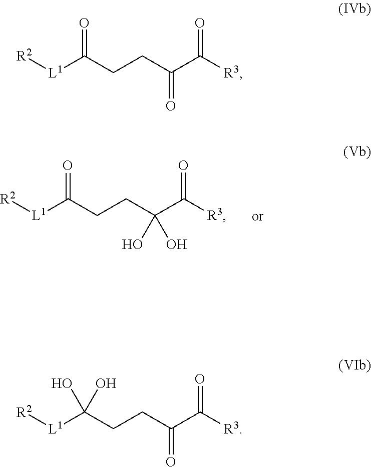

Embodiment P63. The method of embodiment P55, wherein the compound has the formula:

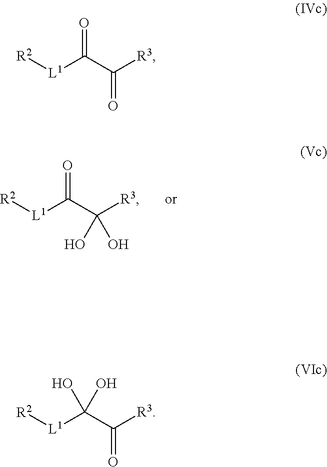

Embodiment P64. The method of one of embodiments P55 to P63, wherein R³ is hydrogen or unsubstituted C₁-C₄ alkyl.

Embodiment P65. The method of one of embodiments P55 to P63, wherein R³ is hydrogen or unsubstituted methyl.

Embodiment P66. The method of one of embodiments P55 to P65, wherein

L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NHI)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P67. The method of one of embodiments P55 to P65, wherein L¹ is a bond, substituted or unsubstituted 3 to 8 membered heterocycloalkylene, or substituted or unsubstituted phenylene.

Embodiment P68. The method of one of embodiments P55 to P65, wherein L¹ is

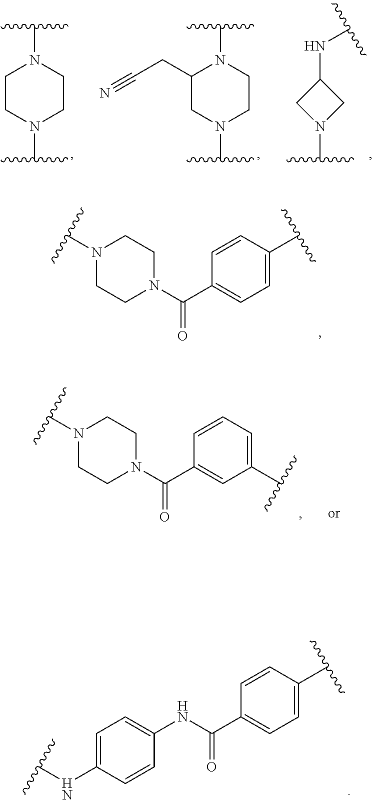

Embodiment P69. The method of one of embodiments P55 to P68, wherein R² is -L²⁰-R²⁰.

L²⁰ is a bond, —C(O)—, —C(O)O—, —OC(O)—, —O—, —S—, —NR²⁰⁰—, —C(O)NR²⁰⁰—, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is hydrogen, halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, —N(O)ₘ₂₀, —NR^{20A}R^{20B}, —C(O)R^{20C}, —C(O)OR^{20C}, —C(O)NR^{20A}R^{20B}, —OR^{20D}, —SR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
each X²⁰ is independently —F, —Cl, —Br, or —I;
n20 is an integer from 0 to 4; and
m20 and v20 are independently 1 or 2.

Embodiment P70. The method of one of embodiments P55 to P68, wherein R² is

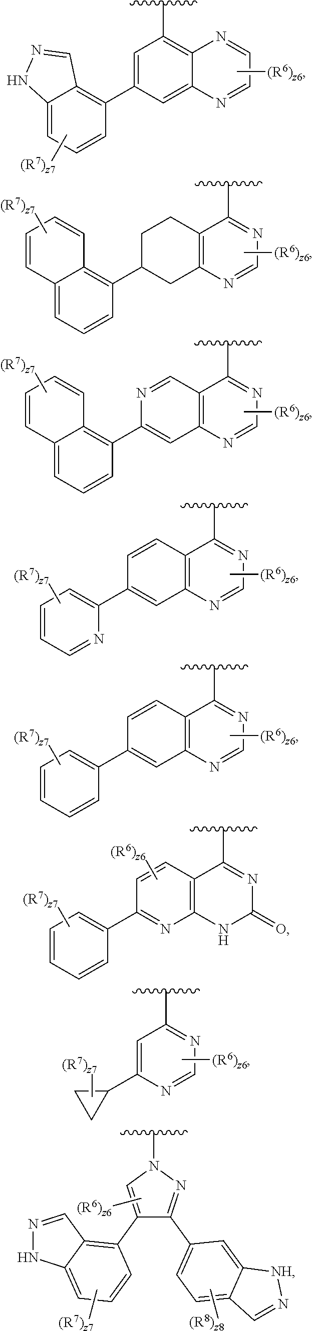

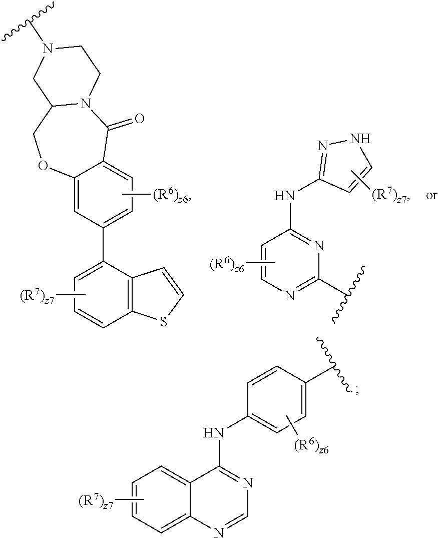

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and z8 is an integer from 0 to 5.

Embodiment P71. The method of embodiment P70, wherein R⁶ is independently a halogen, —OH, unsubstituted C₁-C₄ alkyl, substituted 2 to 6 membered heteroalkyl, or substituted 5 to 6 membered heteroaryl.

Embodiment P72. The method of embodiment P70, wherein R⁶ is independently —F, —Cl, —OH, or unsubstituted methyl.

Embodiment P73. The method of embodiment P70, wherein R⁶ is independently a 2 to 6 membered heteroalkyl, substituted with substituted heterocycloalkyl or unsubstituted fused heterocycloalkyl.

Embodiment P74. The method of embodiment P70, wherein R⁶ is independently a substituted pyridyl.

Embodiment P75. The method of one of embodiments P70 to P74, wherein z6 is 1, 2, or 3.

Embodiment P76. The method of one of embodiments P70 to P75, wherein R⁷ is independently a halogen, —CF₃, —CN, —OH, —NH₂, unsubstituted C₁-C₄ alkyl, unsubstituted C₂-C₄ alkynyl, unsubstituted 2 to 6 membered heteroalkyl, or unsubstituted C₃-C₈ cycloalkyl.

Embodiment P77. The method of one of embodiments P70 to P75, wherein R⁷ is independently —F, —Cl, —CF₃, —CN, —OH, —NH₂, unsubstituted methyl, unsubstituted ethynyl, unsubstituted methoxy, or unsubstituted cyclopropyl.

Embodiment P78. The method of one of embodiments P70 to P77, wherein z7 is 1, 2, or 3.

Embodiment P79. The method of one of embodiments P70 to P78, wherein R⁸ is independently a halogen or unsubstituted C₁-C₄ alkyl.

Embodiment P80. The method of one of embodiments P70 to P78, wherein R⁸ is independently —Cl or unsubstituted methyl.

Embodiment P81. The method of one of embodiments P70 to P80, wherein z8 is 1.

Embodiment P82. The method of one of embodiments P55 to P68, wherein R² is

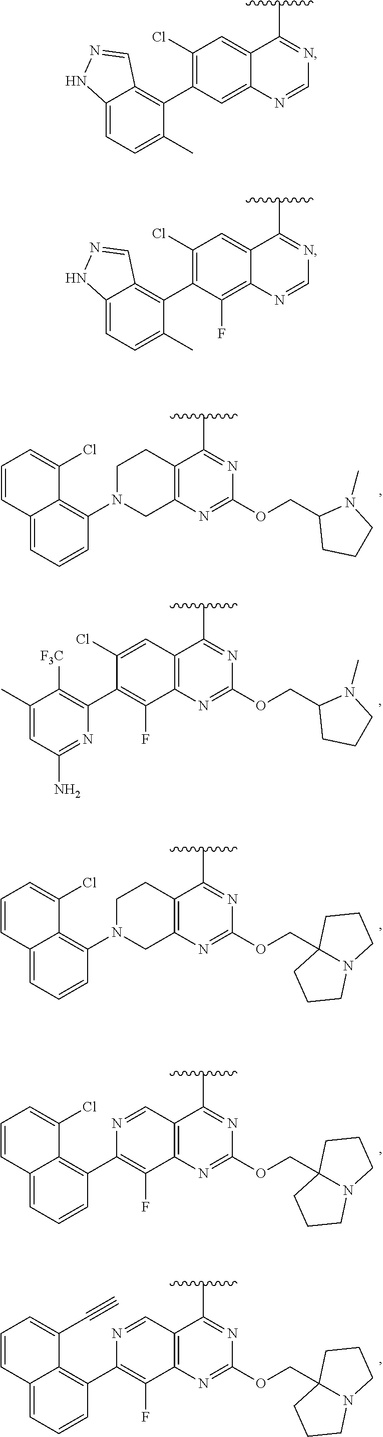

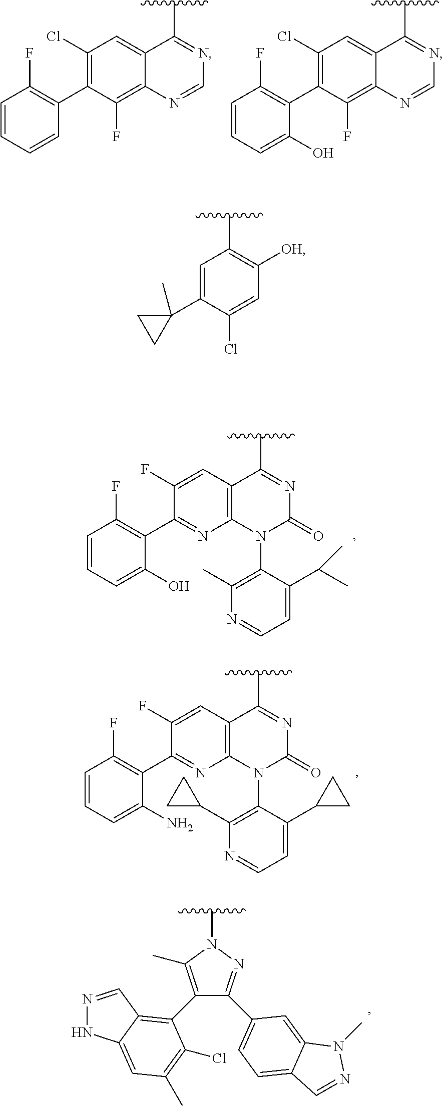

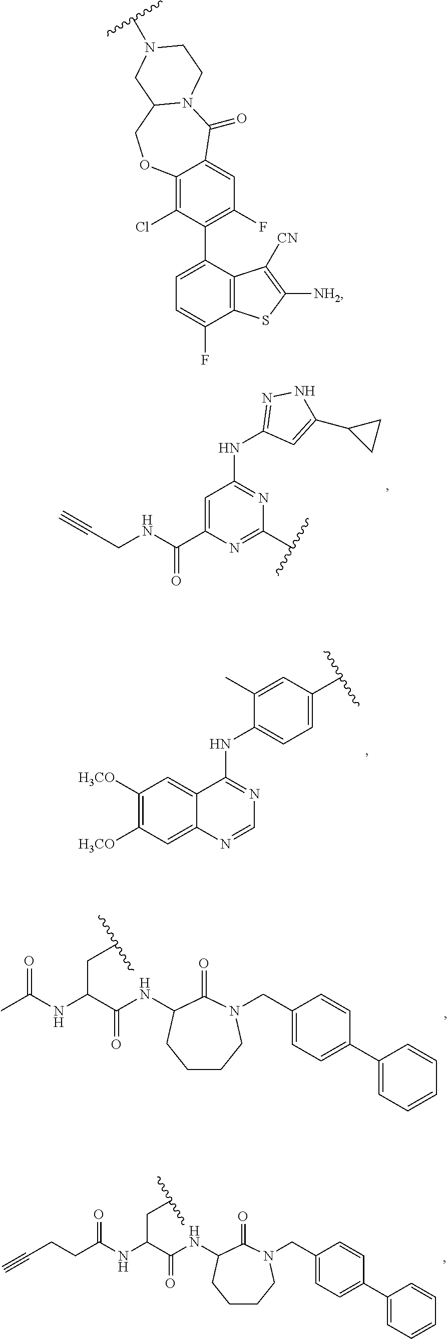 [SMILES: *N1CCN2C(=O)c3cc(F)c(-c4ccc(F)c5sc(N)c(C#N)c45)c(Cl)c3OCC2C1 ; *c1nc(Nc2cc(C3CC3)[nH]n2)cc(C(=O)NCC#C)n1 ; *c1ccc(Nc2ncnc3cc(OC)c(OC)cc23)c(C)c1 ; *CC(NC(C)=O)C(=O)NC1CCCCN(Cc2ccc(-c3ccccc3)cc2)C1=O ; *CC(NC(=O)CCC#C)C(=O)NC1CCCCN(Cc2ccc(-c3ccccc3)cc2)C1=O]

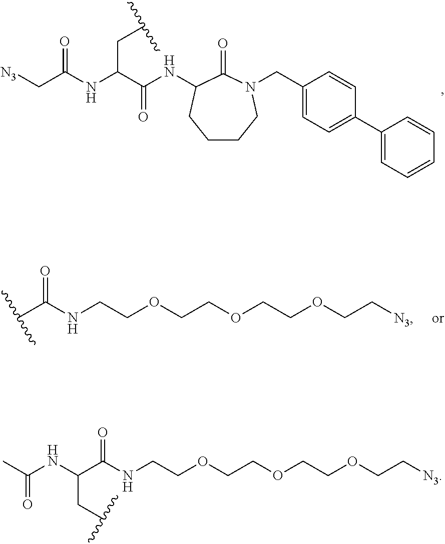 [SMILES: *CC(NC(=O)CN=[N+]=[N-])C(=O)NC1CCCCN(Cc2ccc(-c3ccccc3)cc2)C1=O ; *C(=O)NCCOCCOCCOCCN=[N+]=[N-] ; *CC(NC(C)=O)C(=O)NCCOCCOCCOCCN=[N+]=[N-]]

Embodiment P83. A method of attaching a compound to an arginine residue, said method comprising contacting said compound with said arginine residue, wherein said compound has the formula:

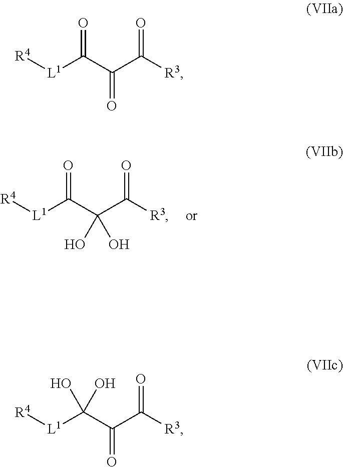

or a salt thereof; wherein
L¹ is a bond or divalent linker;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —N^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{3A}, R^{3B}, R^{3C}, R^{3D}, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X³ and X⁴ is independently —Cl, —Br, —I, or —F;
n3 and n4 are independently an integer from 0 to 4; and m3, m4, v3, and v4 are independently 1 or 2.

Embodiment P84. The method of embodiment P83, wherein said arginine residue forms part of a protein.

Embodiment P85. The method of one of embodiments P83 to P84, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P86. The method of one of embodiments P83 to P85, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P87. The method of one of embodiments P83 to P85, wherein R⁴ is a biomolecular moiety.

Embodiment P88. A compound covalently bound to an arginine residue, having the formula:

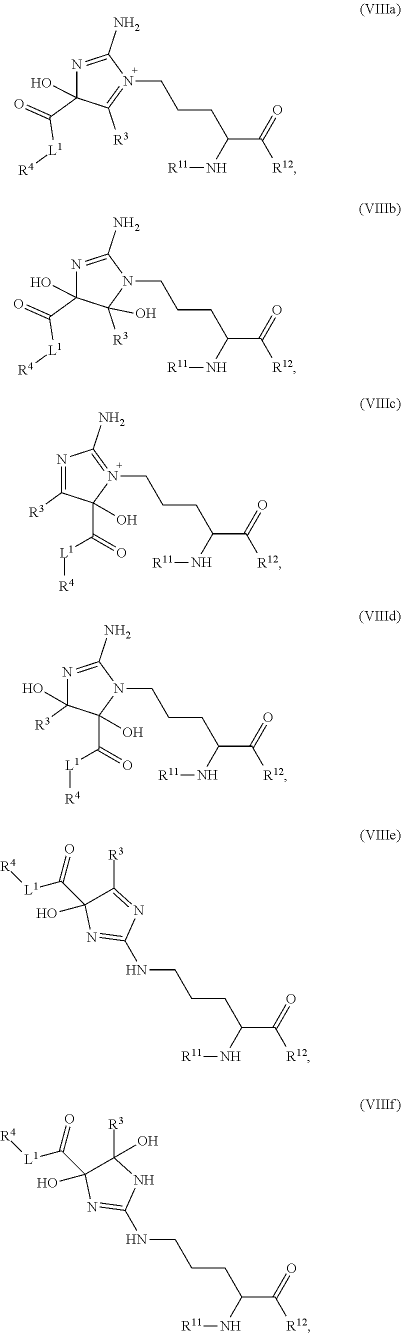

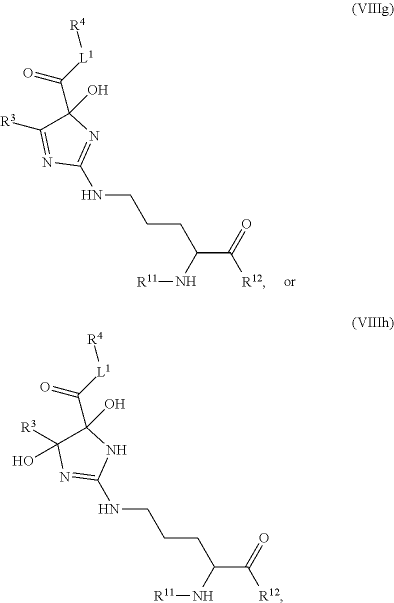

or a salt thereof; wherein
L¹ is a bond or divalent linker;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —N^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —N^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{3A}, R^{3B}, R^{3C}, R^{3D}, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X³ and X⁴ is independently —Cl, —Br, —I, or —F;
n3 and n4 are independently an integer from 0 to 4;
m3, m4, v3, and v4 are independently 1 or 2;
R¹¹ is hydrogen, —C(O)CH₃, or a first protein moiety; and
R¹² is —OH or a second protein moiety.

Embodiment P89. The covalently bound compound of embodiment P88, wherein the first protein moiety and the second protein moiety together form a single protein.

Embodiment P90. The covalently bound compound of embodiment P89, wherein the single protein comprises additional arginine residues that are not covalently bound to a compound to form a reacted arginine having the formula (VIa), (VIb), (VIc), (VId), (VIe), (VIf), (VIg), or (VIh).

Embodiment P91. The covalently bound compound of one of embodiments P88 to P90, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(o)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P92. The covalently bound compound of one of embodiments P88 to P91, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P93. The covalently bound compound of one of embodiments P88 to P91, wherein R⁴ is a biomolecular moiety.

### VIII. Additional Embodiments

Embodiment 1. A compound, or a pharmaceutically acceptable salt thereof, having the formula:

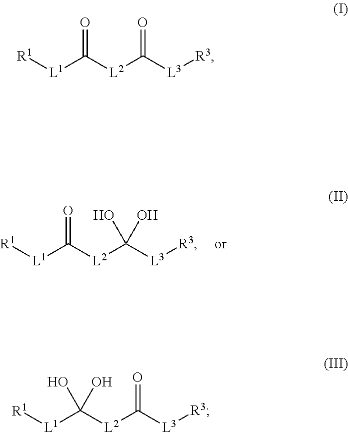

wherein
R¹ is a Switch II Binding Pocket binding moiety;
L¹ is a bond or divalent linker;
L² is a bond or substituted or unsubstituted alkylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A} and R^{3B} substituents bonded to the same nitrogen atom may optionally be joined to form a substituted or unsubstituted heterocycloalkyl or substituted or unsubstituted heteroaryl;
each X³ is independently —Cl, —Br, —I, or —F;
n3 is an integer from 0 to 4; and
m3 and v3 are independently 1 or 2.

Embodiment 2. The compound of embodiment 1, wherein L² is a bond or unsubstituted C₁-C₄ alkylene.

Embodiment 3. The compound of one of embodiments 1 to 2, wherein L³ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 4. The compound of one of embodiments 1 to 2, wherein L³ is a bond.

Embodiment 5. The compound of one of embodiments 1 to 2, wherein L³ is —C(O)—.

Embodiment 6. The compound of embodiment 1, having the formula:

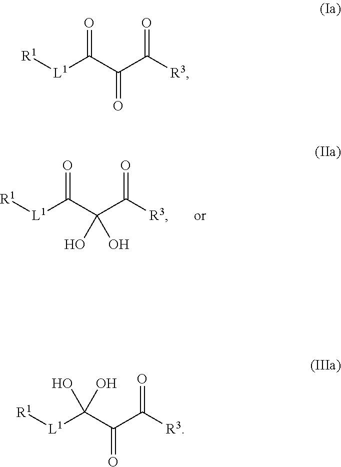

Embodiment 7. The compound of embodiment 1, having the formula:

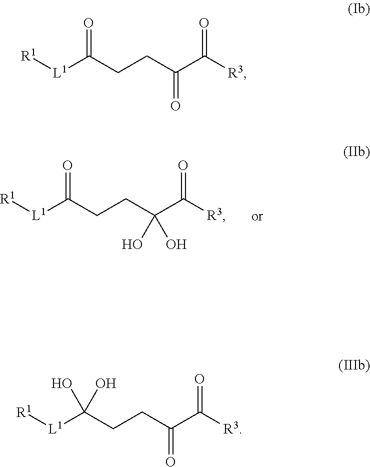

Embodiment 8. The compound of embodiment 1, having the formula:

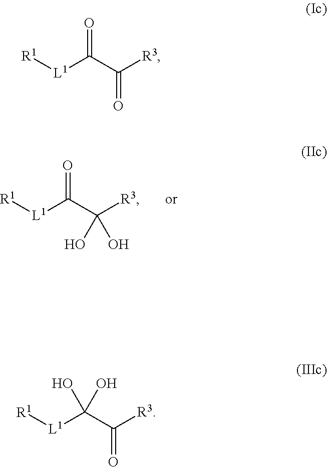

Embodiment 9. The compound of one of embodiments 1 to 8, wherein R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 10. The compound of one of embodiments 1 to 8, wherein R³ is hydrogen or unsubstituted C₁-C₄ alkyl.

Embodiment 11. The compound of one of embodiments 1 to 8, wherein R³ is hydrogen or unsubstituted methyl.

Embodiment 12. The compound of one of embodiments 1 to 11, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(o)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 13. The compound of one of embodiments 1 to 12, wherein L¹ is substituted or unsubstituted 3 to 8 membered heterocycloalkylene.

Embodiment 14. The compound of one of embodiments 1 to 12, wherein L¹ is

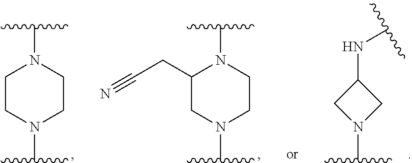

Embodiment 15. The compound of one of embodiments 1 to 14, wherein R¹ is -L¹⁰-R¹⁰;
L¹⁰ is a bond, —C(O)—, —C(O)O—, —OC(O)—, —O—, —S—, —NR¹⁰⁰—, —C(O)NR¹⁰⁰—, —NR¹⁰⁰C(O)—, —NR¹⁰⁰C(O)O—, —OC(O)NR¹⁰⁰—, —NR¹⁰⁰C(O)NR¹⁰⁰—, —NR¹⁰⁰C(NH)NR¹⁰⁰—, —S(O)₂—, —NR¹⁰⁰S(O)₂—, —S(O)₂NR¹⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰², —CN, —SOₙ₁₀R^{10D}, —SOᵥ₁₀NR^{10A}R^{10B}, —NR^{10C}NR^{10A}R^{10B}, —NR^{10A}R^{10B}, —NHC(O)NR^{10C}NR^{10A}R^{10B}, —NHC(O)NR^{10A}R^{10B, —N(O)}ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
each X¹⁰ is independently —F, —Cl, —Br, or —I;
n10 is an integer from 0 to 4; and
m10 and v10 are independently 1 or 2.

Embodiment 16. The compound of one of embodiments 1 to 14, wherein R¹ is

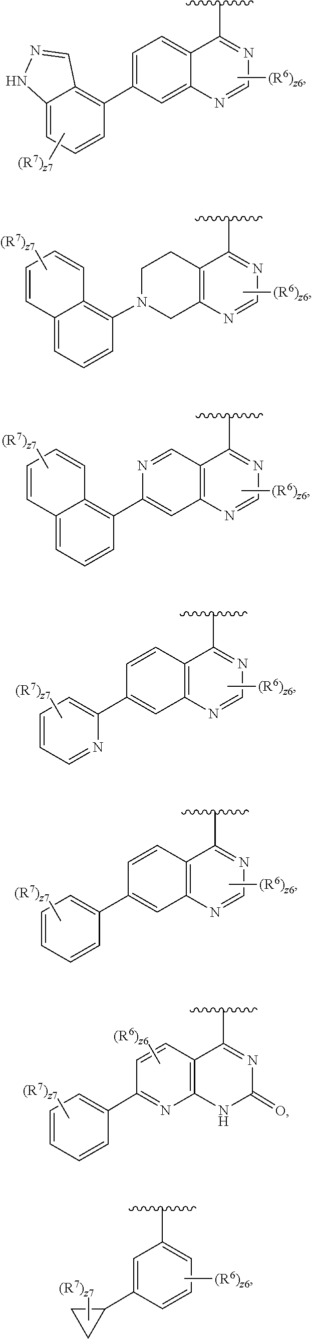

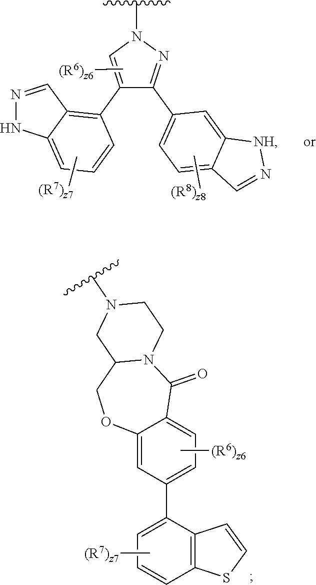

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

Embodiment 17. The compound of embodiment 16, wherein R⁶ is independently a halogen, —OH, unsubstituted C₁-C₄ alkyl, substituted 2 to 6 membered heteroalkyl, or substituted 5 to 6 membered heteroaryl.

Embodiment 18. The compound of embodiment 16, wherein R⁶ is independently —F, —Cl, —OH, or unsubstituted methyl.

Embodiment 19. The compound of embodiment 16, wherein R⁶ is independently a 2 to 6 membered heteroalkyl, substituted with substituted heterocycloalkyl or unsubstituted fused heterocycloalkyl.

Embodiment 20. The compound of embodiment 16, wherein R⁶ is independently a substituted pyridyl.

Embodiment 21. The compound of one of embodiments 16 to 20, wherein z6 is 1, 2, or 3.

Embodiment 22. The compound of one of embodiments 16 to 21, wherein R⁷ is independently a halogen, —CF₃, —CN, —OH, —NH₂, unsubstituted C₁-C₄ alkyl, or unsubstituted C₂-C₄ alkynyl.

Embodiment 23. The compound of one of embodiments 16 to 21, wherein R⁷ is independently —F, —Cl, —CF₃, —CN, —OH, —NH₂, unsubstituted methyl, or unsubstituted ethynyl.

Embodiment 24. The compound of one of embodiments 16 to 23, wherein z7 is 1, 2, or 3.

Embodiment 25. The compound of one of embodiments 16 to 24, wherein R⁸ is independently a halogen or unsubstituted C₁-C₄ alkyl.

Embodiment 26. The compound of one of embodiments 16 to 24, wherein R⁸ is independently —Cl or unsubstituted methyl.

Embodiment 27. The compound of one of embodiments 16 to 26, wherein z8 is 1.

Embodiment 28. The compound of one of embodiments 1 to 14, wherein R¹ is

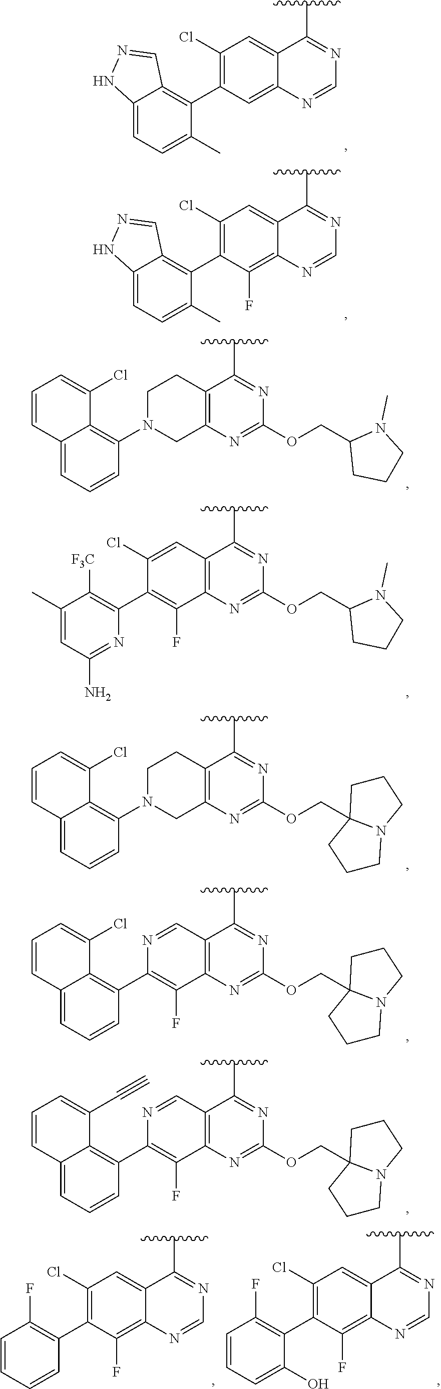

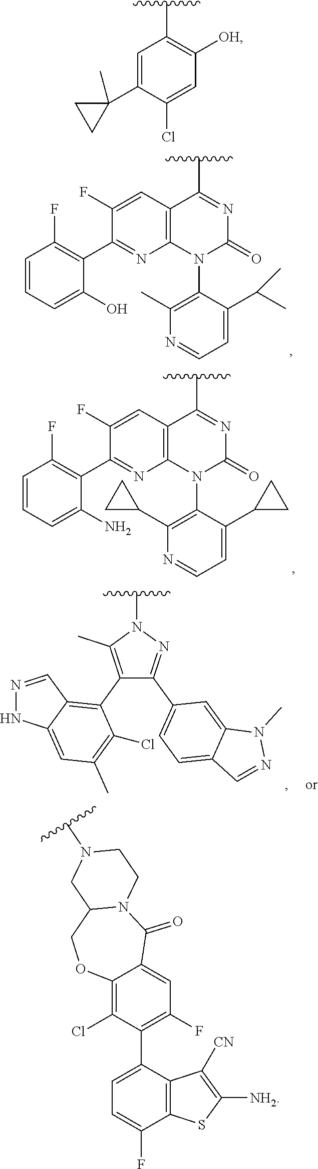

Embodiment 29. The compound of embodiment 1, having the formula:

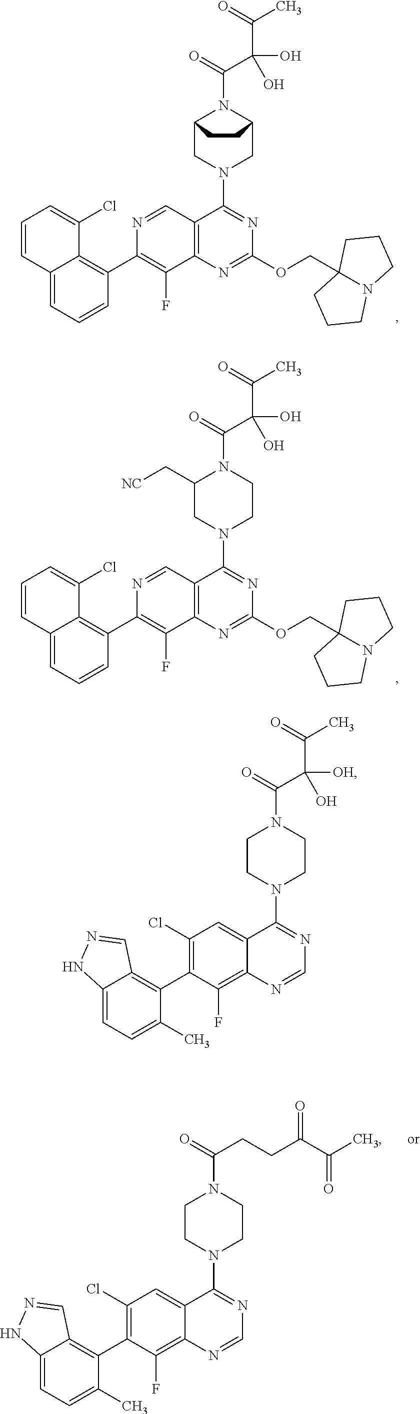

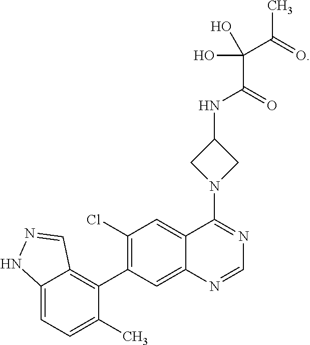 [SMILES: CC(=O)C(O)(O)C(=O)NC1CN(c2ncnc3cc(-c4c(C)ccc5[nH]ncc45)c(Cl)cc23)C1]

Embodiment 30. A pharmaceutical composition comprising a pharmaceutically acceptable excipient and a compound of one of embodiments 1 to 29, or a pharmaceutically acceptable salt thereof.

Embodiment 31. A method of treating cancer in a subject in need thereof, said method comprising administering to the subject in need thereof a therapeutically effective amount of a compound of one of embodiments 1 to 29, or a pharmaceutically acceptable salt thereof.

Embodiment 32. The method of embodiment 31, wherein the cancer is pancreatic cancer, lung cancer, or colorectal cancer.

Embodiment 33. A method of reducing Ras protein-mediated activity in a cell, said method comprising contacting the cell with an effective amount of a compound of one of embodiments 1 to 29, or a pharmaceutically acceptable salt thereof.

Embodiment 34. The method of embodiment 33, wherein said Ras protein is a human K-Ras protein.

Embodiment 35. The method of embodiment 34, wherein said human K-Ras protein contains a G12R mutation, a G13R mutation, or a Q61R mutation.

Embodiment 36. The method of embodiment 33, wherein said Ras protein is a human H-Ras protein.

Embodiment 37. The method of embodiment 36, wherein said human H-Ras protein contains a G12R mutation, a G13R mutation, or a Q61R mutation.

Embodiment 38. The method of embodiment 33, wherein said Ras protein is a human N-Ras protein.

Embodiment 39. The method of embodiment 38, wherein said human N-Ras protein contains a G12R mutation, a G13R mutation, or a Q61R mutation.

Embodiment 40. A Switch II GTPase protein covalently bound to a compound of one of embodiments 1 to 29, or a salt thereof, wherein said compound is covalently bound to an arginine residue of said Switch II GTPase protein.

Embodiment 41. The covalently modified Switch II GTPase protein of embodiment 40, wherein said compound is reversibly covalently bound to an arginine residue of said Switch II GTPase protein.

Embodiment 42. The covalently modified Switch II GTPase protein of embodiment 40, wherein said compound is irreversibly covalently bound to an arginine residue of said Switch II GTPase protein.

Embodiment 43. The covalently modified Switch II GTPase protein of one of embodiments 40 to 42, wherein said Switch II GTPase protein is a human K-Ras protein.

Embodiment 44. The covalently modified Switch II GTPase protein of embodiment 43, wherein said human K-Ras protein contains a G12R mutation.

Embodiment 45. The covalently modified Switch II GTPase protein of embodiment 44, wherein said compound is covalently bonded to arginine residue 12.

Embodiment 46. The covalently modified Switch II GTPase protein of one of embodiments 40 to 42, wherein said Switch II GTPase protein is a human H-Ras protein.

Embodiment 47. The covalently modified Switch II GTPase protein of embodiment 46, wherein said human H-Ras protein contains a G12R mutation.

Embodiment 48. The covalently modified Switch II GTPase protein of embodiment 47, wherein said compound is covalently bonded to arginine residue 12.

Embodiment 49. The covalently modified Switch II GTPase protein of one of embodiments 40 to 42, wherein said Switch II GTPase protein is a human N-Ras protein.

Embodiment 50. The covalently modified Switch II GTPase protein of embodiment 49, wherein said human N-Ras protein contains a G12R mutation.

Embodiment 51. The covalently modified Switch II GTPase protein of embodiment 50, wherein said compound is covalently bonded to arginine residue 12.

Embodiment 52. A method of attaching a compound to an arginine residue of a protein, said method comprising contacting said compound with said arginine residue, wherein said compound has the formula:

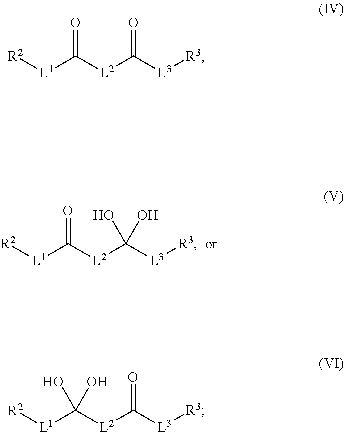

or a salt thereof, wherein
R² is a Switch II Binding Pocket binding moiety, a phosphatase PTP domain binding moiety, an SH2 domain binding moiety, a pseudokinase KSR domain binding moiety, or a pseudokinase STRADα domain binding moiety;
L¹ is a bond or divalent linker; and
L² is a bond or substituted or unsubstituted alkylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A} and R^{3B} substituents bonded to the same nitrogen atom may optionally be joined to form a substituted or unsubstituted heterocycloalkyl or substituted or unsubstituted heteroaryl;
each X³ is independently —Cl, —Br, —I, or —F;
n3 is an integer from 0 to 4; and
m3 and v3 are independently 1 or 2.

Embodiment 53. The method of embodiment 52, wherein the protein further comprises additional arginine residues and none of the additional arginine residues react with the compound.

Embodiment 54. The method of one of embodiments 52 to 53, wherein L² is a bond or unsubstituted C₁-C₄ alkylene.

Embodiment 55. The method of one of embodiments 52 to 54, wherein L³ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —NHC(NH)NH—, —S(O)₂—, —NHS(O)₂—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 56. The method of one of embodiments 52 to 54, wherein L³ is a bond.

Embodiment 57. The method of one of embodiments 52 to 54, wherein L³ is —C(O)—.

Embodiment 58. The method of embodiment 52, wherein the compound has the formula:

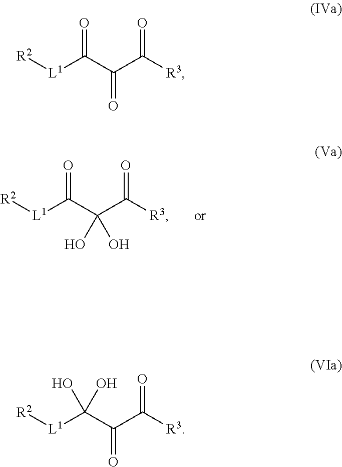

Embodiment 59. The method of embodiment 52, wherein the compound has the formula:

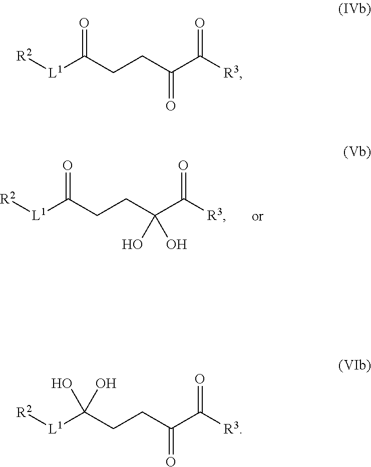

Embodiment 60. The method of embodiment 52, wherein the compound has the formula:

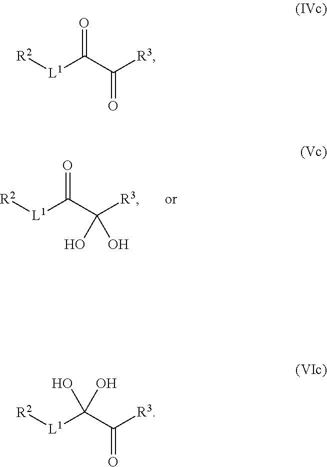

Embodiment 61. The method of one of embodiments 52 to 60, wherein R³ is hydrogen or unsubstituted C₁-C₄ alkyl.

Embodiment 62. The method of one of embodiments 52 to 60, wherein R³ is hydrogen or unsubstituted methyl.

Embodiment 63. The method of one of embodiments 52 to 62, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 64. The method of one of embodiments 52 to 62, wherein L¹ is a bond, substituted or unsubstituted 3 to 8 membered heterocycloalkylene, or substituted or unsubstituted phenylene.

Embodiment 65. The method of one of embodiments 52 to 62, wherein L¹ is

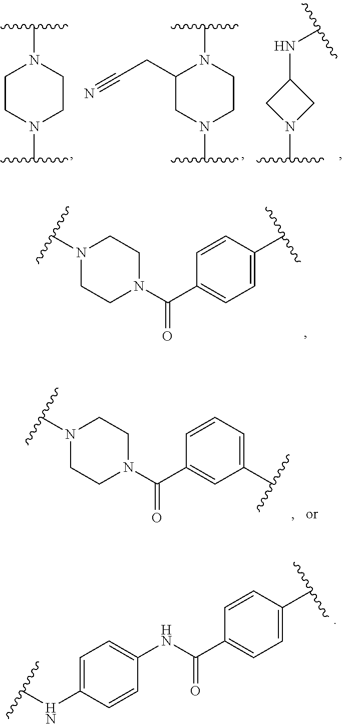

Embodiment 66. The method of one of embodiments 52 to 65, wherein R² is -L²⁰-R²⁰;
L²⁰ is a bond, —C(O)—, —C(O)O—, —OC(O)—, —O—, —S—, —NR²⁰⁰—, —C(O)NR²⁰⁰—, —NR²⁰⁰C(O)—, —NR²⁰⁰C(O)O—, —OC(O)NR²⁰⁰—, —NR²⁰⁰C(O)NR²⁰⁰—, —NR²⁰⁰C(NH)NR²⁰⁰—, —S(O)₂—, —NR²⁰⁰S(O)₂—, —S(O)₂NR²⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is hydrogen, halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, N(O)ₘ₂₀, —NR^{20A}R^{20B}, —C(O)R^{20C}, —C(O)OR^{20C}, —C(O)NR^{20A}R^{20B}, —OR^{20D}, —SR^{20D}, —OR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
each X²⁰ is independently —F, —Cl, —Br, or —I;
n20 is an integer from 0 to 4; and m20 and v20 are independently 1 or 2.

Embodiment 67. The method of one of embodiments 52 to 65, wherein R² is

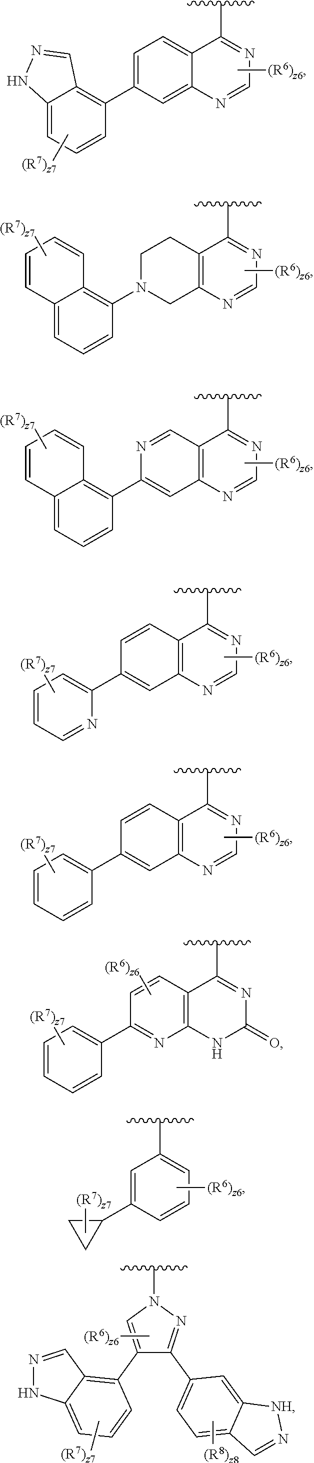

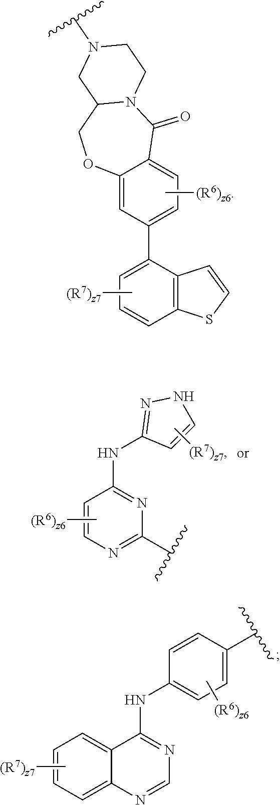

R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

Embodiment 68. The method of embodiment 67, wherein R⁶ is independently a halogen, —OH, unsubstituted C₁-C₄ alkyl, substituted 2 to 6 membered heteroalkyl, or substituted 5 to 6 membered heteroaryl.

Embodiment 69. The method of embodiment 67, wherein R⁶ is independently —F, —Cl, —OH, or unsubstituted methyl.

Embodiment 70. The method of embodiment 67, wherein R⁶ is independently a 2 to 6 membered heteroalkyl, substituted with substituted heterocycloalkyl or unsubstituted fused heterocycloalkyl.

Embodiment 71. The method of embodiment 67, wherein R⁶ is independently a substituted pyridyl.

Embodiment 72. The method of one of embodiments 67 to 71, wherein z6 is 1, 2, or 3.

Embodiment 73. The method of one of embodiments 67 to 72, wherein R⁷ is independently a halogen, —CF₃, —CN, —OH, —NH₂, unsubstituted C₁-C₄ alkyl, unsubstituted C₂-C₄ alkynyl, unsubstituted 2 to 6 membered heteroalkyl, or unsubstituted C₃-C₈ cycloalkyl.

Embodiment 74. The method of one of embodiments 67 to 72, wherein R⁷ is independently —F, —Cl, —CF₃, —CN, —OH, —NH₂, unsubstituted methyl, unsubstituted ethynyl, unsubstituted methoxy, or unsubstituted cyclopropyl.

Embodiment 75. The method of one of embodiments 67 to 74, wherein z7 is 1, 2, or 3.

Embodiment 76. The method of one of embodiments 67 to 75, wherein R⁸ is independently a halogen or unsubstituted C₁-C₄ alkyl.

Embodiment 77. The method of one of embodiments 67 to 75, wherein R⁸ is independently —Cl or unsubstituted methyl.

Embodiment 78. The method of one of embodiments 67 to 77, wherein z8 is 1.

Embodiment 79. The method of one of embodiments 52 to 65, wherein R² is

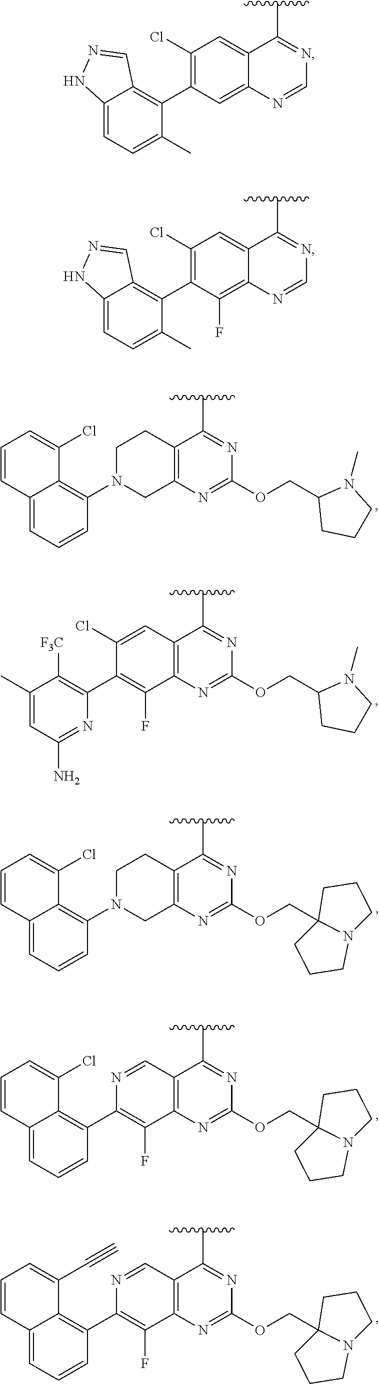

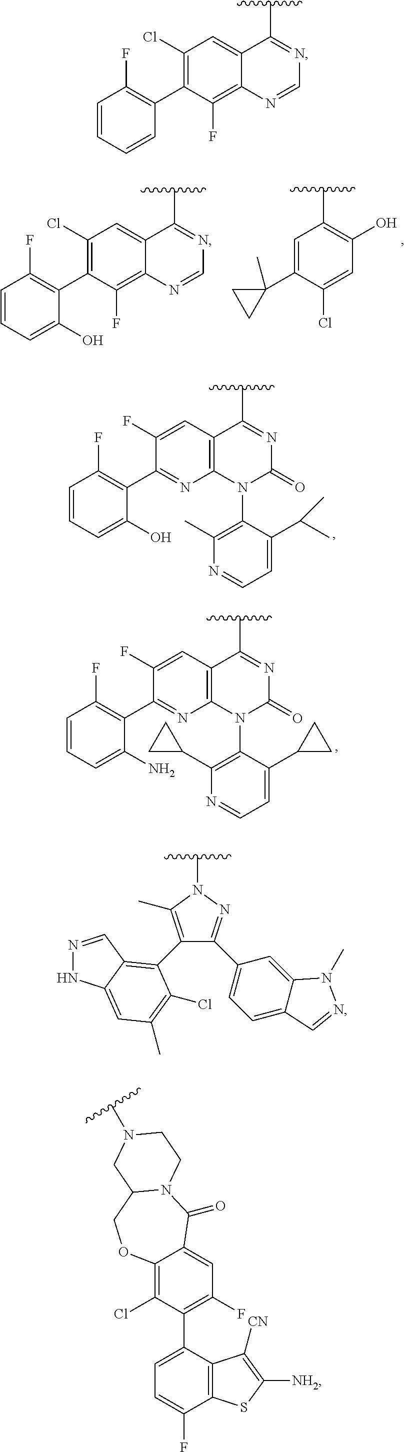

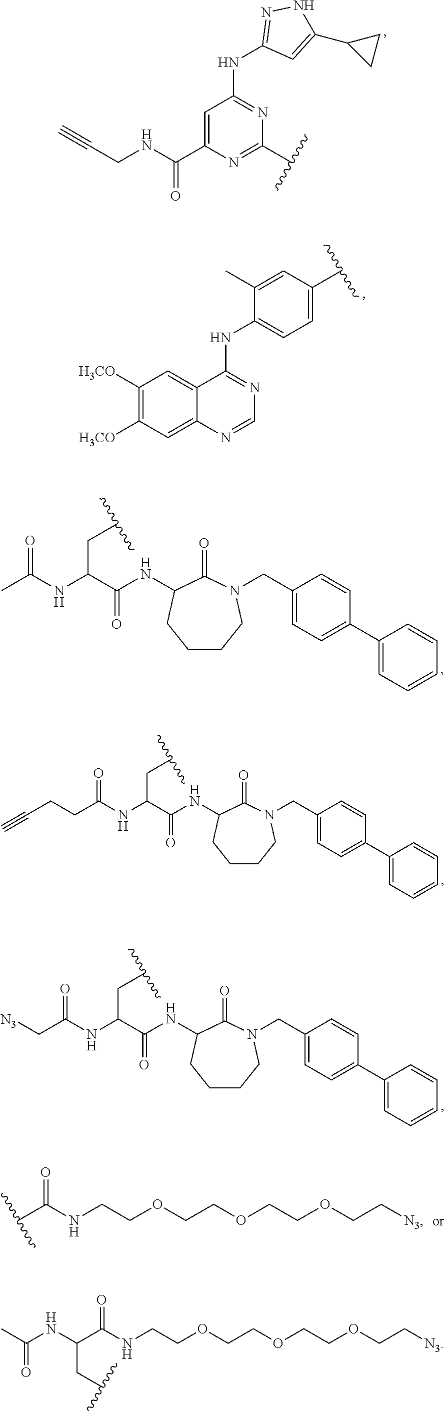

Embodiment 80. A method of attaching a compound to an arginine residue, said method comprising contacting said compound with said arginine residue, wherein said compound has the formula:

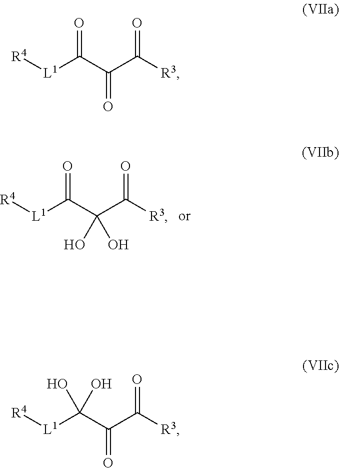

or a salt thereof; wherein
L¹ is a bond or divalent linker;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —N^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —N^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{3A}, R^{3B}, R^{3C}, R^{3D}, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X³ and X⁴ is independently —Cl, —Br, —I, or —F;
n3 and n4 are independently an integer from 0 to 4; and
m3, m4, v3, and v4 are independently 1 or 2.

Embodiment 81. The method of embodiment 80, wherein said arginine residue forms part of a protein.

Embodiment 82. The method of one of embodiments 80 to 81, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(o)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(o)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 83. The method of one of embodiments 80 to 82, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 84. The method of one of embodiments 80 to 82, wherein R⁴ is a biomolecular moiety.

Embodiment 85. A compound covalently bound to an arginine residue, having the formula:

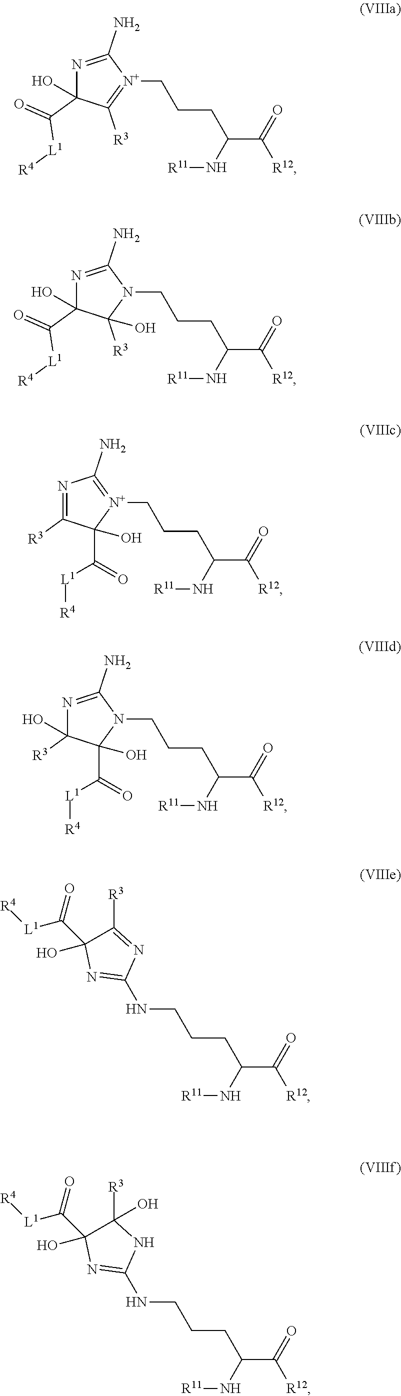

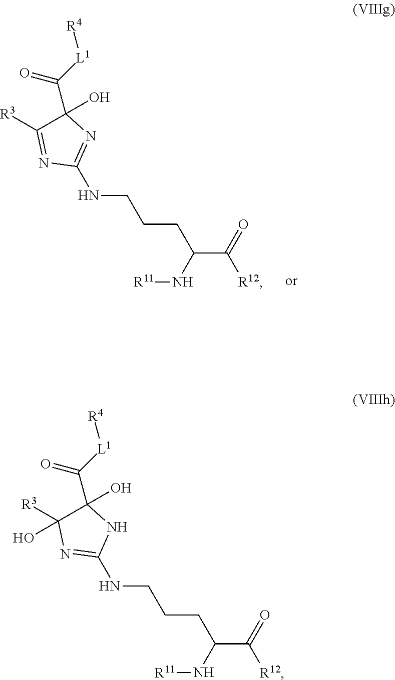

or a salt thereof; wherein
L¹ is a bond or divalent linker;
R³ is hydrogen, halogen, —CX³₃, —CHX³₂, —CH₂X³, —OCX³₃, —OCH₂X³, —OCHX³₂, —CN, —SOₙ₃R^{3D}, —SOv₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —N^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{3A}, R^{3B}, R^{3C}, R^{3D}, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X³ and X⁴ is independently —Cl, —Br, —I, or —F;
n3 and n4 are independently an integer from 0 to 4;
m3, m4, v3, and v4 are independently 1 or 2;
R¹¹ is hydrogen, —C(O)CH₃, or a first protein moiety; and
R¹² is —OH or a second protein moiety.

Embodiment 86. The covalently bound compound of embodiment 85, wherein the first protein moiety and the second protein moiety together form a single protein.

Embodiment 87. The covalently bound compound of embodiment 86, wherein the single protein comprises additional arginine residues that are not covalently bound to a compound to form a reacted arginine having the formula (VIa), (VIb), (VIc), (VId), (VIe), (VIf), (VIg), or (VIh).

Embodiment 88. The covalently bound compound of one of embodiments 85 to 87, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, —NR¹⁰²C(O)O—, —OC(O)NR¹⁰²—, —NR¹⁰²C(O)NR¹⁰²—, —NR⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 89. The covalently bound compound of one of embodiments 85 to 88, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 90. The covalently bound compound of one of embodiments 85 to 88, wherein R⁴ is a biomolecular moiety.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Chemoselective Covalent Modification of K-Ras(G12R) with a Small Molecule Electrophile

We report, inter alia, the discovery of covalent chemical ligands for the common oncogenic mutant K-Ras(G12R). These ligands bind in the Switch II pocket and irreversibly react with the mutant arginine residue. An X-ray crystal structure reveals an imidazolium condensation product formed between the α,β-diketoamide ligand and the ε- and η-nitrogens of arginine 12. Our results show that arginine residues can be selectively targeted with small molecule electrophiles despite their weak nucleophilicity and provide the basis for the development of mutant-specific therapies for K-Ras(G12R)-driven cancer.

Somatic mutations of the KRAS proto-oncogene are the predominant oncogenic lesions in human cancer (1,2). Although KRAS was historically considered an “undruggable” target, the recent successful development of covalent K-Ras(G12C) ligands has demonstrated remarkable clinical benefits of direct, allele-specific K-Ras inhibition (3, 4). These K-Ras(G12C) inhibitors exploit the strong nucleophilicity of the mutant cysteine and irreversibly bind in the Switch II region of K-Ras (5-11). However, many frequently occurring somatic mutations of K-Ras do not yield cysteine residues, and selective targeting of these mutants remains an unmet challenge. One of such hot spot mutations, KRAS p·G12R, is found in 17% of pancreatic ductal adenocarcinoma (PDAC) patients, accounting for more than 9,000 new cancer patients per year in the U.S. alone (4,12). We reasoned that covalent engagement of the acquired arginine (Arg12) could impart both potency and selectivity and enable the direct targeting of K-Ras(G12R).

Fully protonated at physiological pH (pKa₃=12.5), the guanidium group of arginine is weakly nucleophilic. To achieve chemoselective targeting of the mutant arginine, we first considered functional groups privileged to react with guanidines or amidines. Following the discovery of the reaction between 2,3-butanedione and benzamidine by Diels and Schleich in 1916 (13), a variety of vicinal dicarbonyl compounds (14-20) (e.g. phenylglyoxal (14), methylglyoxal (20-22), 2,3-butanedione (15), 1,2-cyclohexadionone (23)) have been found to selectively modify arginine residue on proteins. While these reagents have been employed to study protein glycation (16,22,24-28), non-specifically modify proteins (23,29,30) and perform bioconjugation reactions (19,31-33), such reactivity has not been exploited in the design of targeted covalent ligands.

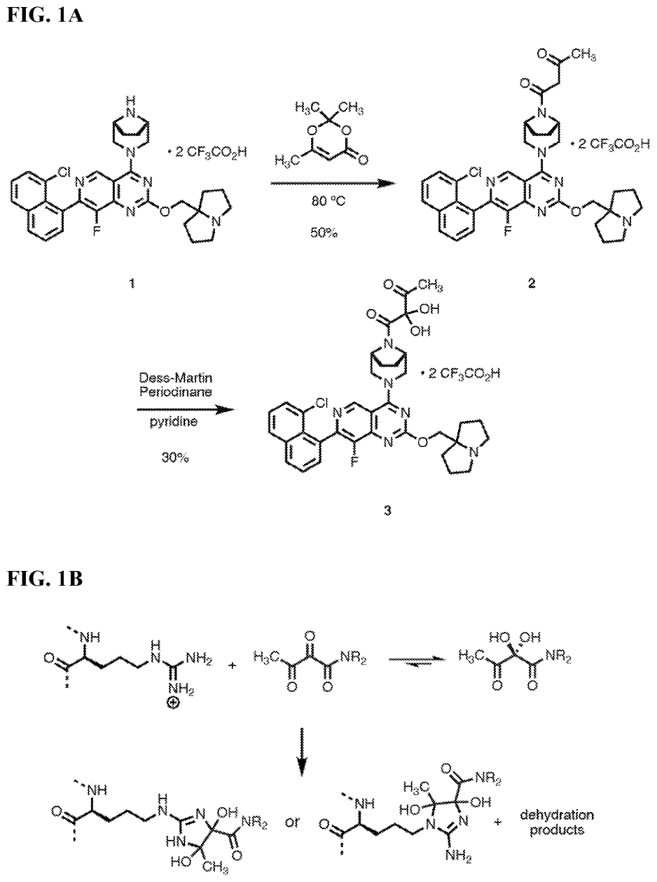

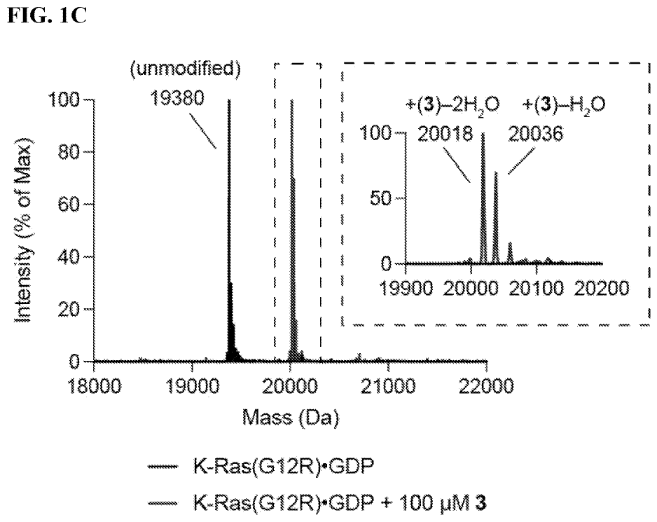

We asked whether a K-Ras Switch-II ligand, when equipped with a vicinal dicarbonyl system, would react with Arg12 in K-Ras(G12R). We synthesized compound 3 (FIG. 1A) by acetoacetylation of piperazine 1 followed by a-oxidation of the resulting acetoacetamide 2 with Dess-Martin periodinane (34). Compound 3 possesses an α,β-diketoamide, a rare but naturally occurring function present in FK506 and rapamycin. Likely due to the strong electrophilicity of the a-ketone, compound 3 could only be isolated as a hydrate. Compound 3 was stable in aqueous buffers over a range of pH and did not react with common thiol-containing nucleophiles (BME, DTT). However, when we incubated 100 μM compound 3 (MW=674 Da) with recombinant K-Ras(G12R)·GDP at pH 7.5 at 23° C. and monitored the reaction by intact protein mass spectrometry, we observed the formation of two new protein species with molecular weights consistent with a stoichiometric covalent adduct with the loss of one or two equivalents of water (+656 Da and +638 Da, respectively, FIG. 1C). Such reactivity is unique to the α,β-diketoamide 3, as compound 2 did not form any detectable covalent adduct under the identical reaction conditions. We hypothesized that the products of this reaction included imidazoline adducts with Arg12 and their dehydration products (FIG. 1B) (35), although their chemical identity remained to be determined.

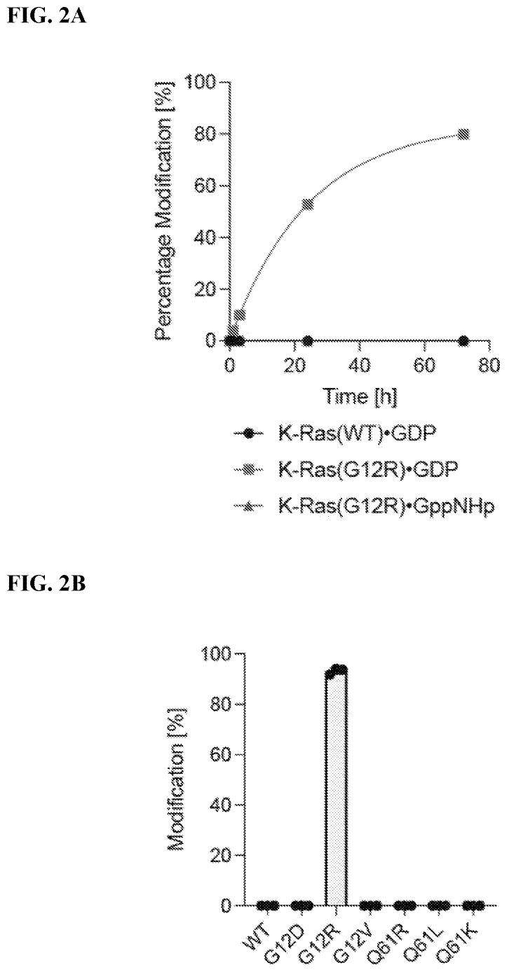

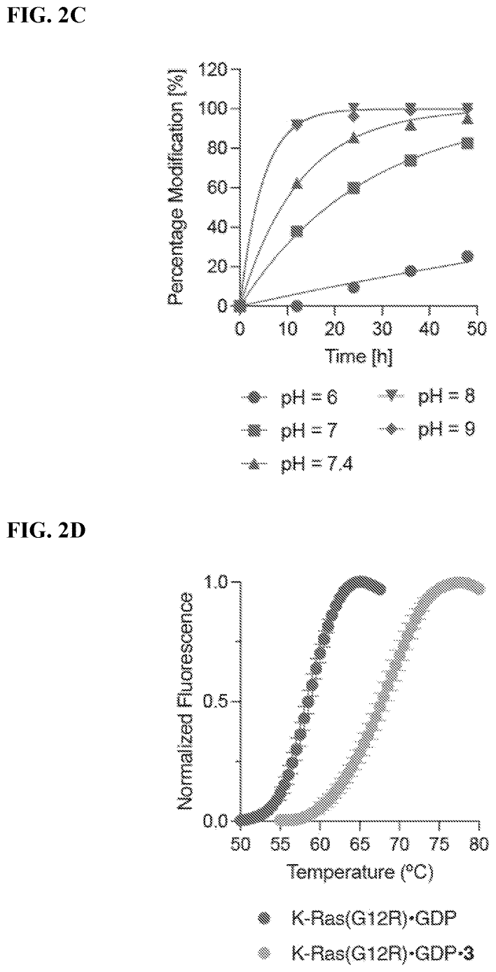

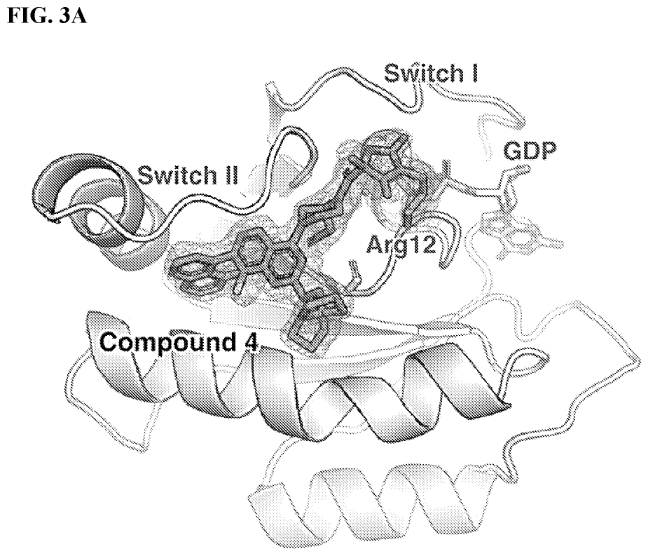

Compound 3 did not modify wildtype (WT) K-Ras·GDP (FIG. 2A) or other hotspot mutants including G12D, G12V, Q61R, Q61K and Q61L (FIG. 2B) upon extended incubation at pH 7.5, confirming the specificity of its reaction with Arg12. Additionally, K-Ras contains several surface exposed arginines, which were not modified by compound 3, further supporting its selectivity for the mutant arginine. Consistent with the preference of this ligand scaffold for the GDP-bound state of the protein, compound 3 reacted much slower with K-Ras(G12R)·GppNHp, with <5% modification after 72 h (FIG. 2A). The reaction was pH-dependent and proceeded at greatly reduced rate at pH 6. The reaction rate reached maximum at pH 8 and did not further increase at pH 9 (FIG. 2C). The K-Ras(G12R)·GDP·3 adduct exhibited markedly increased thermostability compared to unmodified K-Ras(G12R)·GDP (FIG. 2D), with an increase of melting temperature by 9.1° C. The formation of this adduct also appeared irreversible—we did not observe any reversal to the unmodified protein after incubation of the purified K-Ras(G12R)·GDP·3 adduct at pH 7.5 for 7 days at 23° C.

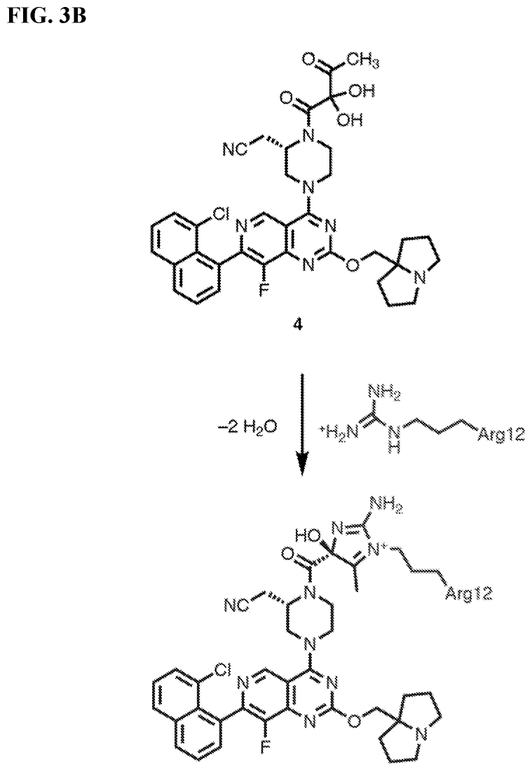

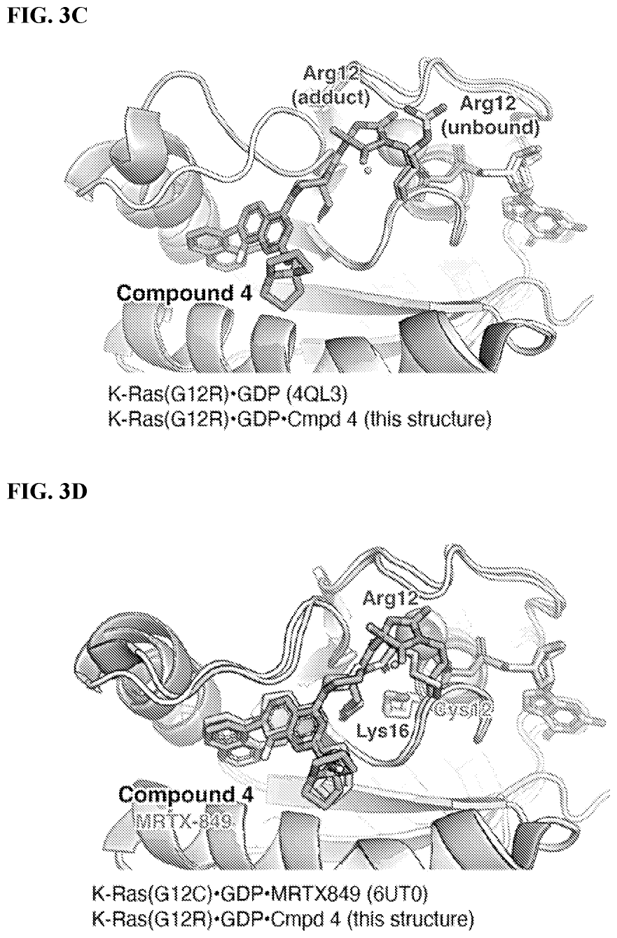

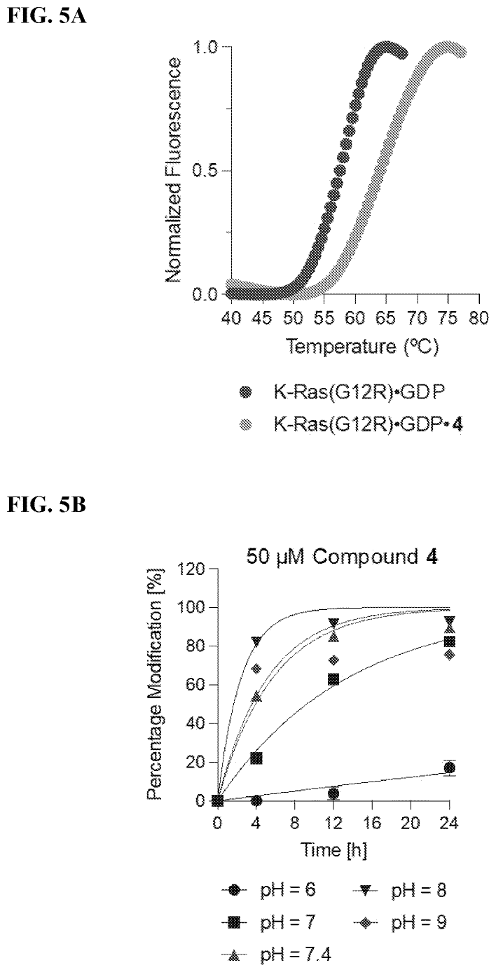

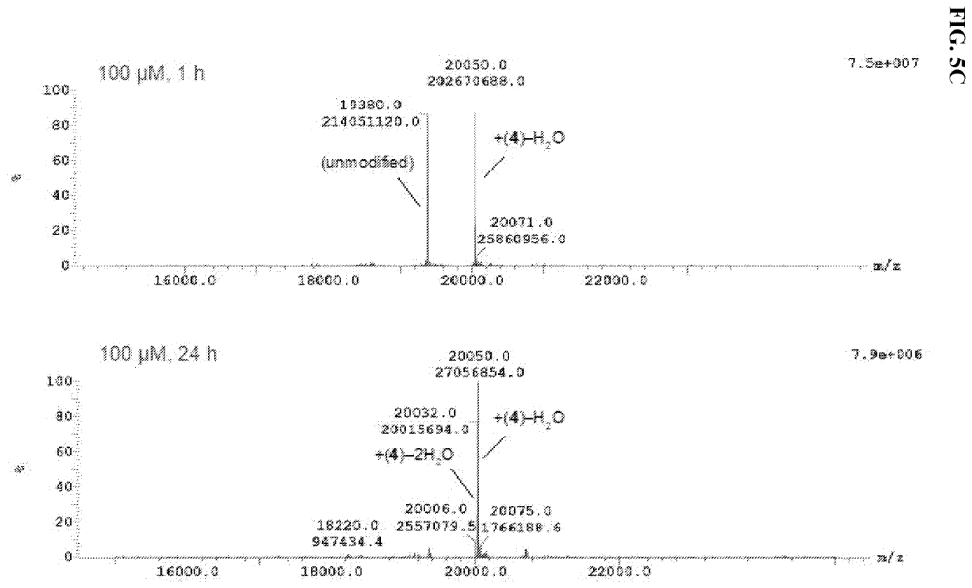

To understand the chemical nature of the adduct formed between Arg12 and α,β-diketoamides, we obtained a co-crystal of K-Ras(G12R)·GDP and compound 4 (a structural analog of compound 3, see FIGS. 5A-5C for its biochemical characterization) in the space group P2₁, which diffracted to 1.7 Å (FIG. 3A). Compound 4 was bound in the Switch II pocket of K-Ras, with well-defined electron density for the covalent bonds between the ligand and the protein (FIG. 3A). Surprisingly, we observed an imidazolium structure where Arg12 participated in a “side-on” orientation with its ε and η nitrogens. Such an imidazolium adduct is consistent with the loss of two water molecules revealed by whole protein mass spectrometry (FIG. 1C). The nucleophilic addition from the η nitrogen also appeared to be stereoselective, as the electron density clearly indicates the tertiary alcohol to be of S-configuration (FIGS. 3A-3B). Compared with unliganded K-Ras(G12R)·GDP, the side chain of Arg12 moved closer to the Switch II region, and the C_{β}—C_{γ}—C_{δ}—N_{ε} dihedral angle shifted from anti to gauche (FIG. 3C). These are likely energetically costly movements compensated by the reaction with the α,β-diketoamide and represent opportunities for future ligand optimization. The conformation of our adduct also differs from that seen with covalent K-Ras(G12C) ligands such as MRTX849 (FIG. 3D): the adduct is formed further away from the protein surface, and the amide carbonyl in our structure did not participate in a hydrogen bond interaction with Lys16.

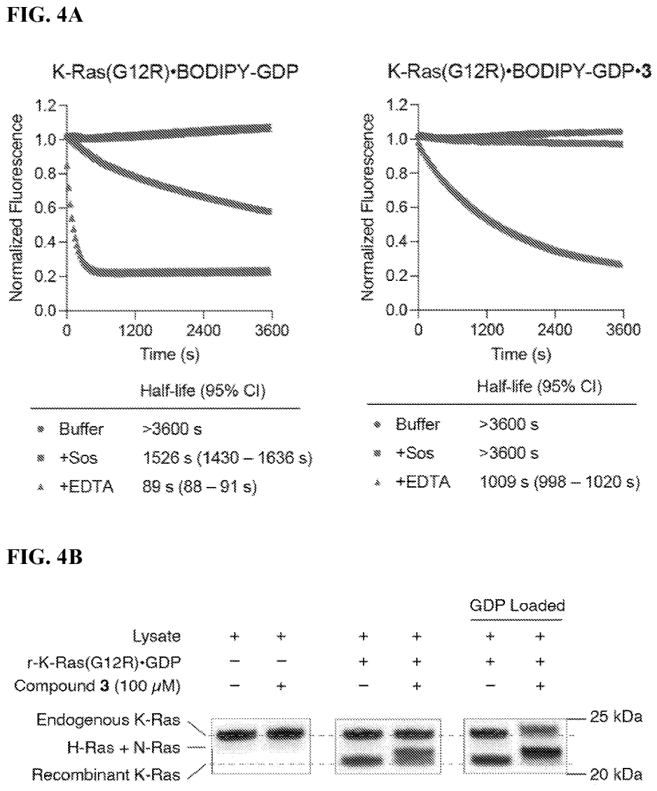

Asking whether the reaction between 3 and Arg12 confers functional inhibition of K-Ras(G12R), we tested the nucleotide exchange activity of unliganded and compound 3-bound K-Ras(G12R), where a fluorescent GDP analog (BODIPY-GDP) was exchanged for unlabeled GDP in the presence of Sos or EDTA (FIG. 4A). Compound 3 inhibited Sos-mediated exchange and significantly reduced the rate of EDTA-mediated exchange, consistent with previous observation with G12C-targeted covalent ligands (5).

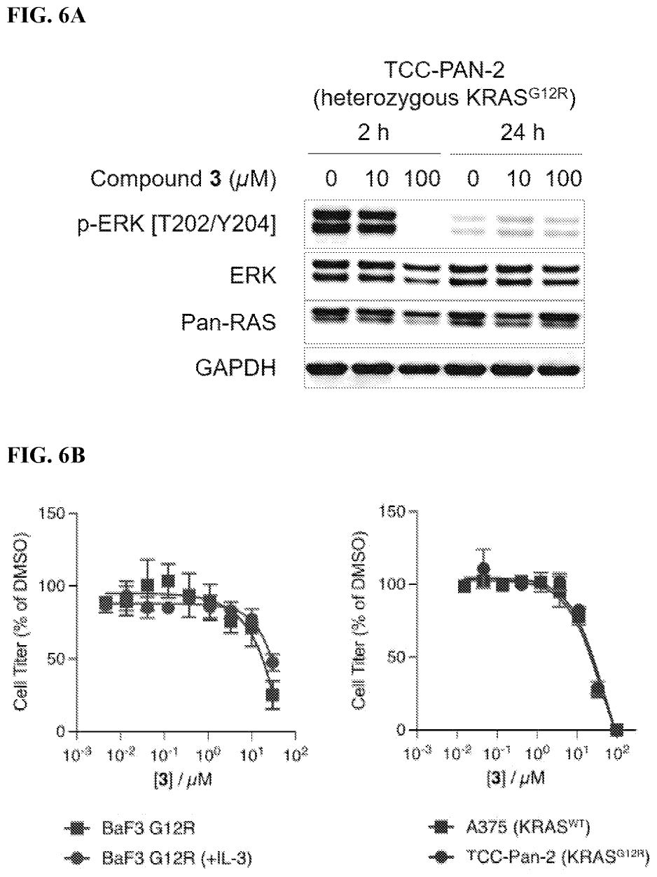

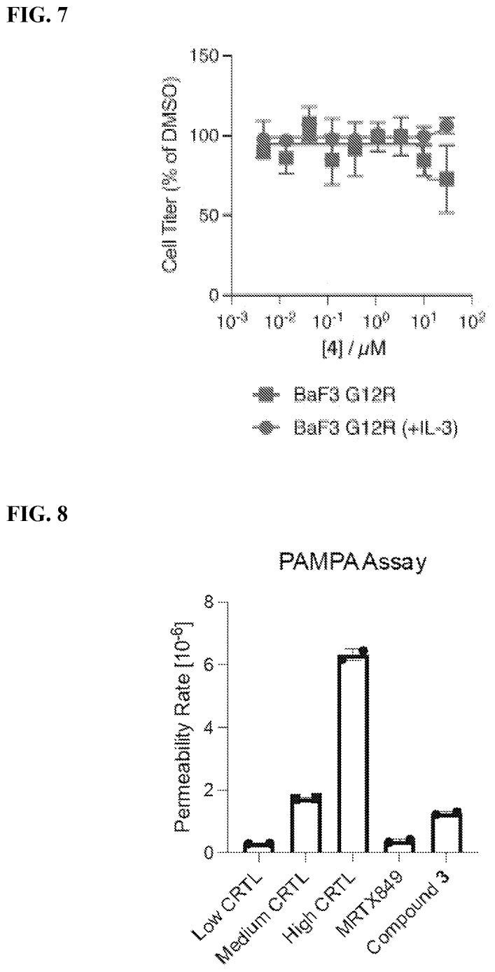

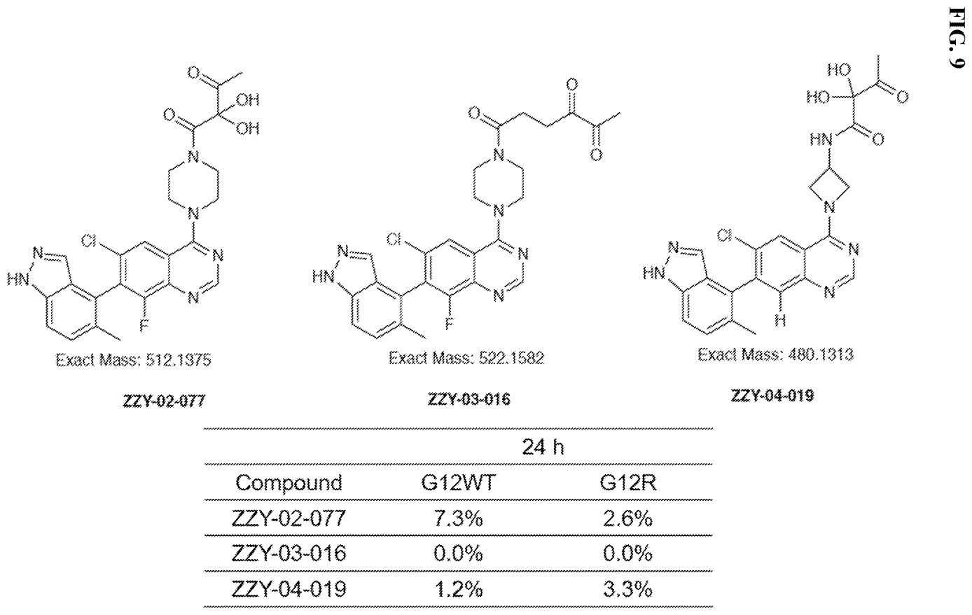

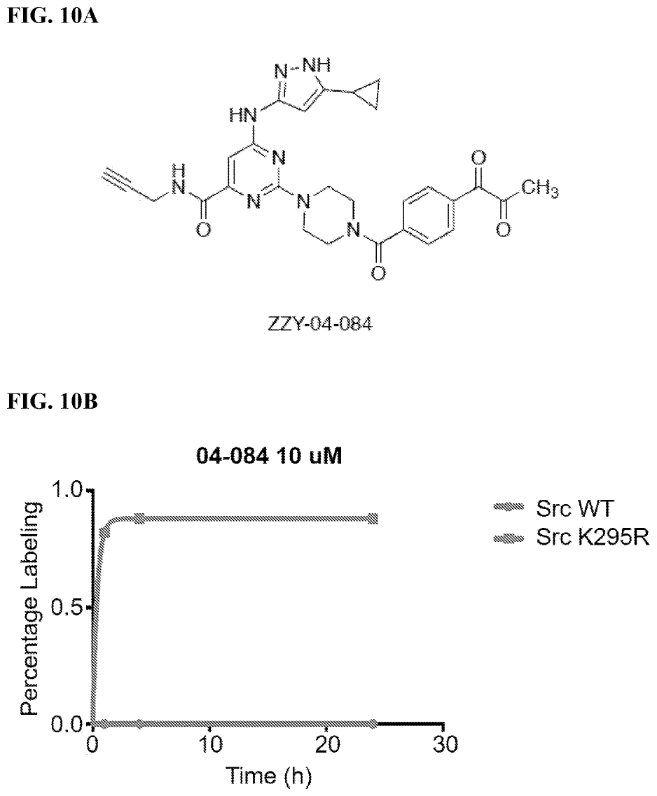

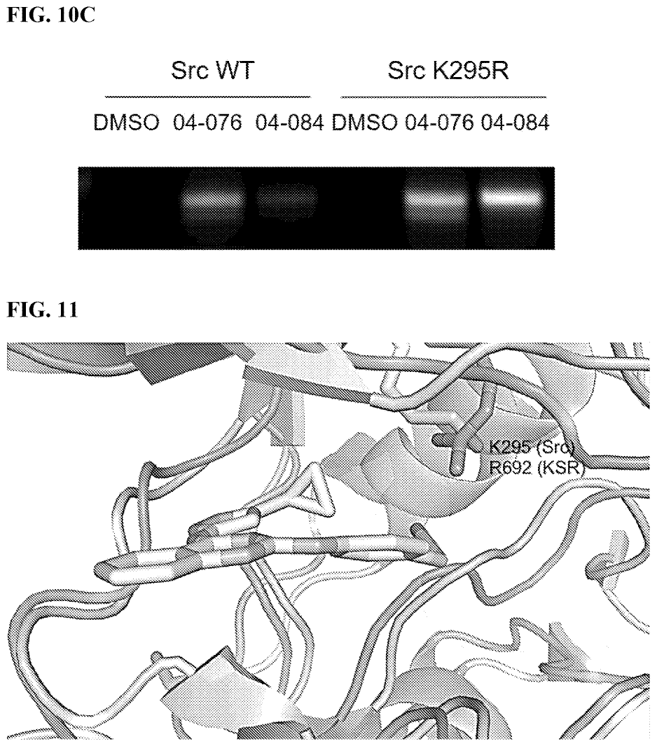

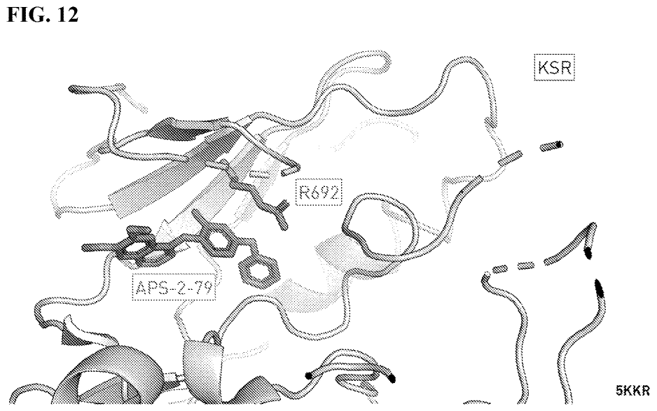

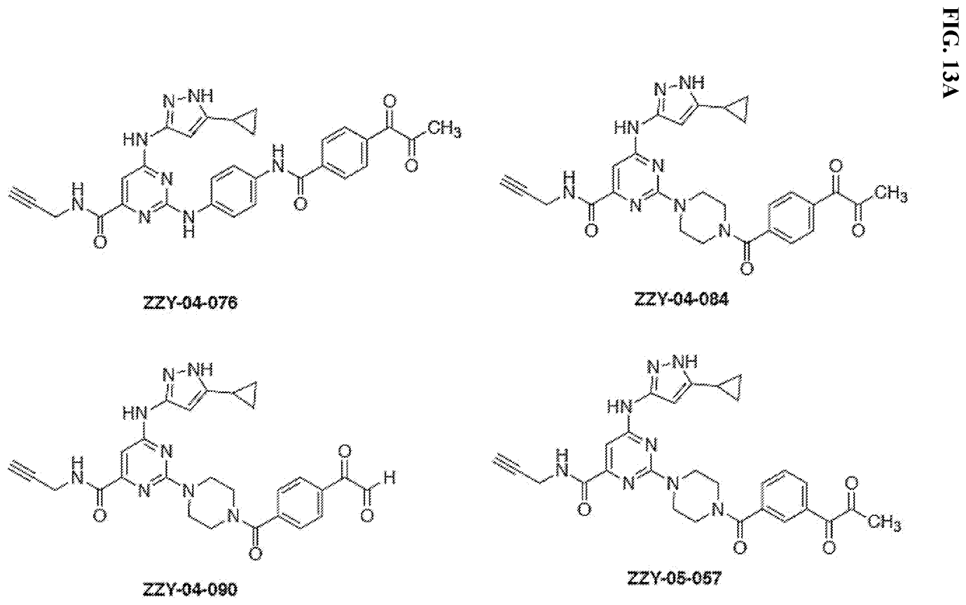

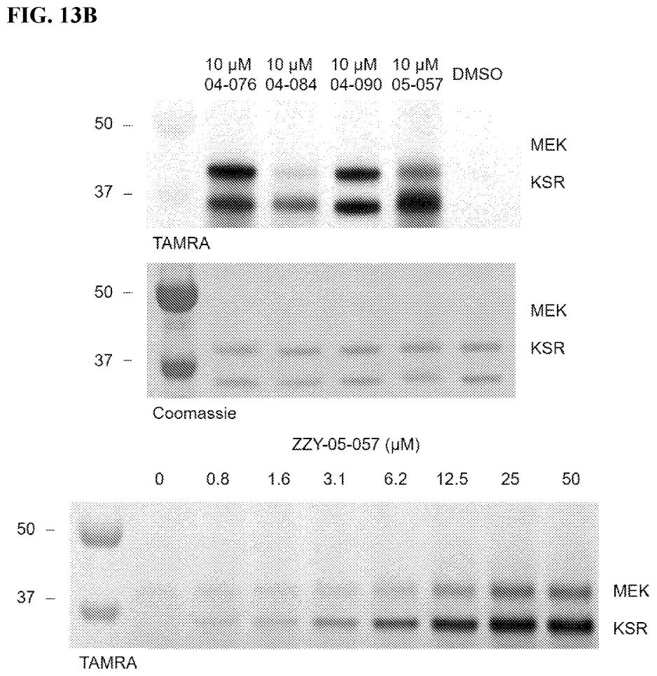

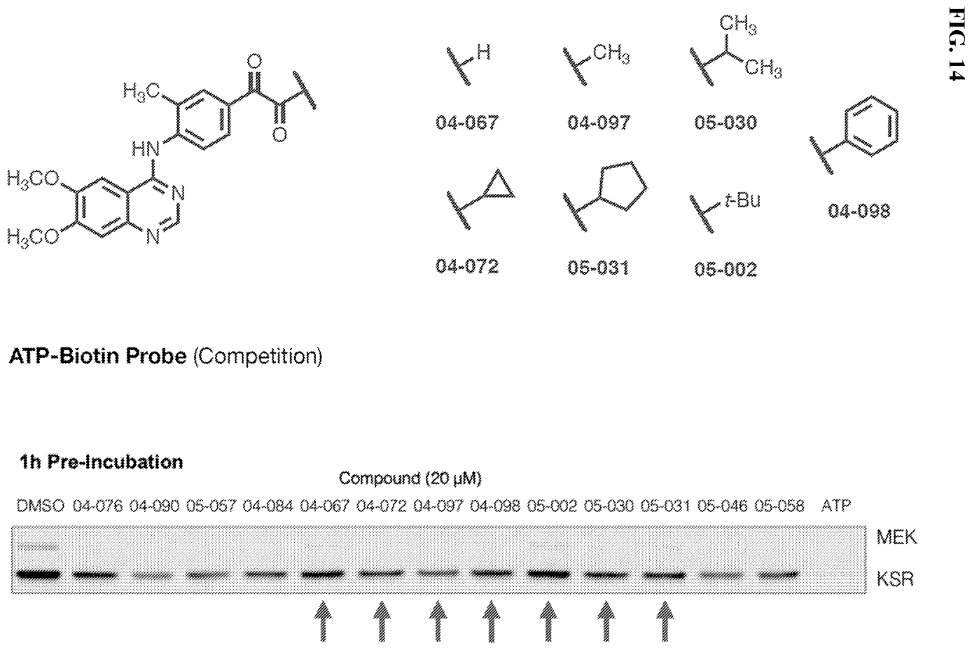

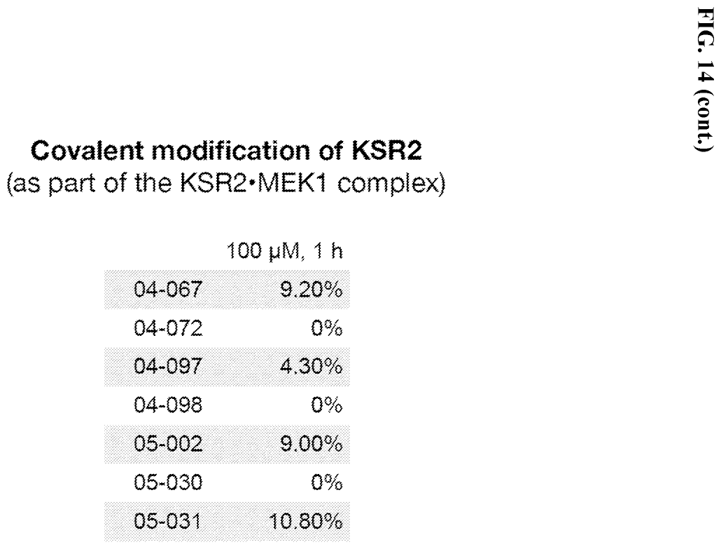

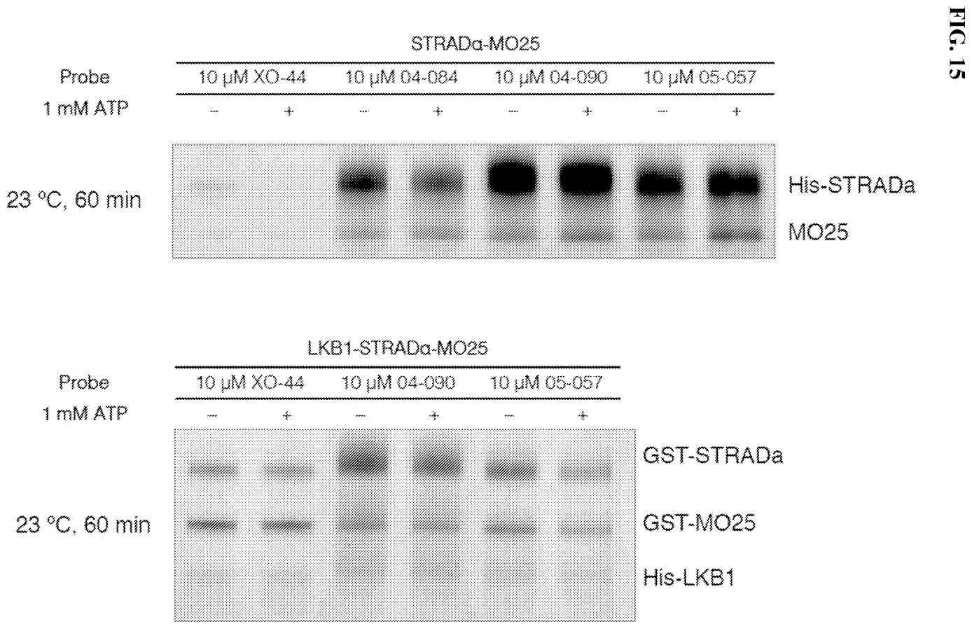

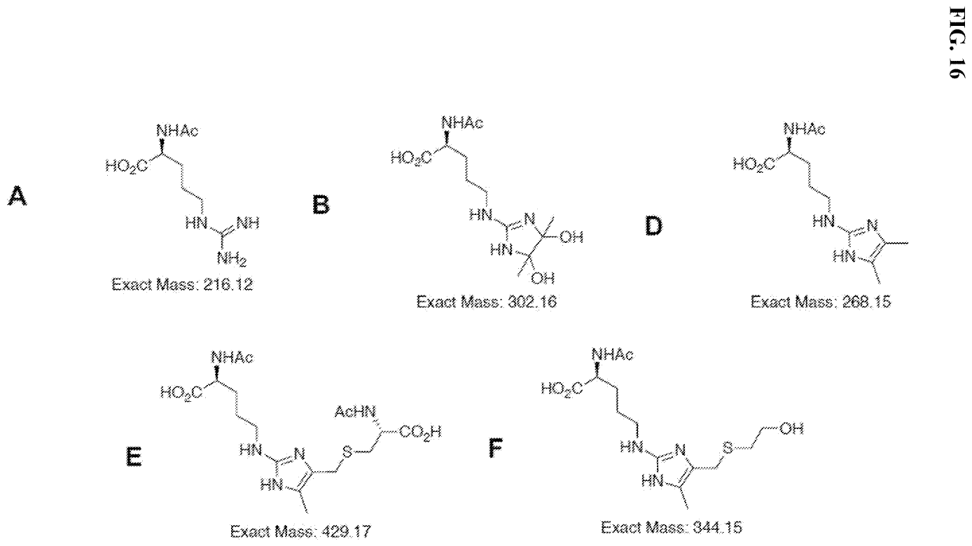

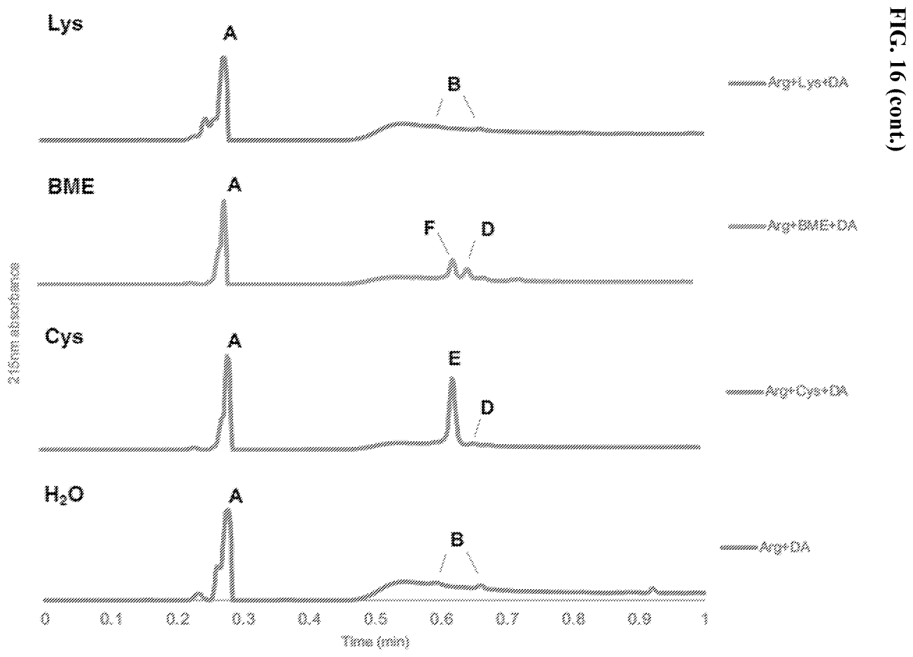

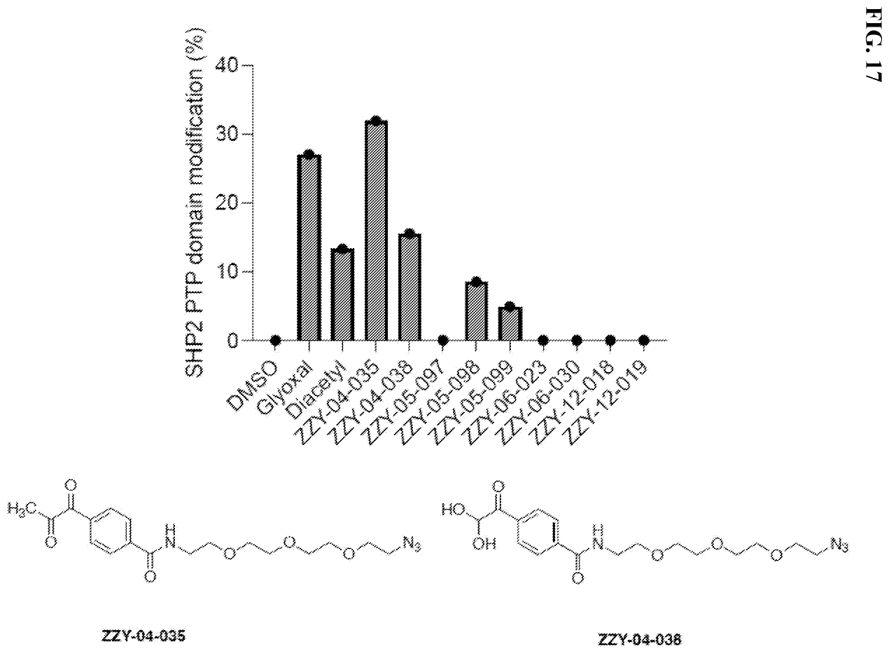

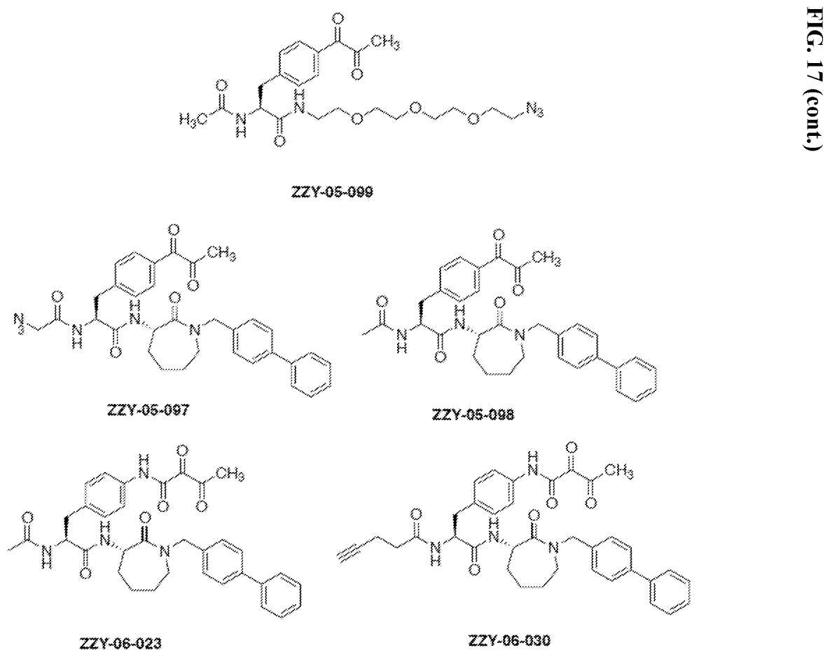

To probe the covalent modification of K-Ras by our arginine-targeted electrophile in a complex proteome, we took advantage of the molecular weight increase upon the reaction between K-Ras and 3, which is large enough to provide a direct visualization of target engagement on the anti-Ras immunoblot (FIG. 4B). We treated the lysates of BaF3/K-Ras(G12R), a mouse cell line engineered to express the mutant form of K-Ras, with compound 3 and monitored the Ras molecular weight change. We did not detect any modification of the endogenous K-Ras(G12R). However, when we added recombinant K-Ras(G12R)·GDP to the lysate, we observed complete modification of this exogenous protein after 16 h of incubation (FIG. 4B). The difference in reactivity suggested that compound 3 remained active in cell lysates but was unable to engage endogenous K-Ras(G12R). This was consistent with our observation that compounds 3 and 4 did not show cellular activity at concentrations below 100 μM (FIGS. 6A-6B and FIG. 7). Unlike K-Ras(G12C) which has been successfully targeted by GDP-state selective ligands, K-Ras(G12R) is known to have severely compromised GTPase activity (12,36) preventing its conversion into the susceptible GDP-bound state. We hypothesized that endogenous K-Ras(G12R) may exist predominantly in the GTP-bound state and therefore is not susceptible to 3 engagement. To test this hypothesis, we pre-loaded BaF3/K-Ras(G12R) lysates with excess GDP and repeated our treatments as above. Under these conditions, we observed covalent modification of the endogenous K-Ras(G12R), as evidenced by the upward shift of the Ras band (FIG. 4B), confirming that the predominant GTP nucleotide state of K-Ras(G12R) can be a challenge for its therapeutic targeting. This represents an opportunity for future optimization—either by identifying ligand scaffolds that recognize K-Ras in the GTP-bound state, or by shifting K-Ras to its GDP-bound state with agents targeting upstream signaling nodes (e.g. RTK, SHP2 or Sos inhibitors) (37).

In conclusion, we have identified the first mutant-selective covalent ligands of K-Ras(G12R) using α,β-diketoamides as a privileged arginine-reactive functional group. We found that these ligands give rise to stable imidazolium adducts with K-Ras(G12R) and directly observed the structure of one such adduct using X-ray crystallography. Our discovery expands our ability to selectively target a recurrent oncogenic mutant, K-Ras(G12R), for which no direct inhibitors have been reported. The chemistry reported here may also serve as the basis for the therapeutic targeting of other acquired arginine residues in human diseases.

### Example 2: Experimental Methods

### Cell Culture

Ba/F3 cells were a gift from Dr. Trevor Bivona (UCSF) and were maintained in RPMI 1640 (Gibco 11875093) supplemented with 10% heat-inactivated fetal bovine serum (Axenia Biologix) and 10 ng/mL recombinant mouse interleukin-3 (Gibco PMC0031). Cells were passed for at least two generations after cryorecovery before they were used for assays. All cell lines were tested mycoplasma negative using MycoAlert™ Mycoplasma Detection Kit (Lonza).

When indicated, cells were treated with drugs at 40-60% confluency at a final DMSO concentration of 1%. At the end of treatment period, cells were placed on ice. Unless otherwise indicated, adherent cells were washed once with ice-cold PBS (1 mL), scraped with a spatula, and pelleted by centrifugation (500×g, 5 min). Suspension cells were pelleted by centrifugation (500×g, 5 min), washed with 1 mL ice-cold PBS, and pelleted again. Cells were lysed in RIPA buffer supplemented with protease and phosphatase inhibitors (cOmplete and phosSTOP, Roche) on ice for 10 min. Lysates were clarified by high-speed centrifugation (19,000×g, 10 min). Concentrations of lysates were determined with protein BCA assay (Thermo Fisher) and adjusted to 2 mg/mL with additional RIPA buffer (or Co-IP Lysis Buffer). Samples were mixed with 5×SDS Loading Dye and heated at 95° C. for 5 min.

### Lysate Target Engagement Assay

For co-treatment with recombinant K-Ras(G12R), Ba/F3(G12R) cells were lysed with NP-40 buffer and adjusted to 1.5 mg/mL. A 0.1 μM solution of recombinant K-Ras(G12R) was prepared in NP-40 buffer. 18 μL of Ba/F3 lysate were added to 4 μL of a 0.5 mM solution of 3 containing 10% DMSO in NP-40 buffer or DMSO as control. 18 μL of recombinant K-Ras(G12R) solution or NP-40 was added and the mixtures were incubated for 16 h at 23° C. Subsequently samples were mixed with 5×SDS Loading Dye and heated at 95° C. for 5 min.

### Gel Electrophoresis and Immunoblot

Unless otherwise noted, SDS-PAGE was run with Novex 12% Bis-Tris gel (Invitrogen) in MES running buffer (Invitrogen) at 200 V for 60 min following the manufacturer's instructions. Protein bands were transferred onto 0.2-μm nitrocellulose membranes (Bio-Rad) using a wet-tank transfer apparatus (Bio-Rad Criterion Blotter) in 1×TOWBIN buffer with 10% methanol at 75V for 45 min. Membranes were blocked in 5% BSA-TBST for 1 h at 23° C. Primary antibody binding was performed with the indicated antibodies diluted in 5% BSA-TBST at 4° C. for at least 16 h. After washing the membrane three times with TBST (5 min each wash), secondary antibodies (goat anti-rabbit IgG-IRDye 800 and goat anti-mouse IgG-IRDye 680, Li-COR) were added as solutions in 5% skim milk-TBST at the dilutions recommended by the manufacturer. Secondary antibody binding was allowed to proceed for 1 h at 23° C. The membrane was washed three times with TBST (5 min each wash) and imaged on a Li-COR Odyssey fluorescence imager.

### Preparation of Mouse Stem Cell Virus (MSCV)

pMSCV-Puro plasmids containing full length human KRAS genes (G12R) were constructed using standard molecule biology techniques by inserting the KRAS gene fragment between the BamHI and XhoI sites. Transfection-grade plasmids were prepared using ZymoPure II Plasmid Midiprep kit. EcoPack 293 cells (Takara Bio) were plated in 6-well plates (3×10⁵/mL, 2 mL). The next day, cells were transfected with 2.5 μg pMSCV plasmid using lipofectamine 3000 following the manufacturer's instructions. The cells were incubated for 66 h, and then the virus-containing supernatants were collected and passed through a 0.22-μm syringe filter. The harvested virus was used immediately for spinfection of Ba/F3 cells or stored at −80° C.

### Generation of Stable Ba/F3 Transductants

1 mL of MSCV-containing supernatant (vide supra) was added to one well of a 6-well plate containing 1×10⁶ Ba/F3 cells in 1 mL of media comprised of 60% RMPI 1640, 40% heat-inactivated FBS, 10 ng mouse IL-3 and 4 μg polybrene. Cells were spinfected by centrifugation at 2,000 g for 90 minutes at room temperature and then placed in the incubator for 24 hours. After 1 day, the cells were diluted into 10 mL culture medium (RPMI 1640+10% heat-inactivated FBS, 10 ng/mL mouse IL-3) and recovered for a second day after spinfection. On the third day after spinfection, cells were pelleted at 500×g for 5 min and resuspended in 10 mL selection medium (RPMI 1640+10% heat-inactivated FBS, 10 ng/mL mouse IL-3, 1.25 μg/mL puromycin). Cells were maintained under puromycin selection for 4-7 days, splitting as required to maintain density <2×10⁶ cells/mL. After 7 days, cells were pelleted, washed once with IL-3 free culture medium (RPMI 1640+10% heat-inactivated FBS) and pelleted again before resuspending at 2-4×10⁵ cells/mL in IL-3 free culture medium. Cells were maintained under these conditions for 7 days, passaging as needed to maintain density <2×10⁶ cells/mL. Growth was monitored (Countess II Cell Counter) over these 7 days to confirm that an IL-3 independent population has been achieved.

### Intact Protein Mass Spectrometry

Purified K-Ras variants (4 μM final) were incubated with compounds at 50 or 100 μM (1% v/v DMSO final) in 20 mM HEPES pH 7.5, 150 mM NaCl, 1 mM MgCl₂ in a total volume of 150 μL. After the noted time, the samples were analyzed by intact protein LC/MS using a Waters Xevo G2-XS system equipped with an Acquity UPLC BEH C4 1.7 μm column. The mobile phase was a linear gradient of 5-95% acetonitrile/water+0.05% formic acid. The spectra were processed using QuantLynx, giving the ion counts observed for the most abundant species. For assays conducted under an alternate pH, the HEPES buffer was replaced with one of the following buffers (maintaining 150 mM NaCl and 1 mM MgCl₂): 20 mM MES pH 6 or 20 mM Tris pH 9.

### Sos- or EDTA-Mediated Nucleotide Exchange Assay

This assay was performed as previously reported (38-41) with slight modifications. BODIPY-GDP-loaded K-Ras proteins were prepared freshly as follows. To a 10 μM solution of K-Ras(G12R)·GDP or K-Ras(G12R)·GDP·3 in SEC Buffer (1 mL) was added sequentially BODIPY-GDP (5 mM, 40 μL, Thermo Fisher, final concentration 200 μM) and Na-EDTA pH 8.0 (0.5 M, 5 μL, final concentration 2.5 mM). The mixture was incubated at 23° C. for 1 h, and a solution of MgCl₂ (1.0 M, 20 μL, final concentration 10 mM) was added to the reaction mixture. The protein solution was run through a PD-10 column to remove the excess nucleotide following the manufacturer's protocol. Briefly, sample (˜1.0 mL) and excess buffer (1.5 mL) were loaded onto the column (equilibrated with NucEx Buffer), and desalted protein was eluted with NucEx Buffer (3.5 mL). Desalted protein was diluted 1:1 to a concentration of roughly 1.5 μM in NucEx Buffer. 12 μL of this solution (triplicate for each condition) was added to wells of a black 384-well low-volume assay plate (Corning 4514). 3 μL of either 1 mM GDP, 1 mM GDP+5 μM Sos, or 1 mM GDP+40 mM EDTA (all prepared in NucEx Buffer) was added via a multichannel pipette rapidly to the wells. This should take less than 15 s to finish. The plate was immediately placed in a TECAN Spark 20M plate reader, and fluorescence for BODIPY (excitation 488 nm, emission 520 nm) was read every 30 s over 1 h. Fluorescence intensity was normalized to values at time 0 and plotted again time. Observed rate constant (k_{obs}) was derived by fitting the curve to first-order kinetic equation

$$F = ( {F}_{0} - {F}_{∞} ) exp [ - {k}_{o b s} t ] + {F}_{∞}$$

and plotted against time.

### Differential Scanning Fluorimetry

The protein of interest was diluted with SEC Buffer [20 mM HEPES 7.5, 150 mM NaCl, 1 mM MgCl₂] to 8 μM. This solution was dispensed into wells of a white 96-well PCR plate in triplicate (25 μL/well). Fluorescence was measured at 0.5° C. temperature intervals every 30 s from 25° C. to 95° C. on a Bio-Rad CFX96 qPCR system using the FRET setting. Each data set was normalized to the highest fluorescence and the normalized fluorescence reading was plotted against temperature in GraphPad Prism 8.0. Tm values were determined as the temperature(s) corresponding to the maximum(ma) of the first derivative of the curve.

### Cell Viability Assay

Cells were seeded into 96-well white flat bottom plates (1,000 cells/well) (Corning) and incubated overnight. Cells were treated with the indicated compounds in a nine-point threefold dilution series (100 μL final volume) and incubated for 72 h. Cell viability was assessed using a commercial CellTiter-Glo (CTG) luminescence-based assay (Promega). Briefly, the 96-well plates were equilibrated to room temperature before the addition of diluted CTG reagent (100 μL) (1:4 CTG reagent:PBS). Plates were placed on an orbital shaker for 30 min before recording luminescence using a Spark 20M (Tecan) plate reader.

### Recombinant Protein Expression and Purification

### K-Ras (Wildtype), K-Ras (G12R) and K-Ras (G12R) Cyslight

DNA sequences encoding human K-Ras (wildtype, aa 1-169), human K-Ras (G12R, aa 1-169), human K-Ras G12R Cyslight (G12R/C51S/C80L/C118S, aa 1-169), human NF1-GRD (aa 1203-1530) were codon optimized, synthesized by Twist Biosciences and cloned into pJExpress411 vector using the Gibson Assembly method (42). The resulting construct contains N-terminal 6×His tag and a TEV cleavage site (ENLYFQG (SEQ ID NO:8)). The proteins were expressed and purified following previously reported protocols (38,43). Briefly, chemically competent BL21(DE3) cells were transformed with the corresponding plasmid and grown on LB agar plates containing 50 μg/mL kanamycin. A single colony was used to inoculate a culture at 37° C., 220 rpm in terrific broth containing 50 μg/mL kanamycin. When the optical density reached 0.6, the culture temperature was reduced to 20° C., and protein expression was induced by the addition of IPTG to 1 mM. After 16 h at 20° C., the cells were pelleted by centrifugation (6,500×g, 10 min) and lysed in lysis buffer [20 mM Tris 8.0, 500 mM NaCl, 5 mM imidazole] with a high-pressure homogenizer (Microfluidics, Westwood, MA). The lysate was clarified by high-speed centrifugation (19,000×g, 15 min) and the supernatant was used in subsequent purification by immobilized metal affinity chromatography (IMAC). His-TEV tagged protein was captured with Co-TALON resin (Clonetech, Takara Bio USA, 2 mL slurry/liter culture) at 4° C. for 1 h with constant end-to-end mixing. The loaded beads were then washed with lysis buffer (50 mL/liter culture) and the protein was eluted with elution buffer [20 mM Tris 8.0, 300 mM NaCl, 300 mM imidazole]. To this protein solution was added His-tagged TEV protease (0.05 mg TEV/mg Ras protein) and GDP (1 mg/mg Ras protein), and the mixture was dialyzed against TEV Cleavage Buffer [20 mM Tris 8.0, 300 mM NaCl, 1 mM EDTA, 1 mM DTT] at 4° C. using a 10K MWCO dialysis cassette until LC-MS analysis showed full cleavage (typically 16-24 h). MgCl₂ was added to a final concentration of 5 mM, and the mixture was incubated with 1 mL Ni-NTA (Qiagen) beads at 4° C. for 1 h to remove TEV protease, any residual His-tagged proteins and peptides. The protein solution was diluted 1:10 v/v with 20 mM Tris 8.0 and further purified with anion exchange chromatography (HiTrapQ column, GE Healthcare Life Sciences) using a NaCl gradient of 50 mM to 500 mM in 20 mM Tris 8.0. Nucleotide loading was performed by mixing the ion exchange-purified protein with an excess of GDP (5 mg/liter culture) or GppNHp (5 mg/liter culture) and 5 mM EDTA at 23° C. for 30 min. The reaction was stopped by the addition of MgCl₂ to 10 mM. For GppNHp, an additional calf intestine phosphatase treatment was performed as follows to ensure high homogeneity of the loaded nucleotide. The protein buffer was exchanged into Phosphatase Buffer [32 mM Tris 8.0, 200 mM ammonium sulfate, 0.1 mM ZnCl₂] with a HiTrap Desalting Column (GE Healthcare Life Sciences). To the buffer-exchanged protein solutions, GppNHp was added to 5 mg/mL, and Calf Intestine Phosphatase (NEB) was added to 10 U/mL. The reaction mixture was incubated on ice for 1 h, and MgCl₂ was added to a final concentration of 20 mM. After nucleotide loading, the protein was concentrated using an 10K MWCO centrifugal concentrator (Amicon-15, Millipore) to 20 mg/mL and purified by size exclusion chromatography on a Superdex 75 10/300 GL column (GE Healthcare Life Sciences). Fractions containing pure biotinylated Ras protein were pooled and concentrated to 20 mg/mL and stored at −78° C. In our hands, this protocol gives a typical yield of 5-15 mg/liter culture.

### Sos^{cat}

The catalytic domain of Sos (residues 466-1049, Sos^{cat}) was expressed and purified following a published protocol (44).

### Crystallization

K-Ras(G12R) Cyslight (G12S/C51S/C80L/C118S) bound by GDP purified by size exclusion chromatography was diluted to 100 μM in Reaction Buffer (20 mM HEPES 7.5, 150 mM NaCl, 1 mM MgCl₂). Compound 4 was added as a 10 mM solution in DMSO to a final concentration of 200 μM. The mixture was allowed to stand at 23° C. until LC-MS analysis of the reaction mixture showed full conversion to a single covalent adduct. The reaction mixture was purified by size exclusion chromatography (Superdex75, 20 mM HEPES 7.5, 150 mM NaCl, 1 mM MgCl₂) and concentrated to 20 mg/mL. For crystallization, 0.1 μL of the protein was mixed with 0.1 μL well buffer containing 0.1 M MES pH 6.5, 30% w/v PEG 4K. Crystals were grown at 20° C. in a 96-well plate using the hanging-drop vapor diffusion method. Maximal crystal growth was achieved after 7 days. The crystals were transferred to a cryoprotectant solution (0.1 M MES pH 6.5, 30% w/v PEG 4K, 15% glycerol) and flash-frozen in liquid nitrogen.

### X-Ray Data Collection and Structure Determination

Dataset was collected at the Advanced Light Source beamline 8.2.1 with X-ray at a wavelength of 0.999907 Å. The dataset was indexed and integrated using iMosfim (Battye et al., 2011), scaled with Scala (Evans, 2006) and solved by molecular replacement using Phaser (McCoy et al., 2007) in CCP4 software suite (Winn et al., 2011). The crystal structure of GDP-bound K-Ras(G12C)-MRTX849 adduct (PDB code: 6UT0) was used as the initial model. The structure was manually refined with Coot (Emsley et al., 2010) and PHENIX (Adams et al., 2010). Data collection and refinement statistics are listed in Table S1. In the Ramachandran plot of the final structure, 96.13% and 3.87% of the residues are in the favored regions and allowed regions, respectively.

### Parallel Artificial Membrane Permeability Assay (PAMPA)

Parallel Artificial Membrane Permeability Assay was performed by BioAssay Systems Inc. (Hayward, CA). A 25 μL aliquot of each test compound was diluted into 475 μL PBS (pH 7.4). A 84 μL aliquot of the diluted compound was further diluted with 126 μL of PBS (pH 7.4) to serve as the Equilibrium Standard. A 4% lecithin lipid mixture was solubilized in 100% dodecane then 5 μL of the lecithin/dodecane solution was applied to the PAMPA Donor plate membrane. In duplicate, 200 μL of the initial compound dilution was applied to the PAMPA plate Donor well. A 300 μL aliquot of PBS (pH 7.4) was added to each Acceptor well then the PAMPA system was assembled. The PAMPA plate was incubated for 18 hours at room temperature. Following the incubation, 100 μL of each Acceptor solution was transferred to a 96-well UV plate. In parallel, each compound's Equilibrium solution was also added to the plate, in duplicate. If an Equilibrium solution showed precipitate, it was clarified by centrifugation at 13,000×g for 10 minutes. The Blank was DMSO in PBS (pH 7.4) minus compound at the same final dilution (2% (v/v)). As a positive control, Chloramphenicol (high), Diclofenac (medium), and Theophylline (low) were run in parallel at 500 M for the Donor solution and 200 M for the Equilibrium solution. The permeability rate of each compound was determined using the following equation:

$$\text{?} = C \times - ln ( 1 - \frac{{OD}_{A}}{O \text{?}} ) cm / \text{?}$$

$$\text{?} \text{indicates text missing or illegible when filed}$$

where Pe is the Permeability Rate, ODA is the absorbance of the Acceptor solution minus the Blank absorbance, ODE is the absorbance of the Equilibrium solution minus the Blank absorbance, and C=7.72×10⁻⁶. The Peak of absorbance used for each compound was compound dependent and based upon peak of maximal absorbance in the Equilibrium solution.

### Chemical Synthesis

### General Experiment Procedure

All reactions were performed in oven-dried glassware fitted with rubber septa under a positive pressure of argon, unless otherwise noted. Air- and moisture-sensitive liquids were transferred via syringe. Solutions were concentrated by rotary evaporation at or below 40° C. Analytical thin-layer chromatography (TLC) was performed using glass plates pre-coated with silica gel (0.25-mm, 60-Å pore size, 230-400 mesh, Merck KGA) impregnated with a fluorescent indicator (254 nm). TLC plates were visualized by exposure to ultraviolet light (UV), then were stained by submersion in a 10% solution of phosphomolybdic acid (PMA) in ethanol or an 2% aqueous solution of potassium permanganate followed by brief heating on a hot plate. Flash column chromatography was performed with Teledyne ISCO CombiFlash EZ Prep chromatography system, employing pre-packed silica gel cartridges (Teledyne ISCO RediSep).

### Solvents and Reagents

Anhydrous solvents were purchased from Acros Organics. Unless specified below, all chemical reagents were purchased from Sigma-Aldrich, AK Scientific or Chemscene.

### Instrumentation

Proton nuclear magnetic resonance (¹H NMR) spectra, carbon nuclear magnetic resonance (¹³C NMR) spectra, and fluorine nuclear magnetic resonance (¹⁹F NMR) spectra were recorded on Bruker AvanceIII HD instrument (400 MHz/100 MHz/376 MHz) at 23° C. operating with the Bruker Topspin 3.1. NMR spectra were processed using Mestrenova (version 14.1.2). Proton chemical shifts are expressed in parts per million (ppm, 6 scale) and are referenced to residual protium in the NMR solvent (CHCl₃: δ 7.26, D₂HCOD: δ 3.31). Carbon chemical shifts are expressed in parts per million (ppm, δ scale) and are referenced to the carbon resonance of the NMR solvent (CDCl₃: δ 77.0, CD₃OD: δ 49.0). Fluorine chemical shifts are expressed in parts per million (ppm, δ scale) and are referenced to an external standard of trifluoroacetic acid (−76.55 ppm). Data are represented as follows: chemical shift, multiplicity (s=singlet, d=doublet, t=triplet, q=quartet, dd=doublet of doublets, dt=doublet of triplets, m=multiplet, br=broad, app=apparent), integration, and coupling constant (J) in Hertz (Hz). High-resolution mass spectra were obtained using a Waters Xevo G2-XS time-of-flight mass spectrometer operating with Waters MassLynx software (version 4.2).

### Monitoring Reaction Progress by LC-MS

When LC-MS analysis of the reaction mixture is indicated in the procedure, it was performed as follows. An aliquot (1 μL) of the reaction mixture (or the organic phase of a mini-workup mixture) was diluted with 100 μL 1:1 acetonitrile:water. 1 μL of the diluted solution was injected onto a Waters Acquity UPLC BEH C18 1.7 pm column and eluted with a linear gradient of 5-95% acetonitrile/water (+0.1% formic acid) over 3.0 min. Chromatograms were recorded with a UV detector set at 254 nm and a time-of-flight mass spectrometer (Waters Xevo G2-XS).

### 1-((1R,5S)-3-(7-(8-chloronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)butane-1,3-dione (2)

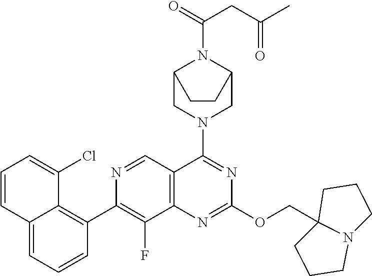 [SMILES: CC(=O)CC(=O)N1C2CCC1CN(c1nc(OCC34CCCN3CCC4)nc3c(F)c(-c4cccc5cccc(Cl)c45)ncc13)C2]

(1R,5S)-3-(7-(8-chloronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-ium-di-2,2,2-trifluoroacetate (50.0 mg, 0.064 mmol, 1.0 equiv) was dissolved in dry dichloroethane (1 mL). DIPEA (28.1 mg, 0.218 mmol, 3.4 equiv) and 2,2,6-trimethyl-4H-1,3-dioxin-4-one (12.4 mg, 0.087 mmol, 1.4 equiv) were subsequently added and the solution was stirred at 80° C. for 1 h. The reaction mixture was partitioned between saturated aqueous sodium bicarbonate solution and dichloromethane. The layers were separated, the organic phase was extracted with dichloromethane, dried over Na₂SO₄, filtered, and concentrated. The crude mixture was purified by reverse-phase HPLC to yield (1R,5S,8s)-3-(7-(8-chloronaphthalen-1-yl)-8-fluoro-2-((hexahydropyrrolizin-4-ium-7a(1H)-yl)methoxy)pyrido[4,3-d]pyrimidin-4-yl)-8-(3-oxobutanoyl)-3,8-diazabicyclo[3.2.1]octan-8-ium-di-2,2,2-trifluoroacetate as colorless solid (27.8 mg, 0.032 mmol, 50%). ¹H NMR (600 MHz, d6-Acetone) δ 9.25-9.20 (m, 1H), 8.20 (d, J=7.7 Hz, 1H), 8.09 (d, J=8.0 Hz, 1H), 7.76-7.71 (m, 1H), 7.65 (dd, J=7.2, 2.6 Hz, 2H), 7.57 (t, J=7.8 Hz, 1H), 4.80 (td, J=13.2, 8.3 Hz, 5H), 4.50 (d, J=5.9 Hz, 1H), 3.91-3.69 (m, 5H), 3.40 (s, 2H), 2.43-2.36 (m, 2H), 2.33-2.27 (m, 2H), 2.26 (s, 3H), 2.24-2.17 (m, 4H), 1.97-1.81 (m, 4H). ¹⁹F NMR (600 MHz, d6-Acetone) 6-140.4. HRMS: m/z calcd. for C₃₅H₃₇ClFN₆O₃⁺ ([M+H]⁺): 643.2594, found: 643.2711.

### 1-((1R,5S)-3-(7-(8-chloronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)-2,2-dihydroxybutane-1,3-dione (3)

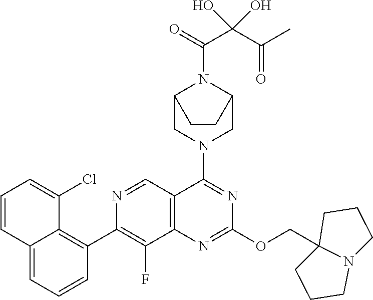 [SMILES: CC(=O)C(O)(O)C(=O)N1C2CCC1CN(c1nc(OCC34CCCN3CCC4)nc3c(F)c(-c4cccc5cccc(Cl)c45)ncc13)C2]

(1R,5S,8s)-3-(7-(8-chloronaphthalen-1-yl)-8-fluoro-2-((hexahydropyrrolizin-4-ium-7a(1H)-yl)methoxy)pyrido[4,3-d]pyrimidin-4-yl)-8-(3-oxobutanoyl)-3,8-diazabicyclo[3.2.1]octan-8-ium-di-2,2,2-trifluoroacetate (22.7 mg, 0.026 mmol, 1.0 equiv.) was dissolved in wet CH₂Cl₂ (1 mL). Pyridine (8.24 mg, 0.104 mmol, 4.0 equiv.) and dess-martin periodinane (44.2 mg, 0.104 mmol, 4.0 equiv.) were added and the mixture was stirred for 1 h at room temperature. Solvents were removed under reduced pressure. The residue was dissolved in MeCN:H2O (1:1) and purified by reverse phase HPLC (C18) to yield 1-((1R,5S)-3-(7-(8-chloronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)-2,2-dihydroxybutane-1,3-dione-di-2,2,2-trifluoroacetate as colorless solid (7.1 mg, 0.079 mmol, 30%). ¹H NMR (400 MHz, d6-Acetone) δ 9.22-9.17 (m, 1H), 8.20 (dd, J=8.2, 1.1 Hz, 1H), 8.13-8.06 (m, 1H), 7.78-7.71 (m, 1H), 7.68-7.63 (m, 2H), 7.58 (t, J=7.8 Hz, 1H), 4.92-4.68 (m, 6H), 3.90-3.80 (m, 4H), 3.38 (s, 2H), 2.46-2.39 (m, 2H), 2.32 (s, 3H), 2.23 (ddt, J=25.6, 12.8, 6.8 Hz, 6H), 1.94 (t, J=13.5 Hz, 4H). ¹⁹F NMR (376 MHz, d6-Acetone) δ −76.2, -140.6. HRMS: m/z calcd. for C₃₅H₃₇ClFN₆O₅⁺ ([M+H]⁺): 675.2493, found: 675.2487.

### (S)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(3-oxobutanoyl)piperazin-2-yl)acetonitrile

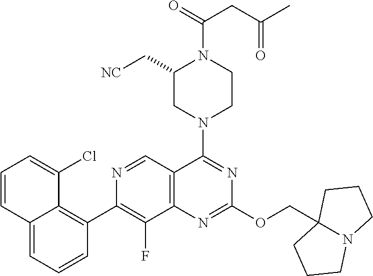 [SMILES: CC(=O)CC(=O)N1CCN(c2nc(OCC34CCCN3CCC4)nc3c(F)c(-c4cccc5cccc(Cl)c45)ncc23)C[C@@H]1CC#N]

(S)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)piperazin-2-yl)acetonitrile (50.0 mg, 0.073 mmol, 1.0 equiv) was dissolved in dry toluene (1 mL). DIPEA (28.3 mg, 0.219 mmol, 3 equiv) and 2,2,6-trimethyl-4H-1,3-dioxin-4-one (12.4 mg, 0.087 mmol, 1.2 equiv) were subsequently added and the solution was stirred at 80° C. for 1 h. The reaction mixture was partitioned between saturated aqueous sodium bicarbonate solution and dichloromethane. The layers were separated, the organic phase was extracted with dichloromethane, dried over Na₂SO₄, filtered, and concentrated. The crude mixture was purified by reverse-phase HPLC to yield (S)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(3-oxobutanoyl)piperazin-2-yl)acetonitrile as colorless solid (27.4 mg, 0.042 mmol, 57%). HRMS: m/z calcd. for C₃₅H₃₆ClFN₇O₃⁺ ([M+H]⁺): 656.2547, found: 656.2547.

### (S)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(2,2-dihydroxy-3-oxobutanoyl)piperazin-2-yl)acetonitrile (4)

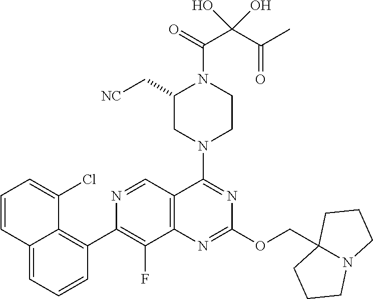 [SMILES: CC(=O)C(O)(O)C(=O)N1CCN(c2nc(OCC34CCCN3CCC4)nc3c(F)c(-c4cccc5cccc(Cl)c45)ncc23)C[C@@H]1CC#N]

(S)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(3-oxobutanoyl)piperazin-2-yl)acetonitrile (20.0 mg, 0.030 mmol, 1.0 equiv.) was dissolved in wet CH₂Cl₂ (0.2 mL). Pyridine (9.6 mg, 0.122 mmol, 4.0 equiv.) and dess-martin periodinane (51.7 mg, 0.122 mmol, 4.0 equiv.) were added and the mixture was stirred for 1 h at room temperature. Solvents were removed under reduced pressure. The residue was dissolved in MeCN:H2O (1:1) and purified by reverse phase HPLC (C18) to yield (S)-2-(4-(7-(8-chloronaphthalen-1-yl)-8-fluoro-2-((tetrahydro-1H-pyrrolizin-7a(5H)-yl)methoxy)pyrido[4,3-d]pyrimidin-4-yl)-1-(2,2-dihydroxy-3-oxobutanoyl)piperazin-2-yl)acetonitrile (4) as colorless solid (11.2 mg, 0.016 mmol, 53%). HRMS: m/z calcd. for C₃₅H₃₆ClFN₇O₅⁺ ([M+H]⁺): 688.2445, found: 688.2444.

### REFERENCES

1. Simanshu, D. K.; Nissley, D. V.; McCormick, F. Cell 2017, 170 (1), 17-33. 2. Prior, I. A.; Hood, F. E.; Hartley, J. L. Cancer Res. 2020, 80 (14), 2969-2974. 3. Ostrem, J. M. L.; Shokat, K. M. Nat. Rev. Drug Discov. 2016, 15 (11), 771-785. 4. Moore, A. R.; Rosenberg, S. C.; McCormick, F.; Malek, S. Nat. Rev. Drug Discov. 2020, 19, 533-552. 5. Ostrem, J. M.; Peters, U.; Sos, M. L.; Wells, J. A.; Shokat, K. M. Nature 2013, 503 (7477), 548-551. 6. Patricelli, M. P.; Janes, M. R.; Li, L. S.; Hansen, R.; Peters, U.; Kessler, L. V; Chen, Y.; Kucharski, J. M.; Feng, J.; Ely, T.; Chen, J. H.; Firdaus, S. J.; Babbar, A.; Ren, P.; Liu, Y. Cancer Discov. 2016, 6 (3), 316-329. 7. Hansen, R.; Peters, U.; Babbar, A.; Chen, Y.; Feng, J.; Janes, M. R.; Li, L. S.; Ren, P.; Liu, Y.; Zarrinkar, P. P. Nat. Struct. Mol. Biol. 2018, 25 (6), 454-462. 8. Janes, M. R.; Zhang, J.; Li, L.-S.; Hansen, R.; Peters, U.; Guo, X.; Chen, Y.; Babbar, A.; Firdaus, S. J.; Darjania, L.; Feng, J.; Chen, J. H.; Li, S.; Li, S.; Long, Y. O.; Thach, C.; Liu, Y.; Zarieh, A.; Ely, T.; Kucharski, J. M.; Kessler, L. V.; Wu, T.; Yu, K.; Wang, Y.; Yao, Y.; Deng, X.; Zarrinkar, P. P.; Brehmer, D.; Dhanak, D.; Lorenzi, M. V.; Hu-Lowe, D.; Patricelli, M. P.; Ren, P.; Liu, Y. Cell 2018, 172 (3), 578-589.e17. 9. Fell, J. B.; Fischer, J. P.; Baer, B. R.; Blake, J. F.; Bouhana, K.; Briere, D. M.; Brown, K. D.; Burgess, L. E.; Burns, A. C.; Burkard, M. R.; Chiang, H.; Chicarelli, M. J.; Cook, A. W.; Gaudino, J. J.; Hallin, J.; Hanson, L.; Hartley, D. P.; Hicken, E. J.; Hingorani, G. P.; Hinklin, R. J.; Mejia, M. J.; Olson, P.; Otten, J. N.; Rhodes, S. P.; Rodriguez, M. E.; Savechenkov, P.; Smith, D. J.; Sudhakar, N.; Sullivan, F. X.; Tang, T. P.; Vigers, G. P.; Wollenberg, L.; Christensen, J. G.; Marx, M. A. J. Med. Chem. 2020, 63 (13), 6679-6693. 10. Lanman, B. A.; Allen, J. R.; Allen, J. G.; Amegadzie, A. K.; Ashton, K. S.; Booker, S. K.; Chen, J. J.; Chen, N.; Frohn, M. J.; Goodman, G.; Kopecky, D. J.; Liu, L.; Lopez, P.; Low, J. D.; Ma, V.; Minatti, A. E.; Nguyen, T. T.; Nishimura, N.; Pickrell, A. J.; Reed, A. B.; Shin, Y.; Siegmund, A. C.; Tamayo, N. A.; Tegley, C. M.; Walton, M. C.; Wang, H. L.; Wurz, R. P.; Xue, M.; Yang, K. C.; Achanta, P.; Bartberger, M. D.; Canon, J.; Hollis, L. S.; McCarter, J. D.; Mohr, C.; Rex, K.; Saiki, A. Y.; San Miguel, T.; Volak, L. P.; Wang, K. H.; Whittington, D. A.; Zech, S. G.; Lipford, J. R.; Cee, V. J. J. Med. Chem. 2020, 63 (1), 52-65. 11. Kwan, A. K.; Piazza, G. A.; Keeton, A. B.; Leite, C. A. J. Exp. Clin. Cancer Res. 2022, 41 (1), Article number: 27. 12. Hobbs, G. A.; Baker, N. M.; Miermont, A. M.; Thurman, R. D.; Pierobon, M.; Tran, T. H.; Anderson, A. O.; Waters, A. M.; Diehl, J. N.; Papke, B.; Hodge, R. G.; Klomp, J. E.; Goodwin, C. M.; DeLiberty, J. M.; Wang, J.; Ng, R. W. S.; Gautam, P.; Bryant, K. L.; Esposito, D.; Campbell, S. L.; Petricoin, E. F.; Simanshu, D. K.; Aguirre, A. J.; Wolpin, B. M.; Wennerberg, K.; Rudloff, U.; Cox, A. D.; Der, C. J. Cancer Discov. 2020, 10 (1), 104-123. 13. Diels, O.; Schleich, K. Ober Bildung Und Eigenschaften Der Aus 1.2-Diketonen Entstehenden Verbindungen. I. Diacetyl Und Benzamidin. Berichte der Dtsch. Chem. Gesellschaft 1916, 49 (2), 1711-1721. 14. Takahashi, K. J. Biol. Chem. 1968, 243 (23), 6171-6179. 15. Yankeelov, J. A.; Mitchell, C. D.; Crawford, T. H. Journal of the American Chemical Society. 1968, pp 1664-1666. 16. Zheng, Q.; Omans, N. D.; Leicher, R.; Osunsade, A.; Agustinus, A. S.; Finkin-Groner, E.; D'Ambrosio, H.; Liu, B.; Chandarlapaty, S.; Liu, S.; David, Y. Nat. Commun. 2019, 10 (1), Article number: 1289. 17. Huang, W. Y.; Tang, J. J. Biol. Chem. 1972, 247 (9), 2704-2710. 18. Thompson, D. A.; Ng, R.; Dawson, P. E. J. Pept. Sci. 2016, 22 (5), 311-319. 19. Jones, A. X.; Cao, Y.; Tang, Y. L.; Wang, J. H.; Ding, Y. H.; Tan, H.; Chen, Z. L.; Fang, R. Q.; Yin, J.; Chen, R. C.; Zhu, X.; She, Y.; Huang, N.; Shao, F.; Ye, K.; Sun, R. X.; He, S. M.; Lei, X.; Dong, M. Q. Nat. Commun. 2019, 10 (1), Article number: 3911. 20. Oya, T.; Hattori, N.; Mizuno, Y.; Miyata, S.; Maeda, S.; Osawa, T.; Uchida, K. J. Biol. Chem. 1999, 274 (26), 18492-18502. 21. Takahashi, K. J. Biochem. 1977, 81 (2), 403-414. 22. Lo, T.; Westwood, M.; McLellan, A.; Selwood, T.; Thornalley, P. J. Biol. Chem. 1994, 51, 32299-32305. 23. Suckau, D.; Mak, M.; Przybylski, M. Proc. Natl. Acad. Sci. U.S.A. 1992, 89 (12), 5630-5634. 24. Ahmed, N.; Thornalley, P. J. Ann. N.Y. Acad. Sci. 2005, 1043, 260-266. 25. Mostafa, A. A.; Randell, E. W.; Vasdev, S. C.; Gill, V. D.; Han, Y.; Gadag, V.; Raouf, A. A.; El Said, H. Mol. Cell. Biochem. 2007, 302 (1-2), 35-42. 26. Gao, Y.; Wang, Y. Biochemistry 2006, 45 (51), 15654-15660. 27. Chumsae, C.; Gifford, K.; Lian, W.; Liu, H.; Radziejewski, C. H.; Zhou, Z. S. Anal. Chem. 2013, 85 (23), 11401-11409. 28. Galligan, J. J.; Wepy, J. A.; Streeter, M. D.; Kingsley, P. J.; Mitchener, M. M.; Wauchope, O. R.; Beavers, W. N.; Rose, K. L.; Wang, T.; Spiegel, D. A.; Marnett, L. J. Proc. Natl. Acad. Sci. 2018, 115 (37), 9228-9233. 29. Vanoni, M. A.; Pilone Simonetta, M.; Curti, B.; Negri, A.; Ronchi, S. Eur. J. Biochem. 1987, 167 (2), 261-267. 30. Stipani, I.; Mangiullo, G.; Stipani, V.; Daddabbo, L.; Natuzzi, D.; Palmieri, F. Arch. Biochem. Biophys. 1996, 331 (1), 48-54. 31. Gauthier, M. A.; Klok, H. A. Biomacromolecules 2011, 12 (2), 482-493. 32. Thompson, D. A.; Ng, R.; Dawson, P. E. J. Pept. Sci. 2016, 22 (5), 311-319. 33. Dovgan, I.; Erb, S.; Hessmann, S.; Ursuegui, S.; Michel, C.; Muller, C.; Chaubet, G.; Cianférani, S.; Wagner, A. Org. Biomol. Chem. 2018, 16 (8), 1305-1311. 34. Batchelor, M. J.; Gillespie, R. J.; Golec, J. M. C.; Hedgecock, C. J. R. Tetrahedron Lett. 1993, 34 (1), 167-170. 35. Mathews, J. M.; Watson, S. L.; Snyder, R. W.; Burgess, J. P.; Morgan, D. L. J. Agric. Food Chem. 2010, 58 (24), 12761-12768. 36. Hunter, J. C.; Manandhar, A.; Carrasco, M. A.; Gurbani, D.; Gondi, S.; Westover, K. D. Mol. Cancer Res. 2015, 13 (9), 1325-1335. 37. Lou, K.; Steri, V.; Ge, A. Y.; Hwang, Y. C.; Yogodzinski, C. H.; Shkedi, A. R.; Choi, A. L. M.; Mitchell, D. C.; Swaney, D. L.; Hann, B.; Gordan, J. D.; Shokat, K. M.; Gilbert, L. A. Sci. Signal. 2019, 12 (583), Article number: eaaw9450. 38. Ostrem, J. M.; Peters, U.; Sos, M. L.; Wells, J. A.; Shokat, K. M. Nature 2013, 503 (7477), 548-551. 39. Ahmadian, M. R.; Zor, T.; Vogt, D.; Kabsch, W.; Selinger, Z.; Wittinghofer, a; Scheffzek, K. Proc. Natl. Acad. Sci. U.S.A. 1999, 96 (12), 7065-7070. 40. Huehls, A. M.; Coupet, T. A.; Sentman, C. L. Immunol. Cell Biol. 2014, 93 (10), 290-296. 41. Maurer, T.; Garrenton, L. S.; Oh, A.; Pitts, K.; Anderson, D. J.; Skelton, N. J.; Fauber, B. P.; Pan, B.; Malek, S.; Stokoe, D.; Ludlam, M. J. C.; Bowman, K. K.; Wu, J.; Giannetti, A. M.; Starovasnik, M. A.; Mellman, I.; Jackson, P. K.; Rudolph, J.; Wang, W.; Fang, G. Proc. Natl. Acad. Sci. U.S.A. 2012, 109 (14), 5299-5304. 42. Gibson, D. G.; Young, L.; Chuang, R. Y.; Venter, J. C.; Hutchison, C. A.; Smith, H. O. Nat. Methods 2009, 6 (5), 343-345. 43. Gentile, D. R.; Rathinaswamy, M. K.; Jenkins, M. L.; Moss, S. M.; Siempelkamp, B. D.; Renslo, A. R.; Burke, J. E.; Shokat, K. M. Cell Chem. Biol. 2017, 24 (12), 1455-1466.e14. 44. Sondermann, H.; Soisson, S. M.; Boykevisch, S.; Yang, S. S.; Bar-Sagi, D.; Kuriyan, J. Cell 2004, 119 (3), 393-405.

1. A compound, or a pharmaceutically acceptable salt thereof, having the formula: 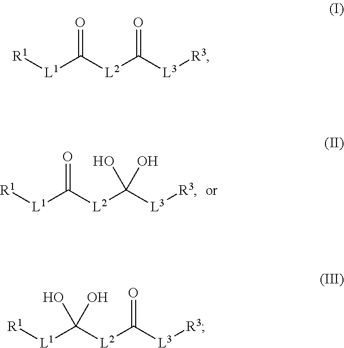
wherein
R¹ is a Switch II Binding Pocket binding moiety;
L¹ is a bond or divalent linker;
L² is a bond or substituted or unsubstituted alkylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl;
each X3 is independently —Cl, —Br, —I, or —F;
n3 is an integer from 0 to 4; and
m3 and v3 are independently 1 or 2.

2. (canceled)

3. (canceled)

4. The compound of claim 1, wherein L³ is a bond or —C(O)—.

5. (canceled)

6. The compound of claim 1, having the formula:

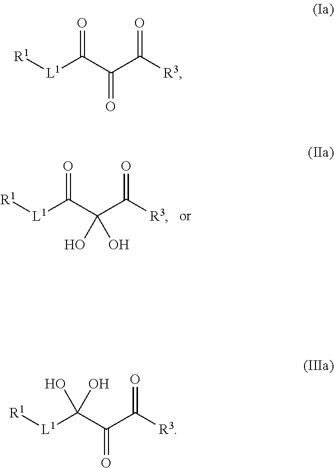

7. The compound of claim 1, having the formula:

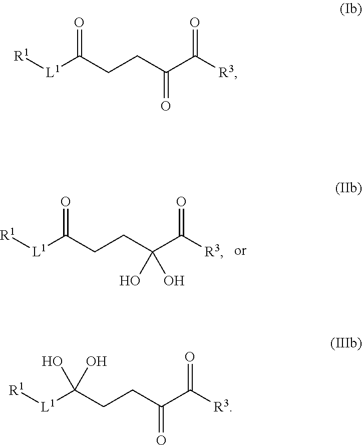

8. The compound of claim 1, having the formula:

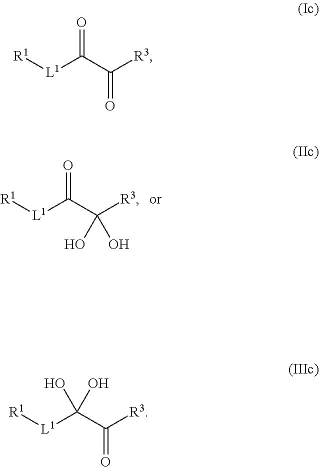

9. (canceled)

10. The compound of claim 1, wherein R³ is hydrogen or unsubstituted C₁-C₄ alkyl.

11. (canceled)

12. The compound of claim 1, wherein
L¹ is -L¹⁰¹-L¹⁰²-L¹⁰³-L¹⁰⁴-L¹⁰⁵-;
L¹⁰¹ is a bond, —C(O)—, —C(O)O—, —OC(O)—, —O—, —S—, —NR¹⁰¹—, C(O)NR¹⁰¹—, —NR¹⁰¹C(O)—, —NR¹⁰¹C(O)O—, —OC(O)NR¹⁰¹—, —NR¹⁰¹C(O)NR¹⁰¹—, —NR¹⁰¹C(NH)NR¹⁰¹—, —S(O)₂—, —NR¹⁰¹S(O)₂—, —S(O)₂NR¹⁰¹—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, C(O)NR¹⁰²—, —NR¹⁰²C(O)—, —NR¹⁰²C(O)O—, —OC(O)NR¹⁰²—, —NR¹⁰²C(O)NR¹⁰²—, —NR¹⁰²C(NH)NR¹⁰²—, —S(O)₂—, —NR¹⁰²S(O)₂—, —S(O)₂NR¹⁰²—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, —NR¹⁰³C(O)O—, —OC(O)NR¹⁰³—, —NR¹⁰³C(O)NR¹⁰³—, —NR¹⁰³C(NH)NR¹⁰³—, —S(O)₂—, —NR¹⁰³S(O)₂—, —S(O)₂NR¹⁰³—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, —NR¹⁰⁴C(O)O—, —OC(O)NR¹⁰⁴—, —NR¹⁰⁴C(O)NR¹⁰⁴—, —NR¹⁰⁴C(NH)NR¹⁰⁴—, —S(O)₂—, —NR¹⁰⁴S(O)₂—, —S(O)₂NR¹⁰⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, —NR¹⁰⁵C(O)O—, —OC(O)NR¹⁰⁵—, —NR¹⁰⁵C(O)NR¹⁰⁵—, —NR¹⁰⁵C(NH)NR¹⁰⁵—, —S(O)₂—, —NR¹⁰⁵S(O)₂—, —S(O)₂NR¹⁰⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
each R¹⁰¹, R¹⁰², R¹⁰³, R¹⁰⁴, and R¹⁰⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

13. (canceled)

14. The compound of claim 1, wherein L¹ is

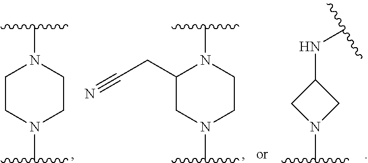

15. The compound of claim 1, wherein R¹ is -L¹⁰-R¹⁰;
L¹⁰ is a bond, —C(O)—, —C(O)O—, —OC(O)—, —O—, —S—, —NR¹⁰⁰—, —C(O)NR¹⁰⁰—, —NR¹⁰⁰C(O)—, —NR¹⁰⁰C(O)O—, —OC(O)NR¹⁰⁰—, —NR¹⁰⁰C(O)NR¹⁰⁰—, —NR¹⁰⁰C(NH)NR¹⁰⁰—, —S(O)₂—, —NR¹⁰⁰S(O)₂—, —S(O)₂NR¹⁰⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A} and R^{10B} substituents bonded to the same nitrogen atom may optionally be joined to form a substituted or unsubstituted heterocycloalkyl or substituted or unsubstituted heteroaryl;
each X¹⁰ is independently —F, —Cl, —Br, or —I;
n10 is an integer from 0 to 4; and
m10 and v10 are independently 1 or 2.

16. The compound of claim 1, wherein R¹ is 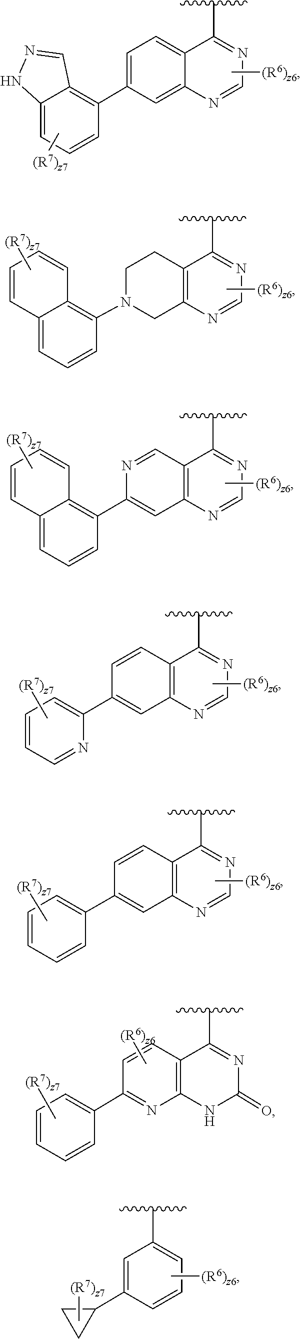 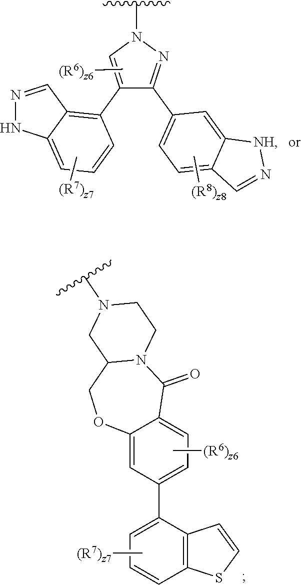
R⁶ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z6 is an integer from 0 to 7;
z7 is an integer from 0 to 7; and
z8 is an integer from 0 to 5.

17.-27. (canceled)

28. The compound of claim 1, wherein R¹ is

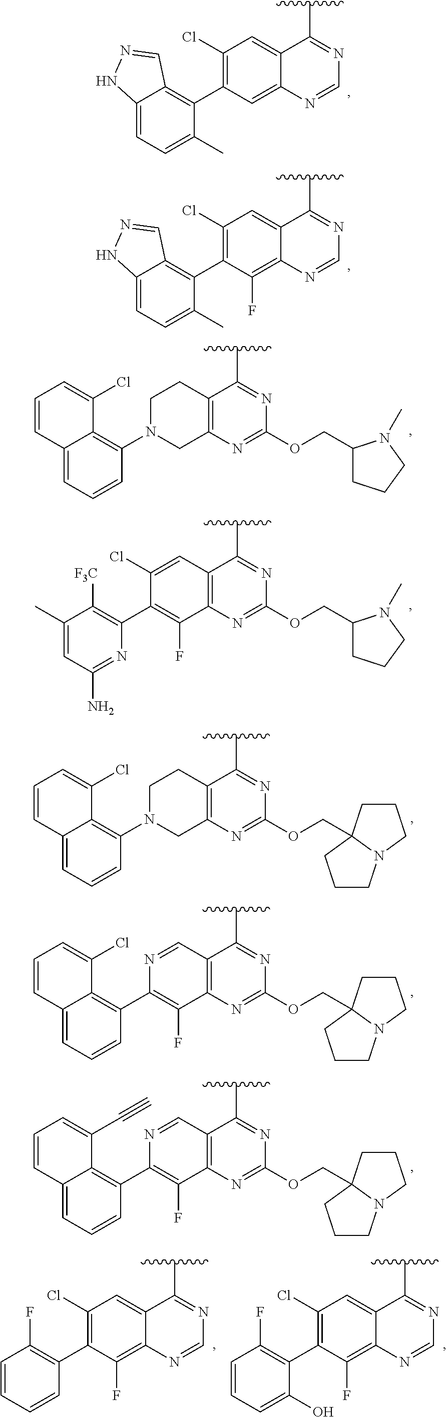

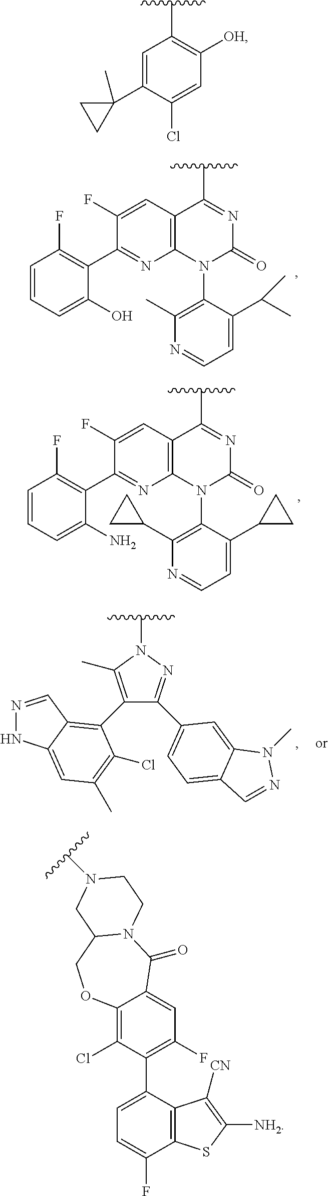

29. The compound of claim 1, having the formula:

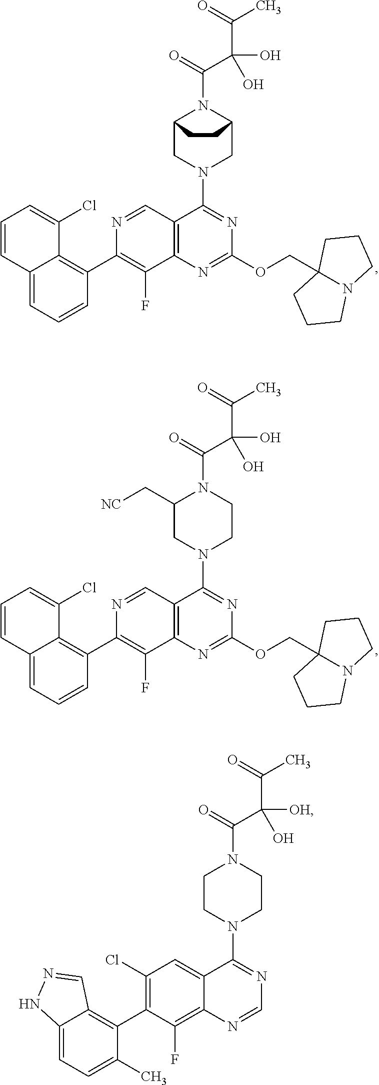

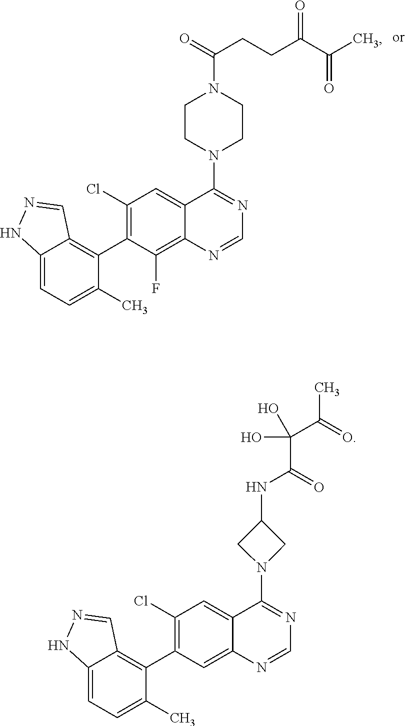 [SMILES: CC(=O)C(=O)CCC(=O)N1CCN(c2ncnc3c(F)c(-c4c(C)ccc5[nH]ncc45)c(Cl)cc23)CC1 ; "or" ; CC(=O)C(O)(O)C(=O)NC1CN(c2ncnc3cc(-c4c(C)ccc5[nH]ncc45)c(Cl)cc23)C1]

30. A pharmaceutical composition comprising a pharmaceutically acceptable excipient and the compound of claim 1, or a pharmaceutically acceptable salt thereof.

31. A method of treating cancer in a subject in need thereof, said method comprising administering to the subject in need thereof a therapeutically effective amount of the compound of claim 1, or a pharmaceutically acceptable salt thereof.

32. The method of claim 31, wherein the cancer is pancreatic cancer, lung cancer, or colorectal cancer.

33. A method of reducing Ras protein-mediated activity in a cell, said method comprising contacting the cell with an effective amount of the compound of claim 1, or a pharmaceutically acceptable salt thereof.

34.-39. (canceled)

40. A Switch II GTPase protein covalently bound to the compound of claim 1, or a salt thereof, wherein said compound is covalently bound to an arginine residue of said Switch II GTPase protein.

41.-51. (canceled)

52. A method of attaching a compound to an arginine residue of a protein, said method comprising contacting said compound with said arginine residue, wherein said compound has the formula:
or a salt thereof, wherein 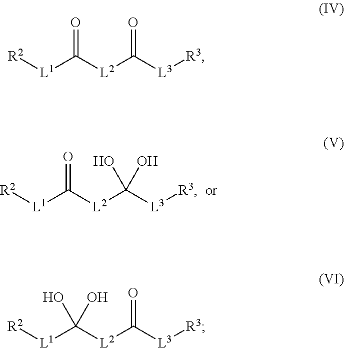
R² is a Switch II Binding Pocket binding moiety, a phosphatase PTP domain binding moiety, an SH2 domain binding moiety, a pseudokinase KSR domain binding moiety, or a pseudokinase STRADα domain binding moiety;
L¹ is a bond or divalent linker; and
L² is a bond or substituted or unsubstituted alkylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is hydrogen, halogen, —CX³₃, —CHX³₂, —CH₂X³, —OCX³₃, —OCH₂X³, —OCHX³₂, —CN, —SOₙ₃R^{3D}, —SOv₃NR^{3A}R^{3B}, —NR³CNR^{3A}R^{3B}, —ONR^{3A}R^{3B}, —NR^{3C}C(O)NR^{3A}R^{3B}, N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl;
each X³ is independently —Cl, —Br, —I, or —F;
n3 is an integer from 0 to 4; and
m3 and v3 are independently 1 or 2.

53.-79. (canceled)

80. A method of attaching a compound to an arginine residue, said method comprising contacting said compound with said arginine residue, wherein said compound has the formula: 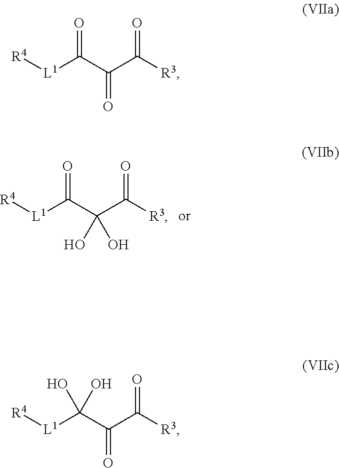
or a salt thereof; wherein
L¹ is a bond or divalent linker;
R³ is hydrogen, halogen, —CX³₃, —CHX³₂, —CH₂X³, —OCX³₃, —OCH₂X³, —OCHX³₂, —CN, —SOₙ₃R^{3D}, —SOv₃NR^{3A}R^{3B}, —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOn₄R^{4D}, —SOv₄NR^{4A}R^{4B}, —NR⁴CNR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{3A}, R^{3B}, R^{3C}, R^{3D}, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X³ and X⁴ is independently —Cl, —Br, —I, or —F;
n3 and n4 are independently an integer from 0 to 4; and
m3, m4, v3, and v4 are independently 1 or 2.

81.-84. (canceled)

85. A compound covalently bound to an arginine residue, having the formula: 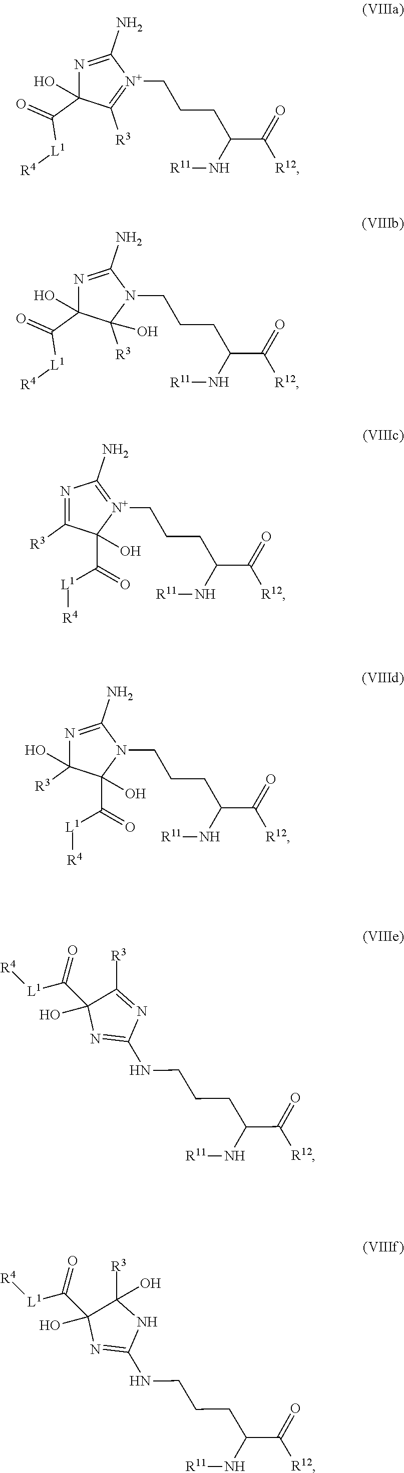 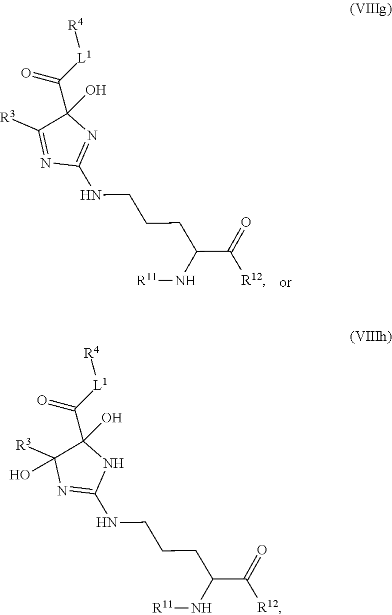
or a salt thereof; wherein
L¹ is a bond or divalent linker;
R³ is hydrogen, halogen, —CX³₃, —CHX³₂, —CH₂X³, —OCX³₃, —OCH₂X³, —OCHX³₂, —CN, —SOₙ₃R^{3D}, —SOv₃NR^{3A}R^{3B}, —NR³CNR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —OC(O)R^{3C}, —OC(O)OR^{3C}, —C(O)NR^{3A}R^{3B}, —OC(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{3A}, R^{3B}, R^{3C}, R^{3D}, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X³ and X⁴ is independently —Cl, —Br, —I, or —F;
n3 and n4 are independently an integer from 0 to 4;
m3, m4, v3, and v4 are independently 1 or 2;
R¹¹ is hydrogen, —C(O)CH₃, or a first protein moiety; and
R¹² is —OH or a second protein moiety.

86.-90. (canceled)


SEQUENCE LISTING (8 sequences)

SEQ ID NO: 1 - protein, 189 aa, synthetic construct [sequence omitted]

SEQ ID NO: 2 - protein, 189 aa, synthetic construct
  REGION 1..189: note = Synthetic polypeptide
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - protein, 189 aa, synthetic construct
  REGION 1..189: note = Synthetic polypeptide
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 4 - protein, 435 aa, synthetic construct
  REGION 1..435: note = Synthetic polypeptide
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGKCREFFPNHQWVKEETQEDKDCPIKEEKGSPLNAAPYGIESMSQDTEVRSRVVGGSLRGAQAASPAKGEPSLPEKDEDHALSYWKPFLVNMCVATVLTAGAYLCYRFLFNSNT

SEQ ID NO: 5 - protein, 536 aa, synthetic construct
  REGION 1..536: note = Synthetic polypeptide
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 6 - protein, 950 aa, synthetic construct [sequence omitted]

SEQ ID NO: 7 - protein, 431 aa, synthetic construct
  REGION 1..431: note = Synthetic polypeptide
MSFLVSKPERIRRWVSEKFIVEGLRDLELFGEQPPGDTRRKTNDASSESIASFSKQEVMSSFLPEGGCYELLTVIGKGFEDLMTVNLARYKPTGEYVTVRRINLEACSNEMVTFLQGELHVSKLFNHPNIVPYRATFIADNELWVVTSFMAYGSAKDLICTHFMDGMNELAIAYILQGVLKALDYIHHMGYVHRSVKASHILISVDGKVYLSGLRSNLSMISHGQRQRVVHDFPKYSVKVLPWLSPEVLQQNLQGYDAKSDIYSVGITACELANGHVPFKDMPATQMLLEKLNGTVPCLLDTSTIPAEELTMSPSRSVANSGLSDSLTTSTPRPSNGDSPSHPYHRTFSPHFHHFVEQCLQRNPDARPSASTLLNHSFFKQIKRRASEALPELLRPVTPITNFEGSQSQDHSGIFGLVTNLEELEVDDWEF

SEQ ID NO: 8 - protein, 7 aa, synthetic construct
  REGION 1..7: note = Synthetic polypeptide
ENLYFQG